(19) United States
(12) Patent Application Publication
(10) Pub. No.: US 2024/0344116 A1
(43) Pub. Date: Oct. 17, 2024

(54) TRANSFER OF NANOPARTICLES TO ARRAY SURFACES

(71) Applicant: Nautilus Subsidiary, Inc., Seattle, WA (US)

(72) Inventors: Shubhodeep PAUL, Millbrae, CA (US); Christina E. INMAN, San Mateo, CA (US); Maryam JOUZI, Hayward, CA (US); Rukshan PERERA, Fremont, CA (US); Pierre INDERMUHLE, Berkeley, CA (US); Hongji QIAN, Sunnyvale, CA (US); Ilaria SANDEI, Redwood City, CA (US)

(21) Appl. No.: 18/613,879

(22) Filed: Mar. 22, 2024

Related U.S. Application Data
(60) Provisional application No. 63/492,004, filed on Mar. 24, 2023.
(60) Provisional application No. 63/582,805, filed on Sep. 14, 2023.

(51) Int. Cl. C12Q 1/6837

(52) U.S. Cl. CPC C12Q 1/6837

(57) ABSTRACT

Methods and systems are provided for forming arrays of analytes, in which the arrays are characterized by a high site occupancy and optionally a low site co-occupancy, and in which the arrays are configured for single-analyte processes. Methods for effecting transport of binding entities to array sites are also provided.

### CROSS-REFERENCE TO RELATED APPLICATIONS

This application claims priority to U.S. Provisional Applications No. 63/492,004, filed on Mar. 24, 2023, and 63/582,805, filed on Sep. 14, 2023, which applications are incorporated herein by reference in their entireties.

### BACKGROUND

Certain chemical and biochemical assays may utilize arrays to provide a degree of spatial control and orientation of array components. An array can comprise a plurality of sites, with each site intended to bind or hold one or more molecules or moieties at a fixed, observable address on the array. In particular, single-analyte arrays, such as single polypeptide arrays and single nucleic acid arrays, can be configured to provide a spatially-ordered collection of individual analytes such that each analyte affixed to the array is individually interrogable.

For certain applications, an array (such as a single-analyte array) may be disposed in a microfluidic device. A microfluidic device can provide an apparatus for controlled delivery of analytes and other reagents to an array disposed within the microfluidic device. Moreover, a microfluidic device can provide a degree of environmental control to an array by limiting fluidic or physical contact with an external environment. In some instances, a microfluidic device can be characterized by fluidic passages (e.g., channels, reservoirs, or chambers) with micron-scale characteristic dimensions, while an array disposed within the microfluidic device can be characterized by array features (e.g., array sites or analytes) with nanometer-scale characteristic dimensions.

Mass transfer in microfluidic devices can be affected by certain physical phenomena. Depending upon characteristic dimensions of fluidic passages, fluid flow can be limited to laminar or transitional flow regimes. Moreover, length scales of boundary layer effects can be large relative to the length scales of array components (e.g., analytes) within fluidic passages of a microfluidic device. In some instances, mass diffusion can be nearly equal or dominate over bulk transfer of array components to an array surface within a microfluidic device.

### SUMMARY

In an aspect, provided herein is a method of forming an array of analytes, comprising: a) providing a solid support comprising a population of sites, wherein the population of sites is characterized by an array site binding competency profile, b) providing a fluidic medium comprising a population of analytes, wherein the composition of the population of analytes is based upon the array site binding competency profile, and c) contacting the fluidic medium to the solid support, thereby binding analytes of the population of analytes to sites of the population of sites to form the array of analytes, wherein the array of analytes has a site occupancy of at least 80%, and wherein the array of analytes has a site co-occupancy of no more than 10%.

In another aspect, provided herein is a method of forming an array of analytes, comprising: a) providing a solid support comprising a population of sites, wherein the population of sites has a characterized site binding competency profile, b) providing a fluidic medium comprising a population of analytes, wherein the population of analytes has a characterized analyte binding competency profile, and wherein the characterized analyte binding competency profile is a function of an analyte quality control parameter, and c) contacting the fluidic medium to the solid support, thereby binding analytes of the population of analytes to sites of the population of sites to form the array of analytes, wherein the array of analytes has a site occupancy of at least 80%, and wherein binding the analytes of the population of analytes to sites of the population of sites occurs in no more than 1 hour.

In another aspect, provided herein is a method of preparing a population of anchoring moieties, comprising: a) providing a solid support comprising a population of sites, b) measuring a quality control parameter for each site of the population of sites, c) based upon the quality control parameter, determining a site binding competency profile for the population of sites, and d) based upon the site binding competency profile of the population of sites, preparing a population of anchoring moieties, wherein the population of anchoring moieties has an anchoring moiety binding competency profile, and wherein the anchoring moiety binding competency profile is complementary to the site binding competency profile of the population of sites.

In another aspect, provided herein is a method of forming an array of analytes, comprising: a) providing a solid support with a surface, wherein the surface is substantially planar, and wherein a population of sites is disposed on the surface, b) contacting the array with a fluidic medium comprising a population of anchoring moieties, wherein each anchoring moiety is attached to an analyte, and c) after contacting the array with the fluidic medium, providing a first analyte association condition, wherein the first analyte association condition forms a concentration gradient of anchoring moieties in the fluidic medium, and wherein the concentration gradient of anchoring moieties is characterized by an increasing concentration of anchoring moieties as distance to the surface of the solid support decreases in a direction orthogonal to the surface of the solid support, whereby anchoring moieties of the plurality of anchoring moieties bind to sites of the population of sites, wherein the single-pass site occupancy is at least 75% of the population of sites within no more than 30 minutes of contacting the solid support with the fluidic medium.

In another aspect, provided herein is a method of forming an array of analytes on a solid support, comprising: a) providing a solid support comprising a surface, wherein the surface is substantially planar, wherein a quantity of sites is disposed on the surface, wherein each site is separated from each other site of the quantity of sites by an interstitial region, and wherein each site of the quantity of sites is optically resolvable from each other site, b) contacting the array with a fluidic medium comprising a plurality of nanoparticles, wherein each nanoparticle is attached to an analyte, c) after contacting the array with the fluidic medium, providing a first analyte association condition, wherein the first analyte association condition forms a concentration gradient of nanoparticles in the fluidic medium, and wherein the concentration of nanoparticles increases as a distance from the surface of the solid support decreases in a direction orthogonal to the surface of the solid support, d) after contacting the array with the fluidic medium, providing a first analyte association condition, wherein the first analyte association condition forms a concentration gradient of nanoparticles in the fluidic medium, and wherein the concentration of nanoparticles increases as a distance from the surface of the solid support decreases in a direction orthogonal to the surface of the solid support, and e) after contacting the array with the fluidic medium, providing a second analyte association condition, wherein the second analyte association condition forms an anisotropic surface density of nanoparticles on the interstitial region of the solid support, whereby nanoparticles of the plurality of nanoparticles bind to sites of the quantity of sites, wherein the single-pass global site occupancy is at least 75% of the quantity of sites within no more than 15 minutes of contacting the solid support with the fluidic medium.

In another aspect, provided herein is a method of forming an array, comprising: a) providing an array comprising a plurality of sites, wherein the sites are substantially devoid of analytes, wherein the plurality of sites has a characteristic property, and wherein the characteristic binding property of the plurality of sites has a standard deviation with a magnitude of at least 10% of the characteristic property, b) binding a plurality of analytes to sites of the plurality of sites, and c) after binding the plurality of analytes to the sites, detecting a presence or absence of an analyte at each site of the plurality of sites, wherein at least 70% of sites of the active fraction of sites comprise an analyte of the plurality of analytes, and wherein no more than 10% of sites of the active fraction of sites comprises two or more analytes of the plurality of analytes.

In another aspect, provided herein is a method, comprising: a) providing a first array of a plurality of arrays, wherein each array of the plurality of arrays comprises a plurality of sites, and wherein the plurality of sites for each array has a known binding characteristic, b) binding analytes to the plurality of sites of the first array with a first analyte association condition, c) after binding the analytes to the first array, determining a first analyte site occupancy for the first array, d) after determining the first analyte site occupancy for the first array, determining a second analyte association condition for a second array of the plurality of arrays based upon a comparison of a first known binding characteristic of the first array to a second known binding characteristic of the second array, and e) binding analytes to the plurality of sites of the second array with the second analyte association condition.

In another aspect, provided herein is a method of forming an array, comprising: a) contacting an array comprising a plurality of sites with a layer of a fluidic medium, wherein the fluidic medium comprises a plurality of analytes, wherein the layer of the fluidic medium has an average thickness, wherein sites of the plurality of sites comprise filamentous moieties, and wherein the filamentous moieties have an average length of at least 10% the thickness of the fluidic medium, and b) binding analytes to at least 70% of sites of the plurality of sites within 15 minutes of contacting the array with the fluidic medium.

In another aspect, provided herein is a method, comprising: a) contacting a fluidic medium comprising a plurality of particles to a solid support, wherein the solid support comprises a plurality of sites, wherein the spatial distribution of the plurality of particles in the fluidic medium is substantially homogeneous, and wherein each individual particle of the plurality of particles is coupled to a plurality of macromolecules, b) concentrating the plurality of particles adjacent to a surface of the solid support, c) after concentrating the plurality of particles adjacent to the surface of the solid support, separating macromolecules from particles of the plurality of particles, d) binding the macromolecules to sites of the plurality of sites of the solid support, and e) after separating the macromolecules from the plurality of particles, dispersing particles of the plurality of particles from adjacent to the surface of the solid support.

### INCORPORATION BY REFERENCE

All publications, items of information available on the internet, patents, and patent applications cited in this specification are herein incorporated by reference to the same extent as if each individual publication, patent, or patent application was specifically and individually indicated to be incorporated by reference. To the extent publications, items of information available on the internet, patents, or patent applications incorporated by reference contradict the disclosure contained in the specification, the specification is intended to supersede and/or take precedence over any such contradictory material.

### BRIEF DESCRIPTION OF DRAWINGS

FIGS. 1A and 1B illustrate different aspects of subarray layout for arrays containing populations of array sites, in accordance with some embodiments.

FIG. 2 depicts aspects of length scales for components of an array of analytes, in accordance with some embodiments.

FIG. 3 shows spatial arrangements of binding entities (e.g., analytes, anchoring moieties or other particles) with respect to a common coordinate system, in accordance with some embodiments.

FIGS. 4A and 4B display aspects of describing length scales for mass transfer of binding entities in an array-based system, in accordance with some embodiments.

FIG. 5A illustrates aspects of array site binding availability and array site binding competency. FIG. 5B illustrates aspects of analyte or anchoring moiety binding availability and analyte or anchoring moiety binding competency, in accordance with some embodiments.

FIG. 6 depicts an exemplary overlay of an array site binding competency profile with an analyte binding competency profile to estimate a maximum array site occupancy, in accordance with some embodiments.

FIG. 7 shows a schematic workflow for identifying conditions for an array formation process, in accordance with some embodiments, in accordance with some embodiments.

FIGS. 8A, 8B, and 8C display the effect of decreasing binding entity concentration on binding entity deposition rate, in accordance with some embodiments.

FIG. 9 illustrates aspects of binding entity mass transfer across a surface of a solid support, in accordance with some embodiments.

FIGS. 10A, 10B, 10C, and 10D depict aspects of mass transfer of binding entities toward a surface of a solid support containing array sites, in accordance with some embodiments.

FIGS. 11A, 11B, and 11C show aspects of mass transfer of binding entities toward a surface of a solid support containing array sites in the presence of a multi-phase interface, in accordance with some embodiments.

FIGS. 12A, 12B, and 12C display a method of resuspending binding entities to increase loading of array sites, in accordance with some embodiments.

FIGS. 13A, 13B, 13C, and 13D illustrate mass transfer of binding entities along filamentous moieties toward array sites, in accordance with some embodiments. FIGS. 13E, 13F, and 13G illustrate advantageous configurations of filamentous moieties for mass transfer of binding entities, in accordance with some embodiments.

FIGS. 14A, 14B, and 14C depict differing configurations of filamentous moieties for coupling binding entities to array sites, in accordance with some embodiments.

FIGS. 15A, 15B, and 15C show different methods of generating near-surface flow patterns utilizing particles, in accordance with some embodiments.

FIGS. 16A and 16B display development of an anisotropic binding entity distribution on a surface of a solid support, in accordance with some embodiments.

FIGS. 17A and 17B illustrate utilization of wave-based phenomena to affect mass transfer of binding entities across a surface of a solid support, in accordance with some embodiments.

FIGS. 18A and 18B depict deposition of a material or fluid at an interstitial region to affect mass transfer of binding entities across a surface of a solid support, in accordance with some embodiments.

FIG. 19 shows a system for continuous deposition of analytes on an array, in accordance with some embodiments.

FIGS. 20A, 20B, and 20C display different binding entities configurations to achieve loading of array sites with differing binding competencies, in accordance with some embodiments.

FIG. 21 illustrates aspects of binding entity binding phenomena, in accordance with some embodiments.

FIG. 22 depicts differences in binding behavior for binding entities comprising or not comprising non-rigid linkers, in accordance with some embodiments.

FIGS. 23A, 23B, and 23C show differing configuration of coupling moieties on a face of an anchoring moiety, in accordance with some embodiments.

FIG. 24 displays impact of array site binding properties on optimal configuration of binding entities to achieve polyvalent binding, in accordance with some embodiments.

FIGS. 25A and 25B illustrate array site occupancy and array site co-occupancy data for a multi-cycle anchoring moiety deposition method. FIG. 25C displays a fluorescence microscopy image for an array after a multi-cycle anchoring moiety deposition method.

FIGS. 26A and 26B depict differing orientations of surface-coupling oligonucleotides on a face of an anchoring moiety. FIG. 26C depicts incorporation of a PEG linker into a surface-coupling oligonucleotide of an anchoring moiety. FIG. 26D depicts array site occupancy fraction for differing designs of anchoring moieties.

FIG. 27A shows differing array site configurations, as distinguished by incorporation of varying PEG linkers into surface-coupled oligonucleotides. FIG. 27B shows array site occupancy fraction for arrays with varying array site configurations.

FIGS. 28A and 28B display differing configurations of an array system that utilizes volumetric expanders to concentrate particle adjacent to an array surface, in accordance with some embodiments.

FIGS. 29A and 29B illustrate the effect of differing particle surface charge on relative proximity between array-bound moieties and mobile particles, in accordance with some embodiments.

FIG. 30 depicts merging of two fluidic stream to induce mass transfer toward an array surface, in accordance with some embodiments.

FIGS. 31A, 31B, and 31C show steps of a method for depositing analytes at array sites of an array utilizing carrier particles, in accordance with some embodiments. FIGS. 31D, 31E, 31F, and 31G show a method of loading utilized carrier particles with additional moieties, in accordance with some embodiments. FIGS. 31H, 31I, and 31J show a method of utilizing re-loaded carrier particles to deliver affinity agents to a surface of an array, in accordance with some embodiments.

FIG. 32A displays a top-down view of an array surface comprising array sites in a hexagonal pattern, in accordance with some embodiments. FIG. 32B displays a top-down view of the array of FIG. 32A with single-layer electrical-current loops for generating a magnetic field at each array site, in accordance with some embodiments.

FIG. 32C displays a cross-sectional view of the array of FIG. 32B, in accordance with some embodiments. FIG. 32D displays a top-down view of the array of FIG. 32A with double-layer electrical-current loops for generating a magnetic field at each array site, in accordance with some embodiments. FIG. 32E displays a cross-sectional view of the array of FIG. 32D, in accordance with some embodiments.

FIGS. 33A and 33B illustrate configurations of an array with well features that are configured to generate an electric field in each well feature, in accordance with some embodiments.

FIG. 34 depicts a schematic of a method for forming an array of analytes, in accordance with some embodiments.

FIGS. 35A and 35B show plots of observed single and multiple occupancy of array sites as a function of total occupancy of an array for two different types of anchoring moieties.

FIG. 36A displays an image of anchoring moiety separation by gel electrophoresis with monomer and multimer or aggregate bands present. FIGS. 36B and 36C display intensity histograms of observed arrays of anchoring moieties formed by anchoring moieties from the monomer band and anchoring moieties from an unpurified control sample, respectively.

### DETAILED DESCRIPTION

Assays, including genomic, transcriptomic, and proteomic assays, are often configured in array-based fashions due to the advantage of spatial ordering that arrays can provide. Arrays typically contain a solid support with a plurality of discrete sites, with each site being configured to contain analytes or other assay reagents. A solid support of an array may be patterned to provide a regular, uniform, or predictable spatial ordering of array sites, or may be unpatterned. As the length-scale of spatial ordering of an array decreases toward the nanoscale, arrays may be configured to display collections of particles such as nanoparticles (e.g., polypeptides, nucleic acids, polysaccharides, metabolites, non-biological nanoparticles, etc.) in clusters or single-analyte fashion. Single-analyte arrays may be particularly advantageous by providing individual analytes at fixed array addresses with sufficient spatial separation between analytes to permit separate optical interrogation of each analyte.

For single-analyte applications, it can be preferable to provide arrays that have a maximal single-analyte occupancy (i.e., arrays with a maximized number of array sites containing one and only one analyte). Ideally, a single-analyte array would be characterized as having 100% of the array sites occupied with one and only one analyte. Given the vagaries of nanoscale phenomena, the probability of achieving ideal loading of a single-analyte array decreases as the length-scale of array sites and/or analytes decreases. Accordingly, array occupancy becomes governed in part by stochastic phenomena at nanometer length-scales. In specific cases, it is preferable to provide “super Poisson loading,” of a single-analyte array, meaning that the single-analyte occupancy of the array exceeds the theoretical loading predicted by a statistical Poisson distribution. Namely, super Poisson loading of an array with an expected site occupancy of one analyte may be characterized as having: i)>37% of array sites containing one and only one molecule, ii) optionally <37% of sites containing no analytes, and iii) optionally <26% of sites containing two or more analytes.

Arrays configured for super Poisson loading will typically have tailored interactions with analytes that facilitate analyte deposition at array sites and inhibit analyte deposition away from arrays sites (e.g., at interstitial regions between array sites). Moreover, arrays configured for super Poisson loading may utilize mechanisms such as size exclusion or steric hindrance to inhibit co-occupancy of array sites by multiple analytes. Given natural variability during manufacturing of arrays, as well as possible heterogeneity of analytes contacted with an array, some fraction of sites is likely to remain unoccupied after analyte loading, and some fraction of sites is likely to be co-occupied by multiple analytes. Moreover, although it may be possible to maximize single-analyte array occupancy given infinite time, real world processes typically impose a limit on the time afforded to achieve maximized single-analyte array loading.

Accordingly, the present disclosure provides methods and system for achieving improved single-analyte occupancy of arrays. Methods for improving mass transfer of nanoparticles to array sites are provided. Also provided are methods for improving binding specificity and decreasing loading time, thereby facilitating improved super Poisson loading of single-analyte arrays. Also provided are systems that are configured to implement methods of single-analyte array loading, as set forth herein.

An array of analytes formed by a method, as set forth herein, may facilitate an outcome of an assay or other process performed on the array of analytes. An array of analytes may be formed by a method, as set forth herein, that provides a sufficient quantity and/or diversity of analytes to facilitate an outcome of an assay or other process performed on the array of analytes. An assay or other process performed on an array of analytes formed by a method, as set forth herein, may facilitate identification, characterization, or any other conceivable form of interrogation of a sufficient quantity and/or diversity of analytes. Accordingly, some methods of forming arrays of analytes with sufficient quantity and/or diversity of analytes to facilitate an assay or process outcome are provided herein.

A formed array of analytes may comprise a bound quantity of analytes, wherein the bound quantity of analytes is a percentage of a total quantity of analytes of a population of analytes, such as at least about 0.00001%, 0.0001%, 0.001%, 0.01%, 0.1%, 0.5%, 1%, 5%, 10%, 15%, 20%, 25%, 30%, 35%, 40%, 45%, 50%, 55%, 60%, 65%, 70%, 75%, 80%, 85%, 90%, 95%, 99%, 99.9%, 99.99%, 99.999%, 99.9999%, 99.999990%, or more than 99.99999% of a total quantity of analytes of a population of analytes. Alternatively or additionally, a bound quantity of analytes may be a percentage of a total quantity of analytes of a population of analytes, such as no more than about 99.99999%, 99.9999%, 99.999%, 99.99%, 99.9%, 99%, 95%, 90%, 85%, 80%, 75%, 70%, 65%, 60%, 55%, 50%, 45%, 40%, 35%, 30%, 25%, 20%, 15%, 10%, 5%, 1%, 0.5%, 0.1%, 0.01%, 0.001%, 0.0001%, 0.00001%, or less 0.00001% of a total quantity of analytes of a population of analytes. Accordingly, an assay or array process utilizing an array of analytes may identify, characterize, or otherwise interrogate a bound quantity of analytes, wherein the identified, characterized, or otherwise interrogated analytes is a percentage of a total quantity of analytes of a population of analytes, such as at least about 0.00001%, 0.0001%, 0.001%, 0.01%, 0.1%, 0.5%, 1%, 5%, 10%, 15%, 20%, 25%, 30%, 35%, 40%, 45%, 50%, 55%, 60%, 65%, 70%, 75%, 80%, 85%, 90%, 95%, 99%, 99.9%, 99.99%, 99.999%, 99.9999%, 99.99999%, or more than 99.99999% of a total quantity of analytes of a population of analytes. Alternatively or additionally, an identified, characterized, or otherwise interrogated percentage of analytes may be a percentage of a total quantity of analytes of a population of analytes, such as no more than about 99.99999%, 99.9999%, 99.999%, 99.99%, 99.9%, 99%, 95%, 90%, 85%, 80%, 75%, 70%, 65%, 60%, 55%, 50%, 45%, 40%, 35%, 30%, 25%, 20%, 15%, 10%, 5%, 1%, 0.5%, 0.1%, 0.01%, 0.001%, 0.0001%, 0.00001%, or less 0.00001% of a total quantity of analytes of a population of analytes.

A formed array of analytes may comprise a bound quantity of analytes, wherein the bound quantity of analytes contains a total percentage of analyte diversity (e.g., species diversity, isoform diversity, proteomic diversity, microbiomic diversity, or combinations thereof), such as at least about 0.00001%, 0.0001%, 0.001%, 0.01%, 0.1%, 0.5%, 1%, 5%, 10%, 15%, 20%, 25%, 30%, 35%, 40%, 45%, 50%, 55%, 60%, 65%, 70%, 75%, 80%, 85%, 90%, 95%, 99%, 99.9%, 99.99%, 99.999%, 99.9999%, 99.999990%, or more than 99.99999% of analyte diversity. Alternatively or additionally, a bound quantity of analytes may contain a percentage of analyte diversity, such as no more than about 99.99999%, 99.9999%, 99.999%, 99.99%, 99.9%, 99%, 95%, 90%, 85%, 80%, 75%, 70%, 65%, 60%, 55%, 50%, 45%, 40%, 35%, 30%, 25%, 20%, 15%, 10%, 5%, 1%, 0.5%, 0.1%, 0.01%, 0.001%, 0.0001%, 0.00001%, or less 0.00001% of analyte diversity. Accordingly, an assay or array process utilizing an array of analytes may identify, characterize, or otherwise interrogate a bound quantity of analytes, wherein the bound quantity of analytes contains a percentage of analyte diversity of analytes of a population of analytes, such as at least about 0.00001%, 0.0001%, 0.001%, 0.01%, 0.1%, 0.5%, 1%, 5%, 10%, 15%, 20%, 25%, 30%, 35%, 40%, 45%, 50%, 55%, 60%, 65%, 70%, 75%, 80%, 85%, 90%, 95%, 99%, 99.9%, 99.99%, 99.999%, 99.9999%, 99.99999%, or more than 99.99999% of analyte diversity of a total quantity of analytes of a population of analytes. Alternatively or additionally, an identified, characterized, or otherwise interrogated percentage of analytes may contain a percentage of analyte diversity of analytes of a population of analytes, such as no more than about 99.99999%, 99.9999%, 99.999%, 99.99%, 99.9%, 99%, 95%, 90%, 85%, 80%, 75%, 70%, 65%, 60%, 55%, 50%, 45%, 40%, 35%, 30%, 25%, 20%, 15%, 10%, 5%, 1%, 0.5%, 0.1%, 0.01%, 0.001%, 0.0001%, 0.00001%, or less 0.00001% of analyte diversity of a total quantity of analytes of a population of analytes.

A formed array of analytes may comprise a bound quantity of analytes, wherein the bound quantity of analytes contains a dynamic range of analyte species (e.g., a ratio of a quantity of a most common analyte species to a least common analyte species, a ratio of a first analyte species to a second analyte species, etc.), such as at least about 1, 2, 5, 10, 10², 10³, 10⁴, 10⁵, 10⁶, 10⁷, 10⁸, 10⁹, 10¹⁰, 10¹¹, 10¹², or more than 10¹² of analyte dynamic range. Alternatively or additionally, a bound quantity of analytes may contain a dynamic range of analyte species, such as no more than about 10¹², 10¹¹, 10¹⁰, 10⁹, 10⁸, 10⁷, 10⁶, 10⁵, 10⁴, 10¹, 10², 10, 5, 2, or less than 2 of analyte dynamic range. Accordingly, an assay or array process utilizing an array of analytes may identify, characterize, or otherwise interrogate a bound quantity of analytes, wherein the bound quantity of analytes contains a dynamic range of analyte species, such as at least about 1, 2, 5, 10, 10², 10³, 10⁴, 10⁵, 10⁶, 10⁷, 10⁸, 10⁹, 10¹⁰, 10¹¹, 10¹², or more than 10¹² of analyte dynamic range. Alternatively or additionally, an identified, characterized, or otherwise interrogated percentage of analytes may contain a dynamic range of analyte species, such as no more than about 10¹², 10¹¹, 10¹⁰, 10⁹, 10⁸, 10⁷, 10⁶, 10⁵, 10⁴, 10³, 10², 10, 5, 2, or less than 2 of analyte dynamic range.

A skilled person in the art will readily recognize that the methods and systems disclosed herein may be exemplified in the context of effecting transfer of analytes or other moieties (e.g., anchoring moieties) to array surfaces, but may also be useful for the delivery of other reagents, molecules, macromolecules, or moieties to array surfaces as well. The methods and systems set forth herein may be especially useful for the delivery of any particles or macromolecules to array surfaces. For example, methods are set forth herein for performing assays of analytes on arrays by contacting the analytes with detection reagents comprising one or more affinity reagents (e.g., antibodies, antibody fragments, aptamers, peptamers, avimers, etc.) coupled to a nanoparticle (e.g., a nucleic acid nanoparticle, a fluorescent nanoparticle, a polymeric nanoparticle, etc.). The methods may also be useful for delivery of assay reagents such as enzymes or detection reagents (e.g., fluorescently-labeled molecules, particles, or moieties, luminescent molecules, particles or moieties, etc.). Accordingly, it may be advantageous to provide a method of mass transfer to an array surface or across an array surface that facilitates mass transfer of a detection reagent or any other conceivable assay reagent to an array site. In some cases, a method set forth herein may comprise a first step of providing a first method of mass transfer, as set forth herein, for transferring an analyte or anchoring moiety to an array site, and a second step of providing a second method of mass transfer, as set forth herein, for transferring a reagent, particle, or macromolecule to the array site.

### Definitions

Terms used herein will be understood to take on their ordinary meaning in the relevant art unless specified otherwise. Several terms used herein and their meanings are set forth below.

As used herein, the term “array site binding availability,” when used in reference to a single array site, refers to the single array site possessing sufficient structural features to form an intended and/or designed binding interaction with a complementary component (e.g., binding between the array site and an analyte and/or an anchoring moiety). Sufficient structural features of an array site for forming an intended and/or designed binding interaction with a complementary component can include presence of a coupling moiety or a plurality thereof at the array site, optional presence of a covalent attachment between a coupling moiety and a solid support on which the array site is disposed, optional presence of a passivating moiety or a plurality thereof at the array site, and physical accessibility of the coupling moiety (i.e., the coupling moiety is not blocked, sequestered, or occluded by a blocking moiety). Array site binding availability for a single array site may be categorized according to a binary classification (e.g., AVAILABLE/NOT AVAILABLE or ACTIVE/INACTIVE) or a polynary classification (e.g., AVAILABLE, NOT AVAILABLE, UNKNOWN, etc.). As used herein, the term “array site binding availability,” when used in reference to a population of array sites, refers to a fraction or percentage of single array sites that are classified as possessing single array site binding availability, as set forth above. For example, the array site binding availability of an array can be 0.95 or 95% of the array sites if all but 0.05 or 5% of the sites of the array are classified as being available for binding of an analyte or an anchoring moiety.

As used herein, the term “analyte binding availability” or “anchoring moiety binding availability,” when used in reference to a single analyte or anchoring moiety, respectively, refers to the single analyte or anchoring moiety possessing sufficient structural features to form an intended and/or designed binding interaction with a complementary component (e.g., binding between the analyte and/or anchoring moiety and an array site). Sufficient structural features of an analyte or anchoring moiety for forming an intended and/or designed binding interaction with a complementary component can include presence of a coupling moiety or a plurality thereof on the analyte and/or anchoring moiety, optional presence of a covalent attachment between a coupling moiety and the analyte or anchoring moiety, optional presence of a passivating moiety or a plurality thereof on the analyte and/or anchoring moiety, and physical accessibility of the coupling moiety (i.e., the coupling moiety is not blocked, sequestered, or occluded by a blocking moiety). Analyte or anchoring moiety binding availability for a single analyte or a single anchoring moiety may be categorized according to a binary classification (e.g., AVAILABLE/NOT AVAILABLE, or ACTIVE/NOT ACTIVE) or a polynary classification (e.g., AVAILABLE, NOT AVAILABLE, UNKNOWN, etc.). As used herein, the term “analyte binding availability” or “anchoring moiety binding availability,” when used in reference to a population of analytes or anchoring moieties, refers to a fraction or percentage of single analytes or single anchoring moieties that are classified as possessing single analyte or single anchoring moiety binding availability, as set forth above. For example, the analyte binding availability of a fluidic medium containing a plurality of analytes can be 0.95 or 95% of the analytes within the fluidic medium if all but 0.05 or 5% of the analytes with the fluidic medium are classified as being available for binding to an array site.

As used herein, the term “array site binding competency,” when used in reference to a single array site, refers to a deterministic, probabilistic, or stochastic characterization of binding likelihood for an available single array site (per the above-described definition of array site availability) with respect to an analyte, an anchoring moiety, or a population thereof. Accordingly, two differing single array sites of a population of array sites may have differing array site binding competencies, respectively, thereby indicating a tendency to preferentially bind differing types of analytes or anchoring moieties, respectively, or differing likelihoods of binding an analyte or anchoring moiety, respectively. Array site binding competency for a single array site may be determined, in whole or in part, by a structural feature or a combination of structural features of the single array site. Relevant structural features that may influence array binding site competency include, but are not limited to, array site dimension (e.g., width, length, height or diameter), array site 2-dimensional shape or morphology (e.g., triangular, ovaline, rectangular, polygonal, amorphous, etc.), array site 3-dimensional shape or morphology (e.g., raised, inset, combinations thereof, etc.), quantity of coupling moieties, surface density of coupling moieties, quantity of passivating moieties, surface density of passivating moieties, overall surface density of surface-bound moieties, spatial variability of surface-bound moieties, chemical composition of coupling moieties (e.g., oligonucleotides, receptor-ligand components, chemically reactive moieties, etc.), variability of composition of coupling moieties (e.g., frequency of incorrect oligonucleotide sequences, chemical impurities, degraded or damaged moieties, etc.), chemical composition of passivating moieties (e.g., hydrophobic polymers, hydrophilic polymers, electrically-charged polymers, zwitterionic polymers, biopolymers, etc.), variability of composition of passivating moieties (e.g., chemical impurities, degraded or damaged moieties, etc.), net surface physical properties (e.g., electrical charge, polarity, hydrophobicity, hydrophilicity, nucleophilicity, electrophilicity, etc.), physical property isotropy or anisotropy, or combinations thereof. Array site binding competency for a single array site may be determined empirically or theoretically. Array site binding competency of a single array site may be determined as a function of one or more structural features of the single array site. Characterization of a structural feature of an array site may occur by a bulk or single-site physical characterization process (e.g., a quality control measurement).

As used herein, the term “array site binding competency profile,” refers to a qualitative or quantitative representation of array site binding competency for a population of single array sites. An array site binding competency profile may be formulated or presented as a discrete or continuous statistical distribution (e.g., a normal distribution, a Gaussian distribution, a Poisson distribution, a monomodal distribution, a polymodal distribution, etc.). An array site binding competency profile may provide information on site population size as a function of a structural feature for array sites. An array site binding competency profile may provide information on site population size as a function of two or more structural features for array sites. An array site binding competency profile may provide information on site population size as a function of a structural feature for an analyte or an anchoring moiety. An array site binding competency profile may be a qualitative metric or parameter that corresponds to a characterized quantitative assessment of array site binding competency. For example, an array may be provided with a single-parameter array site binding competency profile (e.g., High/Medium/Low; Type A/Type B/Type C/ . . . ; Type 1/Type 2/Type 3/ . . . ; etc.) or a multi-parameter array site binding competency profile (e.g., Type 1A/Type 1B/Type 2A/ . . . ; etc.), in which the provided metric(s) or parameter(s) correspond to quantitative aspects of array site binding competency, as set forth herein. A population of array sites may be divided into subpopulations of array sites (e.g., each subpopulation comprising a differing species of array site), in which each subpopulation has an array site binding competency profile, and in which an overall array site binding competency profile for the population of array sites is an aggregate or weighted profile based upon the profiles of each subpopulation (e.g., based upon relative quantities of subpopulations).

As used herein, the term “analyte binding competency” or “anchoring moiety binding competency,” when used in reference to a single analyte or anchoring moiety, refers to a deterministic, probabilistic, or stochastic characterization of binding likelihood for an available single analyte or anchoring moiety (per the above-described definitions of analyte and anchoring moiety availability) with respect to an array site, or a population thereof. Accordingly, two differing single analytes or anchoring moieties of a population of analytes or anchoring moieties may have differing analyte or anchoring moiety binding competencies, respectively, thereby indicating a tendency to preferentially bind differing types of array sites, respectively, or differing likelihoods between the two unique analytes or anchoring moieties of binding to an array site, respectively. Analyte or anchoring moiety binding competency for a single analyte or anchoring moiety may be determined, in whole or in part, by a structural feature or a combination of structural features of the single analyte or anchoring moiety. Relevant structural features that may influence array binding site competency include, but are not limited to, analyte or anchoring moiety dimension (e.g., width, length, height, diameter or hydrodynamic radius), analyte or anchoring moiety 2-dimensional shape or morphology (e.g., triangular, ovaline, rectangular, polygonal, amorphous, etc.), analyte or anchoring moiety 3-dimensional shape or morphology, quantity of coupling moieties, surface density of coupling moieties, quantity of passivating moieties, surface density of passivating moieties, overall surface density of surface-bound moieties, spatial variability of surface-displayed moieties, chemical composition of coupling moieties (e.g., oligonucleotides, receptor-ligand components, chemically reactive moieties, etc.), variability of composition of coupling moieties (e.g., frequency of incorrect oligonucleotide sequences, chemical impurities, degraded or damaged moieties, etc.), chemical composition of passivating moieties (e.g., hydrophobic polymers, hydrophilic polymers, electrically-charged polymers, zwitterionic polymers, biopolymers, etc.), variability of composition of passivating moieties (e.g., chemical impurities, degraded or damaged moieties, etc.), net physical properties (e.g., electrical charge, polarity, hydrophobicity, hydrophilicity, nucleophilicity, electrophilicity, etc.), physical property isotropy or anisotropy, or combinations thereof. Analyte or anchoring moiety binding competency for a single analyte or anchoring moiety may be determined empirically or theoretically. Analyte or anchoring moiety binding competency of a single analyte or anchoring moiety may be determined as a function of one or more structural features of the single analyte or anchoring moiety. Characterization of a structural feature of an analyte or anchoring moiety may occur by a bulk or single-molecule physical characterization process (e.g., a quality control measurement).

As used herein, the term “analyte binding competency profile” or “anchoring moiety binding competency profile” refers to a qualitative or quantitative representation of analyte or anchoring moiety binding competency for a population of single analyte or anchoring moieties. An analyte binding competency profile or anchoring moiety binding competency profile may be formulated or presented as a discrete or continuous statistical distribution (e.g., a normal distribution, a Gaussian distribution, a Poisson distribution, a monomodal distribution, a polymodal distribution, etc.). An analyte binding competency profile or anchoring moiety binding competency profile may provide information on analyte or anchoring moiety population size as a function of a structural feature for analytes or anchoring moieties. An analyte or anchoring moiety binding competency profile may provide information on site population size as a function of two or more structural features for analytes or anchoring moieties. An analyte or anchoring moiety binding competency profile may provide information on analyte or anchoring moiety population size as a function of a structural feature for an array site. An analyte binding competency profile or an anchoring moiety binding competency profile may be a qualitative metric or parameter that corresponds to a characterized quantitative assessment of array site binding competency. For example, a population of analytes may be provided with a single-parameter analyte binding competency profile (e.g., High/Medium/Low; Type A/Type B/Type C/ . . . ; Type 1/Type 2/Type 3/ . . . ; etc.) or a multi-parameter array site binding competency profile (e.g., Type 1A/Type 1B/Type 2A/ . . . ; etc.), in which the provided metric(s) or parameter(s) correspond to quantitative aspects of analyte binding competency, as set forth herein. In some cases, metrics or parameters of a qualitative analyte or anchoring moiety binding competency profile may correspond to metrics or parameters of a qualitative array site binding competency profile. For example, a population of analytes with a Type 1A analyte binding competency profile may be optimal for binding to an array with a Type 1A array site binding competency profile. A population of analyte or anchoring moieties may be divided into subpopulations of analytes or anchoring moieties (e.g., each subpopulation comprising a differing species of analyte or anchoring moiety), in which each subpopulation has an analyte or anchoring moiety binding competency profile, and in which an overall analyte or anchoring moiety binding competency profile for the population of analytes or anchoring moieties is an aggregate or weighted profile based upon the profiles of each subpopulation (e.g., based upon relative quantities of subpopulations).

As used herein, the term “nucleic acid nanoparticle,” refers to a single- or multi-chain polynucleotide molecule comprising a compacted three-dimensional structure. The compacted three-dimensional structure can optionally have a characteristic tertiary structure. An exemplary nucleic acid nanostructure is a structured nucleic acid particle (SNAP). A SNAP can be configured to have an increased number of interactions between regions of a polynucleotide strand, less distance between the regions, increased number of bends in the strand, and/or more acute bends in the strand, as compared to the same nucleic acid molecule in a random coil or other non-structured state. Alternatively or additionally, the compacted three-dimensional structure of a nucleic acid nanostructure can optionally have a characteristic quaternary structure. For example, a nucleic acid nanostructure can be configured to have an increased number of interactions between polynucleotide strands or less distance between the strands, as compared to the same nucleic acid molecule in a random coil or other non-structured state. In some configurations, the tertiary structure (i.e. the helical twist or direction of the polynucleotide strand) of a nucleic acid nanostructure can be configured to be more dense than the same nucleic acid molecule in a random coil or other non-structured state. Nucleic acid nanostructures may include deoxyribonucleic acid (DNA), ribonucleic acid (RNA), peptide nucleic acid (PNA), other nucleic acid analogs, and combinations thereof. Nucleic acid nanostructures may have naturally-arising or engineered secondary, tertiary, or quaternary structures. A structured nucleic acid particle can contain at least one of: i) a moiety that is configured to couple an analyte to the nucleic acid nanostructure, ii) a moiety that is configured to couple the nucleic acid nanostructure to another object such as another SNAP, a solid support or a surface thereof, iii) a moiety that is configured to provide a chemical or physical property or characteristic to a nucleic acid nanostructure, or iv) a combination thereof. Exemplary SNAPs may include nucleic acid nanoballs (e.g. DNA nanoballs), nucleic acid nanotubes (e.g. DNA nanotubes), and nucleic acid origami (e.g. DNA origami). A SNAP may be functionalized to include one or more reactive handles or other moieties. A SNAP may comprise one or more incorporated residues that contain reactive handles or other moieties (e.g., modified nucleotides).

As used herein, the term “primary structure,” when used in reference to a nucleic acid, refers to a residue sequence of a single-stranded nucleic acid. As used herein, the term “secondary structure,” when used in reference to a nucleic acid, refers to the base-pairing interactions within a single nucleic acid polymer or between two polymers. Secondary structure may include multi-stranded nucleic acids formed by self-complementarity of a single oligonucleotide, such as stems, loops, bulges, and junctions. As used herein, the term “tertiary structure,” when used in reference to a nucleic acid, refers to the three-dimensional conformation of a nucleic acid, such as the overall three-dimensional shape of a single-stranded nucleic acid or multi-stranded nucleic acid.

As used herein, the term “residue,” when used in reference to a polymer, refers to a monomeric unit of a polymer structure. When used in reference to a nucleic acid, a residue may refer to a nucleotide, nucleoside, or a synthetic, modified, or non-natural analogue thereof. When used in reference to a polypeptide, a residue may refer to an amino acid or a synthetic, modified, or non-natural analogue thereof.

As used herein, the terms “type” or “species,” when used in reference to a molecule, particle, or moiety, refer to a molecule, particle, or moiety with a unique, distinguishable chemical structure. As used herein, the term “type of anchoring moiety” refers to an anchoring moiety with a unique, distinguishable binding characteristic, for example, as characterized by an anchoring moiety binding availability or anchoring moiety binding competency. A first anchoring moiety may have one or more structural dissimilarities, such as an absence of a detectable label or a damaged moiety, with respect to a second anchoring moiety and still be of the same type of anchoring moiety if the structural dissimilarities do not result in a difference in binding characteristic between the first anchoring moiety and the second anchoring moiety. Anchoring moiety variants with differences in quantity, location, orientation, and types of coupling moieties are different species from each other if the differences result in differences in a binding characteristic. For example, members of a “type of anchoring moiety” can have a unique, distinguishable structure that is common to the members compared to other anchoring moieties that lack the unique, distinguishable structure. Anchoring moiety types may be identified, for example, by common shape and/or conformation, number of coupling moieties, or type of coupling moieties.

As used herein, the terms “click reaction,” “click-type reaction,” or “bioorthogonal reaction” refer to single-step, thermodynamically-favorable conjugation reaction utilizing biocompatible reagents. A click reaction may be configured to not utilize toxic or biologically incompatible reagents (e.g., acids, bases or heavy metals) or to not generate toxic or biologically incompatible byproducts. A click reaction may utilize an aqueous solvent or buffer (e.g., phosphate buffer solution, Tris buffer, saline buffer, MOPS, etc.). A click reaction may be thermodynamically favorable if it has a negative Gibbs free energy of reaction, for example a Gibbs free energy of reaction of less than about −5 kiloJoules/mole (kJ/mol), −10 kJ/mol, −25 kJ/mol, −50 kJ/mol, −100 kJ/mol, −200 kJ/mol, −300 kJ/mol, −400 kJ/mol, or less than −500 kJ/mol. Exemplary bioorthogonal and click reactions are described in detail in WO 2019/195633A1, which is herein incorporated by reference in its entirety. Exemplary click reactions may include metal-catalyzed azide-alkyne cycloaddition, strain-promoted azide-alkyne cycloaddition, strain-promoted azide-nitrone cycloaddition, strained alkene reactions, thiol-ene reaction, Diels-Alder reaction, inverse electron demand Diels-Alder reaction, [3+2] cycloaddition, [4+1] cycloaddition, nucleophilic substitution, dihydroxylation, thiol-yne reaction, photoclick, nitrone dipole cycloaddition, norbornene cycloaddition, oxanobornadiene cycloaddition, tetrazine ligation, and tetrazole photoclick reactions. Exemplary functional groups or reactive handles utilized to perform click reactions may include alkenes, alkynes, azides, epoxides, amines, thiols, nitrones, isonitriles, isocyanides, aziridines, activated esters, and tetrazines. Other well-known click conjugation reactions may be used having complementary bioorthogonal reaction species, for example, where a first click component comprises a hydrazine moiety and a second click component comprises an aldehyde or ketone group, and where the product of such a reaction comprises a hydrazone functional group or equivalent.

As used herein, the term “array” refers to a population of sites that provide spatial separation of molecules, moieties, or analytes that are resolved such that the sites can be distinguished from each other. Accordingly, molecules, moieties or analytes at one site of an array can be resolved from molecules, moieties or analytes at other sites of the array. The sites can function as unique identifiers and/or the sites can be attached to unique identifiers. The term “array of analytes” refers to an array with a population of sites, in which a plurality of sites of the population of sites is occupied by analytes.

As used herein, the term “unique identifier” refers to a solid support (e.g., particle or bead), spatial address in an array, tag, label (e.g., luminophore), or barcode (e.g., nucleic acid barcode) that is attached to an analyte and that is distinct from other identifiers, throughout one or more steps of a process. The process can be an analytical process such as a method for detecting, identifying, characterizing or quantifying an analyte. Attachment to a unique identifier can be covalent or non-covalent (e.g., ionic bond, hydrogen bond, van der Waals forces etc.). A unique identifier can be exogenous to the analyte, for example, being synthetically attached to the analyte. Alternatively, a unique identifier can be endogenous to the analyte, for example, being attached or associated with the analyte in the native milieu of the analyte. An array can include different analytes that are each attached to different unique identifiers. For example, an array can include different molecules or analytes that are each located at different addresses on a solid support. Alternatively, an array can include separate solid supports each functioning as an address that bears a different molecule or analyte, where the different molecules or analytes can be identified according to the locations of the solid supports on a surface to which the solid supports are attached, or according to the locations of the solid supports in a liquid such as a fluid stream. The molecules or analytes of the array can be, for example, nucleic acids such as SNAPs, polypeptides, proteins, peptides, oligopeptides, enzymes, ligands, or receptors such as antibodies, functional fragments of antibodies or aptamers. The addresses of an array can optionally be optically observable and, in some configurations, adjacent addresses can be optically distinguishable when detected using a method or apparatus set forth herein.

As used herein, the term “address” refers to a physical location on a surface of a solid support and/or an array, as set forth herein. An address may be definable with respect to a coordinate system that provides a uniform frame of reference for defining each individual address (e.g., an x-y-z axis coordinate system). On a substantially flat or planar solid support or array, an address may be provided with respect to a two-dimensional coordinate system, such as a rectangular coordinate system (e.g., x-y axis) or a polar coordinate system. For a non-planar or curved solid support or array, an address may be provided with respect to a three-dimensional coordinate system, such as spherical coordinates or a rectangular coordinate system (x-y-z axis).

As used herein, the terms “binding site,” “array site,” and “site,” when used in reference to an array, means an address in an array where a particular molecule or analyte is present. A site can contain only a single molecule or analyte, or it can contain a population of several molecules or analytes of the same species (i.e. an ensemble of the molecules). Alternatively, a site can include a plurality of molecules or analytes that are different species. Sites of an array are typically discrete or spatially separated from each other array site (i.e., two discrete array sites will have no overlapping addresses). Sites can be optically resolvable. The discrete sites can be contiguous, or they can have interstitial spaces between each other. An array useful herein can have, for example, sites that are separated by less than 100 microns, 10 microns, 1 micron, 500 nm, 100 nm, 10 nm or less. Alternatively or additionally, an array can have sites that are separated by at least 10 nm, 100 nm, 500 nm, 1 micron, 5 microns, 10 microns, 50 microns, 100 microns or more. An individual site can contain a plurality of addresses (i.e., multiple addresses can be located within a single array site); accordingly, an individual site can have a measurable surface area. The sites can each have a surface area of less than 1 square millimeter, 500 square microns, 100 square microns, 25 square microns, 1 square micron or less. An array can include at least about 1×10⁴, 1×10⁵, 1×10⁶, 1×10⁸, 1×10¹⁰, 1×10¹², or more sites.

As used herein, the term “solid support” refers to a substrate that is insoluble in aqueous liquid. Optionally, the substrate can be rigid. The substrate can be non-porous or porous. The substrate can optionally be capable of taking up a liquid (e.g., due to porosity) but will typically, but not necessarily, be sufficiently rigid that the substrate does not swell substantially when taking up the liquid and does not contract substantially when the liquid is removed by drying. A nonporous solid support is generally impermeable to liquids or gases. Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefins, polyimides etc.), nylon, ceramics, resins, Zeonor™, silica or silica-based materials including silicon and modified silicon, carbon, metals, metal oxides (e.g., zirconia, titania, alumina, etc.), inorganic glasses, optical fiber bundles, gels, and polymers.

As used herein, the terms “group” and “moiety” are intended to be synonymous when used in reference to the structure of a molecule. The terms refer to a component or part of the molecule. The terms do not necessarily denote the relative size of the component or part compared to the rest of the molecule, unless indicated otherwise. A group or moiety can contain one or more atom. As used herein, the term “coupling moiety” refers to a component or part of a molecule or particle that is configured to couple the molecule or particle to a second molecule or particle, or that couples the molecule or particle to the second molecule or particle. A coupling moiety may couple a molecule or particle to a second molecule or particle reversibly or irreversibly. A coupling moiety may couple a molecule or particle to a second molecule or particle covalently or non-covalently.

As used herein, the term “face” refers to a portion of a molecule, particle, or complex (e.g., a SNAP or a SNAP complex) that contains one or more moieties with substantially similar orientation and/or function. For example, a substantially rectangular or square SNAP may have a coupling face that comprises one or more coupling moieties, with each coupling moiety having a substantially similar orientation to each other coupling moiety (e.g., oriented about 180° from a display moiety that is configured to be coupled to an analyte). In another example, a spherical nanoparticle may have a coupling face comprising a coupled plurality of coupling moieties confined to a hemisphere of the particle (i.e., a plurality of coupling moieties having similar function but differing orientations). In some cases, a face may be defined by an imaginary plane relative to which a moiety or a portion thereof may have a spatial proximity or angular orientation when the plane is contacted with a point or portion of a molecule, particle, or complex. A moiety or a portion thereof may have a spatial separation from an imaginary plane defining a face of a molecule, particle, or complex of no more than about 100 nanometers (nm), 90 nm, 80 nm, 70 nm, 60 nm, 50 nm, 40 nm, 30 nm, 25 nm 20 nm, 15 nm, 10 nm, 9 nm, 8 nm, 7 nm, 6 nm, 5 nm, 4 nm, 3 nm, 2 nm, 1 nm, 0.5 nm, 0.1 nm, or less than 0.1 nm. A moiety or a portion thereof may have an angular orientation relative to a normal vector of an imaginary plane of no more than about 90°, 85°, 80°, 75°, 70°, 65°, 60°, 55°, 50°, 45° 40°, 35° 30°, 25°, 20°, 15°, 10°, 5° 1°, or less than 1°. A molecule, particle, or complex may comprise a first face and a second face that are substantially opposed. For example, a molecule, particle, or complex may comprise a first face that is attached to an analyte and a substantially opposed second face that is attached to one or more coupling moieties. Accordingly, attachment of the second face to a solid support can orient the analyte away from the solid support.

As used herein, the terms “analyte” or “analyte of interest,” refers to a molecule, particle, or complex of molecules or particles that is provided to an array for identification, characterization, modification, or any other form of interrogation. An analyte may comprise a target for an analytical method (e.g., sequencing, identification, quantification, etc.) or may comprise a functional element such as a binding ligand or a catalyst. An analyte may comprise a biomolecule, such as a polypeptide, polysaccharide, nucleic acid, lipid, metabolite, enzyme cofactor or a combination thereof. An analyte may comprise a non-biological molecule, such as a polymer, metal, metal oxide, ceramic, semiconductor, mineral, or a combination thereof. As used herein, the terms “sample analyte” refers to an analyte derived from a sample collected from a biological or non-biological system. A sample analyte may be purified, partially purified or unpurified. As used herein, the term “control analyte” refers to an analyte that is provided as a positive or negative control for comparison to a sample analyte. A control analyte may be derived from the same source as a sample analyte, or derived from a differing source from the sample analyte. As used herein, the term “standard analyte” refers to a known or characterized analyte that is provided as a physical or chemical reference to a process. A standard analyte may comprise the same type of analyte as a sample analyte, or may differ from a sample analyte. For example, a polypeptide analyte process may utilize a polypeptide standard analyte with known characteristics. In another example, a polypeptide analyte process may utilize a non-polypeptide standard analyte with known characteristics. As used herein, the term “inert analyte” refers to an analyte with no expected function in a process or system.

As used herein, the terms “linker,” “linking group,” or “linking moiety” refer to a molecule or molecular chain that is configured to attach a first molecule to a second molecule. A linker, linking group, or linking moiety may be configured to provide a chemical or mechanical property to a region separating a first molecule from a second molecule, such as hydrophobicity, hydrophilicity, electrical charge, polarity, rigidity, or flexibility. A linker, linking group, or linking moiety may comprise two or more functional groups that facilitate the coupling of the linker, linking group, or linking moiety to the first and second molecule. A linker, linking group, or linking moiety may include polyfunctional linkers such as homobifunctional linkers, heterobifunctional linkers, homopolyfunctional linkers, and heteropolyfunctional linkers. The molecular chain may be characterized by a minimum size such as, for example, at least about 100 Daltons (Da), 200 Da, 300 Da, 400 Da, 500 Da, 600 Da, 700 Da, 800 Da, 900 Da, 1 kiloDalton (kDa), 2 kDa, 3 kDa, 4 kDa, 5 kDa, 10 kDa, 15 kDa, 20 kDa or more than 20 kDa. Alternatively or additionally, a molecular chain may be characterized by a maximum size such as, for example, no more than about 20 kDa, 15 kDa, 10 kDa, 5 kDa, 4 kDa, 3 kDa, 2 kDa, 1 kDa, 900 Da, 800 Da, 700 Da, 600 Da, 500 Da, 400 Da, 300 Da, 200 Da, 100 Da, or less than 100 Da. A linking moiety may have a length of at least about 1 nanometers (nm), 5 nm, 10 nm, 15 nm, 20 nm, 30 nm, 40 nm, 50 nm, or more than 50 nm. Alternatively or additionally, a linking moiety may have a length of no more than about 50 nm, 40 nm, 30 nm, 20 nm, 15 nm, 10 nm, 5 nm, 1 nm, or less than 1 nm. A linking moiety may be provided to increase a separation distance between a first entity and a second entity (e.g., separating an analyte from a surface of a solid support). A linking moiety may comprise a rigid moiety (e.g., a double-stranded nucleic acid, an alkenyl moiety, an alkynyl moiety). A linking moiety may comprise a non-rigid or flexible moiety (e.g., an alkyl moiety, a PEG moiety, a single-stranded nucleic acid, a peptide, a polysaccharide). Exemplary molecular chains may comprise a synthetic polymer (e.g., polyethylene glycol (PEG), polyethylene oxide (PEO), alkane chains, fluorinated alkane chains), a polysaccharide (e.g., dextrans, methyl cellulose), polypeptides, polynucleotides, or combinations thereof. In some cases, a linking moiety may be substantially devoid of a nucleic acid.

As used herein, the terms “reversible” and “reversibility” are used in reference to a chemical or physical coupling of two entities (e.g., molecules, analytes, functional groups, or moieties) that has a substantial likelihood of uncoupling under one or more conditions of use, such as, one or more conditions of a method or step set forth herein. Reversibility may consist of thermodynamic reversibility, kinetic reversibility, or a combination thereof. Reversible coupling of a first entity to a second entity may be characterized by a temporary change to the structure or function of the first and/or second entity when coupled to each other. Reversing the coupling can optionally revert the structure or function of the first and/or second entity to the same state as it was prior to the temporary change. The context for determining reversibility may comprise the likelihood of detecting a reversed coupling given the specific spatial, temporal, and physical environment in which two coupled molecules are located. For example, in a population of one million streptavidin-biotin coupled pairs, a detectable number of reversed couplings may be predicted thermodynamically, however the slow kinetic reversal of the binding reaction may make such decouplings not detectable above detection noise if the detection time scale is on the order of seconds or minutes. In this context, the streptavidin-biotin coupling would be described as irreversible. The context of reversibility may be process-dependent for a system that undergoes multiple processes. For example, measurable de-coupling of coupled molecules may occur during months of storage but a subsequent process utilizing the coupled molecules may occur in minutes. In this context, the coupled molecules may be reversibly coupled with respect to storage but irreversibly coupled with respect to utilization. Measures of reversibility may include use of quantitative measures such as equilibrium constants or kinetic on-rates and/or off-rates. Reversibility may be directly measured by an equilibrium assay. Reversibility may vary with changes in a chemical system, such as changes in temperature or solvent composition. A reversible coupling may include meta-stable couplings that remain coupled until a change in physical environment. For example, complementary nucleic acids may remain stably coupled at 20° C. but may rapidly decouple above 75° C. A reversible coupling may remain coupled for a time period of at least about 1 second (s), 1 minute (min), 5 min, 10 min, 15 min, 30 min, 1 hour (hr), 2 hr, 3 hr, 4 hr, 5 hr, 6 hr, 12 hr, 18 hr, 1 day, 1 week, 1 month, 6 months, 1 year, or more than 1 year. Alternatively or additionally, a reversible coupling may become decoupled in a time period of no more than about 1 year, 6 months, 1 month, 1 week, 1 day, 18 hrs, 12 hrs, 6 hrs, 5 hrs, 4 hrs, 3 hrs, 2 hrs, 1 hr, 30 min, 15 min, 10 min, 5 min, 1 min, 1 s, or less than 1 s. A time period of reversible coupling can be less than a time period of a method, as set forth herein, or a step thereof.

As used herein, terms “irreversible” and “irreversibility” are used in reference to a chemical or physical coupling of two entities (e.g., molecules, analytes, functional groups, or moieties) that has a likelihood of remaining coupled under one or more conditions of use, such as, one or more conditions of a method or step set forth herein. A system that is determined to not be reversible as described above may be described as irreversible. For example, irreversible coupling of a first entity to a second entity may be characterized by a permanent change to the structure or function of the first and/or second entity after being coupled to each other. Uncoupling can cause substantial change to the structure or function of one or both of the entities compared to the structure or function of the respective entity or entities prior to the coupling. An irreversible coupling may remain coupled for a time period of at least about 1 second (s), 1 minute (min), 5 min, 10 min, 15 min, 30 min, 1 hour (hr), 2 hr, 3 hr, 4 hr, 5 hr, 6 hr, 12 hr, 18 hr, 1 day, 1 week, 1 month, 6 months, 1 year, or more than 1 year. A time period of irreversible coupling can be greater than or equal to a time period of a method, as set forth herein, or a step thereof.

As used herein, the terms “affinity reagent” or “affinity agent” refer to a molecule or other substance that is capable of specifically or reproducibly binding to a binding partner or other substance. Binding can optionally be used to identify, track, capture, alter, or influence the binding partner. The binding partner can optionally be larger than, smaller than or the same size as the affinity reagent. An affinity reagent may form a reversible or irreversible interaction with a binding partner. An affinity reagent may bind with a binding partner in a covalent or non-covalent manner. An affinity reagent may be configured to perform a chemical modification (e.g., ligation, cleavage, concatenation, etc.) that produces a detectable change in the larger molecule, thereby permitting observation of the interaction that occurred. Affinity reagents may include chemically reactive affinity reagents (e.g., kinases, ligases, proteases, nucleases, etc.) and chemically non-reactive affinity reagents (e.g., antibodies, antibody fragments, aptamers, DARPins, peptamers, etc.). An affinity reagent may comprise one or more known and/or characterized binding components or binding sites (e.g., complementarity-defining regions) that mediate or facilitate binding with a binding partner. Accordingly, an affinity reagent can be monovalent or multivalent (e.g. bivalent, trivalent, tetravalent, etc.). An affinity reagent is typically non-reactive and non-catalytic, thereby not permanently altering the chemical structure of a substance it binds in a method set forth herein.

As used herein, the terms “protein” and “polypeptide” are used interchangeably to refer to a molecule or analyte comprising two or more amino acids joined by a peptide bond. A polypeptide may refer to a peptide (e.g., a polypeptide with less than about 200, 150, 100, 75, 50, 40, 30, 20, 15, 10, or less than about 10 linked amino acids). A polypeptide may refer to a naturally-occurring molecule, or an artificial or synthetic molecule. A polypeptide may include one or more non-natural, modified amino acids, or non-amino acid linkers. A polypeptide may contain D-amino acid enantiomers, L-amino acid enantiomers or both. A polypeptide may be modified naturally or synthetically, such as by post-translational modifications.

As used herein, the term “detectable label” refers to a moiety of an affinity reagent or other substance that provides a detectable characteristic. The detectable characteristic can be, for example, an optical signal such as absorbance of radiation, luminescence or fluorescence emission, luminescence or fluorescence lifetime, luminescence or fluorescence polarization, or the like; Rayleigh and/or Mie scattering; binding affinity for a ligand or receptor; magnetic properties; electrical properties; charge; mass; radioactivity or the like. A label component can be a detectable chemical entity that is conjugated to or capable of being conjugated to another molecule or substance. Exemplary molecules that can be conjugated to a label component include an affinity reagent or a binding partner. A label component may produce a signal that is detected in real-time (e.g., fluorescence, luminescence or radioactivity). A label component may produce a signal that is detected off-line (e.g., a nucleic acid barcode) or in a time-resolved manner (e.g., time-resolved fluorescence). A label component may produce a signal with a characteristic frequency, intensity, polarity, duration, wavelength, sequence, or fingerprint. Exemplary labels include, without limitation, a fluorophore, luminophore, chromophore, nanoparticle (e.g., gold, silver or carbon nanotubes), heavy atom, radioactive isotope, mass label, charge label, spin label, receptor, ligand, nucleic acid barcode, polypeptide barcode, polysaccharide barcode, or the like.

As used herein, the term “nucleic acid origami” refers to a nucleic acid construct comprising an engineered secondary, tertiary or quaternary structure. A nucleic acid origami may include DNA, RNA, PNA, LNAs, other nucleic acid analog, modified or non-natural nucleic acids, or combinations thereof. A nucleic acid origami may comprise a plurality of oligonucleotides that hybridize via sequence complementarity to produce the engineered structuring of the origami particle. A nucleic acid origami may comprise sections of single-stranded or double-stranded nucleic acid, or combinations thereof. A nucleic acid origami may comprise one or more tertiary structures of a nucleic acid, such as A-DNA, B-DNA, C-DNA, L-DNA, M-DNA, Z-DNA, etc. A nucleic acid origami may comprise single-stranded nucleic acid, double-stranded nucleic acid, multi-stranded nucleic acid, or combinations thereof. Exemplary nucleic acid origami structures may include nanotubes, nanowires, cages, tiles, nanospheres, blocks, and combinations thereof.

As used herein, the term “nucleic acid nanoball” refers to a globular or spherical nucleic acid structure. A nucleic acid nanoball may comprise a concatemer of oligonucleotides that arranges in a globular structure. A nucleic acid nanoball may comprise one or more oligonucleotides, including oligonucleotides comprising self-complementary nucleic acid sequences. A nucleic acid nanoball may comprise a palindromic nucleic acid sequence. A nucleic acid nanoball may include DNA, RNA, PNA, LNAs, other nucleic acid analog, modified or non-natural nucleic acids, or combinations thereof.

As used herein, the term “oligonucleotide” refers to a molecule comprising two or more nucleotides joined by a phosphodiester bond or analog thereof. An oligonucleotide may comprise DNA, RNA, PNA, LNAs, other nucleic acid analog, modified nucleotides, non-natural nucleotides, or combinations thereof. An oligonucleotide may include a limited number of bonded nucleotides, such as, for example, less than about 10000, 8000, 6000, 5000, 4000, 3000, 2000, 1000, 750, 500, 400, 300, 250, 200, 190, 180, 170, 160, 150, 140, 130, 120, 110, 100, 90, 80, 70, 60, 50, 40, 30, 25, 20, 15, 10, or less than 5 nucleotides. An oligonucleotide may include a linking group or linking moiety at a terminal or intermediate position. For example, an oligonucleotide may comprise two nucleic acid strands that are joined by an intermediate PEG molecule. In another example, an oligonucleotide may comprise a cleavable linker (e.g., a photocleavable linker, an enzymatically-cleavable linker, a restriction site, etc.) that joins two portions of the oligonucleotide. The terms “polynucleotide” and “nucleic acid” are used herein synonymously with the term “oligonucleotide.”

As used herein, the term “binding specificity” refers to the tendency of an affinity reagent to preferentially interact with a binding partner, affinity target, or target moiety relative to other binding partners, affinity targets, or target moieties. An affinity reagent may have a calculated, observed, known, or predicted binding specificity for any possible binding partner, affinity target, or target moiety. Binding specificity may refer to selectivity for a single binding partner, affinity target, or target moiety in a sample over at least one other analyte in the sample. Moreover, binding specificity may refer to selectivity for a subset of binding partners, affinity targets, or target moieties in a sample over at least one other analyte in the sample.

As used herein, the term “binding affinity” or “affinity” refers to the strength or extent of binding between an affinity reagent and a binding partner, affinity target or target moiety. In some cases, the binding affinity of an affinity reagent for a binding partner, affinity target, or target moiety may be vanishingly small or effectively zero. A binding affinity of an affinity reagent for a binding partner, affinity target, or target moiety may be qualified as being a “high affinity,” “medium affinity,” or “low affinity.” A binding affinity of an affinity reagent for a binding partner, affinity target, or target moiety may be quantified as being “high affinity” if the interaction has a dissociation constant of less than about 100 nM, “medium affinity” if the interaction has a dissociation constant between about 100 nM and 1 mM, and “low affinity” if the interaction has a dissociation constant of greater than about 1 mM. Binding affinity can be described in terms known in the art of biochemistry such as equilibrium dissociation constant (K_{D}), equilibrium association constant (K_{A}), association rate constant (kₒₙ), dissociation rate constant (k_{off}) and the like. See, for example, Segel, Enzyme Kinetics John Wiley and Sons, New York (1975), which is incorporated herein by reference in its entirety.

As used herein, the term “promiscuity,” when used in reference to binding, may refer to affinity reagent properties of 1) binding to a plurality of binding partners due to the presence of a particular affinity target or target moiety, regardless of the binding context of the affinity target or target moiety; or 2) binding to a plurality of affinity targets or target moieties within the same or differing binding partners; or 3) a combination of both properties. With regard to the first form of binding promiscuity, “binding context” may refer to the local chemical environment surrounding an affinity target or target moiety, such as flanking, adjacent, or neighboring chemical entities (e.g., for a polypeptide epitope, flanking amino acid sequences, or adjacent or neighboring non-contiguous amino acid sequences relative to the epitope). With regard to the second form of binding promiscuity, the definition may refer to an affinity reagent or probe binding to structurally- or chemically-related affinity targets or target moieties despite differences between the affinity targets or target moieties. For example, an affinity reagent may be considered promiscuous if it possesses a binding affinity for trimer peptide sequences having the form WXK, where W is tryptophan, K is lysine and X is any possible amino acid. Additional concepts pertaining to binding promiscuity are discussed in WO 2020106889A1, which is incorporated herein by reference in its entirety.

As used herein the term “tunable”, when used in reference to an analyte or anchoring moiety, refers to the specific, precise, and/or rational location of components, such as coupling moieties or passivating moieties, or attachment sites for components with an assembly or structure. Tunable retaining components may refer to the ability to couple or conjugate components at specific sites or within specific regions of the retaining component structure, or to generate attachment sites for the coupling or conjugation of probe components at specific sites or specific regions of the retaining component structure.

As used herein, the term “functional group” refers to a group of atoms in a molecule that confer a chemical property, such as reactivity, polarity, hydrophobicity, hydrophilicity, solubility, etc., on the molecule. Functional groups may comprise organic moieties or may comprise inorganic atoms. Exemplary functional groups may include alkyl, alkenyl, alkynyl, phenyl, halide, hydroxyl, carbonyl, aldehyde, acyl halide, ester, carboxylate, carboxyl, carboalkoxy, methoxy, hydroperoxyl, ether, hemiacetal, hemiketal, acetal, ketal, orthoester, epoxide, carboxylic anhydride, carboxamide, amine, ketimine, aldimine, imide, azide, azo, cyanate, isocyanate, nitrate, nitrile, isonitrile, nitrosoxy, nitro, nitroso, oxime, pyridyl, carbamate, sulfhydryl, sulfide, disulfide, sulfinyl, sulfonyl, sulfinom, sulfo, thiocyanate, isothiocyanate, carbonothioyl, thioester, thionoester, phosphino, phosphono, phosphonate, phosphate, borono, boronate, and borinate functional groups.

As used herein, the term “functionalized” refers to any material or substance that has been modified to include a functional group. A functionalized material or substance may be naturally or synthetically functionalized. For example, a polypeptide can be naturally functionalized with a phosphate, oligosaccharide (e.g., glycosyl, glycosylphosphatidylinositol or phosphoglycosyl), nitrosyl, methyl, acetyl, lipid (e.g., glycosyl phosphatidylinositol, myristoyl or prenyl), ubiquitin or other naturally occurring post-translational modification. A functionalized material or substance may be functionalized for any given purpose, including altering chemical properties (e.g., altering hydrophobicity or changing surface charge density) or altering reactivity (e.g., capable of reacting with a moiety or reagent to form a covalent bond to the moiety or reagent).

Other than in the operating examples, or where otherwise indicated, all numbers expressing quantities of ingredients or reaction conditions used herein should be understood as modified in all instances by the term “about.” As used herein, the term “about,” when used in connection with percentages, may mean a variance of at most ±5% of the value being referred to. For example, about 90% may mean from 85% to 95%. In some cases, “about” may mean a variance of at most ±4%, ±3%, ±2%, ±1%, ±0.5% or less of the value being referred to. As used herein, the term “substantially,” when used in reference to a measurable quantity or property, refers to the quantity or property having a value within +10% of a reference value. For example, a first value may be substantially the same as a second value if the first value is within +10% of the second value. In another example, a shape may be substantially square if a ratio of side lengths of a rectangle is within a range between 0.90 and 1.10, inclusive. In some cases, “substantially” may mean a quantity or property having a value within at most ±9%, ±8%, 7%, ±6%, ±5%, ±4%, ±3%, ±2%, +1%, +0.5%, or less of a reference value.

As used herein, the terms “attached” or “coupled” refer to the state of two things being joined, fastened, adhered, connected or bound to each other. Attachment can be covalent or non-covalent. For example, a particle can be attached or coupled to a protein by a covalent or non-covalent bond. Similarly, a first nucleic acid can be attached or coupled to a second nucleic acid via hybridization or Watson-Crick base pairing. A covalent bond is characterized by the sharing of pairs of electrons between atoms. A non-covalent bond is a chemical bond that does not involve the sharing of pairs of electrons and can include, for example, hydrogen bonds, ionic bonds, van der Waals forces, hydrophilic interactions, adhesion, adsorption, and hydrophobic interactions.

As used herein, the term “population” refers to a total quantity of items, such as array sites, analytes, anchoring moieties, etc. For example, a population of array sites of an array would refer to all array sites on the array without exclusion. Likewise, a population of array sites of a subarray would refer to all array sites of the subarray without exclusion. As used herein, the term “plurality” refers to two or more items. A plurality of items can include any plural quantity of items up to and including a total quantity of items. Accordingly, a population of items would be the largest possible size of a plurality of items.

As used herein, the term “assay agent” or “assay reagent” refers to matter that is introduced into an array system for the purpose of performing an assay, such as array-based assay as set forth herein. Assay agents include analytes, anchoring moieties, other particles, and chemical reagents (including small molecule and macromolecule chemical reagents). Assay agents can include liquids, gases, solids, and combinations thereof. Assay agents may be directly or indirectly contacted to an array, as set forth herein. An assay agent may be indirectly coupled to an array if it does not contact a surface containing array sites of an array, but is contacted to a medium or material that is in contact with the surface. For example, a multi-phase fluid may be introduced into a flow cell containing an array, in which a first assay agent is solubilized in a first fluidic phase that contacts the array, and a second assay agent is solubilized in a second fluidic phase that does not contact the array. In this example, the first assay agent is directly contacted to the array, and the second assay agent is indirectly contacted to the array.

As used herein, the term “anchoring moiety” or “anchoring group” refers to a molecule or particle that serves as an intermediary attaching an analyte to a surface (e.g., on a solid support or a microbead). An anchoring group may be covalently or non-covalently attached to a surface and/or a polypeptide. An anchoring group may be a biomolecule, polymer, particle, nanoparticle, or any other entity that is capable of attaching to a surface or analyte. An anchoring moiety may provide physical separation between a surface of a solid support and an analyte that is attached to the anchoring moiety. An anchoring moiety may comprise a linking moiety (e.g., a rigid linker, a flexible linker) that attaches an analyte to the anchoring moiety, and optionally provides physical separation between the analyte and a surface of a solid support. An anchoring moiety may comprise a particle or a bead. In some cases, an anchoring group may be a nucleic acid nanoparticle such as a SNAP (e.g., a nucleic acid origami, a nucleic acid nanoball). In some cases, an anchoring group may comprise a non-nucleic acid nanoparticle, such as a polymer nanoparticle, a branched polymer nanoparticle, or a dendrimeric nanoparticle.

As used herein, the term “binding entity” refers to a molecule, particle, or moiety that is configured to be bound to an array site by a non-orthogonal binding interaction, or is bound to an array site by a non-orthogonal binding interaction. A binding entity may comprise one or more coupling moieties. A binding entity may comprise an analyte, an anchoring moiety, or an analyte attached to an anchoring moiety.

As used herein, the term “unbound,” when used in reference to a molecule, particle or moiety that is contacted with an array, refers to the molecule, particle, or moiety not being attached or bound at an array site in an initial configuration. An unbound assay agent may include a molecule, particle, or moiety that is solvated, suspended, or otherwise mobile within a fluidic medium at the instant it is contacted with an array. As used herein, the term “bound” when used in reference to a molecule, particle or moiety that is contacted with an array, refers to the molecule, particle, or moiety being attached or coupled at an array site. A bound molecule, particle, or moiety may be covalently or non-covalently coupled to an array site.

As used herein, the terms “passivate” or “passivating,” when used in reference to a moiety, particle, or molecule, or a collection thereof (e.g., a layer or coating), refers to inhibition of unwanted or orthogonal binding of an initially unbound entity to the array or a surface thereof. A layer, coating, molecule, particle, or moiety can be considered passivating with respect to a binding context to which the passivating entity is exposed. For example, a surface comprising a plurality of attached oligonucleotides with uniform nucleotide sequences would be expected to specifically bind oligonucleotide assay agents with complementary sequences to the attached oligonucleotides (i.e., a wanted or intended binding interaction), but would inhibit binding of oligonucleotide assay agents with partially or completely non-complementary nucleotide sequences (i.e., unwanted or unintended binding interactions). Likewise, a surface comprising a layer of positively-charged amine moieties (i.e., a positively charged surface) can be expected to form binding interactions with negatively-charged assay agents, and would be expected to inhibit binding of positively-charged assay agents (i.e., the layer is passivating with respect to positively-charged assay agents). A layer, coating, molecule, particle, or moiety with passivating properties can be hydrophobic, hydrophilic, polar, non-polar, positively-charged, negatively-charged, linear, branched, dendrimeric, or a combination thereof, depending upon a binding context or a chemical property of an assay agent. Exemplary passivating molecules or moieties can include, but are not limited to, polyethylene glycol molecules (PEG), alkyl moieties, halogenated alkyl moieties, polyvinylpyrrolidines, polyalcohols, oligosaccharide molecules (e.g., dextrans, glucopyranosides or methyl cellulose), polypeptides (e.g., albumins), and oligonucleotides.

As used herein, the term “optically resolvable distance” refers to a distance on an array or a surface thereof at which two separate objects can be optically distinguished with respect to each other. The threshold for an optically resolvable distance can vary based upon a mechanism of detection and/or the physical apparatus used to perform an optical detection as well as a detectable species utilized for detection (e.g., single fluorophores, multiple fluorophores, nanoparticles, intercalated dyes, etc.). For example, when detecting two fluorescent objects on a surface via optical microscopy, an optically resolvable distance may depend upon an excitation wavelength of fluorophores, an emission wavelength of fluorophores, and optical characteristics of an optical microscope utilized to image the objects. An optically resolvable distance may be at least about 1 nanometer (nm), 5 nm, 10 nm, 25 nm, 50 nm, 75 nm, 100 nm, 125 nm, 150 nm, 200 nm, 300 nm, 400 nm, 500 nm, or more than 500 nm. Alternatively or additionally, an optically resolvable distance may be no more than about 500 nm, 400 nm, 300 nm, 200 nm, 150 nm, 125 nm, 100 nm, 75 nm, 50 nm, 25 nm, 10 nm, 5 nm, 1 nm, or less than 1 nm. In some cases, an optically resolvable distance may be determined with respect to a detection method (e.g., a pixel-based sensor). For example, two objects may be considered to be separated by an optically resolvable distance if a sensor-based detection produces two optical signal intensity maxima (corresponding to the two objects) and an optical signal intensity minimum between the two maxima, in which the optical signal intensity minimum has a magnitude that is no more than half of the average signal-to-noise ratio of the two optical signal intensity maxima. As used herein, the term “optically non-resolvable distance” refers to a distance on an array or a surface thereof which is less than an optically resolvable distance, as set forth herein. An optically non-resolvable distance may be a distance at which an optical signal from a first object can not be distinguished from an optical signal from a second object. For example, a first optical signal from a first object may be optically non-resolvable from a second optical signal from a second object if the first optical signal and the second optical signal are respectively detected by adjacent pixels of a pixel-based sensor.

As used herein, the term “substantially uniform,” when used in reference to a plurality of molecules, moieties, or particles, refers to the plurality of molecules, moieties, or particles as containing a vast majority (e.g., at least about 90%, 95%, 96%, 97%, 98%, 99%, 99.5%, 99.9%, 99.99%, 99.999%, or more than 99.999% on a molar or mass basis) of molecules, moieties, or particles with uniform structural or physical properties. A plurality of molecules, moieties, or particles may be substantially uniform with respect to a structural property, a physical property, or both a structural and a physical property. For example, a plurality of polyethylene glycol (PEG) molecules containing a molecular weight distribution may be considered substantially uniform with respect to chemical structure (i.e., repeating polymerized ethylene glycol monomers). In another example, a plurality of PEG molecules comprising a mixture of linear and branched molecular chains may be considered substantially uniform with respect to a physical property if all molecules of the mixture are configured to inhibit binding of a particular assay agent, as set forth herein.

As used herein, the term “orthogonal binding phenomena”, when used in reference to an array or a molecule, moiety, or particle contacted thereto, refers to any unwanted, unexpected, or contrary-to-design binding interactions that occur between an array surface or array feature and an unbound moiety that may become contacted with the array surface or array feature. Orthogonal binding phenomena may be qualitatively characterized as a binding interaction that occurs in a system that has been engineered to prevent such a binding interaction (e.g., a hydrophilic molecule binding to a putatively hydrophobic surface). Orthogonal binding phenomena may be quantitatively characterized as measurable binding interactions occurring between an array surface or array feature (e.g., an interstitial region or an analyte binding site) and an unbound moiety that may become contacted with the array surface or feature, in which the measurable binding interactions occur at a rate and/or to an extent that exceeds a predicted rate and/or extent, such as a thermodynamic or kinetic prediction (e.g., a dissociation constant, a binding on-rate, a binding off-rate, etc.). For example, if an unbound moiety is characterized to bind to a surface-coupled passivating moiety (e.g., polyethylene glycol) with a kilomolar dissociation constant (a very weak binding interaction), then observing a millimolar binding dissociation constant between the unbound moiety and an array surface that is provided with a uniform layer of the surface-coupled passivating moiety would indicate an orthogonal binding phenomena (i.e., binding due to a mechanism other than the specific binding of the unbound moiety to the surface-coupling passivating moiety). Orthogonal binding phenomena may be characterized based upon a stochastic measure, such as spatial and/or temporal variations in unwanted, unexpected, or contrary-to-design binding phenomena. As used herein, the term “non-orthogonal binding phenomena,” when used in reference to an array or a molecule, moiety, or particle contacted thereto, refers to any wanted, expected, or designed binding interactions that occur between an array surface or array feature and an unbound moiety that may become contacted with the array surface or array feature. Examples of non-orthogonal binding interactions can include binding of an oligonucleotide to a complementary oligonucleotide, binding of a receptor to a ligand, binding of an affinity agent to an epitope for which the affinity agent has a binding specificity, and covalent binding of a Click-type reagent to a complementary Click-type reagent.

As used herein, the term “transportate,” when used in reference to a mass transfer process or result of a mass transfer process, refers to a discrete object that is preferentially transported toward or along a surface of a solid support during the mass transfer process. A transportate can include a macromolecule, particle, or other moiety. A transportate may be an assay agent, such as an analyte, an anchoring moiety, an affinity agent, a binding reagent, a detectable probe, or a complex thereof. Alternatively, a transportate may be a small molecule compound. A transportate may be soluble or suspendable within a fluidic medium, as set forth herein. A transportate may be insoluble or sedimentable within a fluidic medium, as set forth herein. A transportate may comprise a plurality of smaller molecules, particles, or moieties that are temporarily or permanently coupled in a single discrete entity (i.e., each individual molecule, particle, or moiety of the transportate co-transports with each other molecule, particle, or moiety).

As used herein, the term “macromolecule” refers to a molecule containing a large number of covalently-bonded atoms, such as at least about 100, 250, 500, 1000, or more than 1000 covalently-bonded atoms. Alternatively or additionally, a macromolecule can refer to a molecule having a large molecular weight, such as at least about 1000 Daltons (Da), 2000 Da, 5000 Da, 10000 Da, or more than 10000 Da. In some cases, a macromolecule may comprise a biomolecule, such as a polypeptide, polynucleotide, polysaccharide, or lipid. A macromolecule may have an ordered primary, secondary, or tertiary structure. A macromolecule may have an amorphous structure.

As used herein, the term “particle” refers to a discrete, solid object with a characteristic dimension (e.g., length, width, diameter, hydrodynamic radius, etc.) of no more than 1 millimeter (mm), 500 microns (μm), 250 μm, 100 μm, 50 μm, 25 μm, 10 μm, 5 μm, 1 μm, or less than 1 μm. A particle can comprise a cluster or complex of molecules, provided the molecules remain associated throughout transport of the particle. Molecules of a particle may be covalently or non-covalently bonded. Molecules of a particle may be associated by an interaction such as a magnetic or electrostatic interaction. A particle may comprise a macromolecule. A particle may comprise a plurality of associated small molecules and/or macromolecules. A particle may comprise a microparticle (i.e., a particle having a micron-scale characteristic dimension) or a nanoparticle (i.e., a particle having a nanometer-scale characteristic dimension). A particle may comprise a first particle with a larger characteristic dimension coupled to a second particle with a smaller characteristic dimension. For example, a particle (e.g., a microparticle) may be coupled to a plurality of nucleic acid nanoparticle or a plurality of magnetic nanoparticles.

The term “comprising” is intended herein to be open-ended, including not only the recited elements, but further encompassing any additional elements.

As used herein, the term “each,” when used in reference to a collection of items, is intended to identify an individual item in the collection but does not necessarily refer to every item in the collection. Exceptions can occur if explicit disclosure or context clearly dictates otherwise.

The embodiments set forth below and recited in the claims can be understood in view of the above definitions.

### Arrays and Array Characterization

### Array Components

Described embodiments of the present invention may include an array, as set forth herein. Arrays can include numerous arrangements of solid supports with discrete analyte-binding sites such that deposition of analytes at the analyte-binding sites gives rise to spatially-ordered distributions of analytes. In some embodiments, arrays may give rise to spatially-ordered distributions of analytes such that analytes are bound at known and/or predictable addresses of a solid support. In some embodiments, arrays may give rise to spatially-ordered distributions of analytes such that any analyte or a cluster thereof is optically resolvable from every other analyte or cluster thereof on the array. In a preferable embodiment, an array can be a single-analyte array. A single-analyte array may comprise a plurality of sites, in which each site of the plurality of sites comprises one and only one analyte. In other embodiments, an array can be a clustered array. A clustered array may comprise a plurality of sites, in which each site of the plurality of sites comprises 2, 3, 5, 10, 25, 50, 100, 500, 1000, or more than 1000 analytes.

In some embodiments, an array or a subarray thereof may contain a population of sites. A population of sites of an array or a subarray thereof may have a total quantity of sites, such as at least about 10 sites, 10² sites, 10³ sites, 10⁴ sites, 10⁵ sites, 10⁶ sites, 10⁷ sites, 10⁸ sites, 10⁹ sites, 10¹⁰ sites, 10¹¹ sites, 10¹² sites, or more than 10¹² sites. Alternatively or additionally, a population of sites of an array or a subarray thereof may have a total quantity of sites, such as no more than about 10¹² sites, 10¹¹ sites, 10¹⁰ sites, 10⁹ sites, 10⁸ sites, 10⁷ sites, 10⁶ sites, 10⁵ sites, 10⁴ sites, 10³ sites, 10² sites, 10 sites, or less than 10 sites.

In some embodiments, arrays or subarrays thereof can contain at least one site or a plurality thereof whose analyte-binding behavior is non-ideal (e.g., binding too few analytes, binding too many analytes or binding no analytes). For example, a single-analyte array may be expected to contain a first fraction of non-ideal sites that do not bind analytes, and optionally a second fraction of non-ideal sites that bind two or more analytes. Similarly, a single-analyte array may contain a first fraction of non-ideal sites that are attached to analytes, and optionally a second fraction of non-ideal sites that are attached to two or more analytes. In another example, a single-analyte array may be expected to contain a first fraction of non-ideal sites that bind two or more analytes, and optionally a second fraction of non-ideal sites that do not bind analytes. In yet another example, a clustered array may contain a site or a plurality thereof that binds (or is attached to) too few or too many analytes, such as at least about 10%, 20%, 30%, 40%, 50%, 75%, or 90% too few or too many analytes.

An array may comprise a plurality of sites in which each site is located on a solid support at a unique address. In some embodiments, an array may comprise a plurality of sites, in which each site is separated by an optically resolvable distance from each other site of the plurality of sites. Accordingly, a single-analyte array may comprise a plurality of sites, in which each site comprises one and only one bound analyte of a plurality of analytes, and in which each analyte is optically resolvable from each other analyte of the plurality of analytes. Accordingly, site competency may further include optical resolvability as a criterium for calculating the site competency C, as set forth in Equation 1. For example, N_{competent} may be determined as the characterized total number of optically-resolvable sites that are competent to bind an analyte on the array or subarray.

An array site can be configured to bind an analyte or an anchoring moiety coupled to an analyte. Accordingly, an array site may comprise one or more coupling moieties that are configured to bind the analyte and/or the anchoring moiety to the site. A coupling moiety may be configured to form a non-covalent binding interaction with an analyte and/or an anchoring moiety (e.g., nucleic acid hybridization, electrostatic binding, hydrogen bonding, van der Waals bonding, receptor-ligand binding, etc.). Exemplary non-covalent coupling moieties can include oligonucleotides, receptor-ligand binding components (e.g., streptavidin/biotin, SpyCatcher-SpyTag, SnoopCatcher-SnoopTag, etc.). A coupling moiety may be configured to form a covalent binding interaction with an analyte and/or an anchoring moiety (e.g., a Click-type reaction, a cross-linking reaction, a polymerization reaction, etc.). Exemplary covalent binding moieties may include, epoxides, amines, imines, imides, carboxylates, alkenyls, alkynyls, aryls, esters, azides, thiols, etc. In some cases, an array site may comprise a covalent binding moiety and a non-covalent binding moiety. Optionally, an array site may further comprise one or more passivating moieties. A passivating moiety may be configured to inhibit a binding interaction between a molecule or moiety and the array site. A passivating moiety may comprise a hydrophobic moiety. A passivating moiety may comprise a hydrophilic moiety. A passivating moiety may comprise an electrically-charged moiety. A passivating moiety may comprise an electrically-neutral moiety. A passivating moiety may comprise a polymeric moiety (e.g., polyethylene glycol, polyethylene, polypropylene, a cationic polymer, an anionic polymer, a zwitterionic polymer, etc.) or a biopolymeric moiety (e.g., a dextran, cellulose, hemicellulose, a protein, a nucleic acid, etc.). In some embodiments, an array site may comprise a coupling moiety coupled to a passivating moiety (e.g., a polyethylene chain containing a terminal oligonucleotide).

In some cases, an array site may comprise a blocking material or blocking moiety. A blocking material or moiety can comprise any conceivable material that inhibits or prevents an intended or designed binding interaction of an analyte and/or anchoring moiety with the array site, and is optionally releasable from the array site. A blocking material may be covalently or non-covalently attached to a surface of an array site, or to a moiety attached thereto (e.g., a coupling moiety or a passivating moiety). A blocking material or blocking moiety may bind to a coupling moiety, thereby preventing the coupling moiety from forming a binding interaction with an analyte or an anchoring moiety. A blocking material or blocking moiety may occlude contact between a coupling moiety and a complementary coupling moiety, thereby inhibiting the coupling moiety from forming a binding interaction with an analyte or an anchoring moiety. A blocking material or moiety may be selectively released from an array site (e.g., chemically released, photochemically released, enzymatically released, etc.), thereby unblocking the array site. Examples of blocking materials are given, for example, in U.S. Pat. No. 7,736,906, which is herein incorporated by reference in its entirety.

In some embodiments, array sites may be spatially separated by one or more interstitial regions. An interstitial region may be configured to inhibit a binding interaction between a molecule or moiety and the interstitial region. In some embodiments, an interstitial region may be configured to inhibit a binding interaction between an analyte or an anchoring moiety and the interstitial region. For example, an interstitial region of an array can inhibit binding to an analyte that binds to (or is configured to bind to) a site of the array. An interstitial region may comprise one or more passivating moieties. A passivating moiety may be configured to inhibit a binding interaction between a molecule or moiety and the interstitial region. A passivating moiety may comprise a hydrophobic moiety. A passivating moiety may comprise a hydrophilic moiety. A passivating moiety may comprise an electrically-charged moiety. A passivating moiety may comprise an electrically-neutral moiety. A passivating moiety may comprise a polymeric moiety (e.g., polyethylene glycol, polyethylene, polypropylene, a cationic polymer, an anionic polymer, a zwitterionic polymer, etc.) or a biopolymeric moiety (e.g., a dextran, cellulose, hemicellulose, a protein, a nucleic acid, etc.). In some embodiments, an interstitial region may comprise a coating or layer (e.g., a monolayer, a self-assembled monolayer, etc.). In some cases, an interstitial region may comprise a coating or layer deposited during an array formation method (e.g., a resist material or an adhesion promoter such as hexamethyldisilazane (HMIDS)). An interstitial region may be continuous or non-continuous. An array may comprise a plurality of subarrays, in which a first subarray is separated from a second subarray by an interstitial region. Additional advantageous aspects of arrays and array formation are presented in U.S. Pat. Nos. 11,203,612 and 11,505,796, and U.S. patent application Ser. Nos. 17/513,877, 17/772,484, 63/387,322, and 63/387,555, each of which in herein incorporated by reference in its entirety.

In some embodiments, an array may comprise one or more fiducial elements. A fiducial element may comprise any marking or object that provides a spatial reference point on an array. A fiducial element may be formed from a solid support, for example by patterning or etching. A fiducial element may be disposed on a surface of a solid support, for example by deposition of a metal or metal oxide. A fiducial element may comprise a detectable particle (e.g., a fluorescent nanoparticle, a quantum dot, etc.) that is disposed on an array. A fiducial element may be disposed on an interstitial region of an array. A fiducial element may be disposed on a site of an array. Additional advantageous aspects of arrays comprising fiducial elements are disclosed in US. Patent Application No. 63/485,835, which is herein incorporated by reference in its entirety.

### Array Site Binding Availability and Array Site Binding Competency

Arrays produced by an array formation process can be expected to contain a fraction of sites that are non-functional for the purpose of binding an analyte or an anchoring moiety. Non-functional array sites may arise due to manufacturing error, manufacturing variability, degradation during storage or usage, or combinations thereof. In particular, lithographic processes for array formation may produce unavailable binding sites due to unintended manufacturing error and/or variability such as incomplete stripping of resist materials, cross-reactivity of resist materials, incomplete or variable deposition of surface chemistry, spatial variability of lithographic patterning (e.g., via irradiation, via imprinting, etc.), and spatial variability of intermediate processing (e.g., plasma treatments, resist and/or adhesion promoter deposition, thermal oxide layer development, etc.). Formation of non-functional array sites can impact an array-based process by inhibiting deposition of an analyte at the non-functional sites. Further, non-functional sites can impact an array-based process by inhibiting subsequent array steps (e.g., binding of assay agents to analytes) or facilitating orthogonal binding of assay agents to array site defects that render the sites non-functional.

Array site binding availability may comprise a measure of the fraction or percentage of total sites on an array or a subarray thereof that contain a minimum set of structural features for forming an intended or designed binding interaction with an analyte or anchoring moiety (i.e., a non-orthogonal binding interaction). Array site binding availability may provide an upper bound to a maximum total occupancy of an array or a subarray thereof. For example, if an array containing a total of 10⁶ sites has an array site binding availability of 95%, it would be expected that a maximum number of analytes or anchoring moieties bound to an array would be limited to no more than 9.5×10⁵ analytes or anchoring moieties. Array binding site availability may be characterized before an array-based assay or other process, for example by a quality control process. Array binding site availability may be characterized during or after an array-based assay or other process, for example by sequential measurement of array occupancy to identify an asymptotic limit of occupancy. Array binding site availability may be characterized by a bulk-scale or single-molecule physical measurement process, as provided herein.

An array or a subarray thereof may have an array site binding availability of at least about 10%, 20%, 30%, 40%, 50%, 55%, 60%, 65%, 70%, 75%, 80%, 85%, 90%, 95%, 96%, 97%, 98%, 99%, 99.9%, 99.99%, 99.999%, or more than 99.999%. Alternatively or additionally, an array or a subarray thereof may have an array site binding availability of no more than about 99.999%, 99.99%, 99.9%, 99%, 98%, 97%, 96%, 95%, 90%, 85%, 80%, 75%, 70%, 65%, 60%, 55%, 50%, 40%, 30%, 20%, 10%, or less than 10%.

Array site binding availability may provide a site-specific or population specific characterization of analyte-binding capacity of an array. An array site or a population thereof may be further characterized with respect to array site binding competency. Array site binding competency may provide a measure of binding propensity. In other words, each available array site of a population of array sites may comprise all necessary structural features to form intended or designed binding interactions with analytes or anchoring moieties, but individual sites may display varying likelihood of binding analytes or anchoring moieties due to variations in site structure unique to each array site of the population of array sites. Without wishing to be bound by theory, array site binding competency may depend upon thermodynamic and/or kinetic aspects of an intended or designed binding interaction between a site and an analyte or anchoring moiety.

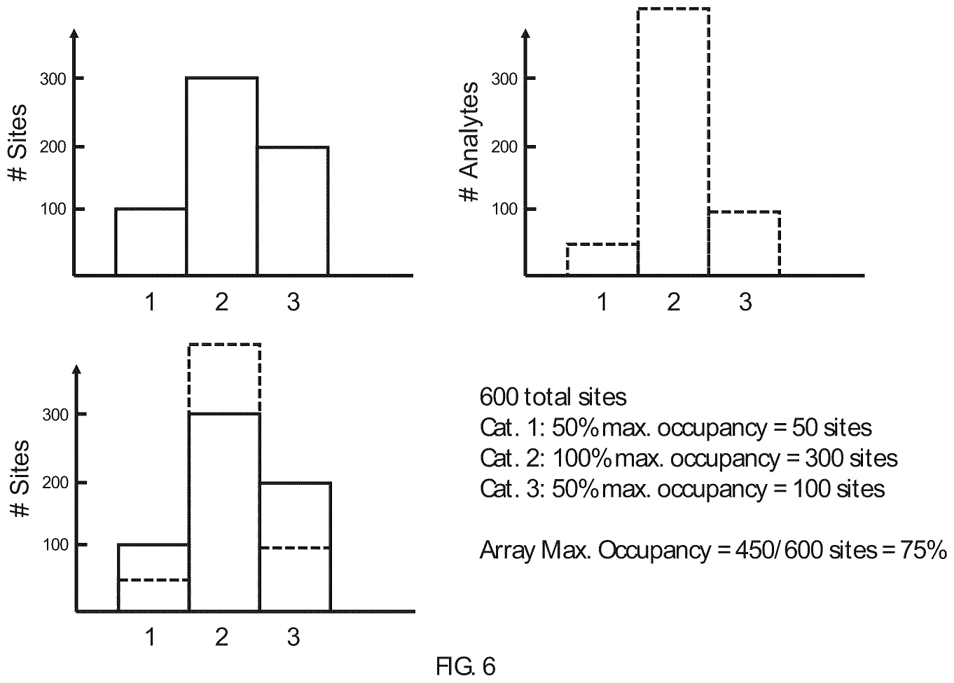

FIGS. 5A-5B illustrate exemplary aspects of array structure and analyte or anchoring moiety structure that can impact array site binding competency. FIG. 5A depicts four sites (A, B, C, and D) with varying structure that are contacted with a single species of analytes 510, in which the analytes 510 are attached to an anchoring moiety 515 containing an anchoring moiety-linked coupling moiety 518. Sites A-D contain an optional surface layer 501 and an array site-linked coupling moiety 505, in which the array site-linked coupling moiety 505 is complementary to the anchoring moiety-linked coupling moiety 518. Site A is configured with a favorable site size and coupling moiety surface density, so the site is competent to bind the analyte by forming a binding interaction to the anchoring moiety. Site B further comprises a blocking material 502 that occludes contact between the array site-linked coupling moiety 505 and the anchoring moiety-linked coupling moiety 518, so the site is not available for binding to an analyte 510. Site C further comprises a 3-dimensional material 503 adjacent to the surface layer 501 that occludes contact between the array site-linked coupling moiety 505 and the anchoring moiety-linked coupling moiety 518. Site C is not competent to bind the analyte 510, but may be competent to bind an analyte 510 with an anchoring moiety comprising a smaller diameter. Site D comprises a larger surface layer 501, thereby leading to a lower surface density of the array site-linked coupling moiety 505. Site D is competent but may have a lower array site binding competency for the analyte 510 relative to Site A due to a decreased likelihood of a binding interaction forming between array site-linked coupling moiety 505 and the anchoring moiety-linked coupling moiety 518.

FIG. 5B illustrates aspects of array site binding competency related to heterogeneous populations of analytes and/or anchoring moieties. Site A of FIG. 5B is structurally equivalent to Site A of FIG. 5A, but the site is contacted with an analyte 510 that is linked to an anchoring moiety 515 that lacks an anchoring-moiety linked coupling moiety 518. Site A is not competent to form a binding interaction due to the absence of a complementary pair of coupling moieties. Site B of FIG. 5B is structurally equivalent to Site A of FIG. 5A, but the site is contacted with an anchoring moiety 515 comprising an anchoring-moiety linked coupling moiety 518 but lacking an attached analyte 510. Site B is competent to bind the anchoring moiety, but will not become occupied by an analyte 510 if bound to the adjacent anchoring moiety 515. Site C is structurally equivalent to Site C of FIG. 5A, but is contacted with two differing species of analytes 510 (a first analyte 510 attached to a larger anchoring moiety 515, and a second analyte 510 attached to a smaller anchoring moiety 516). Site C is competent to bind an analyte 510 attached to anchoring moiety 516, but not competent to bind an analyte 510 attached to anchoring moiety 515.

Array site binding competency can provide a qualitative and/or quantitative characterization of array site binding propensity for a single array site or a population thereof. Characterization of array site binding competency may be presented in several fashions, including analyte-facing descriptions (i.e., characterizing array site binding competency in terms of propensity to bind certain analytes and/or anchoring moieties) or site-facing descriptions (i.e., characterizing array site binding competency in terms of array site structural characteristics that give influence propensity for analyte and/or anchoring moiety binding).

In some cases, array site binding competency may be characterized in a deterministic fashion. A deterministic characterization of array site binding competency can comprise any characterization of which analytes and/or anchoring moieties an array can bind according to the structural properties of the array site or the structural characteristics of the analytes and/or anchoring moieties. For example, amongst a population of anchoring moieties containing species A, B, and C, an array site may be configured to bind anchoring moiety species A, but not species B and C. Accordingly, a deterministic characterization of the array site can include information on competence for binding species A and/or incompetence for binding species B and C. A deterministic characterization of array site binding competency for an array site may be an analyte-facing characterization, such as an identification of whether an analyte and/or anchoring moiety will or will not bind to the array site, or which analyte species and/or anchoring moiety species amongst a variety of analyte species and/or anchoring moiety species will or will not bind to the array site. A deterministic characterization of array site binding competency for an array site may be a site-facing characterization, such as a site categorization based upon one or more site structural features. For example, a site may be categorized with respect to binding competency according to its characteristic dimension, species of coupling moiety, surface density of coupling moieties, or combinations thereof.

In some cases, array site binding competency may be characterized in a probabilistic or stochastic fashion. A probabilistic or stochastic characterization of array site binding competency can comprise any characterization of a likelihood of an array site binding an analyte and/or anchoring moiety. For example, amongst a population of anchoring moieties containing species A, B, and C, an array site may be best configured to bind anchoring moiety species A, and less optimally configured to bind species B and C. Accordingly, a probabilistic characterization of the array site can include information on a higher binding probability for binding species A, and a lower probability for binding species B and species C. A probabilistic characterization of array site binding competency for an array site may be an analyte-facing characterization, such as a probability of binding an analyte and/or anchoring moiety based upon a structural characteristic of the analyte and/or anchoring moiety. A probabilistic characterization of array site binding competency for an array site may be a site-facing characterization, such as a probability of binding an analyte and/or anchoring moiety based upon a structural characteristic of the array site.

Information on array site binding competency for individual array sites may be coalesced or aggregated into an array site binding competency profile. An array site binding competency profile may provide a qualitative or quantitative estimate or measure of a range of binding competencies present on an array or a subarray thereof. For example, for a population of sites of an array, as set forth herein, a range of binding competencies can be expected due to variability in one or more structural features of each site of the population of sites. Accordingly, an array site binding competency profile can include a quantitative measure of number of sites in subpopulations grouped by one or more structural features that influence array site binding propensity. In some cases, an array site binding competency profile may comprise a quantitative measure of number of sites in subpopulations grouped by a single parameter model or a multi-parameter model of analyte and/or anchoring moiety binding.

In some cases, an array site binding competency profile can comprise a qualitative characterization of binding competency for a population of array sites. An array can be assigned a qualitative characterization of binding competency (e.g., High/Medium/Low; Type 1, Type 2, Type 3, etc . . . ; Type A, Type B, Type C, etc. . . . ) based upon an estimated or measured range of binding competencies. In some cases, analytes and/or anchoring moieties may be contacted to an array with a known or characterized qualitative array site binding competency profile, in which the analytes and/or anchoring moieties have a corresponding analyte binding competency profile or anchoring moiety binding competency profile. For example, a Type 1 array may be contacted with Type 1 anchoring moieties to achieve a maximized array site occupancy. In some cases, a population of analytes and/or anchoring moieties may be formulated based upon an array site binding competency profile. For example, two or more species of anchoring moieties may be combined in a ratio based upon a correlated ratio of two or more subpopulations of array sites, in which the array site subpopulations are formed by grouping sites of alike binding competencies.

### Array Patterning, Structure, and Geometry

Arrays may be formed by a lithographic process, such as photolithography, Dip-Pen nanolithography, nanoimprint lithography, nanosphere lithography, cluster lithography, nanopillar arrays, nanowire lithography, scanning probe lithography, thermochemical lithography, thermal scanning probe lithography, local oxidation nanolithography, molecular self-assembly, stencil lithography, or electron-beam lithography. A lithographic process may be utilized to form an ordered array of sites. An ordered array of sites may refer to sites being located at predetermined addresses on a solid support, for example at positions determined by positioning of a reticle relative to a solid support surface during photolithography. In a particular embodiment, an ordered array may comprise a patterned array if the array sites are positioned in a regular and/or repeating order such as a gridded pattern (e.g., a rectangular grid, a hexagonal grid, a circular grid, etc.). In some embodiments, an array may comprise an unordered array or a random array.

An array can comprise a plurality of sites, in which each site comprises a characteristic dimension (e.g., diameter, width or length). Sites of an array or a subarray thereof may have an average characteristic dimension of at least about 1 nanometer (nm), 5 nm, 10 nm, 25 nm, 50 nm, 100 nm, 150 nm, 200 nm, 250 nm, 300 nm, 400 nm, 500 nm, 1 micron (μm), or more than 1 μm. Alternative or additionally, sites of an array or a subarray thereof may have an average characteristic dimension of no more than about 1 μm, 500 nm, 400 nm, 300 nm, 250 nm, 200 nm, 150 nm, 100 nm, 50 nm, 25 nm, 10 nm, 5 nm, 1 nm, or less than 1 nm. The average characteristic dimension of sites of an array or subarray may be further characterized by a statistical measure of size deviation (e.g., variance or standard deviation). A measure of size deviation of sites of an array or subarray thereof may be no more than about 99%, 95%, 90%, 75%, 50%, 40%, 30%, 25%, 20%, 15%, 10%, 5%, 1%, or less than 1% of the magnitude of an average characteristic dimension of the sites. Alternatively or additionally, a measure of size deviation of sites of an array or subarray thereof may be at least about 1%, 5%, 10%, 15%, 20%, 25%, 30%, 40%, 50%, 75%, 90%, 95%, 99%, or more than 99% of the magnitude of an average characteristic dimension of the sites.

Array sites may have a morphology or shape (e.g., oval, circular, rectangular, square, triangular, polygonal, etc.). The morphology or shape of an array site may be characterized by an aspect ratio. An aspect ratio may be calculated as the ratio of the largest characteristic dimension to the smallest characteristic dimension. For example, ideal circles (largest diameter/smallest diameter) and ideal squares (longest edge length/shortest edge length) would have an aspect ratio of 1. Sites of an array or a subarray thereof may have an average aspect ratio of at least about 1, 1.1, 1.25, 1.5, 2, 3, 4, 5, 10, 20, 50, 100, or more than 100. Alternatively or additionally, sites of an array or a subarray thereof may have an average aspect ratio of no more than about 100, 50, 20, 10, 5, 4, 3, 2, 1.5, 1.25, 1.1, or less than 1.1. The average aspect ratio of sites of an array or subarray may be further characterized by a statistical measure of size deviation (e.g., variance or standard deviation). A measure of aspect ratio deviation of sites of an array or subarray thereof may be no more than about 99%, 95%, 90%, 75%, 50%, 40%, 30%, 25%, 20%, 15%, 10%, 5%, 1%, or less than 1% of the magnitude of an average characteristic dimension of the sites. Alternatively or additionally, a measure of aspect ratio deviation of sites of an array or subarray thereof may be at least about 1%, 5%, 10%, 15%, 20%, 25%, 30%, 40%, 50%, 75%, 90%, 95%, 99%, or more than 99% of the magnitude of an average characteristic dimension of the sites.

Array sites of an array or subarray may be characterized as having a pitch (i.e., an inter-site spacing, as measured centerpoint to centerpoint). Sites of an array or subarray thereof may have an average pitch of at least about 10 nm, 25 nm, 50 nm, 100 nm, 200 nm, 250 nm, 300 nm, 400 nm, 500 nm, 600 nm, 700 nm, 800 nm, 900 nm, 1 μm, 1.1 μm, 1.2 μm, 1.3 μm, 1.4 μm, 1.5 μm, 2 μm, 3 μm, 4 μm, 5 μm, or more than 5 μm. Alternatively or additionally, sites of an array or subarray thereof may have an average pitch of no more than about 5 μm, 4 μm, 3 μm, 2 μm, 1.5 μm, 1.4 μm, 1.3 μm, 1.2 μm, 1.1 μm, 1 μm, 900 nm, 800 nm, 700 nm, 600 nm, 500 nm, 400 nm, 300 nm, 250 nm, 200 nm, 100 nm, 50 nm, 25 nm, 10 nm, or less than 10 nm. The average pitch of sites of an array or subarray may be further characterized by a statistical measure of size deviation (e.g., variance or standard deviation). A measure of pitch deviation of sites of an array or subarray thereof may be no more than about 99%, 95%, 90%, 75%, 50%, 40%, 30%, 25%, 20%, 15%, 10%, 5%, 1%, or less than 1% of the magnitude of an average characteristic dimension of the sites. Alternatively or additionally, a measure of pitch deviation of sites of an array or subarray thereof may be at least about 1%, 5%, 10%, 15%, 20%, 25%, 30%, 40%, 50%, 75%, 90%, 95%, 99%, or more than 99% of the magnitude of an average characteristic dimension of the sites.

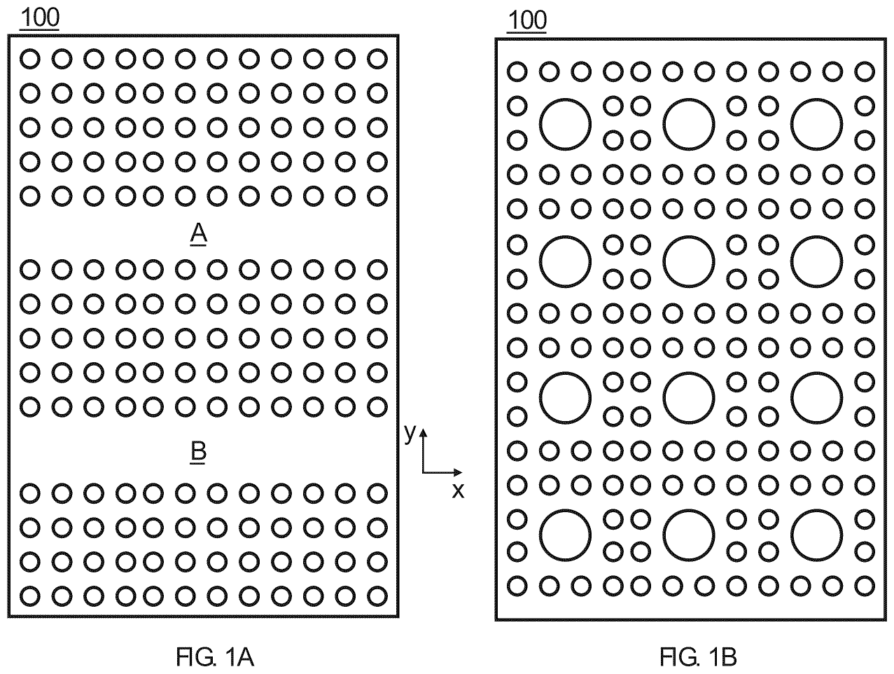

Sites of an array may be subdivided into subarrays. A subarray may comprise a plurality of sites that are clustered according to a shared characteristic (e.g., spatial contiguity, site dimension, site pitch, site shape and/or aspect ratio, site surface chemistry, etc.). In some embodiments, a subarray may comprise a plurality of sites, in which each site of the plurality of sites is spatially adjacent to at least one or more other sites of the plurality of sites. FIG. 1A illustrates a top-down view of an array on a solid support 100 comprising 3 subarrays of circular sites arranged in rectangular grids. Subarrays are spatially separated from adjacent subarrays by wide interstitial regions A and B. In other embodiments, a subarray may comprise a plurality of sites, in which a site of the plurality of sites is not spatially adjacent to any other site of the plurality of sites. FIG. 1B illustrates a top-down view of an array on a solid support 100 comprising a first subarray of contiguous small circular sites and a second subarray of non-contiguous large circular sites disposed within the first subarray.

Accordingly, in some embodiments, subarrays may provide a spatial reference for analytes and other array components on an array. For example, an analyte being bound to a site of the center subarray of FIG. 1A provides a narrower set of addresses for locating the analyte than the total quantity of possible addresses on the array. In other embodiments, subarrays may provide spatial ordering to differing array components. For example, a first subarray may be configured to bind analytes from a first sample and a second subarray may be configured to bind analytes from a second sample, thereby providing identifiable and/or predictable addresses for analytes of the first and second samples. Above-described aspects of arrays (e.g., site size, site aspect ratio, site surface chemistry, site pitch, site competency, and statistical variations in populations thereof) can be applied to sites of subarrays, collections of subarrays, and complete arrays.

In some embodiments, a solid support of an array may comprise a surface (e.g., a surface comprising an array) that is substantially planar. In other embodiments, a solid support of an array may comprise a surface (e.g., a surface comprising an array) that is non-planar or curved. In some embodiments, a solid support of an array may comprise a layer (e.g., a self-assembled monolayer) or a coating (e.g., a deposited metal, metal oxide, semiconductor, glass, polymer, dielectric material, etc.) that is disposed on a surface of the solid support. A layer or coating disposed on a surface of a solid support may comprise a distal surface relative to the surface of the solid support that is substantially planar. A layer or coating disposed on a surface of a solid support may comprise a distal surface relative to the surface of the solid support that is non-planar or curved.

In some embodiments, an array site or interstitial region may comprise a 3-dimensional structure. An array site or interstitial region may comprise a 3-dimensional structure that protrudes from a surface of a solid support (e.g., a hemisphere, a post, pillar, or pad). An array site or interstitial region may comprise a 3-dimensional structure that is recessed into a surface of a solid support (e.g., a well, pore, trench, or channel). In some embodiments, a 3-dimensional structure of an array site or interstitial region may be considered substantially 2-dimensional if a length scale of the 3-dimensional structure is much smaller than a length scale of another component of an array system. For example, an array site having an array site that projects about 5 nanometers from a solid support surface within a 50 micron fluidic channel may be considered substantially 2-dimensional relative to the fluidic channel.

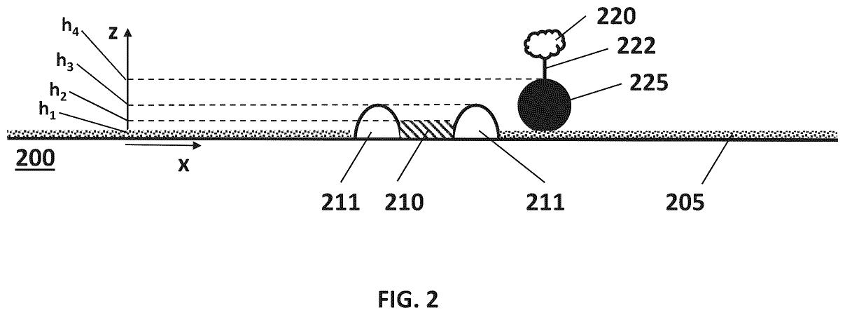

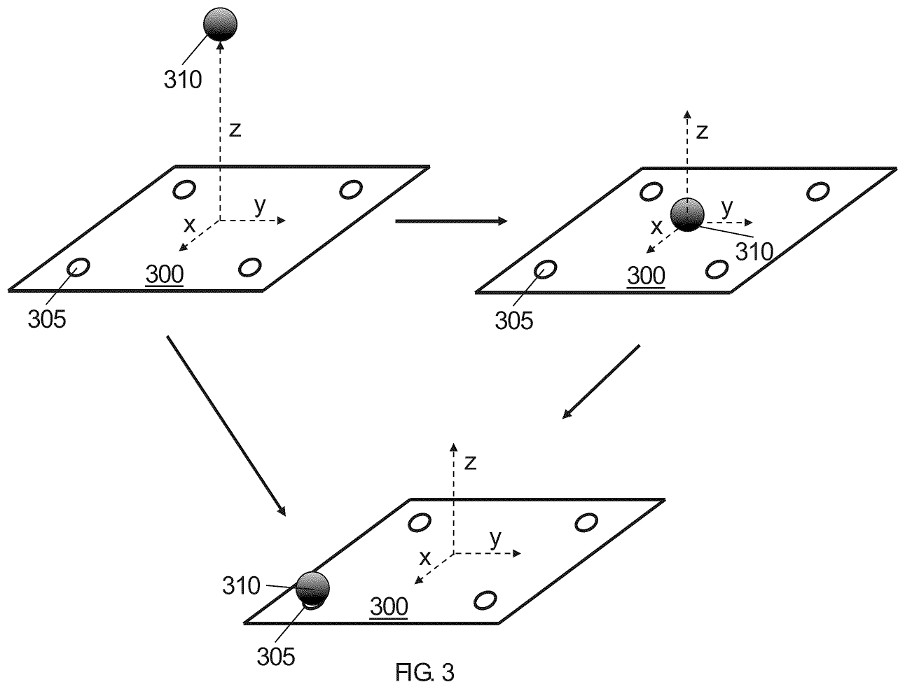

FIG. 2 illustrates a cross-sectional view of an array comprising 3-dimensional structures, in which the length scale of 3-dimensional structures of an array site are similar to the length scales of another array component (an analyte). A solid support 200 with a substantially planar surface is provided. Disposed on the upward surface of the solid support 200 is an array site 210 comprising a monolayer of molecules (e.g., coupling moieties, passivating moieties, etc.) with a height h₂ relative to the surface of the solid support 200. The outer edge of the array site 210 is surrounded by a boundary material 211 with a height of h₃ relative to the surface of the solid support 200. The array site 210 is further surrounded by an interstitial region comprising a thin layer or coating (e.g., an adhesion promoter) with a height of h₁ relative to the surface of the solid support 200. An analyte 220 (e.g., a polypeptide, a nucleic acid, a polysaccharide, a metabolite, etc.) is adjacent to the array site 210. The analyte 220 is coupled to an anchoring moiety 225 by a linker 222. The anchoring moiety 225 is configured to bind to the array site 210 and has a diameter of h₄. In the embodiment depicted in FIG. 2, the array site 210 may be considered 3-dimensional because the length scales of the array site 210 and boundary material 211 are similar to the length scale of the analyte 220 and/or anchoring moiety 225.

Accordingly, absolute or relative positions of array components (e.g., array sites, interstitial regions, analytes, anchoring moieties, etc.) may be described by a coordinate system. For a substantially planar solid support, array sites may be advantageously described by a 2-dimensional rectangular coordinate system (e.g., sites at [−1, 1], [−1, −1], [0, 1], etc.). As it pertains to certain aspects of array systems, it may be preferable to describe positions of array components relative to each other by a 3-dimensional rectangular coordinate system (i.e., as described relative to x-, y-, and z-axes).

An array or a subarray thereof can comprise sites and one or more interstitial regions, in which the sites have a characterized or known total site surface area, Aₛᵢₜₑ, (i.e., a cumulative surface area that combines the surface areas of each individual site), and in which the one or more interstitial regions have a characterized or known total interstitial surface area, Aᵢₙₜₑᵣₛₜᵢₜᵢₐₗ, (i.e., a cumulative surface area that combines the surface areas of each individual interstitial region). In some cases, Aᵢₙₜₑᵣₛₜᵢₜᵢₐₗ may be greater than Aₛᵢₜₑ. In other cases, Aᵢₙₜₑᵣₛₜᵢₜᵢₐₗ may be less than Aₛᵢₜₑ. In some cases, Aᵢₙₜₑᵣₛₜᵢₜᵢₐₗ may not be greater than Aₛᵢₜₑ. An array may be provided with a known ratio of Aᵢₙₜₑᵣₛₜᵢₜᵢₐₗ/Aₛᵢₜₑ, such as a ratio of at least about 0.001, 0.01, 0.1, 0.5, 1, 2, 5, 10, 20, 30, 40, 50, 75, 100, 125, 150, 200, 250, 300, 400, 500, 1000, 10000, 100000, 1000000, or more than 1000000. Alternatively or additionally, an array may be provided with a known ratio of Aᵢₙₜₑᵣₛₜᵢₜᵢₐₗ/Aₛᵢₜₑ, such as a ratio of no more than about 1000000, 100000, 10000, 1000, 500, 400, 300, 250, 200, 150, 125, 100, 75, 50, 40, 30, 20, 10, 5, 2, 1, 0.5, 0.1, 0.01, 0.001, 0.001, or less than 0.001.

### Analytes, Analyte Mass Transfer, and Analyte Competency

In some embodiments, an array or a subarray thereof may be substantially devoid of analytes. For example, an array may be provided to a method, as set forth herein, in which the array is substantially devoid of analytes. In some embodiments, a site of a plurality of sites of an array may be devoid of an analyte.

In some cases, an array may be contacted with a population of analytes. In some cases, a population of analytes may be bound to sites of an array. A population of analytes may comprise a biomolecule or bioactive molecule (e.g., a polypeptide, a polynucleotide, a polysaccharide, a metabolite, a lipid, a small molecule or pharmaceutical compound, or a combination thereof) or a nanoparticle (e.g., an organic nanoparticle, a polymeric nanoparticle, an inorganic nanoparticle, a semiconductor nanoparticle, a metal nanoparticle, etc.). An analyte of a population of analytes may be derived, separated, extracted, or otherwise obtained from a biological or a non-biological source. An analyte of a population of analytes may be derived, separated, extracted, or otherwise obtained from a manufactured, engineered, or naturally occurring source. A population of analytes may further comprise a standard or control moiety. A standard or control moiety may provide a qualitative or quantitative measure of a property of a population of analytes (e.g., purity, separation or extraction efficiency, coupling efficiency, analyte size, analyte species diversity, analyte chemical diversity, etc.). A population of analytes may comprise a quantity of standard or control moieties, such as at least about 0.000001%, 0.00001%, 0.0001%, 0.001%, 0.01%, 0.1%, 0.5%, 1%, 5%, 10%, or more than 10% of the population of analytes on a per analyte basis. Alternatively or additionally, a population of analytes may comprise a quantity of standard or control moieties, such as no more than about 10%, 5%, 1%, 0.5%, 0.1%, 0.01%, 0.001%, 0.0001%, 0.00001%, 0.000001%, or less than 0.000001% of the population of analytes on a per analyte basis

In some embodiments, an array may be contacted with a plurality of analytes. In particular embodiments, an array may be contacted with a fluidic medium comprising a plurality of analytes. In other embodiments, a plurality of analytes may be coupled to a plurality of sites of an array. Some methods set forth herein facilitate mass transfer of analytes through a fluidic medium to array sites of an array. Without wishing to be bound by theory, mass transfer of analytes to array sites may be resolved into: 1) mass transfer through a fluidic medium, and 2) mass transfer across an array surface. Mass transfer through a fluidic medium can be effectively 1-dimensional, 2-dimensional, or 3-dimensional depending on the context. For example, a steady-state concentration gradient in a fluidic medium relative to a surface can be considered 1-dimensional if the concentration gradient is the same relative to substantially every point on the surface. Mass transfer across an array surface may be effectively 1-dimensional or 2-dimensional depending on the context. Mass transfer across an array surface can include any mechanism of transfer in which the length scale of displacement of a transported species (e.g., an analyte) is very small compared to the length scale of displacement across the surface of the array. Displacement of a transported species away form a surface of an array may be limited for example by boundary layer effects or weak binding interactions of the transportee with the surface (e.g., electrostatic binding or van der Waals interactions). Mass transfer across an array surface may occur when transported species have sedimented, settled, or diffused to a surface of an array.

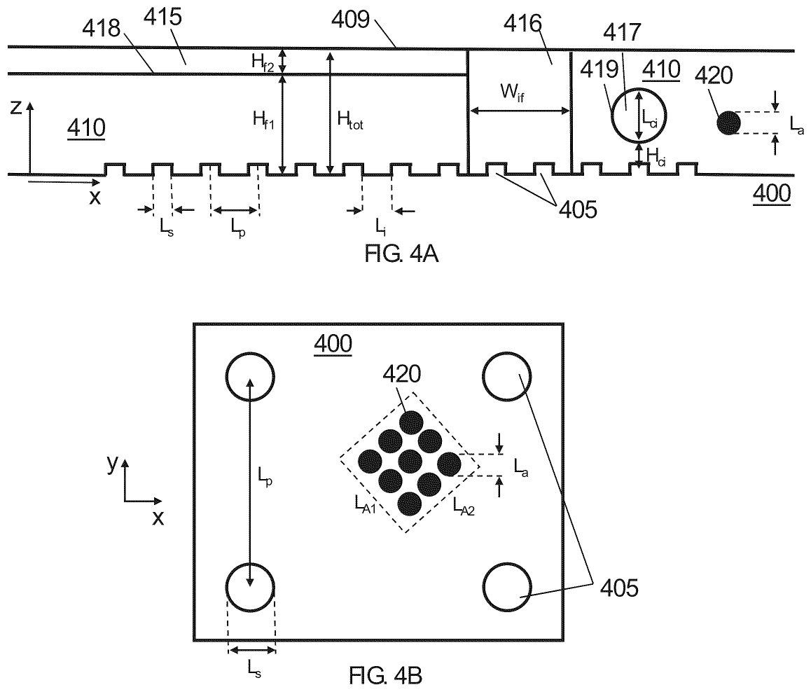

FIG. 3 provides an isometric view of an analyte 310 being transported to an array site 305 of a solid support 300. Two non-limiting pathways for mass transfer of analyte 310 to array site 305 are depicted. In a first mass transfer pathway, the analyte 310 initially transports to the surface of the solid support 300 (the surface coplanar with the x-axis and y-axis at a height z=0), then transports to the array site 305 across the surface. In a second mass transfer pathway, the analyte 310 transports directly to the array site 305 without surface transport (i.e., simultaneous transport relative to all 3 axes of the provided coordinate system).

If the array of FIG. 3 contains 3-dimensional array sites (e.g., like an array site 210 of FIG. 2), the z-axis dimensionality of an array site may inhibit the complete transport of a transported species to the array site. For example, transport of the analyte complex (containing analyte 220 and anchoring moiety 225) of FIG. 2 along the x-axis direction with limited z-axis displacement (e.g., displacement<<h₃) can inhibit binding of the anchoring moiety 225 to the array site 210.

Accordingly, a method carried out using an array having a surface in an x,y plane of a Cartesian coordinate system may provide one of more steps of: 1) transporting a transported species (e.g., an analyte, an anchoring moiety, or a combination thereof) through a fluidic medium in a direction relative to an x-axis, y-axis, z-axis, or a combination thereof, 2) transporting a transported species across an array surface relative to an x-axis, y-axis, or a combination thereof, and 3) producing a vertical displacement of a transported species away from the array surface along relative to a z-axis.

The skilled person will readily recognize that the 3-axis frame of reference relative to the array surface in the provided figures can be adjusted to any conceivable frame of reference, for example relative to the approximate centerpoint of an analyte or anchoring moiety.

An array may have a characterized site occupancy. A site occupancy may refer to a fraction or percentage of array sites that are bound to an analyte (fractional form is readily converted to percentage by multiplying by 100). Site occupancy may be a global site occupancy if it is determined with respect to a total quantity of array sites of an array or subarray. For example, an array having 10⁶ total array sites, of which 5×10⁵ sites are bound to an analyte, would have a global site occupancy of 0.5 (fractional form) or 50%. Site occupancy may be a relative site occupancy if it is determined with respect to a total quantity of competent array sites of an array or subarray. For example, an array having 10⁶ total array sites and a site competency of 0.9, of which 4.5×10⁵ sites are bound to an analyte, would have a relative site occupancy of 0.5 (fractional form) or 50%.

Moreover, an array may have a characterized site co-occupancy. A site co-occupancy may refer to a fraction or percentage of sites that are bound to two or more analytes. Site co-occupancy may be a global site co-occupancy if it is determined with respect to a total quantity of array sites of an array or subarray. For example, an array having 10⁶ total array sites, of which 1×10⁵ sites are bound to two or more analytes, would have a global site co-occupancy of 0.1 (fractional form) or 10%. Site co-occupancy may be a relative site co-occupancy if it is determined with respect to a total quantity of competent array sites of an array or subarray. For example, an array having 10⁶ total array sites and a site competency of 0.9, of which 0.9×10⁴ sites are bound to two or more analytes, would have a relative site co-occupancy of 0.1 (fractional form) or 10%.

A method, as set forth herein, may comprise a step of determining a site occupancy and/or site co-occupancy of an array. A method, as set forth herein, may comprise two or more steps of determining a site occupancy and/or co-occupancy of an array. In some embodiments, site occupancy and/or site co-occupancy of an array may be determined before contacting the array with a plurality of analytes or providing an analyte association condition to the array or the plurality of analytes contacted thereto. In some embodiments, site occupancy and/or site co-occupancy of an array may be determined after performing one or more steps of a method set forth herein, for example, after contacting the array with a plurality of analytes or providing an analyte association condition to the array or the plurality of analytes contacted thereto. In some embodiments, a method may not comprise determining a site occupancy and/or co-occupancy of an array before contacting the array with a plurality of analytes (i.e., an unused array may be presumed to be substantially devoid of any analytes).

An array may have a site occupancy of 0 (i.e., is substantially devoid of detectable analytes). An array may have a site occupancy (in fractional from) of at least about 0.000000001, 0.00000001, 0.0000001, 0.000001, 0.00001, 0.0001, 0.001, 0.005, 0.01, 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 0.99, 0.999, 0.9999, 0.99999, 0.999999, 0.9999999, 0.99999999, 0.999999999, or more than 0.999999999. Alternatively or additionally, an array may have a site occupancy of no more than about 0.999999999, 0.99999999, 0.9999999, 0.999999, 0.99999, 0.9999, 0.999, 0.99, 0.95, 0.9, 0.85, 0.8, 0.75, 0.7, 0.65, 0.6, 0.55, 0.5, 0.45, 0.4, 0.35, 0.3, 0.25, 0.2, 0.15, 0.1, 0.05, 0.01, 0.005, 0.001, 0.0001, 0.00001, 0.000001, 0.0000001, 0.00000001, 0.000000001, or less than 0.000000001.

An array may have a site co-occupancy of 0 (i.e., is substantially devoid of detectable analytes). An array may have a site co-occupancy (in fractional from) of at least about 0.0000000001, 0.000000001, 0.00000001, 0.0000001, 0.000001, 0.00001, 0.0001, 0.001, 0.005, 0.01, 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 0.99, 0.999, 0.9999, 0.99999, 0.999999, 0.9999999, 0.99999999, 0.999999999, 0.9999999999, or more than 0.9999999999. Alternatively or additionally, an array may have a site co-occupancy of no more than about 0.9999999999, 0.999999999, 0.99999999, 0.9999999, 0.999999, 0.99999, 0.9999, 0.999, 0.99, 0.95, 0.9, 0.85, 0.8, 0.75, 0.7, 0.65, 0.6, 0.55, 0.5, 0.45, 0.4, 0.35, 0.3, 0.25, 0.2, 0.15, 0.1, 0.05, 0.01, 0.005, 0.001, 0.0001, 0.00001, 0.000001, 0.0000001, 0.00000001, 0.000000001, 0.0000000001, or less than 0.0000000001.

After binding analytes to an array or a subarray thereof, the array or subarray may be characterized to determine: 1) a fraction or percentage of sites with no bound analytes, 2) a fraction or percentage of sites with one and only one bound analyte, and 3) a fraction or percentage of sites with two or more analytes. A method may comprise providing an array that is configured for a targeted per site occupancy, for example single-analyte occupancy (one and only one analyte per site), double-analyte occupancy (two and only two analytes per site), etc. A method may further comprise forming an array with super Poisson loading, in which super Poisson loading comprises: i) an increased fraction of sites having a targeted occupancy (e.g., single-analyte occupancy) relative to the occupancy predicted by a Poisson distribution, and ii) a decreased fraction of sites having a non-targeted occupancy relative to the occupancy predicted by a Poisson distribution. For the specific case of a single-analyte array, the single-analyte array and/or a subarray thereof may be determined to have super Poisson occupancy if: i) less than 37% of sites comprise no bound analytes, ii) greater than 37% of sites contain one and only bound analyte, and iii) less than 24% of sites contain two or more bound analytes. In more preferable embodiments, a single-analyte array and/or a subarray thereof may be determined to have super Poisson occupancy if: i) less than 35%, 30%, 25%, 20%, 15%, 10%, 5%, 1%, 0.5%, 0.1%, 0.05, or 0.01% of sites comprise no bound analytes, ii) greater than 40%, 45%, 50%, 55%, 60%, 65%, 70%, 75%, 80%, 85%, 90%, 95%, 99%, 99.9%, or 99.99% of sites contain one and only bound analyte, and iii) less than 20%, 15%, 10%, 5%, 1%, 0.5%, 0.1%, 0.05, or 0.01% of sites contain two or more bound analytes.

In some embodiments, super Poisson loading may be determined relative to a total number of sites of an array and/or a subarray thereof. In other embodiments, super Poisson loading may be determined relative to a total number of competent sites of an array and/or a subarray thereof (i.e., as determined by a known or characterized site competency of the array and/or subarray).

In an advantageous configuration, an analyte may be attached to an anchoring moiety. In a particular configuration, a population of anchoring moieties may be provided, in which each anchoring moiety of the population of anchoring moieties comprises one and only one analyte of a population of analytes. An anchoring moiety may be configured to form one or more binding interactions with an array site, thereby coupling an analyte attached to the anchoring moiety to the array site. In some cases, an anchoring moiety may comprise a nanoparticle, such as a nucleic acid nanoparticle. In some cases, an anchoring moiety may comprise one or more coupling moieties that form a binding interaction with an array site coupling moiety. An anchoring moiety may be further configured to occlude contact between an analyte attached to the anchoring moiety and an array site. In some cases, a method may comprise the steps of: a) attaching a population of analytes to a population of anchoring moieties, and b) attaching the population of anchoring moieties to sites of a populations of sites, thereby forming an array of analytes. Step b) can be carried out before, during or after step a).

An array of sites, as set forth herein, may be contacted with a population of analytes as a step of forming an array of analytes. A population of analytes may include an analyte that is incapable or unavailable for binding to an array or a site thereof. In particular cases, a population of analytes may comprise a population of anchoring moieties, in which each anchoring moiety of the population of anchoring moieties is coupled to an analyte of the population of analytes, and in which an anchoring moiety of the population of anchoring moieties is non-functional for the purpose of binding to an array site. Non-functional analytes and/or anchoring moieties may arise due to manufacturing error, manufacturing variability, variations in methods of analyte preparation (e.g., purification of analytes, separation of analytes, extraction of analytes, attachment of analytes to anchoring moieties, etc.), degradation during storage or usage, or combinations thereof. In particular, methods of functionalizing analytes and/or anchoring moieties for attachment (e.g., of anchoring moieties to analytes, of coupling moieties to analytes, of coupling moieties to anchoring moieties, etc.) may produce a fraction of analytes and/or anchoring moieties that do not comprise a coupling moiety, thereby preventing binding of the coupling moiety to an array site coupling moiety.

Analyte binding availability or anchoring moiety binding availability may comprise a measure of the fraction or percentage of total analytes or anchoring moieties, respectively, that contain a minimum set of structural features for forming an intended or designed binding interaction with an array site (i.e., a non-orthogonal binding interaction). Analyte binding availability or anchoring moiety binding availability may provide an upper bound to a maximum total occupancy of an array or a subarray thereof (e.g., when there are fewer available analytes and/or anchoring moieties relative to array sites). For example, if a population of analytes containing a total of 10⁶ analytes has an analyte binding availability of 95%, it would be expected that a maximum number of analytes or anchoring moieties available to bind to an array would be about 9.5×10⁵ analytes or anchoring moieties. Analyte binding availability or anchoring moiety binding availability may be characterized before an array-based assay or other process, for example by a quality control process. Analyte binding availability or anchoring moiety binding availability may be characterized during or after an array-based assay or other process, for example by sequential measurement of array occupancy to identify an asymptotic limit of occupancy. Analyte binding availability or anchoring moiety binding availability may be characterized by a bulk-scale or single-molecule physical measurement process, as provided herein.

A population of analytes and/or anchoring moieties may have an analyte binding availability or anchoring moiety binding availability of at least about 10%, 20%, 30%, 40%, 50%, 55%, 60%, 65%, 70%, 75%, 80%, 85%, 90%, 95%, 96%, 97%, 98%, 99%, 99.9%, 99.99%, 99.999%, or more than 99.999%. Alternatively or additionally, a population of analytes and/or anchoring moieties may have an analyte binding availability or anchoring moiety binding availability of no more than about 99.999%, 99.99%, 99.9%, 99%, 98%, 97%, 96%, 95%, 90%, 85%, 80%, 75%, 70%, 65%, 60%, 55%, 50%, 40%, 30%, 20%, 10%, or less than 10%.

Analyte binding availability or anchoring moiety binding availability may provide an analyte-specific or anchoring moiety-specific characterization of binding ability, or for populations of analytes or anchoring moieties, a characterization of total analytes and/or anchoring moieties available to bind to an array. An analyte, an anchoring moiety, or a population thereof may be further characterized with respect to analyte binding competency or anchoring moiety binding competency, respectively. Analyte binding competency or anchoring moiety binding competency may provide a measure of binding propensity for an analyte or an anchoring moiety. In other words, each available analyte and/or anchoring moiety of a population of analytes and/or anchoring moieties may comprise all necessary structural features to form intended or designed binding interactions with array sites, but individual analytes and/or anchoring moieties may display varying likelihood of binding particular array sites due to variations in analyte and/or anchoring moiety structure unique to the analyte and/or anchoring moiety. Without wishing to be bound by theory, analyte binding competency or anchoring moiety binding competency may depend upon thermodynamic and/or kinetic aspects of an intended or designed binding interaction between a site and an analyte or anchoring moiety.

Analyte binding competency and/or anchoring moiety binding competency can provide a qualitative and/or quantitative characterization of analyte binding propensity and/or anchoring moiety binding propensity for a single analyte, anchoring moiety, or a population thereof. Characterization of analyte binding competency and/or anchoring moiety binding competency may be presented in several fashions, including analyte-facing or anchoring moiety-facing characterizations (i.e., characterizing analyte binding competency or anchoring moiety binding competency in terms of analyte or anchoring moiety structural characteristics that influence propensity for analyte and/or anchoring moiety binding) or site-facing characterizations (i.e., characterizing analyte binding or anchoring moiety competency in terms of propensity to bind certain array sites).

In some cases, analyte binding competency and/or anchoring moiety binding competency may be characterized in a deterministic fashion. A deterministic characterization of analyte binding competency or anchoring moiety binding competency can comprise any characterization of which array sites an analyte or anchoring moiety can bind according to the structural properties of the array site or the structural characteristics of the analytes and/or anchoring moieties. For example, amongst a population of array sites, wherein each site contains one and only one of structures A, B, and C, an anchoring moiety may be configured to bind array site structure A, but not structures B and C. Accordingly, a deterministic characterization of the anchoring moiety can include information on competence for binding structure A and/or incompetence for binding structures B and C. A deterministic characterization of analyte binding competency and/or anchoring moiety binding competency for an analyte or anchoring moiety may be a site-facing characterization, such as an identification of whether an array site will or will not bind to the analyte or anchoring moiety, or which array site structures amongst a variety of array site structures will or will not bind to the analyte and/or anchoring moiety. A deterministic characterization of analyte binding competency and/or anchoring moiety binding competency for an analyte or anchoring moiety may be an analyte-facing or anchoring moiety-facing characterization, such as an analyte or anchoring moiety categorization based upon one or more site structural features of the analyte or anchoring moiety. For example, an anchoring moiety may be categorized with respect to binding competency according to its characteristic dimension, electrical charge anisotropy, species of coupling moiety, surface density of coupling moieties, or combinations thereof.

In some cases, analyte binding competency and/or anchoring moiety binding competency may be characterized in a probabilistic or stochastic fashion. A probabilistic or stochastic characterization of analyte binding competency or anchoring moiety binding competency can comprise any characterization of a likelihood of an analyte or an anchoring moiety binding an array site. For example, amongst a population of array sites containing one and only one of structures A, B, and C, an anchoring moiety may be best configured to bind array site structure A, and less optimally configured to bind structures B and C. Accordingly, a probabilistic characterization of the anchoring moiety can include information on a higher binding probability for binding structure A, and a lower probability for binding structures B and C. A probabilistic characterization of analyte binding competency and/or anchoring moiety binding competency for an analyte or anchoring moiety may be a site-facing characterization, such as a probability of binding an array site based upon a structural characteristic of the array site. A probabilistic characterization of analyte binding competency and/or anchoring moiety binding competency for an analyte or anchoring moiety may be an analyte-facing characterization, such as a probability of binding an array site based upon a structural characteristic of analyte and/or anchoring moiety.

Information on analyte binding competency or anchoring moiety binding competency for individual analyte or anchoring moieties may be coalesced or aggregated into an analyte binding competency profile or anchoring moiety binding competency profile. An analyte binding competency profile or anchoring moiety binding competency profile may provide a qualitative or quantitative estimate or measure of a range of binding competencies present in a population of unbound analytes or anchoring moieties. For example, for a population of anchoring moieties, a range of binding competencies can be expected due to variability in one or more structural features of each anchoring moiety of the population of anchoring moieties. Accordingly, an anchoring moiety binding competency profile can include a quantitative measure of number of anchoring moieties in subpopulations grouped by one or more structural features that influence anchoring moiety binding propensity. In some cases, an analyte binding competency profile or anchoring moiety binding competency profile may comprise a quantitative measure of number of analyte or anchoring moieties in subpopulations grouped by a single parameter model or a multi-parameter model of array site binding.

In some cases, an analyte binding competency profile or anchoring moiety binding competency profile can comprise a qualitative characterization of binding competency for a population of analytes or anchoring moieties. A population of analytes or anchoring moieties (e.g., in a fluidic medium) can be assigned a qualitative characterization of binding competency (e.g., High/Medium/Low; Type 1, Type 2, Type 3, etc . . . ; Type A, Type B, Type C, etc. . . . ) based upon an estimated or measured range of binding competencies.

In some cases, a method of forming an array of analytes, as set forth herein, may comprise providing a population of binding entities (e.g., analytes, anchoring moieties or analytes attached to anchoring moieties). Providing a population of binding entities may comprise separating from a plurality of binding entities a binding entity to provide the population of binding entities, in which an array containing a population of array sites is substantially devoid of array sites with a binding competency for the binding entity. In some cases, separating a binding entity from a plurality of binding entities may comprise separating a damaged binding entity, a malformed binding entity, a misfolded binding entity, an altered binding entity, or a plurality thereof from the plurality of binding entities. In some cases, separating a binding entity from a plurality of entities may provide a population of binding entities with an improved binding entity competency profile. For example, after separating a binding entity from a plurality of binding entities, a binding entity binding competency profile may be better corresponded to an array site binding competency profile of an array.

Although aspects of the above discussion have been exemplified with respect to analytes, it should be noted that the disclosed concepts can be extended to other transportates, such as macromolecules (e.g., analytes), particles (e.g., anchoring moieties, nanoparticles, or microparticles), or moieties (e.g., small molecules). For example, array site occupancy may be determined with respect to a fraction of array sites comprising an anchoring moiety rather than a fraction of array sites containing an analyte.

### Length Scales in Array Systems

The present disclosure provides numerous systems and methods for preparing and utilizing arrays of analytes. In some aspects, the sizing and formatting of arrays may vary depending upon factors such as analyte size and desired analyte density. Moreover, array methods and/or systems may utilize two or more mechanisms of mass transfer to facilitate formation of high-occupancy arrays of analytes, in which at least two of the mechanisms of mass transfer have differing length scales (e.g., microscale vs. nanoscale). Accordingly, in some cases it may be preferable to describe length scales of arrays and components of arrays systems (e.g., analyte size, anchoring moiety size, site size, site pitch, fluid layer thickness, interface height, interface length, etc.) by aspect ratios rather than absolute lengths.

The present disclosure presents numerous mechanisms for effecting mass transfer of binding entities, such as analytes and/or anchoring moieties, in innumerable configurations and combinations thereof. The skilled person will recognize that the described systems present various elements of transport phenomena, including combined or separate mass transfer, momentum transfer, and heat transfer. Accordingly, obtaining successful outcomes (e.g., arrays with super Poisson loading) for a particular system can require careful design and selection of length scales, or ratios thereof. Provided herein are numerous measurable length scales that may be useful in designing systems and methods that successfully achieve desired array occupancy outcomes.

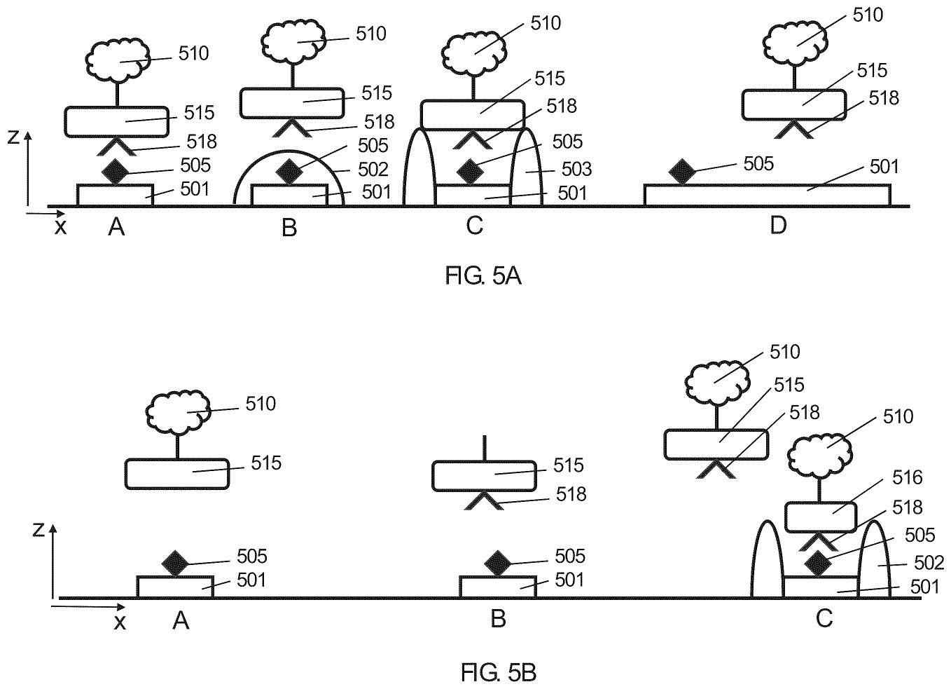

FIG. 4A provides a cross-sectional view of an array system with certain array features and associated length scales highlighted. A first solid support 400 comprises a plurality of sites 405. The sites 405 have a characteristic dimension Lₛ (e.g., length, width, diameter or height) and the sites are spaced on a pitch Lₚ, as optionally measured centerpoint to centerpoint. A bounded volume is formed by providing a second solid support 409 that is substantially parallel and opposed to the first solid support 400, with an average separation distance of Hₜₒₜ between the two solid supports (e.g., a chamber or channel depth of a microfluidic device). The left side of FIG. 4A depicts a first fluid 410 contacted to the surface of the first solid support 400, with the first fluid 410 having an average depth of H_{f1}. An open or unbounded interface 418 is formed between the first fluid 410 and a second fluid 415 (e.g., a gas or liquid). The second fluid 415 has an average depth of H_{f2}, and optionally fills the remaining volume between the open interface 418 and the second solid support 409. The right side of FIG. 4A depicts the first fluid 410 contacted to the surface of the solid support 400, with the first fluid 410 having an average depth of Hot. Contained within the right side volume of the first fluid 410 is a closed or bounded interface 419 surrounding a volume of a third fluid 417 (e.g., a gas, a liquid, or an emulsified liquid, etc.) and an array component (e.g., an analyte or an anchoring moiety) with a characteristic dimension Lₐ. The bubble of the third fluid 417 has a characteristic dimension L_{ci} and the nearest distance between the bubble and the first solid support 400 and/or site 405 has a separation distance of H_{ci}. The left side fluids and the right side fluids are separated by a bolus of a fourth fluid 416. The bolus has a characteristic width W_{if}.

FIG. 4B provides a top-down view of a plurality of sites 405 disposed on a solid support 400. The plurality of sites 405 has an average inter-site pitch Lₚ, and the sites have a characteristic dimension Lₛ. Disposed on an interstitial region of the solid support 400 are a plurality of N analytes 420, with each analyte having an average characteristic dimension Lₐ (e.g., diameter, length, width, hydrodynamic radius, etc.). The N analytes are disposed within a portion of the interstitial region, thereby giving a surface density of analytes within the portion of the interstitial region of N/(L_{A1}*L_{A2}).

FIGS. 4A-4B depict non-exhaustive illustrations of relevant length scales for arrays and array systems, as set forth herein. Table I presents a non-exhaustive list of relevant length scales for various possible array system parameters. Column A of Table I lists parameters for array components, including components bound or affixed to arrays. Column B of Table I lists parameters for system components contacted to an array. In some cases, a method, as set forth herein, may utilize an array with a known or characterized array parameter chosen from Column A of Table I, and a system component with a known or characterized system parameter chosen from Column B of Table I, in which an aspect ratio of the array parameter to the system parameter (when length units are consistent, e.g., meters to meters) is at least about 0.000001, 0.00001, 0.0001, 0.001, 0.01, 0.1, 0.5, 1, 2, 5, 10, 50, 100, 1000, 10000, 100000, 1000000, or more than 1000000. Alternatively or additionally, a method, as set forth herein, may utilize an array with a known or characterized array parameter chosen from Column A of Table I, and a system component with a known or characterized system parameter chosen from Column B of Table I, in which an aspect ratio of the array parameter to the system parameter (when length units are consistent, e.g., meters to meters) is no more than about 1000000, 100000, 10000, 1000, 100, 50, 10, 5, 2, 1, 0.5, 0.1, 0.01, 0.001, 0.0001, 0.00001, 0.000001, or less than 0.000001.

TABLE I

| A: Array Length Parameters (meters) | B: System Length Parameters (meters) |
|---|---|
| Average site length | Average unbound analyte width |
| Average site width | Average unbound analyte height |
| Average site diameter | Average unbound analyte hydrodynamic radius |
| Average site height/depth | Maximum unbound analyte width |
| Average site pitch | Maximum unbound analyte height |
| Maximum site length | Maximum unbound analyte hydrodynamic radius |
| Maximum site width | Minimum unbound analyte width |
| Maximum site diameter | Minimum unbound analyte height |
| Maximum site height/depth | Minimum unbound analyte hydrodynamic radius |
| Maximum site pitch | Average unbound anchoring moiety width |
| Minimum site length | Average unbound anchoring moiety height |
| Minimum site width | Average unbound anchoring moiety hydrodynamic radius |
| Minimum site diameter | Maximum unbound anchoring moiety width |
| Minimum site height/depth | Maximum unbound anchoring moiety height |
| Minimum site pitch | Maximum unbound anchoring moiety hydrodynamic radius |
| Average bound analyte width | Minimum unbound anchoring moiety width |
| Average bound analyte height | Minimum unbound anchoring moiety height |
| Average bound analyte hydrodynamic | Minimum unbound anchoring moiety hydrodynamic radius |
| radius | |
| Maximum bound analyte width | Average fluid layer thickness |
| Maximum bound analyte height | Maximum fluid layer thickness |
| Maximum bound analyte hydrodynamic | Minimum fluid layer thickness |
| radius | |
| Minimum bound analyte width | Average droplet/bubble diameter |
| Minimum bound analyte height | Maximum droplet/bubble diameter |
| Minimum bound analyte hydrodynamic | Minimum droplet/bubble diameter |
| radius | |
| Average bound anchoring moiety width | Average droplet/bubble separation distance |
| Average bound anchoring moiety height | Maximum droplet/bubble separation distance |
| Average bound anchoring moiety | Minimum droplet/bubble separation distance |
| hydrodynamic radius | |
| Maximum bound anchoring moiety width | Average interface separation distance |
| Maximum bound anchoring moiety height | Maximum interface separation distance |
| Maximum bound anchoring moiety | Minimum interface separation distance |
| hydrodynamic radius | |
| Minimum bound anchoring moiety width | Average fluid bolus length |
| Minimum bound anchoring moiety height | Maximum fluid bolus length |
| Minimum bound anchoring moiety | Minimum fluid bolus length |
| hydrodynamic radius | |
| Average site surface-coupled moiety | Average fluid bolus width |
| length | |
| Maximum site surface-coupled moiety | Maximum fluid bolus width |
| length | |
| Minimum site surface-coupled moiety | Minimum fluid bolus width |
| length | |
| Average interstitial surface-coupled | Average fluid bolus height |
| moiety length | |
| Maximum interstitial surface-coupled | Maximum fluid bolus height |
| moiety length | |
| Minimum interstitial surface-coupled | Minimum fluid bolus height |
| moiety length | |
| Average interstitial layer thickness | |
| Maximum interstitial layer thickness | |
| Minimum interstitial layer thickness | |
| Average fluidic channel/chamber length | |
| Maximum fluidic channel/chamber length | |
| Minimum fluidic channel/chamber length | |
| Average fluidic channel/chamber width | |
| Maximum fluidic channel/chamber width | |
| Minimum fluidic channel/chamber width | |
| Average fluidic channel/chamber height | |
| Maximum fluidic channel/chamber height | |
| Minimum fluidic channel/chamber height | |

### Quality Characterization of Arrays and Analytes

A method utilizing an array, as set forth herein, may include one or more array characterization steps. In some cases, an array may be provided for a method, as set forth herein, in which the array has been characterized with respect to an array property (e.g., site dimension, average site dimension, site surface-coupled moiety density, average site surface-coupled moiety density, site coupling moiety density, average site coupling moiety density, site passivating moiety density, average site passivating moiety density, site pitch, average site pitch, etc.). In some cases, an array may be provided for a method, in which the array has a characterized array site binding availability and/or array site binding competency profile. In some cases, a method may comprise a step of characterizing an array site binding availability and/or an array site binding competency profile.

The skilled person will readily recognize numerous appropriate techniques for measuring array properties set forth herein. Exemplary techniques can include bulk characterization methods, including x-ray diffraction, x-ray photoelectron spectroscopy, infrared spectroscopy, Auger electron spectroscopy, Mossbauer spectroscopy, Raman spectroscopy, UV-Vis spectroscopy, energy dispersive x-ray spectroscopy, electron energy loss spectroscopy, atomic fluorescence spectroscopy, attenuated total reflection, BET surface area measurement, coherent diffraction imaging, cyclic voltammetry, contact angle measurement, molecular beam epitaxy, nuclear magnetic resonance, surface interferometry, and small angle x-ray scattering. Other characterization techniques may include optical and/or microscopy techniques, such as confocal scanning microscopy, fluorescence microscopy, fluorescence lifetime imaging, scanning electron microscopy, transmission electron microscopy, scanning probe microscopy, atomic force microscopy, bright-field microscopy, dark-field microscopy, hyperspectral imaging, x-ray diffraction topography, total internal fluorescence microscopy, and field ion microscopy. In some cases, single-site measurements may be obtained by an optical or microscopy technique, including super-resolution microscopy methods. For example, properties such as site dimension, site pitch, and site morphology can be measured for an individual array site. In some cases, an array characterization method may comprise an invasive technique, in which the invasive technique alters one or more array sites during a characterization measurement. In some cases, an array of a population of arrays may be characterized to identify an array property (e.g., array site binding availability, array site binding competency, an array site binding competency profile, etc.), in which the identified array property is subsequently assigned to other arrays of the population of arrays.

A method utilizing a population of analytes and/or anchoring moieties, as set forth herein, may include one or more analyte or anchoring moiety characterization steps. In some cases, an analyte, an anchoring moiety, or a population thereof may be provided for a method, as set forth herein, in which the analyte, the anchoring moiety, or the population thereof has been characterized with respect to an analyte and/or anchoring moiety property (e.g., characteristic dimension, average characteristic dimension, coupling moiety density, average coupling moiety density, hydrodynamic radius, average hydrodynamic radius, isoelectric point, average isoelectric point, etc.). In some cases, an analyte, an anchoring moiety, or a population thereof may be provided for a method, in which the analyte, the anchoring moiety, or the population thereof has a characterized analyte or anchoring moiety binding availability and/or analyte or anchoring moiety binding competency profile. In some cases, a method may comprise a step of characterizing an analyte or anchoring moiety binding availability and/or an analyte or anchoring moiety binding competency profile.

The skilled person will readily recognize numerous appropriate techniques for measuring analyte and/or anchoring moiety properties set forth herein. Exemplary techniques can include bulk characterization methods, including x-ray diffraction, x-ray photoelectron spectroscopy, infrared spectroscopy, Auger electron spectroscopy, Mossbauer spectroscopy, Raman spectroscopy, UV-Vis spectroscopy, energy dispersive x-ray spectroscopy, electron energy loss spectroscopy, atomic fluorescence spectroscopy, attenuated total reflection, coherent diffraction imaging, cyclic voltammetry, Zeta potential measurement, gas chromatography, liquid chromatography, mass spectrometry, nuclear magnetic resonance, dynamic light scattering, particle imaging velocimetry, differential scanning calorimetry, and thermogravimetric analysis. Other characterization techniques may include optical and/or microscopy techniques, such as confocal scanning microscopy, fluorescence microscopy, fluorescence lifetime imaging, scanning electron microscopy, transmission electron microscopy, scanning probe microscopy, atomic force microscopy, bright-field microscopy, dark-field microscopy, hyperspectral imaging, x-ray diffraction topography, and field ion microscopy. In some cases, single-analyte or single-anchoring moiety measurements may be obtained by an optical or microscopy technique, including super-resolution microscopy methods. For example, properties such as characteristic dimension, morphology, and analyte attachment can be measured for an individual anchoring moiety and/or analyte. In some cases, an analyte or anchoring moiety characterization method may comprise an invasive technique, in which the invasive technique alters one or more analytes or anchoring moieties during a characterization measurement (e.g., mass spectrometry). In some cases, an analyte or anchoring moiety of a population of analyte or anchoring moieties may be characterized to identify an analyte or anchoring moiety property (e.g., analyte or anchoring moiety binding availability, analyte or anchoring moiety binding competency, an analyte or anchoring moiety binding competency profile, etc.), in which the identified analyte or anchoring moiety property is subsequently assigned to other analytes or anchoring moieties of the population of analytes or anchoring moieties.

Characterization of an array of sites or a population of analytes and/or anchoring moieties may include a functional characterization. A functional characterization of an array of sites or a population of analytes and/or anchoring moieties can include any method that measures an array, analyte, or anchoring moiety binding property by binding functional entities to an array. A functional characterization method may comprise the steps of: a) contacting a solid support comprising a population of sites with a population of functional entities (e.g., sample analytes, standard or control analytes, anchoring moieties, standard or control anchoring moieties, standard or control analytes attached to anchoring moieties), b) binding functional entities of the population of functional entities to sites of the population of sites, and c) measuring a binding property of the population of sites, optionally in which the measuring comprises a single-site or single-analyte measurement, as set forth herein. In some cases, a method of functional characterization may further comprise the steps of: d) releasing the bound functional entities from the sites of the population of sites, and e) after releasing the bound functional entities, optionally performing an array-based method, as set forth herein.

In some cases, functional characterization may comprise binding functional entities of a population of functional entities to sites of a population of sites of an array. A total quantity of functional entities utilized during functional characterization, or a total quantity of sites of a population of sites bound by functional entities during functional characterization, may comprise a small quantity relative to the total quantity of a population of array sites, such as no more than about 10%, 5%, 1%, 0.5%, 0.1%, 0.05%, 0.01%, 0.001%, 0.0001%, 0.00001%, 0.000001%, or less than 0.000001% of the total quantity of the population of array sites. Alternatively or additionally, a total quantity of functional entities utilized during functional characterization, or a total quantity of sites of a population of sites bound by functional entities during functional characterization, may comprise a quantity relative to the total quantity of a population of array sites of at least about 0.000001%, 0.00001%, 0.0001%, 0.001%, 0.01%, 0.05%, 0.1%, 0.5%, 1%, 5%, 10%, or more than 10% of the total quantity of the population of array sites. In some cases, a method may comprise: i) performing a functional characterization method on a solid support comprising a population of sites, and ii) after performing the functional characterization, performing an array-based method, as set forth herein, in which the bound functional entities are present during one or more steps of the array-based method.

Characterizations of array site and/or binding entity (e.g., analytes or anchoring moieties) behavior, such as array site binding competency profiles or analyte binding competency profiles, may be performed by a method that incorporates a computational algorithm. A computational algorithm may apply any suitable statistical method that identifies relationships between measured characteristics of arrays or binding entities and array site or binding entity behavior, respectively. A computational algorithm may receive a dataset containing a plurality of measurements from array sites or binding entities and, based upon the received measurements, compute a binding competency profile (e.g., an array site binding competency profile or an analyte binding competency profile). In some cases, a computational algorithm may receive a dataset containing two or more pluralities of measurements from array sites or binding entities and, based upon the received measurements, compute a binding competency profile (i.e., a multi-parameter model of binding competency).

A computation algorithm for determining a binding competency profile may receive a plurality of measurements, in which the plurality of measurements comprises a data sample. For example, an array site characteristic may be measured for a fraction of array sites of an array (e.g., no more than 0.1%, 1%, 10%, etc.), and based upon a statistical analysis of the array site characteristic measurements for the fraction of array sites, an array site binding competency profile may be calculated for the array. In another example, for a manufactured batch of arrays, one or more arrays may be selected at random for measurement of array site characteristics, and based upon the measured array site characteristics, one or more array site binding competency profiles may be calculated for the batch of arrays.

In some cases, a computational algorithm may comprise a machine learning algorithm. A machine learning algorithm may incorporate various sources of information to determine binding competency profiles (e.g., an array site binding competency profile or an analyte binding competency profile). The machine learning algorithm can be trained using such sources of information obtained from prior empirical measurements or prior hypothetical models. Sources of information may include measured characteristics of array sites or binding entities, functional characterization results, and outcomes of assays or methods of array utilization. For example, a machine learning model may incorporate, or may be trained using, measurements of array characteristics and single-analyte resolution site occupancy information from an analyte deposition process to form or refine a predictive model for array site binding competency profiles. Such a model could receive subsequent array site characteristic measurements from a given array and use a model trained on prior array measurements to predict an outcome for an analyte deposition process on the given array. A system for array or binding entity characterization may comprise an algorithm that is configured to implement a method such as machine learning, deep learning, statistical learning, supervised learning, unsupervised learning, clustering, expectation maximization, maximum likelihood estimation, Bayesian inference, non-Bayesian inference, linear regression, logistic regression, binary classification, multinomial classification, or other pattern recognition algorithm. Examples of machine learning algorithms may include support vector machines (SVMs), neural networks, convolutional neural networks (CNNs), deep neural networks, cascading neural networks, k-Nearest Neighbor (k-NN) classification, random forests (RFs), and other types of classification and regression trees (CARTs).

A method of determining a binding competency profile (e.g., an array site binding competency profile, an analyte binding competency profile, or an anchoring moiety binding competency profile) may comprise: i) measuring characteristics (e.g., array site characteristics, analyte characteristics, or anchoring moiety characteristics) by a characterization method (including but not limited to characterization methods set forth herein), and ii) based upon the measured characteristics, determining the binding competency profile. Table II provides a list of array site and analyte or anchoring moiety characteristics that may be utilized for determining a binding competency profile. Measured characteristics may include bulk or ensemble measurements, or single-entity measurements. Any characteristic listed in Table II may be provided to a model or algorithm for determining a binding competency profile in several quantitative forms, including mean or average value, median value, maximum value, minimum value, or a distribution of measured values, as well as related statistical measures, such as measures of variance and measures of sample size.

TABLE II

| Array Site Characteristic | Binding Entity Characteristic |
|---|---|
| Site dimension | Binding entity dimension |
| Site surface area | Binding entity face dimension |
| Site aspect ratio | Binding entity effective surface area |
| Site pitch | Binding entity face effective surface area |
| Site depth | Binding entity hydrodynamic radius |
| Site height | Binding entity weight |
| Coating or layer thickness | Total coupling moiety quantity |
| Total molecular surface density | Coupling moiety species quantity |
| Total coupling moiety surface density | Total coupling moiety surface density |
| Coupling moiety species surface density | Coupling moiety species surface density |
| Total passivating moiety surface density | Total passivating moiety surface density |
| Passivating moiety species surface density | Passivating moiety species surface density |
| Total surface moiety quantity | Total coupling moiety quantity |
| Total coupling moiety quantity | Coupling moiety species quantity |
| Coupling moiety species quantity | Total passivating moiety quantity |
| Total passivating moiety quantity | Passivating moiety species quantity |
| Passivating moiety species quantity | Total coupling moiety functional quantity |
| Total coupling moiety functional quantity | Coupling moiety species functional quantity |
| Coupling moiety species functional quantity | Coupling moiety linker length |
| Passivating layer thickness | Coupling moiety linker chemical composition |
| Passivating layer volume | Binding entity net surface electrical charge density |
| Boundary material height | Binding entity surface electrical charge anisotropy |
| Boundary material width | Binding entity isoelectric point |
| Boundary material volume | Binding entity Zeta potential |
| Passivating layer chemical composition | |
| Boundary layer chemical composition | |
| Defect surface density | |
| Array site net surface electrical charge | |
| density | |

### Methods of Forming High-Occupancy Arrays

In an aspect, provided herein are methods for forming arrays of analytes (e.g., single analytes), in which the methods comprise the steps of: a) providing a solid support with a total quantity of sites, in which the solid support has a fractional global site competency of less than 1 (i.e., less than 100% site competency), and in which the solid support is substantially devoid of analytes, b) contacting a fluidic medium comprising a plurality of analytes to the solid support, and c) binding analytes of the plurality of analytes to sites of the total quantity of sites, thereby obtaining the array of analytes that is characterized by super Poisson loading of analytes relative to the total quantity of sites. In some cases, a method may occur within a fixed amount of time, such as no more than about 6 hours, 3 hours, 2 hours, 60 minutes, 45 minutes, 30 minutes, 20 minutes, 15 minutes, 10 minutes, 5 minutes, 1 minute, or less than 1 minute. In some cases, a method may occur with no more than one analyte-contacting step (i.e., a first plurality of analytes is contacted to a solid support and no further analytes exogenous to the first plurality of analytes is subsequently contacted to the solid support). In other cases, a method may occur with more than one analyte-contacting step. In some cases, a method may comprise contacting a solid support comprising a total quantity of sites with a plurality of analytes, in which a total quantity of the plurality of analytes is greater than the total quantity of sites.

In another aspect, provided herein are methods for forming arrays of analytes (e.g., single analytes), in which the methods comprise the steps of: a) providing a solid support comprising a plurality of sites, b) contacting the solid support with a fluidic medium comprising a plurality of analytes, in which a total quantity of analytes of the plurality of analytes is less than a total quantity of sites of the plurality of sites, and c) depositing at least about 50% (e.g., at least about 50%, 55%, 60%, 65%, 70%, 75%, 80%, 85%, 90%, 95%, 96%, 97%, 98%, 99%, 99.9%, 99.99%, 99.999%, 99.9999%, 99.99999%, or more than 99.99999%) of analytes of the total quantity of analytes at sites of the plurality of sites.

FIG. 34 depicts a schematic of a method of preparing an array of analytes, in accordance with some embodiments set forth herein. Steps 3400-3406 depicts a method of preparing a plurality of analytes that may be provided to a solid support. Optionally, the analytes are coupled to particles (e.g., nucleic acid nanoparticles, polymer particles). In a first optional step 3400, a plurality of particles may be provided. In a second step 3401, the plurality of particles may undergo a purification process that separates aggregates or multimeric particles from monomeric particles, thereby providing a plurality of monomeric particles. A purified fraction of monomeric particles may comprise at least about 80% (e.g., at least about 80%, 85%, 90%, 95%, 99%, 99.5%, 99.9%, or more than 99.9%) monomeric particles on a weight or molar basis. In a third step 3405, analytes may be coupled to the monomeric particles, thereby forming analyte-particle complexes. Preferably, one and only analyte is coupled to one and only one particle in each analyte-particle complex. In an optional fourth step 3406, the plurality of analyte-particle complexes may undergo a purification process that separates aggregates or multimeric analyte-particle complexes from monomeric analyte-particle complexes, thereby providing a plurality of monomeric analyte-particle complexes. A purified fraction of monomeric analyte-particle complexes may comprise at least about 80% (e.g., at least about 80%, 85%, 90%, 95%, 99%, 99.5%, 99.9%, or more than 99.9%) monomeric analyte-particle complexes.

Prior to delivering analyte-particle complexes to a solid support, the particles and/or the analyte-particle complexes may be fractionated to provide an enriched fraction of monomeric particles or analyte-particle complexes, respectively. A monomeric particle can refer to a single particle that is substantially devoid of any unwanted binding interactions. A nucleic acid nanoparticle may become aggregated with misfolded or partially-folded nucleic acid nanoparticles, or may become aggregated with nucleic acid nanoparticle components (e.g., oligonucleotides). Alternatively, two or more nucleic acid nanoparticles can become coupled to form a multimeric particle complex. Likewise, a monomeric analyte-particle complex can refer to a single analyte attached to a single particle. An analyte-particle complex may become aggregated with misfolded or partially-folded nucleic acid nanoparticles, or may become aggregated with nucleic acid nanoparticle components (e.g., oligonucleotides), or may become aggregated with additional analytes or components thereof. Alternatively, two or more analyte-particle complexes can become coupled to form a multimeric analyte-particle complex. A plurality of particles or a plurality of analyte-particle complexes may be fractionated to provide an enriched monomeric fraction and an enriched aggregate or multimeric fraction. Monomeric particle or analyte-particle complexes may be fractionated by any suitable method, including affinity chromatography, size exclusion chromatography, high-pressure liquid chromatography, and tangential flow filtration. Alternatively or additionally, aggregates or multimeric particles or analyte-particle complexes may be disrupted by contacting the aggregates or multimeric particles or analyte-particle complexes with a dispersing buffer. Depending upon the type of particle and/or analytes, the dispersing buffer may be formulated with respect to ionic strength, ionic composition, buffer composition, surfactant composition and/or concentration, denaturant composition and/or concentration, and temperature to facilitate dissociation of aggregates or multimers.

Continuing with FIG. 34, a plurality of analytes (e.g., analyte-particle complexes) can be delivered 3410 to a solid support comprising an array of sites. Preferably, the analytes may be delivered in a fluidic medium. After delivering the analytes 3410 to the solid support, the analytes may be incubated 3420 with the solid support for an amount of time, thereby facilitating binding of analytes to sites of the solid support. Optionally, an analyte association condition, as set forth herein, may be provided 3430. Next, unbound analytes (e.g., analyte-particle complexes) may be removed 3440 from the solid support (e.g., by providing a rinsing medium). Optionally, after removing 3440 the analytes, the analytes may be again delivered 3410 to the solid support one or more times. Optionally, additional analytes (e.g., analyte-particle complexes) may be added 3470 before again delivering 3410 them to the solid support. After removing 3440 unbound analytes from the solid support, the solid support may be detected 3450 to determine a presence or absence of a signal associated with an analyte (e.g., a signal from a particle coupled to the analyte) at each array site. Based upon a ratio of sites observed to provide a signal associated with an analyte to a total quantity of detected sites, an array site occupancy may be determined 3460. Steps 3410-3460 may be repeated until a desired occupancy is achieved. In some cases, steps 3410-3460 may be repeated with analytes (e.g., analyte-particle complexes) from a differing source (e.g., a second sample) to form a multiplexed array.

In some cases, a method may utilize a solid support comprising sites and one or more interstitial regions, in which a cumulative surface area of the interstitial region(s) is greater than or equal to a cumulative surface area of the sites. In some cases, a ratio of cumulative surface area of interstitial region(s) to cumulative surface area of sites may be at least about 5, 10, 20, 50, 100, or more than 100. It may be advantageous to increase interstitial surface area relative to site surface area to increase the optical resolvability of individual sites.

In some cases, a method, as set forth herein, may comprise forming an array of analytes, in which the array of analytes is formed on a solid support that is initially substantially devoid of analytes, and in which the array of analytes is formed in no more than about 24 hours, 12 hours, 6 hours, 3 hours, 2 hours, 1 hour, 45 minutes, 30 minutes, 15 minutes, 10 minutes, 5 minutes, 1 minute, 30 seconds, 15 seconds, or less than 15 seconds. Alternatively or additionally, a method, as set forth herein, may comprise forming an array of analytes, in which the array of analytes is formed on a solid support that is initially substantially devoid of analytes, and in which the array of analytes is formed in at least about 15 seconds, 30 seconds, 1 minute, 5 minutes, 10 minutes, 15 minutes, 30 minutes, 45 minutes, 1 hour, 3 hours, 6 hours, 12 hours, 24 hours, or more than 24 hours. In some cases, a step of a method (e.g., contacting with a fluidic medium, performing a mass transfer process, etc.) may occur for at least about 15 seconds, 30 seconds, 1 minute, 5 minutes, 10 minutes, 15 minutes, 30 minutes, 45 minutes, 1 hour, 3 hours, 6 hours, 12 hours, 24 hours, or more than 24 hours. Alternatively or additionally, a step of a method may occur for no more than about 24 hours, 12 hours, 6 hours, 3 hours, 2 hours, 1 hour, 45 minutes, 30 minutes, 15 minutes, 10 minutes, 5 minutes, 1 minute, 30 seconds, 15 seconds, or less than 15 seconds.

A method of forming an array of analytes on a solid support comprising a population of sites, as set forth herein, may comprise a step of contacting the solid support with a population of analytes and/or anchoring moieties. Optionally, the anchoring moieties may be attached to the analytes prior to contact with the solid support. Alternatively, attachment between the anchoring moieties and analytes can occur after contacting the anchoring moieties or analytes to the solid support. In some cases, analytes and/or anchoring moieties may be contacted with an array in a molar excess relative to the population of sites, or an available fraction of the population of sites. For example, analytes and/or anchoring moieties may be provided in a molar excess relative to array sites to ensure a high site occupancy when forming an array of analytes. In other cases, analytes and/or anchoring moieties may be contacted with an array in a molar deficit relative to the population of sites, or an available fraction of the population of sites. For example, analytes and/or anchoring moieties may be provided in a molar deficit relative to array sites due to an insufficient quantity of analytes available in a sample or other source of analytes.

Accordingly, a method, as set forth herein, may comprise providing a population of analytes and/or anchoring moieties (e.g., in a fluidic medium), in which a ratio of a total quantity of the population of analytes or anchoring moieties to the total quantity of a population of array sites of a solid support containing the population of array sites may be at least about 0.000001, 0.00001, 0.0001, 0.001, 0.01, 0.05, 0.1, 0.2, 0.3, 0.4, 0.5, 0.6, 0.7, 0.8, 0.9, 1.0, 1.5, 2, 3, 4, 5, 10, 20, 50, 100, 500, 1000, 10000, 100000, 1000000, or more than 1000000. Alternatively or additionally, a ratio of a total quantity of the population of analytes or anchoring moieties to the total quantity of a population of array sites of a solid support containing the population of array sites may be no more than about 1000000, 100000, 10000, 1000, 500, 100, 50, 20, 10, 5, 4, 3, 2, 1.5, 1.0, 0.9, 0.8, 0.7, 0.6, 0.5, 0.4, 0.3, 0.2, 0.1, 0.05, 0.01, 0.001, 0.0001, 0.00001, 0.000001, or less than 0.000001.

The present disclosure provides methods for forming an array of analytes with high occupancy of analytes at sites of the array. Accordingly, a method may comprise a step of determining an analyte occupancy for an array of analytes. In some cases, a method may comprise detecting at each site of a population of sites of an array of analytes a presence or an absence of an analyte of the population of analytes. In some cases, detecting, at each site of a population of sites of an array of analytes, a presence or an absence of an analyte of the population of analytes may occur before contacting a population of analytes to the array (e.g., to determine analyte occupancy at an initial step or at an intermediate step of an array formation process). In some cases, detecting at each site of a population of sites of an array of analytes a presence or an absence of an analyte of the population of analytes may occur after contacting a population of analytes to the array (e.g., to determine an analyte occupancy at an intermediate step or a final step of an array formation process). In some cases, detecting a presence or an absence of an analyte of a plurality of analytes comprises detecting a signal (e.g., a fluorescent signal, a luminescent signal, a fluorescent or luminescent lifetime signature, a spectroscopic signature, emission of a particle from a radiolabel, etc.) from the analyte or an anchoring moiety attached to the analyte. Detectable labels for providing signals are known in the art, and can include, for example, fluorophores, luminophores, radiolabels, nucleic acid tags, peptide tags, and combinations thereof.

After forming an array of analytes, a method, as set forth herein, may utilize the array of analytes to identify, characterize, or otherwise interrogate analytes of the array of analytes. In some cases, a method may utilize an array of polypeptide analytes to identify, characterize, or otherwise interrogate analytes of the array of analytes. In particular cases, a method may utilize an array of polypeptide analytes to identify or characterize polypeptide analytes by an affinity agent-based assay, such as affinity agent-based sequencing, affinity agent-based identification, or affinity agent-based characterization (e.g., proteoform identification).

A method comprising an affinity agent-based assay may include one or more steps of: i) binding a first plurality of affinity agents to a first fraction of analytes of an array of analytes; ii) detecting affinity agents of the first plurality of affinity agents bound to the first fraction of the analytes at a first fraction of sites of a population of sites of the array of analytes; iii) optionally binding a second plurality of affinity agents to a second fraction of the analytes of the array of analytes, in which the first fraction of analytes differs from the second fraction of analytes; and iv) optionally detecting affinity agents of the second plurality of affinity agents bound to the second fraction of the analytes at a second fraction of sites of the population of sites, in which the first fraction of sites differs from the second fraction of sites. A method comprising an affinity agent-based assay may further comprise: v) identifying an affinity reagent binding profile (e.g., a combination of epitopes recognized by the affinity agents within an analyte or an amino acid sequence of an analyte recognized by the affinity agents, etc.) for analytes of the array of analytes. A method comprising an affinity agent-based assay may further comprise: vi) determining an identity of an analyte based upon an affinity reagent binding profile.

A method comprising a fluorosequencing assay may comprise: vii) fluorescently labeling amino acid residues of analytes of an array of analytes, viii) removing a terminal amino acid residue from the analytes of the array of analytes, and ix) measuring a change or an absence of a change in fluorescence intensity of each analyte of the analytes of the array of analytes. A method comprising a fluorosequencing assay may further comprise: v) identifying a partial amino acid sequence for analytes of the array of analytes. A method comprising a fluorosequencing assay may further comprise: vi) determining an identity of an analyte based upon a partial amino acid sequence.

Additional aspects of methods of analyte characterization are further described below in the section titled “Polypeptide Assays.”

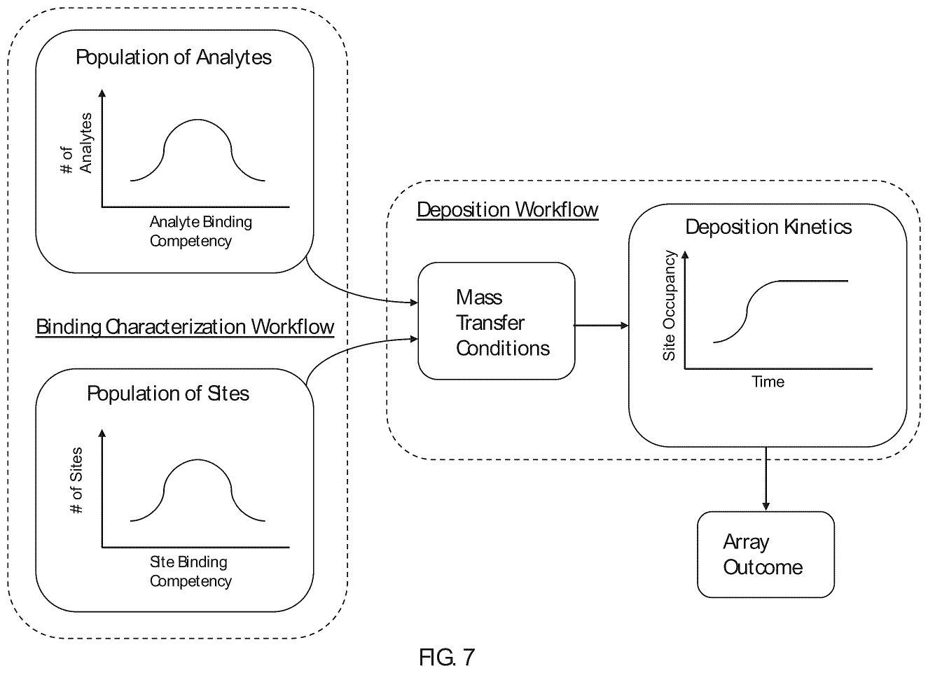

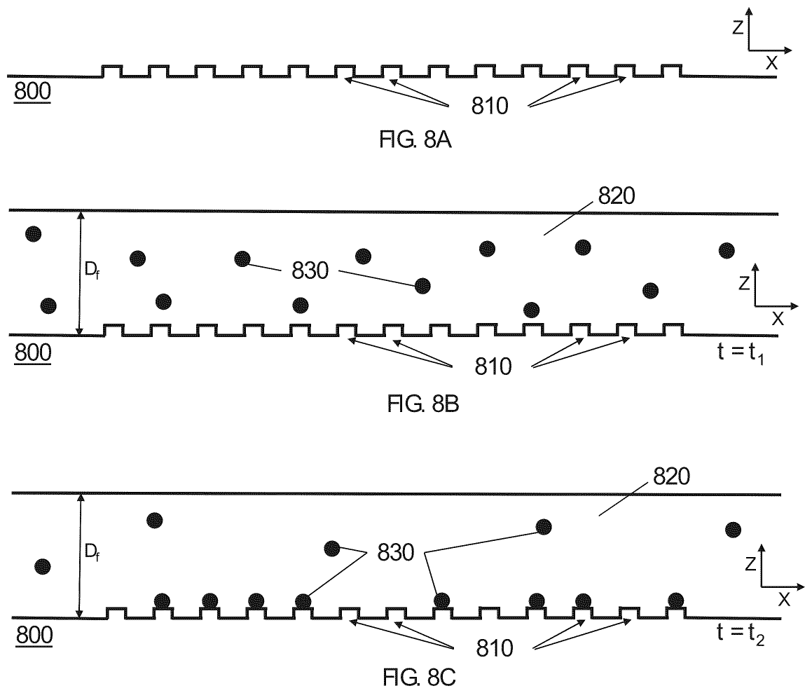

FIGS. 6 and 7 highlight aspects of array formation that can impact the final array occupancy outcome. The present disclosure provides a series of workflows that, alone or in combination, can result in the formation of single-analyte arrays, including in some cases single-analyte arrays that are characterized by super Poisson loading of analytes at array sites. FIG. 6 depicts array site and/or analyte characterization information, for example from a quality control process, that can be combined into a prediction or model of maximum or ideal array occupancy. The upper left chart of FIG. 6 depicts an exemplary array site binding competency profile for a population of about 600 array sites, in which quantities of array sites are provided according to 3 different categories of binding competency. According to the upper left chart, about 100 sites have binding competency 1, 300 sites have binding competency 2, and 200 sites have binding competency 3. The upper right chart of FIG. 6 depicts an exemplary analyte binding competency profile for a population of about 600 analytes, in which quantities of analytes are provided according to 3 different categories of binding competency. The categories of analyte binding competency can be assumed to correspond to the categories of array site binding competency. According to the upper right chart, about 50 analytes have binding competency 1, about 450 analytes have binding competency 2, and about 100 analytes have binding competency 3. The lower left chart of FIG. 6 overlays the two upper charts to demonstrate relative occupancy of sites within each category. Sufficient analytes of category 2 binding competency exist to occupy all array site having category 2 binding competency, but insufficient analytes exist with category 1 or 3 binding competencies to completely occupy all array sites with corresponding binding competencies. As shown on FIG. 6, there are sufficient analytes to achieve a maximum array occupancy of about 75%. This assumes that no analytes co-occupy a single array site, and that no excess analytes (e.g., category 2 analytes) occupy an array site with a category 1 or 3 binding competency.

FIG. 7 extends the array and/or analyte binding characterization workflow to include aspects of analyte deposition workflow in determining array occupancy outcomes. The left side of FIG. 7 depicts elements on a binding characterization workflow, which can include selection or providing of an array with a characterized array site binding competency profile, selection or providing of analytes and/or anchoring moieties with a characterized analyte and/or anchoring moiety binding competency profile, characterization of array site binding competency profile (e.g., by a functional characterization method, as set forth herein), and characterization of analyte and/or anchoring moiety binding competency profile (e.g., by a functional characterization method, as set forth herein). In conjunction with identifying and/or providing an array and population of analytes and/or anchoring moieties for an array formation process, FIG. 7 depicts selection or providing of a deposition workflow, in which the deposition workflow provides sufficient conditions for deposition of analytes of a population of analytes at array sites of an array of sites. The deposition workflow can include method of mass transfer, as set forth herein, including methods for transferring analytes and/or anchoring moieties of a population of analytes and/or anchoring moieties to array sites of a population of array sites. Further, a deposition workflow may include a binding kinetics aspect, in which temporal aspects of forming binding interactions between analytes and/or anchoring moieties and array sites may impact the outcome of an array formation process. Based upon design and selection of the binding characterization workflow and deposition workflow, differing array outcomes can be achieved, including achieved array site occupancy (i.e., percentage of array sites containing at least one analyte), and achieved array site co-occupancy (i.e., percentage of array sites containing at least two analytes).

In another aspect, provided herein is a method, comprising: a) contacting a plurality of analytes to a solid support comprising a plurality of sites, in which each individual analyte is coupled to one and only one particle of a plurality of particles, in which each particle is attached to a plurality of oligonucleotides, and wherein each individual site of the plurality of sites comprises a plurality of complementary oligonucleotides, b) hybridizing oligonucleotides of the plurality of oligonucleotides of particles of the plurality of particles to complementary oligonucleotides of sites of the plurality of sites, thereby coupling analytes of the plurality of analytes to the solid support, and c) after contacting the plurality of analytes to the solid support, detecting a presence of one and only one analyte for at least about 50% (e.g., at least about 55%, 60%, 65%, 70%, 75%, 80%, 85%, 90%, 95%, 99%, 99.9%, or more than 99.9%) of sites of the plurality of sites, and detecting a presence of two or more analytes for no more than about 10% (e.g., no more than about 9%, 8%, 7%, 6%, 5%, 4%, 3%, 2%, 1%, 0.5%, 0.1%, or less than 0.1%) of sites of the plurality of sites, in which the contacting occurs for no more than about 1 hour (e.g., no more than about 45 minutes (min), 30 min, 20 min, 15 min, 10 min, 5 min, 1 min, or less than 1 min), and in which a molar ratio of analytes of the plurality of analytes to sites of the plurality of sites is no more than about 100 (e.g., about 50, 25, 10, 5, 3, 2, 1, 0.5, 0.1, 0.01, or less than 0.01).

Analytes may be attached to a solid support by nucleic acid hybridization between one or more oligonucleotides attached to the analyte (e.g., oligonucleotides attached to an anchoring moiety attached to the analyte) and one or more complementary oligonucleotides attached to an array site. In some cases, oligonucleotides attached to an analyte may be provided with a linking moiety (e.g., a nucleic acid linking moiety, a non-nucleic acid linking moiety). In some cases, complementary oligonucleotides attached to an array site may be provided with a linking moiety (e.g., a nucleic acid linking moiety, a non-nucleic acid linking moiety). Preferably, oligonucleotides attached to an analyte and complementary oligonucleotides attached to an array site may individually be provided with linking moieties. Preferably, oligonucleotides attached to analytes and/or array site may comprise non-rigid linking moieties.

Analytes and/or anchoring moieties may be circulated to facilitate attachment of the analytes and/or anchoring moieties to array sites. Circulation of analytes and/or anchoring moieties can include methods of agitating a fluid, as set forth herein. Circulation of analytes and or anchoring moieties can include repeated delivery and removal of a fluidic medium comprising the analytes and/or anchoring moieties to the solid support. A method may comprise the steps of: (i) removing a fluidic medium comprising analytes and/or anchoring moieties from contact with a solid support; and (ii) after removing the fluidic medium, again contacting the fluidic medium to the solid support. Optionally steps (i) and (ii) can be repeated at least once (e.g., at least 2, 3, 4, 5, 10, 20, 50, 100, or more than 100 times). Optionally, between steps (i) and (ii), a method may further comprise adding additional analytes and/or anchoring moieties to the fluidic medium. For example, analytes and/or anchoring moieties may be added to maintain or increase a concentration of analytes and/or anchoring moieties in the fluidic medium.

A method of array formation may further comprise attaching fiducial elements to array sites. A fiducial element may comprise a detectable address or region of the array that facilitates spatial identification on the array. A fiducial element may provide a landmark or fixed reference for determining position on an array, a measurement of length or distance on the array, a spatial reference for calibrating a detection device (e.g., a sensor or camera), and a spatial reference for registering the addresses of analyte-binding sites consistently over the timespan of an array-based process. In some cases, fiducial elements may be disposed in interstitial regions of a solid support. In other cases, fiducial elements may be disposed at analyte-binding sites of the solid support.

A fiducial element may be formed on a surface of a solid support, preferably at an interstitial region. A fiducial element can be formed by etching the surface of a solid support, thereby providing a fiducial element as a protrusion or depression of the solid support material. Alternatively, a fiducial element can be formed by depositing and/or forming a material (e.g., a metal, metal oxide, or semiconductor) on the surface of the solid support. Alternatively, a particle can be deposited on a surface of the solid support. Various lithographic techniques may be useful for providing fiducial elements on solid support surfaces at fixed addresses and with useful shapes or morphologies. A plurality of fiducial elements can be formed on a solid support at regular or patterned addresses or regions, thereby providing spatial landmarks or a scale for determining relative or absolute distance. Alternatively, fiducial elements may be formed at random locations.

Fiducial elements may be deposited at analyte-binding sites of an array of analytes. It may be preferable to deposit fiducial elements at analyte-binding sites with a random spatial distribution (e.g., providing fiducial elements at a set of analyte-binding sites having no identifiable short-range or long-range spatial pattern). A random spatial distribution of fiducial elements at analyte-binding sites may provide a spatial reference that facilitates analyte-binding site registration when detectable signals are produced from a relatively small quantity of analyte-binding sites (e.g., detectable signals produced from less than about 25%, 20%, 15%, 10%, 5%, 1%, 0.5%, 0.1%, 0.05%, or 0.01% of analyte-binding sites). It may be preferable to provide fiducial elements at at least about 0.01%, 0.05%, 0.1%, 0.5%, 1%, or more than 1% of analyte-binding sites. Alternatively or additionally, fiducial elements may be provided at no more than about 1%, 0.5%, 0.1%, 0.05%, 0.01%, or less than 0.01% of analyte-binding sites. In a preferable configuration, an analyte-binding site may contain one and only one fiducial element. In other configurations, an analyte-binding site may contain more than one fiducial element.

Fiducial elements attached to analyte-binding sites of an array of analytes may comprise a material that provides a detectable signal (e.g., a fluorescent or luminescent signal). Exemplary materials for fiducial elements can include quantum dots or fluorescently-labeled polymer particles. In some cases, a fiducial element may further comprise a coupling moiety that couples to a complementary coupling moiety that is attached to an analyte-binding site. For example, a fiducial element may be functionalized with one or more oligonucleotides that comprise a nucleotide sequence that is complementary to a nucleotide sequence of a site-coupled oligonucleotide. In some cases, a fiducial element and an analyte may be coupled to individual analyte-binding sites by the same type of coupling moiety. For example, fiducial elements and analytes may be attached to individual analyte-binding sites by hybridization of oligonucleotides on the fiducial elements or analytes to complementary oligonucleotides at analyte-binding sites. Alternatively, a fiducial element and an analyte may be coupled to individual analyte-binding sites by a different type of coupling moiety. For example, a fiducial element may be attached electrostatically to a first oligonucleotide-containing analyte-binding site while an analyte may be attached by nucleic acid hybridization to a second oligonucleotide-containing analyte-binding site.

Fiducial elements may be provided at analyte-binding sites to facilitate multiplexed detection of analytes. In configurations utilizing more than one wavelength of light for optical detection, it may be preferable to provide fiducial elements that provide corresponding signals for each detected wavelength of light. For example, in a four-color detection system, it may be preferable to provide fiducial elements that provide signals for each of the four colors of light detected by the system. In some configurations, a fiducial element may be provided on an array that produces signals for each detected wavelength of light (e.g., a multi-color fluorescently-labeled polymer particle).

Fiducial elements may be deposited on an array before or after analytes are deposited on the array. A fluidic medium containing a plurality of fiducial elements may be contacted to a solid support containing a plurality of analyte-binding sites. After contacting the fluidic medium to the solid support, fiducial elements may couple to individual analyte-binding sites, preferably in a random spatial distribution. Alternatively, fiducial elements and analytes may be deposited simultaneously. A fluidic medium comprising a plurality of analyte and a plurality of fiducial elements may be contacted to a solid support containing a plurality of analyte-binding sites. After contacting the fluidic medium to the solid support, fiducial elements and analytes may couple to individual analyte-binding sites, preferably in a random spatial distribution. Additional aspects of arrays containing fiducial elements are described in U.S. Patent Publication No. 2023/0314324 A1, which is herein incorporated by reference in its entirety.

In some cases, a same oligonucleotide may be utilized to attach analytes and/or anchoring moieties and fiducial elements to array sites. In some cases, an analyte or an anchoring moiety may be attached to a first array site by a first oligonucleotide, and a fiducial element may be attached to a second array site by a second oligonucleotide, in which the first oligonucleotide and the second oligonucleotide comprise a same nucleotide sequence (e.g., a nucleotide sequence that is complementary to a nucleotide sequence of oligonucleotides coupled to both the first array site and the second array site).

In some cases, fiducial elements may be contacted to a solid support before analytes and/or anchoring moieties are contacted to the solid support. In some cases, fiducial elements may be contacted to a solid support after analytes and/or anchoring moieties are contacted to the solid support. In some cases, analytes and/or anchoring moieties and fiducial elements may be simultaneously contacted to a solid support. In some cases, analytes and/or anchoring moieties and fiducial elements may be simultaneously delivered to a solid support. Fiducial elements may be contacted to a solid support for at least about 1 second (s), 30 s, 1 minute (min), 5 min, 10 min, 15 min, 30 min, 1 hour (hr), 3 hrs, 6 hrs, 12 hrs, 24 hrs, or more than 24 hrs. Alternatively or additionally, fiducial elements may be contacted to a solid support for no more than about 24 hrs, 12 hrs, 6 hrs, 3 hrs, 1 hr, 30 min, 15 min, 10 min, 5 min, 1 min, 30 s, 1 s, or less than 1 s.

A method may further comprise a step of detecting a presence or absence of a signal from a fiducial element at each array site of a plurality of array sites. Based upon detecting signals at array sites from fiducial elements, a fiducial element array site occupancy may be determined. In some cases, a method may comprise repeating contacting, binding, and/or detection of fiducial elements at array sites until a desired fiducial element array site occupancy is obtained.

An array occupancy that can be achieved in a fixed period of time may be mediated in part by the concentration of available analytes and/or anchoring moieties, and the concentration of unoccupied array sites. Accordingly, it may be advantageous to provide a limited quantity of available array sites at a given time during an array formation method, thereby increasing the quantity of available analytes and/or anchoring moieties relative to the quantity of available array sites.

In some cases, array sites may be provided with blocking groups. A blocking group may comprise a material or moiety disposed at an array site that inhibits binding interactions between coupling moieties attached to analytes and/or anchoring moieties and complementary coupling moieties attached to the array site. Blocking groups may be covalently attached (e.g., photolabile moieties) or non-covalently attached (e.g., binding ligands of the complementary coupling moieties). Blocking groups may be removed by chemical or enzymatic digestion. In a useful configuration, oligonucleotides attached to array sites may comprise double-stranded nucleic acids (e.g., a complementary oligonucleotide hybridized to an array-attached oligonucleotide). The double-stranded nucleic acids may be dehybridized by heating of the array site, thereby providing an available array site for attaching an analyte or anchoring moiety. Localized heating of selected array sites may be provided by a heater coupled to the solid support. Alternatively, localized heating of selected array sites may be provided by illumination of the array sites with a light source. A solid support may comprise a photon-absorbing medium (e.g., an infrared absorber) that produces localized heating when the photon-absorbing medium is illuminated.

Accordingly, a method may comprise a step of removing blocking groups from one or more array sites, then performing a method of array formation, as set forth herein. The step of removing blocking groups may be performed for additional sets of array sites as necessary.

Multiple occupancy or analytes and/or anchoring moieties at array sites may be caused, in part, by formation of multimers of analytes and/or anchoring moieties. If analytes are attached to anchoring moieties, multimers may form before or after the analytes are attached to the anchoring moieties (e.g., two analytes may be attached to a dimer comprising two anchoring moieties, two analyte-coupled anchoring moieties may bind to form a dimer). Accordingly, a method may comprise a step of separating multimers (multimer analytes, multimer anchoring moieties, multimer analyte-anchoring moiety complexes) from a plurality of monomeric analytes, anchoring moieties, or complexes thereof. Multimers may be separated by any suitable method, such as affinity chromatography, size-exclusion chromatography, liquid chromatography, tangential flow filtration, or combinations thereof.

Methods of Forming Arrays with Characterized Binding Competencies

A method of the present disclosure may comprise forming a high-occupancy array of analytes, in which the array of analytes is formed on a solid support comprising a population of sites, and wherein sites of the population of sites have characterized array site binding competencies. In some cases, the solid support comprising the population of sites has a characterized array site binding competency profile, as set forth herein. In some cases, a solid support comprising a plurality of sites may be contacted with a population of analytes and/or anchoring moieties, in which analytes and/or anchoring moieties of the population of analytes and/or anchoring moieties have characterized analyte binding competencies or anchoring moiety binding competencies, as set forth herein. In some cases, the solid support comprising the population of sites may be contacted with a population of analytes and/or anchoring moieties, in which the population of analytes and/or anchoring moieties, in which has a characterized analyte binding competency profile or characterized anchoring moiety binding competency profile.

In an aspect, provided herein is a method of forming an array of analytes, comprising: a) providing a solid support comprising a population of sites, in which the population of sites is characterized by an array site binding competency profile, b) providing a fluidic medium comprising a population of analytes, in which the composition of the population of analytes is based upon the array site binding competency profile, and c) contacting the fluidic medium to the solid support, thereby binding analytes of the population of analytes to sites of the population of sites to form the array of analytes, in which the array of analytes has a site occupancy of at least 50% (e.g., at least 55%, at least 60%, at least 65%, at least 70%, at least 75%, at least 80%, at least 85%, at least 90%, at least 95%, at least 99%), and in which the array of analytes has a site co-occupancy of no more than 10% (e.g., no more than 9%, no more than 8%, no more than 7%, no more than 6%, no more than 5%, no more than 4%, no more than 3%, no more than 2%, no more than 1%, no more than 0.5%, no more than 0.1%).

In some cases, analytes of a population of analytes may be deposited at array sites of a population of array sites. An analyte may be coupled to an array site by a binding interaction between the analyte and the array site. In some cases, an analyte may comprise a coupling moiety that binds to a site-coupled coupling moiety. An analyte may comprise a coupling moiety that is endogenous to the analyte. For example, a polypeptide or polynucleotide analyte may comprise a region of electrical charge density that forms an electrostatic binding interaction with an electrically-charged coupling moiety of an array site. An analyte may comprise a coupling moiety that is exogenous to the analyte (e.g., comprising a residue sequence that does not naturally occur in the analyte, or comprising a functional group or moiety that does not naturally occur in the analyte). For example, a polypeptide analyte may be attached to one or more oligonucleotide coupling moieties, or attached to one or more receptor-ligand binding pair components (e.g., streptavidin or biotin, SpyCatcher or SpyTag, SnoopCatcher or SnoopTag, etc.).

It may be advantageous to couple an analyte of a population of analytes to an anchoring moiety. An anchoring moiety may comprise a moiety, molecule, or particle that mediates a binding interaction between an analyte and an array site. An anchoring moiety may facilitate binding of analytes to array sites by increasing specificity of binding between an analyte and an array site, or providing a more homogeneous binding property to an analyte. For example, a sample of polypeptide analytes may vary in numerous chemical properties (e.g., molecular weight, hydrodynamic radius, net electrical charge, hydrophobicity, etc.), so attaching an anchoring moiety to each polypeptide analyte of the sample of polypeptide analytes may provide a more uniform binding characteristic to the polypeptide analytes. In a particular case, an analyte of a population of analytes can be linked to one and only one anchoring moiety of a population of anchoring moieties. A method may further comprise attaching an analyte of a population of analytes to one and only one anchoring moiety of a population of anchoring moieties. In another particular cases, two or more analytes of a population of analytes can be linked to one and only one anchoring moiety of a population of anchoring moieties. A method may further comprise attaching two or more analytes of a population of analytes to one and only one anchoring moiety of a population of anchoring moieties.

In some cases, binding analytes and anchoring moieties of a population of analytes and anchoring moieties to array sites of a population of array sites comprises binding an anchoring moiety of the population of anchoring moieties to an array site of the population of array sites, wherein the anchoring moiety is attached to an analyte of the population of analytes. In some cases, an anchoring moiety may comprise a non-covalent coupling moiety. In some cases, binding an anchoring moiety of a population of anchoring moieties to an array site of a population of array sites comprises binding a non-covalent coupling moiety of the anchoring moiety to a non-covalent coupling moiety of the array site. In some cases, an anchoring moiety may comprise a covalent coupling moiety. In some cases, binding an anchoring moiety of a population of anchoring moieties to an array site of a population of array sites may comprise binding a covalent coupling moiety of the anchoring moiety to a covalent coupling moiety of the array site.

When analytes are coupled to anchoring moieties, the binding interactions between analytes and array sites may be largely governed by binding interactions between anchoring moieties and array sites. Accordingly, binding behavior of a population of analytes coupled to a population of anchoring moieties may be characterized by an anchoring moiety binding competency profile.

A fluidic medium may comprise a population of analytes, a population of anchoring moieties, or a population of analytes attached to anchoring moieties (e.g., attached covalently or attached non-covalently). In some cases, a fluidic medium may comprise an analyte of a population of analytes that is not attached to an anchoring moiety of a population of anchoring moieties. In some cases, a fluidic medium may comprise a plurality of analytes of a population of analytes, in which the plurality of analytes is not attached to anchoring moieties of a population of anchoring moieties. In some cases, a fluidic medium may comprise an anchoring moiety of a population of anchoring moieties that is not attached to an analyte of a population of analytes. In some cases, a fluidic medium may comprise a plurality of anchoring moieties of a population of anchoring moieties, in which the plurality of anchoring moieties is not attached to analytes of the plurality of analytes.

In some cases, providing a fluidic medium comprising a population of analytes can comprise: e) attaching analytes of the population of analytes to anchoring moieties of a population of anchoring moieties, and f) disposing the population of analytes and the population of anchoring moieties within the fluidic medium. In particular cases, a method may further comprise a purification step, in which, after attaching the analytes of the population of analytes to anchoring moieties of the population of anchoring moieties, an analyte of the population of analytes that is not attached to an anchoring moiety is separated and/or removed from the other analytes. In particular cases, a method may further comprise a purification step, in which, after attaching the analytes of the population of analytes to anchoring moieties of the population of anchoring moieties, an anchoring moiety of the population of anchoring moieties that is not attached to an analyte is separated and/or removed from the other anchoring moieties.

For an array containing a population of sites in which the sites have a range of binding competencies, it can be advantageous to provide a population of binding entities (e.g., analytes, anchoring moieties, or analytes attached to anchoring moieties) with a similar range of binding competencies. Such populations of binding entities may be provided as one or more standardized reagents (e.g., from a commercially available product or kit), in which the one or more standardized reagents have a characterized binding competency profile (e.g., an analyte binding competency profile or an anchoring moiety binding competency profile). A population of binding entities may be selected from one or more standardized reagents by selecting a standardized reagent from the one or more standardized reagents, in which the standardized reagent has a binding competency profile most similar to an array site binding competency profile of an array containing a population of sites.

In some cases, a population of binding entities (e.g., analytes, anchoring moieties, or analytes attached to anchoring moieties) may be prepared or formulated based upon a characterized array site binding competency profile of an array containing a population of sites. For example, a population of analytes and/or anchoring moieties may be formulated by combining two or more subpopulations of analytes and/or anchoring moieties. Two or more subpopulations of analytes or anchoring moieties may be combined, in which a first subpopulation of analytes and/or anchoring moieties comprises a first analyte or first anchoring moiety with a first binding competency, and in which a second subpopulation of analytes and/or anchoring moieties comprises a second analyte or second anchoring moiety with a second binding competency, in which the first binding competency differs from the second binding competency. Two or more subpopulations of analytes or anchoring moieties may be combined, in which a first subpopulation of analytes and/or anchoring moieties comprises a first analyte or first anchoring moiety with a first binding competency, and in which a second subpopulation of analytes and/or anchoring moieties comprises a second analyte or second anchoring moiety with a second binding competency, in which the first binding competency is the same as the second binding competency.

In some cases, a subpopulation or quantity of analytes and/or anchoring moieties may be provided or formulated based upon an array site binding competency profile of an array containing a population of sites. An array site binding competency profile may provide quantitative information, such as array site quantity as a function of various measures or categories of array site binding competency. In some cases, providing a subpopulation or quantity of analytes and/or anchoring moieties of a population of analytes or anchoring moieties can comprise: h) identifying a subpopulation or quantity of sites comprising a same array site binding competency based upon the array site binding competency profile; and i) providing the subpopulation or quantity of analytes to a fluidic medium, in which each analyte of the quantity of analytes comprises a same binding competency for the subpopulation or quantity of sites. In some cases, a provided subpopulation or quantity of analytes and/or anchoring moieties may be greater than a subpopulation or quantity of sites. For example, a subpopulation of analytes may be provided in molar excess relative to a subpopulation of sites to enhance the binding kinetics of the subpopulation of analytes to the subpopulation of sites. In some cases, a provided subpopulation or quantity of analytes and/or anchoring moieties may be less than or equal to a subpopulation or quantity of sites. For example, a subpopulation of analytes may be provided in molar deficit relative to a subpopulation of sites due to an insufficient quantity of analytes in a sample of analytes.

In some cases, providing a population or quantity of analytes and/or anchoring moieties can comprise: j) determining two or more subpopulations of array sites, in which each subpopulation of array sites comprises a quantity of sites comprising a same array site binding competency based upon the array site binding competency profile; and k) providing two or more subpopulations of analytes and/or anchoring moieties to the fluidic medium, in which each subpopulation of analytes and/or anchoring moieties comprises a quantity of analytes and/or anchoring moieties, and in which each analyte or anchoring moiety of a quantity of analytes and/or anchoring moieties of a subpopulation of analytes and/or anchoring moieties comprises a binding competency for the quantity of array sites of a corresponding subpopulation of array sites.

A population of array sites may comprise one and only one species of array site, in which the species of array site has a common array site binding competency amongst all sites of the population of array sites. A population of array sites may comprise two or more species of array sites, in which each species of array site has a subpopulation of the population of array sites, and in which each array site of a subpopulation has a common array site binding competency amongst all sites of the subpopulation of array sites.

A population of analytes may comprise one and only one species of analyte. Each analyte of a population of analytes comprising one and only one species of analyte may have the same analyte binding competency. A population of analytes comprising one and only one species of analyte may comprise a first analyte with a first analyte binding competency and a second analyte with a second analyte binding competency, in which the first analyte binding competency differs from the second analyte binding competency. For example, two isoforms of a same species of polypeptide may comprise differing analyte binding competencies due to structural differences between the isoforms. A population of analytes may comprise two or more species of analytes (e.g., polypeptide species, polynucleotide species, polysaccharide species, etc.). A population of analytes may comprise a first analyte species and a second analyte species, in which an analyte of the first analyte species and an analyte of the second analyte species have a same analyte binding competency. A population of analytes may comprise a first analyte species and a second analyte species, in which an analyte of the first analyte species and an analyte of the second analyte species have a differing analyte binding competency. A population of analytes may comprise a diversity of analyte species, such as a genomic-scale diversity, a proteomic-scale diversity, or a microbiome-scale diversity.

A population of anchoring moieties may comprise one and only one species of anchoring moiety. Each anchoring moiety of a population of anchoring moieties comprising one and only one species of anchoring moiety may have the same anchoring moiety binding competency. A population of anchoring moieties comprising one and only one species of anchoring moiety may comprise a first anchoring moiety with a first anchoring moiety binding competency and a second anchoring moiety with a second anchoring moiety binding competency, in which the first anchoring moiety binding competency differs from the second anchoring moiety binding competency. For example, quantity of coupling moieties on anchoring moieties may vary stochastically when provided from a manufacturing process. A population of anchoring moieties may comprise two or more species of anchoring moieties. A population of anchoring moieties may comprise a first anchoring moiety species and a second anchoring moiety species, in which an anchoring moiety of the first anchoring moiety species and an anchoring moiety of the second anchoring moiety species have a same anchoring moiety binding competency. A population of anchoring moieties may comprise a first anchoring moiety species and a second anchoring moiety species, in which an anchoring moiety of the first anchoring moiety species and an anchoring moiety of the second anchoring moiety species have a differing anchoring moiety binding competency.

A method, as set forth herein, may comprise contacting a fluidic medium comprising a population of analytes and/or anchoring moieties with an array comprising a population of array sites, in which the contacting occurs a particular number of times, such as at least about 1, 2, 3, 4, 5, 6, 7, 8, 9, 10, 15, 20, 25, 50, 100, or more than 100 times. Alternatively or additionally, contacting of a fluidic medium may occur no more than about 100, 50, 25, 20, 15, 10, 9, 8, 7, 6, 5, 4, 3, 2, or no more than 1 time. A method of contacting a fluidic medium containing a population of analytes and/or anchoring moieties with an array more than once may further comprise displacing the fluidic medium from contacting the array with an equivalent volume of a second fluidic medium (e.g., a gaseous fluid or a liquid fluid), in which the second fluidic medium does not comprise the population of analytes and/or anchoring moieties. In some cases, no additional analytes are added to a fluidic medium during contacting of the fluidic medium to the solid support. In some cases, a method may further comprise contacting a second fluidic medium to an array comprising a population of array sites, in which the second fluidic medium comprises a second population of analytes and/or anchoring moieties.

An array of analytes may be formed by depositing two or more species of binding entities (e.g., analytes, anchoring moieties, analytes coupled to anchoring moieties) at sites of a population of sites. In some cases, a first species of binding entity of two or more species of binding entities can differ from a second species of binding entity of the two or more species of binding entities with respect to a binding entity binding property. Exemplary binding entity properties that may distinguish two or more species of binding entities are listed in Table II. In some cases, the first species of binding entity of the two or more species of binding entities differs from a second species of binding entity of the two or more species of binding entities with respect to two or more binding entity binding properties. In some cases, a population of array sites can comprise two or more species of array sites. In some cases, a first species of array site of two or more species of array sites can differ from a second species of array site of the two or more species of array sites with respect to an array site binding property. Exemplary array site properties that may distinguish two or more species of array sites are listed in Table II. In some cases, the first species of array site of the two or more species of array sites can differ from a second species of array site of the two or more species of array sites with respect to two or more array site binding properties

### Methods of Improving Mass Transfer During Array-Based Processes

An array-based method, such as a method of forming an array of analytes or a method of characterizing an array of analytes, as set forth herein, may comprise one or more steps that effect a transfer of transportates, (e.g., macromolecules, particles, analytes, anchoring moieties, affinity agents, detection reagents, etc) from a fluidic medium to an array site. Methods of facilitating mass transfer of transportates during array formation may fall into one or both of two categories: Category 1—transfer of transportates toward a surface of a solid support, in which the solid support comprises a population of array sites, and Category 2—transfer of transportates across the surface of the solid support. In some cases, a method can include both transfer of transportates toward a surface (Category 1), and transfer of transportates across the surface. Referring to FIG. 3, the aforementioned category 1 of mass transfer would be depicted in the predominant motion of the moiety 310 in the z-axis direction toward the surface of the solid support 300, as shown in the upper left to upper right diagrams. The aforementioned category 2 of mass transfer would be depicted in the motion of the analyte in the plane defined by the x-axis and y-axis toward the array site 305, as shown in the upper right and lower center diagrams. Although certain examples and figures set forth herein are exemplified with respect to analytes and/or anchoring moieties, it shall be understood that the concepts of mass transfer disclosed herein may apply to any transportate, as set forth herein.

Mass transfer effects can affect the rate of transportate deposition or binding on solid supports containing populations of sites. Without wishing to be bound by theory, a rate of transportate binding by array sites may be affected by a concentration of unoccupied array sites (e.g., available array sites that do not contain a bound analyte or anchoring moiety) and a concentration of transportates.

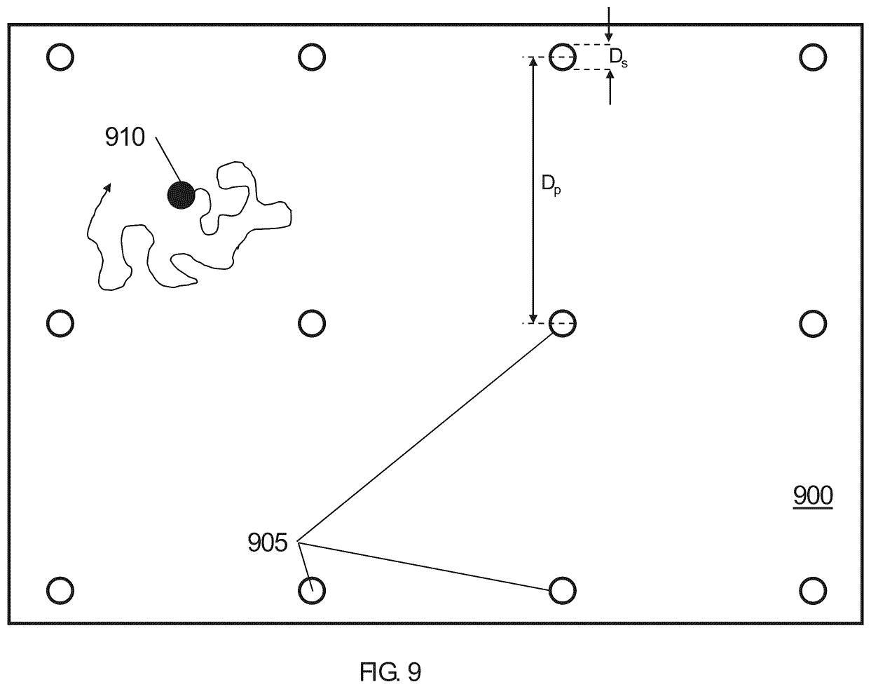

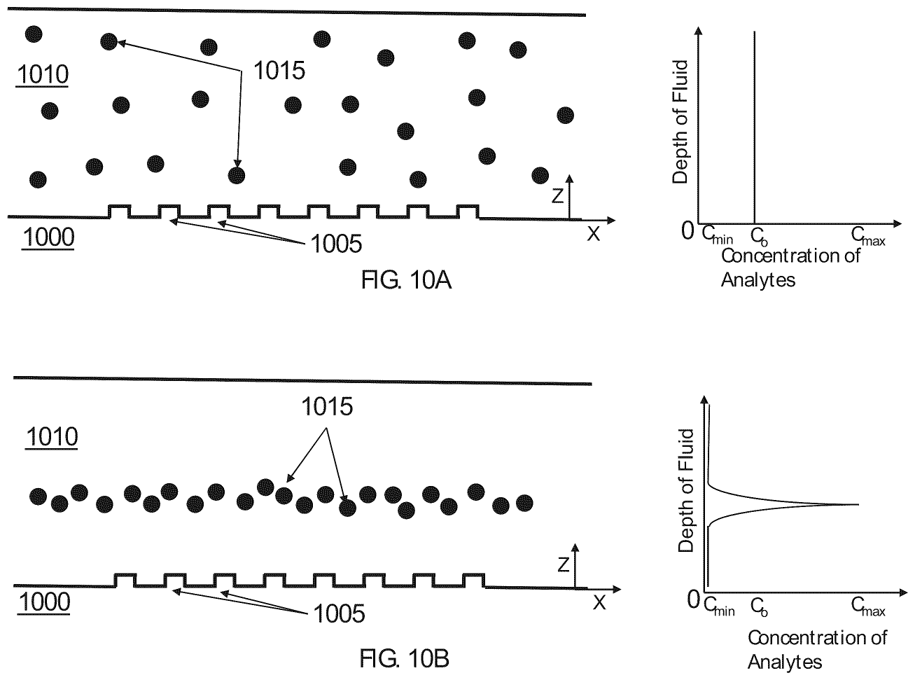

FIGS. 8A-8C depict the affect of array site concentration and analyte concentration on binding rate. FIG. 8A depicts a solid support 800 comprising a population of array sites 810. In FIG. 8B, the solid support 800 comprising the population of array sites 810 is contacted with a fluidic medium 820 of depth D_{f} at an initial time t₁. The fluidic medium 820 comprises a population of analytes 830 that have a substantially uniform concentration in the fluidic medium 820 of N_{A}/(A*D_{f}), wherein N_{A} is the total molar quantity of unbound analytes 830 in the fluidic medium 820, and A is the surface area of the solid support 800, including the surface area of the array sites 810. FIG. 8C depicts the solid support 800 and fluidic medium 820 at a time t₂, in which a fraction of analytes 830 of the population of analytes have deposited at array sites 810 of the solid support 800. Assuming the analytes 830 diffuse freely through the fluidic medium 820, the overall concentration of analytes decreases due to the decrease in unbound analytes 830. Accordingly, the decrease in concentration of unbound analytes 830 and unbound array sites 810 suggests that a likelihood of an unbound analyte 830 approaching an array site 810 at a close enough distance to facilitate a binding interaction has decreased by time t₂ relative to t₁. For array systems in which a fluidic thickness D_{f} is significantly larger than an inter-site pitch or spacing, the decrease in analyte and/or anchoring moiety concentration during analyte and/or anchoring moiety deposition may primarily inhibit Category 1 mass transfer (i.e., transfer in the z-axis direction) as a function of time absent a mechanism of active transport in the z-axis direction toward an array surface.

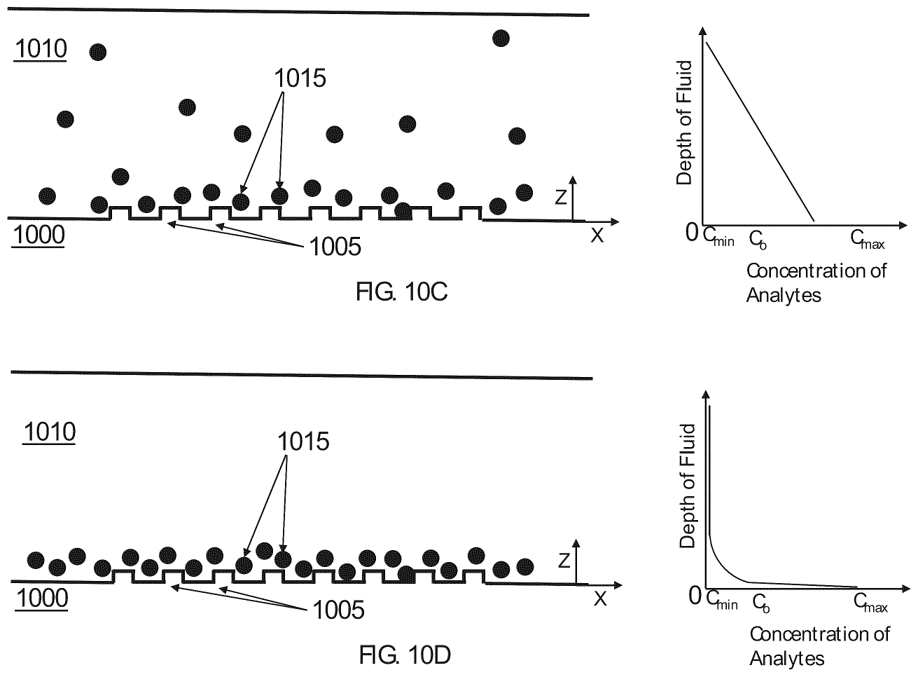

FIG. 9 depicts mass transfer effects of an analyte 910 on a surface of a solid support 900 containing a population of array sites 905. The array sites have a characteristic feature size Dₛ (e.g., a site diameter) and a characteristic inter-site pitch Dₚ. For the array depicted in FIG. 9, the ratio of Dₚ/Dₛ is about 10:1. Accordingly, approximately 99% of the surface of the solid support 900 comprises an interstitial region. For an analyte 910 randomly placed on the surface of the solid support 900, the likelihood of being located on or adjacent to an array site 905 is low, and diffusion by random walk (as shown in FIG. 9) is also unlikely to result in contacting an array site, even if the analyte 910 does not form binding interactions with the interstitial region. Accordingly, Category 2 mass transfer (i.e., transfer within the x-y plane) may be unlikely to result in binding of analytes 910 to array sites 905 absent a mechanism of active transport in the x-y plane.

A method, as set forth herein, may comprise providing one or more transportate association conditions to a solid support comprising a population of sites, or a fluidic medium contacted thereto. An analyte association condition may refer to a step of a method that increases a rate of transport of analytes and/or anchoring moieties to array sites. An analyte association condition may refer to a method that increases a rate of transport toward a surface containing a population of array sites. An analyte association condition may refer to a method that increases a rate of transport across a surface toward array sites. An analyte association condition may comprise a method of active transport of analytes and/or anchoring moieties if an externally-applied force drives mass transfer of analytes and/or anchoring moieties (e.g., by fluid flow, fluid displacement, fluid pressure, electrophoresis, magnetic attraction, magnetic repulsion, centrifugal force, etc.). A transportate association condition may comprise a method of passive transport of transportates during mass transfer of the transportates (e.g., by sedimentation, precipitation, hydrophobic/hydrophilic repulsion, electrostatic repulsion, etc.). In some cases, an array system may comprise two or more surfaces, in which a first surface comprises a population of array sites, in which a second surface comprises no array sites, in which a transportate association condition increases mass transfer of transportates toward the first surface, and in which the transportate association condition decreases mass transfer of transportate toward the second surface. For example, a substantially planar array of sites may be disposed within a chamber of a flow cell or fluidic cartridge, in which the chamber of the flow cell or fluidic cartridge may be enclosed by a substantially planar backer or cover slip, and in which the backer or cover slip contains a surface that is offset and opposed from a surface containing the array sites. In such a case, a transportate association condition may increase a concentration of transportates adjacent to the surface containing the array sites, and may decrease a concentration of transportates adjacent to the surface of the backer or cover slip.

A method, as set forth herein, may comprise providing two or more differing transportate association conditions, as set forth herein, such as 2, 3, 4, 5, or more than 5 transportate association conditions. Two or more transportate association conditions may be performed sequentially. Two or more transportate association conditions may be performed simultaneously. Two or more transportate association conditions may be performed consecutively. Two or more transportate association conditions may be performed non-consecutively.

### Methods of Increasing Mass Transfer to an Array Surface

A method, as set forth herein, may include an analyte association condition that increases mass transfer of transportates (e.g., macromolecules, particles, analytes, anchoring moieties, affinity agents, detection reagents, etc) toward a surface of a solid support, in which the surface contains a population of array sites. A transportate association condition that increases mass transfer of transportates toward a surface of a solid support may be provided to increase the site occupancy of an array of analytes, to decrease formation time of an array of analytes, to increase a fraction of sites to which an assay agent binds, or a combination thereof.

It will be understood by the skilled person that methods of generating a flux of transportates toward a surface of a solid support, as described below, may also produce fluxes of transportates in at least one other direction, including along a surface of a solid support. Methods described in this section should not be construed as necessarily being limited to a one-dimensional or uni-directional flux of transportates, but rather as providing methods that can advantageously increase flux (relative to a diffusive flux) of transportates in a direction toward and substantially orthogonal to array sites on a surface of a solid support.

A method set forth herein may comprise a step of contacting a solid support comprising a population of array sites with a fluidic medium containing a population of transportates. In some cases, a method set forth herein may comprise a step of contacting a solid support comprising a population of array sites with a fluidic medium containing a population of transportates, in which the fluidic medium has a substantially spatially isotropic concentration of the transportates. In other cases, a method set forth herein may comprise a step of contacting a solid support comprising a population of array sites with a fluidic medium containing a population of transportates, in which the fluidic medium has a spatially anisotropic concentration of transportates. For example, a microfluidic effect such as inertial focusing may concentrate particles (e.g., analytes, anchoring moieties or analytes attached to anchoring moieties) along a streamline of a fluidic flow field.

In some cases, after contacting a solid support comprising a population of array sites with a fluidic medium containing a population of transportates, the fluidic medium can have a substantially spatially isotropic concentration of transportates. In other cases, after contacting a solid support comprising a population of array sites with a fluidic medium containing a population of transportates, the fluidic medium can have a substantially spatially anisotropic concentration of transportates.

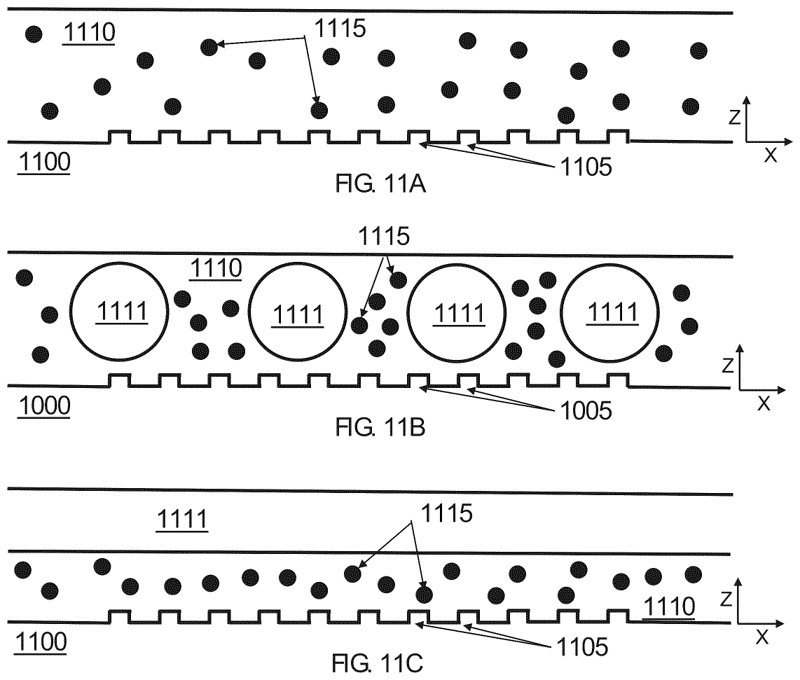

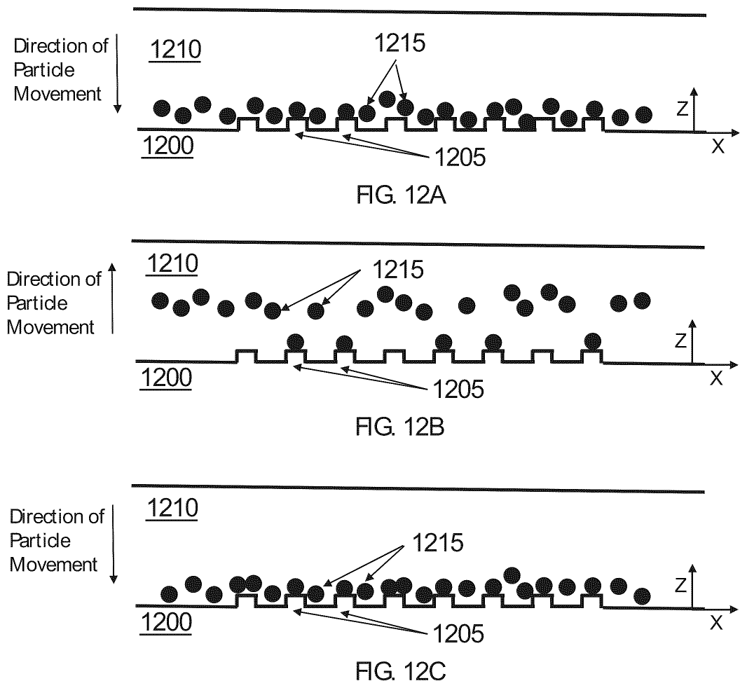

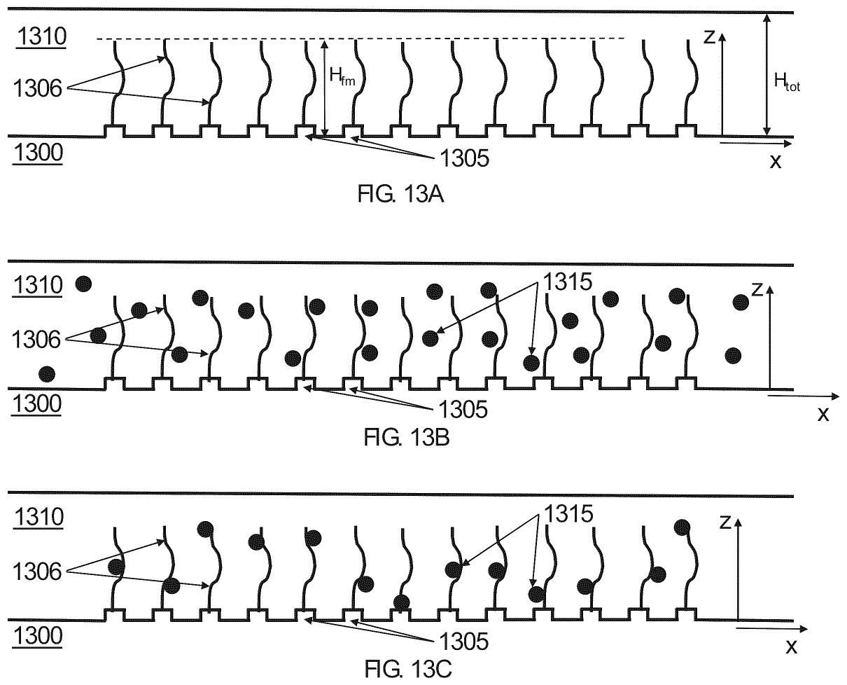

FIGS. 10A-10B depict spatial distributions of analytes and/or anchoring moieties 1015 within a fluidic medium 1010 during or after contacting of the fluidic medium 1010 with a solid support 1000 containing a population of sites 1005. FIG. 10A illustrates a configuration in which the fluidic medium 1010 comprises a substantially homogeneous distribution of analytes and/or anchoring moieties 1015 (i.e., spatially isotropic concentration). To the right of the illustration is an exemplary plot depicting the invariant concentration of analytes and/or anchoring moieties 1015 as a function of depth in the fluidic medium 1010 relative to the surface of the solid support 1000 that is contacted with the fluidic medium 1010. For the configuration of FIG. 10A, a measured concentration of analytes and/or anchoring moieties 1015 in any subvolume of the fluidic medium 1010 would be expected to be about the same as the average overall concentration, Cₒ, for the entire volume of the fluidic medium 1010. FIG. 10B illustrates a configuration in which the fluidic medium 1010 comprises a spatially anisotropic distribution of analytes and/or anchoring moieties 1015. To the right of the illustration is an exemplary plot depicting the varying concentration of analytes and/or anchoring moieties 1015 as a function of depth in the fluidic medium 1010 relative to the surface of the solid support 1000 that is contacted with the fluidic medium 1010. For the configuration of FIG. 10B, a measured concentration of analytes and/or anchoring moieties 1015 in a subvolume of the fluidic medium 1010 taken within a region of concentrated analyte and/or anchoring moieties 1015 would be expected to have a concentration between Cₒ and Cₘₐₓ, a maximal concentration of analytes and/or anchoring moieties 1015. Subvolumes of the fluidic medium away from the region of concentrated analytes and/or anchoring moieties 1015 would be expected to have a concentration of analytes and/or anchoring moieties 1015 that approaches Cₘᵢₙ (which can be a zero or near-zero concentration).

FIGS. 10C-10D illustrates configurations in which the array system has undergone or is undergoing an analyte association condition that increases mass transfer of analytes and/or anchoring moieties 1015 toward the surface of the solid support 1000 containing the population of array sites 1005. As shown in FIG. 10C, the concentration of analytes and/or anchoring moieties 1015 is increasing adjacent to the surface of the solid support 1000. To the right of the illustration is an exemplary plot depicting the varying concentration of analytes and/or anchoring moieties 1015 as a function of depth in the fluidic medium 1010 relative to the surface of the solid support 1000 that is contacted with the fluidic medium 1010. For the configuration of FIG. 10C, a measured concentration of analytes and/or anchoring moieties 1015 has a substantially linear relationship with depth in the fluidic medium 1010, ranging from about Cₘᵢₙ at furthest distances from the surface of the solid support 1000 to a value between Cₒ and Cₘₐₓ adjacent to the surface of the solid support 1010. As shown in FIG. 10D, the analytes and/or anchoring moieties 1015 have become concentrated adjacent to the surface of the solid support 1000 by the analyte association condition. Subvolumes of the fluidic medium 1010 adjacent to the surface of the solid support 1000 would have a concentration of analytes and/or anchoring moieties 1015 approaching Cₘₐₓ, while the concentration of analytes and/or anchoring moieties 1015 at most depths of the fluidic medium beyond depths adjacent to the surface of the solid support 1000 would have a concentration of analytes and/or anchoring moieties 1015 approaching Cₘᵢₙ. Preferably, an analyte association condition would produce a configuration like that of FIG. 10D because the increased relative concentrations of analytes and/or anchoring moieties 1015 and array sites 1005 would increase a likelihood of analytes and/or anchoring moieties 1015 binding to array sites 1005.

In an aspect, provided herein is a method of forming an array of analytes, comprising: a) providing a solid support with a surface, in which the surface is substantially planar, and in which a population of sites is disposed on the surface, b) contacting the array with a fluidic medium comprising a population of anchoring moieties, in which each anchoring moiety is attached to an analyte, c) after contacting the array with the fluidic medium, providing a first analyte association condition, in which the first analyte association condition forms a concentration gradient of anchoring moieties in the fluidic medium, and wherein the concentration gradient of anchoring moieties is characterized by an increasing concentration of anchoring moieties as distance to the surface of the solid support decreases in a direction orthogonal to the surface of the solid support, and d) binding anchoring moieties of the plurality of anchoring moieties to sites of the population of sites, in which the single-pass site occupancy is at least 75% of the population of sites within no more than about 6 hours, 3 hours, 2 hours, 60 minutes, 45 minutes, 30 minutes, 20 minutes, 15 minutes, 10 minutes, 5 minutes, or 1 minute of contacting the solid support with the fluidic medium.

An analyte association condition may comprise altering a fluidic medium comprising a population of transportates. A fluidic medium can be altered in a manner that alters the solubility of transportates (e.g., increasing the solubility of analytes and/or anchoring moieties or decreasing the solubility of analytes and/or anchoring moieties). A fluidic medium can be altered in a manner that decreases the solubility of transportates without causing substantial aggregation or precipitation of the transportates. A fluidic medium can be altered in a manner that increases the concentration of transportates adjacent to a surface of a solid support containing a population of array sites.

An transportate association condition may comprise altering a fluidic medium comprising a population of transportates, in which altering the fluidic medium comprises one or more conditions of: 1) altering a pH of the fluidic medium, 2) altering a quantity of a chemical species of a fluidic medium (e.g., altering ionic strength of an ionic species, altering concentration of a surfactant, altering concentration of a buffering species, etc.), 3) adding a chemical species to a fluidic medium, 4) removing a chemical species from a fluidic medium, and 5) altering a fluidic property of a fluidic medium, such as density or viscosity (e.g., by heating or cooling the fluidic medium).

Altering a fluidic medium may increase mass transfer of transportates toward a surface containing a population of array sites. Altering a fluidic medium can increase mass transfer of transportates toward a surface containing a population of array sites by altering a property of a transportate in the fluidic medium, such as solubility (e.g., increased solubility or decreased solubility), net surface electrical charge (e.g., increased net surface electrical charge or decreased net surface electrical charge), colloidal dispersion stability (e.g., increased stability or decreased stability), hydrodynamic radius (e.g., increased hydrodynamic radius or decreased hydrodynamic radius), sedimentation rate (e.g., increased sedimentation rate or decreased sedimentation rate), or combinations thereof. Altering a fluidic medium can increase mass transfer of transportates toward a surface containing a population of array sites by altering a property of the fluidic medium, such as density (e.g., increased density or decreased density), viscosity (e.g., increased viscosity or decreased viscosity), ionic strength (e.g., increased ionic strength or decreased ionic strength), polarity (e.g., increased polarity or decreased polarity), thermal conductivity (e.g., increased thermal conductivity or decreased thermal conductivity), thermal diffusivity (e.g., increased thermal diffusivity or decreased thermal diffusivity), or combinations thereof. A person skilled in the art will recognize both the impact of described methods of altering fluidic media on fluid and/or transportate properties, as well as methods for altering a fluidic medium to achieve a desired mass transfer result.

A transportate association condition may comprise altering a pH of a fluidic medium. Altering a fluidic medium may comprise altering a pH of the fluidic medium (e.g., increasing the pH or decreasing the pH) by at least about 0.1, 0.2, 0.3, 0.4, 0.5, 0.6, 0.7, 0.8, 0.9, 1.0, 1.2, 1.3, 1.4, 1.5, 1.6, 1.7, 1.8, 1.9, 2.0, 2.5 3.0, 3.5, 4.0, 4.5, 5.0, 6.0, 7.0, 8.0, 9.0, 10.0, or more than 10.0 pH units. Alternatively or additionally, altering a fluidic medium may comprise altering a pH of the fluidic medium (e.g., increasing the pH or decreasing the pH) by no more than about 10.0, 9.0, 8.0, 7.0, 6.0, 5.0, 4.5, 4.0, 3.5, 3.0, 2.5, 2.0, 1.9, 1.8, 1.7, 1.6, 1.5, 1.4, 1.3, 1.2, 1.1, 1.0, 0.9, 0.8, 0.7, 0.6, 0.5, 0.4, 0.3, 0.2, 0.1, or less than 0.1 pH units.

An analyte association condition may comprise altering the quantity of a chemical species of a fluidic medium. Altering the quantity of a chemical species of a fluidic medium can refer to altering a quantity of a chemical species that is present within the fluidic medium (as opposed to adding a previously non-existing chemical species to the fluidic medium). A fluidic medium may comprise one or more chemical species other than transportates. A change in quantity of a chemical species within a fluidic medium may occur by a method such as concentrating the fluidic medium, diluting the fluidic medium, adding an additional quantity of the chemical species to the fluidic medium, or removing a fraction of the quantity of the chemical species from the fluidic medium. In some cases, a transportate association condition may comprise altering quantities of two or more chemical species of a fluidic medium. Measures of chemical species quantity (and changes thereof) are well known in the art and can include concentration, molarity, molality, normality, weight percent or fraction, volume percent or fraction, specific weight, total mass, total weight, or total molar amount.

It will be understood by the skilled person that differing chemical species are provided to a fluidic medium in differing amounts depending upon the formulation and purpose of the fluidic medium. For example, a fluidic medium containing nucleic acids may comprise a magnesium salt to maintain nucleic acid stability, as well as a buffering species and/or other salt species that may be provided at 1 to 2 orders of magnitude greater molar concentration than the magnesium salt. The numerous advantageous chemical species that may be provided in a fluidic medium can each have a unique concentration or range of useful concentrations depending on the purpose of the fluidic medium. Accordingly, due to the many possible units of measure and ranges of quantities of chemical species, it may be useful to represent changes in a quantity of a chemical species in terms of a fractional or percentage change rather than an absolute change in a unit of measure. For example, a change in molar concentration of a chemical species from about 100 milliMolar (mM) to about 200 mM would be about a 100% increase in the concentration of the chemical species.

A transportate association condition may comprise increasing a quantity of a chemical species (e.g., molarity, molality, normality, weight percent or fraction, specific weight, total mass or weight, or total molar amount, etc.) in a fluidic medium by at least about 0.1%, 0.5%, 1%, 5%, 10%, 20%, 30%, 40%, 50%, 60%, 70%, 80%, 90%, 100%, 150%, 200%, 300%, 400%, 500%, 750%, 1000%, or more than 1000%. A transportate association condition may comprise increasing a quantity of a chemical species in a fluidic medium by no more than about 1000%, 750%, 500%, 400%, 300%, 200%, 150%, 100%, 90%, 80%, 70%, 60%, 50%, 40%, 30%, 20%, 10%, 5%, 1%, 0.5%, 0.1%, or less than 0.1%.

A transportate association condition may comprise decreasing a quantity of a chemical species (e.g., molarity, molality, normality, weight percent or fraction, specific weight, total mass or weight, or total molar amount, etc.) in a fluidic medium by at least about 0.1%, 0.5%, 1%, 5%, 10%, 20%, 30%, 40%, 50%, 60%, 70%, 80%, 90%, 95%, 99%, 99.9%, 99.99%, 99.999%, or more than 99.999%. A transportate association condition may comprise decreasing a quantity of a chemical species in a fluidic medium by no more than about 99.999%, 99.99%, 99.9%, 99%, 95%, 90%, 80%, 70%, 60%, 50%, 40%, 30%, 20%, 10%, 5%, 1%, 0.5%, 0.1%, or less than 0.1%.

Altering the quantity of a chemical species of a fluidic medium may comprise diluting the fluidic medium with a volume of a second fluidic medium, in which the second fluidic medium has a lower concentration of the chemical species than the fluidic medium (e.g., substantially devoid of the chemical species). Altering a quantity of a chemical species of a fluidic medium may comprise separating at least a fraction of the chemical species from the fluidic medium. Separating at least a fraction of the chemical species from the fluidic medium may comprise one or more steps of: i) extracting a volume of the fluidic medium from contact with a solid support, ii) separating at least the fraction of the chemical species from the volume of the fluidic medium, and iii) after separating the fraction of the chemical species, contacting the volume of the fluidic medium with the solid support.

Altering the quantity of a chemical species of a fluidic medium may comprise combining the fluidic medium with a volume of a second fluidic medium, in which the second fluidic medium has a higher concentration of the chemical species than the fluidic medium. Altering a quantity of a chemical species of a fluidic medium may comprise concentrating a chemical species within the fluidic medium. Concentrating a chemical species within the fluidic medium may comprise one or more steps of: i) extracting a volume of the fluidic medium from contact with a solid support, ii) separating at least a fraction of a second chemical species (e.g., a solvent species) from the volume of the fluidic medium, thereby forming a remaining volume of the fluidic medium, in which the remaining volume of the fluidic medium has an increased concentration of the chemical species, and iii) after separating the fraction of the second chemical species from the fluidic medium, contacting the remaining volume of the fluidic medium with the solid support.

A transportate association condition may comprise adding a chemical species to a fluidic medium. Adding a chemical species to a fluidic medium can refer to providing a previously non-existing chemical species to the fluidic medium, for example by spike-in of the chemical species to the fluidic medium or by blending a second fluidic medium containing the added chemical species to an existing fluidic medium that is substantially devoid of the chemical species.

Alteration of a fluidic medium may be useful for facilitating mass transfer of transportates to a surface of a solid support, especially in cases where altering the fluidic medium changes a physical property of the transportate such as solubility or surface electrical charge. For certain transportates, such as biomolecules, changes in pH, ionic strength, or chemical composition of a fluidic medium can decrease a solubility or suspendability of the transportate, thereby increasing a concentration of the transportates adjacent to a surface of a solid support in the direction of a gravitational or centripetal force. Likewise, changes in surface tension or wettability of solid particles (e.g., inorganic nanoparticles) due to alteration of a fluidic medium (e.g., changes in surfactant concentration, changes in ionic strength, changes in solvent composition) can increase a sedimentation rate of the solid particles, thereby increasing a concentration of the transportates adjacent to a surface of a solid support in the direction of a gravitational or centripetal force.

A fluidic medium, as set forth herein, may comprise any of a variety of components, such as a solvent species, pH buffering species, a cationic species, an anionic species, a surfactant species, a denaturing species, an antioxidant species, or a combination thereof. A solvent species may include water, acetic acid, methanol, ethanol, n-propanol, isopropyl alcohol, n-butanol, formic acid, ammonia, propylene carbonate, nitromethane, dimethyl sulfoxide, acetonitrile, dimethylformamide, acetone, ethyl acetate, tetrahydrofuran, dichloromethane, chloroform, carbon tetrachloride, dimethyl ether, diethyl ether, 1-4, dioxane, toluene, benzene, cyclohexane, hexane, cyclopentane, pentane, or combinations thereof. A fluidic medium may include a buffering species including, but not limited to, MES, Tris, Bis-tris, Bis-tris propane, ADA, ACES, PIPES, MOPSO, MOPS, BES, TES, HEPES, HEPBS, HEPPSO, DIPSO, MOBS, TAPSO, TAPS, TABS, POPSO, TEA, EPPS, Tricine, Gly-Gly, Bicine, AMPD, AMPSO, AMP, CHES, CAPSO, CAPS, and CABS. A fluidic medium may include cationic species such as Na+, K+, Ag+, Cu+, NH4+, Mg2+, Ca2+, Cu2+, Cd2+, Zn2+, Fe2+, Co2+, Ni2+, Cr2+, Mn2+, Ge2+, Sn2+, A13+, Cr3+, Fe3+, Co3+, Ni3+, Ti3+, Mn3+, Si4+, V4+, Ti4+, Mn4+, Ge4+, Se4+, V5+, Mn5+, Mn6+, Se6+, and combinations thereof. A solvent or solution may include anionic species such as F−, Cl−, Br−, ClO3−, H2PO4−, HCO3−, HSO4−, OH−, I−, NO3−, NO2−, MnO4−, SCN−, CO32−, CrO42−, Cr2O72−, HPO42−, SO42−, SO32−, PO43−, and combinations thereof. A fluidic medium may include a surfactant species (e.g., an anionic surfactant, a cationic surfactant, a zwitterionic surfactant, an amphoteric surfactant or a non-ionic surfactant) including, but not limited to, stearic acid, lauric acid, oleic acid, sodium dodecyl sulfate, sodium dodecyl benzene sulfonate, dodecylamine hydrochloride, hexadecyltrimethylammonium bromide, polyethylene oxide, nonylphenyl ethoxylates, Triton X, pentapropylene glycol monododecyl ether, octapropylene glycol monododecyl ether, pentaethylene glycol monododecyl ether, octaethylene glycol monododecyl ether, lauramide monoethylamine, lauramide diethylamine, octyl glucoside, decyl glucoside, lauryl glucoside, Tween 20, Tween 80, n-dodecyl-o-D-maltoside, nonoxynol 9, glycerol monolaurate, polyethoxylated tallow amine, poloxamer, digitonin, zonyl FSO, 2,5-dimethyl-3-hexyne-2,5-diol, Igepal CA630, Aerosol-OT, triethylamine hydrochloride, cetrimonium bromide, benzethonium chloride, octenidine dihydrochloride, cetylpyridinium chloride, adogen, dimethyldioctadecylammonium chloride, CHAPS, CHAPSO, cocamidopropyl betaine, amidosulfobetaine-16, lauryl-N,N-(dimethylammonio)butyrate, lauryl-N,N-(dimethyl)-glycinebetaine, hexadecyl phosphocholine, lauryldimethylamine N-oxide, lauryl-N,N-(dimethyl)-propanesulfonate, 3-(1-pyridinio)-1-propanesulfonate, 3-(4-tert-butyl-1-pyridinio)-1-propanesulfonate, pluronic F-127, and combinations thereof. A fluidic medium may comprise a denaturing species including, but not limited to, acetic acid, trichloroacetic acid, sulfosalicylic acid, sodium bicarbonate, ethanol, ethylenediamine tetraacetic acid (EDTA), urea, guanidinium chloride, lithium perchlorate, sodium dodecyl sulfate, 2-mercaptoethanol, dithiothreitol, and tris(2-carboxyethyl) phosphine (TCEP).

A layer of fluidic medium may be contacted with a solid support, in which the layer has a depth or thickness. The depth or thickness of a layer of a fluidic medium may be at least about 1 nanometer (nm), 5 nm, 10 nm, 50 nm, 100 nm, 250 nm, 500 nm, 1 micron (μm), 5 μm, 10 μm, 20 μm, 30 μm, 40 μm, 50 μm, 100 μm, or more than 100 μm. Alternatively or additionally, a depth or thickness of a layer of a fluidic medium may be no more than about 100 μm, 50 μm, 40 μm, 30 μm, 20 μm, 10 μm, 5 μm, 1 μm, 500 nm, 250 nm, 100 nm, 50 nm, 10 nm, 5 nm, 1 nm, or less than 1 nm.

A transportate association condition may comprise altering a fluidic property of a fluidic medium, such as density, viscosity, thermal conductivity, thermal diffusivity, mass diffusivity, etc. Altering a fluidic property of a fluidic medium may comprise one or more of: i) altering a chemical composition of the fluidic medium (e.g., adding a chemical species to the fluidic medium, removing a chemical species from the fluidic medium or altering a quantity of a chemical species in the fluidic medium), and ii) altering a temperature of the fluidic medium (e.g., heating the fluidic medium or cooling the fluidic medium).

A temperature of a fluidic medium may be altered when forming an array of analytes by a temperature difference of at least about ±1 degree Celsius (° C.), +2° C., 3° C., ±4° C., ±5° C., ±10° C., ±15° C., ±20° C., ±25° C., ±30° C., ±40° C., ±50° C., or more than ±50° C. Alternatively or additionally, the temperature of a fluidic medium may be altered when forming an array of analytes by a temperature difference of no more than about ±50° C., ±40° C., +30° C., ±25° C., ±20° C., ±15° C., ±10° C., ±5° C., ±4° C., ±3° C., 2° C., ±1° C., or less than ±1° C. In some cases, the temperature of a fluidic medium may be altered to produce convective flow in a fluid, thereby transferring transportates toward or away from a surface of a solid support.

Altering the temperature of a fluidic medium may comprise cyclical changes in temperature (e.g., raising then lowering a temperature of the fluidic medium or lowering then raising the temperature of the fluidic medium). Altering the temperature of a fluidic medium may comprise combining a fluidic medium at a first temperature with a fluidic medium at a second temperature, in which the first temperature differs from the second temperature. Altering a temperature of a fluidic medium may comprise heating or cooling a solid support contacted with the fluidic medium. Altering a temperature of a fluidic medium may comprise irradiating the fluidic medium (e.g., microwave irradiation of an aqueous medium). Altering a temperature of a fluidic medium may comprise performing a non-isothermal chemical reaction in the fluidic medium (e.g., an endothermic or exothermic chemical reaction).

A transportate association condition may comprise a step of forming an interface between a fluidic medium containing a population of transportates and a second fluidic medium. A second fluidic medium contacted with a fluidic medium containing a population of transportates can be any conceivable fluid that is immiscible with the fluidic medium (e.g., a gas contacted with a liquid, a non-polar solvent contacted with a polar solvent, etc.) or has a limited miscibility with the fluidic medium (e.g., a second fluidic medium that solvates into a fluidic medium containing a population of transportates on a time scale longer than a time-scale of contacting the fluidic medium to a solid support). Forming an interface between a fluidic medium containing a population of transportates and a second fluidic medium may comprise flowing bubbles or droplets of the second fluidic medium through the fluidic medium. Forming an interface between a fluidic medium containing a transportates and a second fluidic medium may comprise forming an emulsion of the second fluidic medium in the first fluidic medium, or vice versa.

Forming an interface between a fluidic medium containing a population of transportates and a second fluidic medium may increase the concentration of the transportates in the fluidic medium. Forming an interface between a fluidic medium containing a population of transportates and a second fluidic medium may increase a rate of mass transfer of the transportates to a surface of a solid support containing a population of sites.

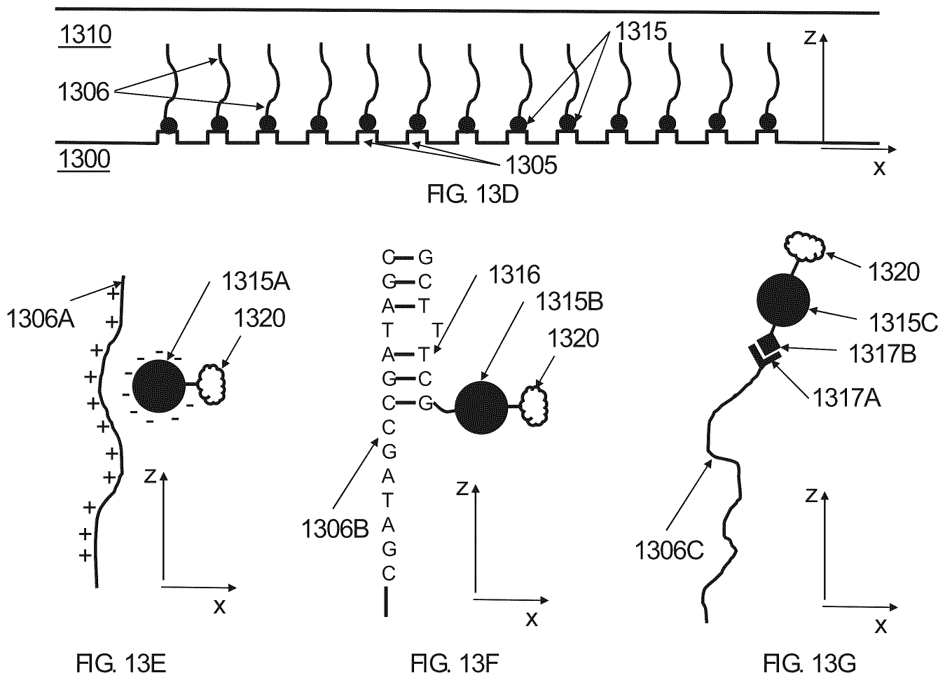

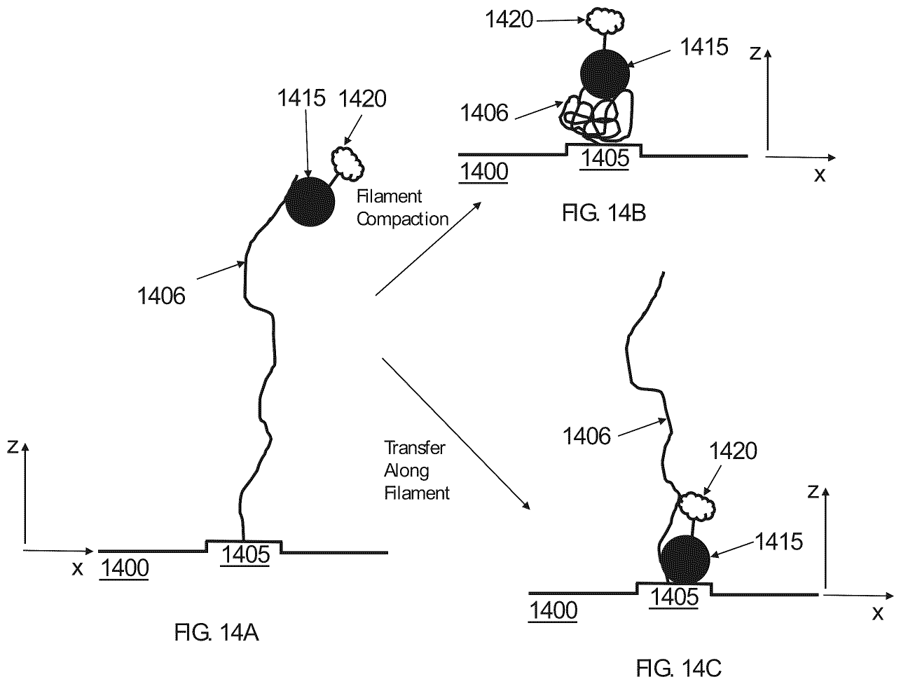

FIGS. 11A-11C illustrate aspects of forming interfaces during formation of arrays of analytes. FIG. 11A depicts a solid support 1100 containing a population of sites 1105, in which the solid support is contacted with a fluidic medium 1110 containing a population of analytes and/or anchoring moieties 1115. The population of analytes and/or anchoring moieties 1115 has a substantially uniform concentration throughout the fluidic medium 1110. FIG. 11B illustrates flow of a series of droplets of a second immiscible fluidic medium 1111 through the fluidic medium 1110 (i.e., a non-continuous set of interfaces). Alternatively, the droplets could be replaced with solid-phase beads (e.g., organic or inorganic nanoparticles) or a semi-solid bead (e.g., a hydrogel bead). The motion of the droplets or beads may generate a circulation of analytes and/or anchoring moieties 1115 around the moving droplets, producing motion of the analytes and/or anchoring moieties 1115 away from or toward the surface of the solid support 1100. Depending upon the velocity of the beads or droplets of the second fluidic medium 1111 through the first fluidic medium 1110, the net flux of analytes and/or anchoring moieties 1115 toward the surface of the solid support 1100 may be greater than a net flux of analytes and/or anchoring moieties 1115 toward the surface of the solid support produced by diffusion of the analytes and/or anchoring moieties 1115. Moreover, a reduction in the volume of the fluidic medium 1110 caused by the introduction of the second fluidic medium may concentrate the analytes and/or anchoring moieties 1115 within the remaining volume of the fluidic medium 1110. FIG. 11C illustrates formation of a continuous interface between a second fluidic medium 1111 and a first fluidic medium 1110 containing a population of analytes and/or anchoring moieties 1115 (e.g., a less dense second fluidic medium 1111 contacted with a more dense fluidic medium 1110). The reduction in the volume of the fluidic medium 1110 caused by the introduction of the second fluidic medium may concentrate the analytes and/or anchoring moieties 1115 within the remaining volume of the fluidic medium 1110, thereby decreasing a distance that analytes and/or anchoring moieties 1115 must diffuse or be transferred through to contact the surface of the solid support containing the population of array sites 1105.

A method of forming an array of analytes may comprise sedimenting transportates within a fluidic medium onto a surface of a solid support, in which the surface contains a population of array sites. In some cases, sedimenting transportates within a fluidic medium may comprise enhancing a natural rate of sedimentation of the transportates. For example, certain analytes within a population of analytes (e.g., very large proteins or nucleic acids) may inherently settle out of solution or suspension at a particular rate due to gravitational settling; enhancing sedimentation of the analytes may comprise setting the analytes at a rate that exceeds the particular rate of gravitational settling.

In some cases, sedimentation of transportates may be enhanced by coupling other particles or moieities to the transportates (i.e., increasing the weight of an analyte or anchoring moiety to enhance gravitational settling). Solid particles (e.g., metal nanoparticles, mineral particles, semiconductor particles, etc.) may be particularly useful due to their high mass density. In particular cases, sedimentation of analytes may be enhanced by coupling an anchoring moiety to an analyte, in which the anchoring moiety has a larger mass than the analyte. In particular cases, sedimentation of affinity agents may be enhanced by coupling a particle (e.g., a nucleic acid nanoparticle) to the affinity agent, in which the particle has a larger mass than the affinity agent. A transporate may be coupled to a particle, in which a ratio of a mass of the particle to a mass of the transportate is at least about 0.01:1, 0.1:1, 0.5:1, 1:1, 1.5:1, 2:1, 3:1, 4:1, 5:1, 10:1, 20:1, 50:1, 100:1, or more than 100:1. Alternatively or additionally, a particle may be coupled to a particle, in which a ratio of a mass of the particle to a mass of the transportate is no more than about 100:1, 50:1, 20:1, 10:1, 5:1, 4:1, 3:1, 2:1, 1.5:1, 1:1, 0.5:1, 0.1:1, 0.01:1, or less than 0.01:1.

A transportate may comprise a high-density or high-weight particle. In some cases, a transportate may comprise an inorganic nanoparticle (e.g., a metal nanoparticle, a metal oxide nanoparticle, a semiconductor nanoparticle, a high-molecular weight polymer, etc.). In some cases, one or more high-density or high-weight particles may be coupled to a transportate (e.g., a nucleic acid nanoparticle), thereby increasing the weight of the transportate. In some cases, one or more high-density or high-weight particles may be coupled to a transportate by a releasable or separable coupling interaction, such as nucleic acid hybridization, a photocleavable linker, a chemically-cleavable linker, or an enzymatically-cleavable linker.

A method of forming an array of analytes may comprise one or more steps of: i) within a fluidic medium, attaching a high-weight or high density particle to an analyte or anchoring moiety, ii) within the fluidic medium, attaching the analyte to the anchoring moiety, iii) contacting the fluidic medium to a solid support comprising a surface containing an array site for a sedimentation time, and iv) optionally removing the high-weight or high-density particle from the analyte or anchoring moiety. A sedimentation time may be a time sufficient for a particle to sediment through a full thickness of a fluid to a surface of a solid support or a surface of a sediment layer, such as at least about 1 second (s), 10 s, 15 s, 30 s, 45 s, 1 minute (min), 5 mins, 10 mins, 15 mins, 20 mins, 30 mins, 60 mins, or more than 60 mins. Alternatively or additionally, a sedimentation time may be no more than about 60 mins, 30 mins, 20 mins, 15 mins, 10 mins, 5 mins, 1 min, 45 s, 30 s, 15 s, 10 s, 1 s, or less than 1 s. A high-weight or high-density particle may be attached to a transportate. In some cases, an analyte may be attached to an anchoring moiety before a high-weight or high-density particle is attached to the analyte or anchoring moiety. In other cases, an analyte may be attached to an anchoring moiety after a high-weight or high-density particle is attached to the analyte or anchoring moiety. In other cases, an analyte and a high-weight or high-density particle may be attached to an anchoring moiety simultaneously.

Sedimentation of transportates toward a surface of a solid support may be further enhanced by centrifugation. In some cases, a method of forming an array of analytes may comprise one or more steps of: i) contacting a solid support containing a surface that comprises a population of sites with a fluidic medium containing a population of analytes and/or anchoring moieties, ii) placing the solid support within a centrifuge device, and iii) centrifuging the solid support, thereby increasing a concentration of analytes and/or anchoring moieties of the population of analytes and/or anchoring moieties adjacent to the surface of the solid support.

Centrifugation may occur at a rotational speed, such as at least about 1 revolution per minute (rpm), 10 rpm, 50 rpm, 100 rpm, 250 rpm, 500 rpm, 1000 rpm, 2500 rpm, 5000 rpm, 10000 rpm, 15000 rpm, 20000 rpm, 25000 rpm, 30000 rpm, 40000 rpm, 50000 rpm, or more than 50000 rpm. Alternatively or additionally, centrifugation may occur at no more than about 50000 rpm, 40000 rpm, 30000 rpm, 25000 rpm, 20000 rpm, 15000 rpm, 10000 rpm, 5000 rpm, 2500 rpm, 1000 rpm, 500 rpm, 250 rpm, 100 rpm, 50 rpm, 10 rpm, 1 rpm, or less than 1 rpm. Centrifugation speed may be chosen based upon a centrifugation apparatus to produce a net acceleration of entities, such as at least about 1.1 g, 1.5 g, 2 g, 5 g, 10 g, 20 g, 25 g, 50 g, 100 g, 500 g, 1000 g, 5000 g, 10000 g, 15000 g, 20000 g, 25000 g, 30000 g, 40000 g, 50000 g, 100000 g, or more than 100000 g. Alternatively or additionally, a centrifugation apparatus may produce a net acceleration of no more than about 100000 g, 50000 g, 40000 g, 30000 g, 25000 g, 20000 g, 15000 g, 10000 g, 5000 g, 1000 g, 500 g, 100 g, 50 g, 25 g, 20 g, 10 g, 5 g, 2 g, 1.5 g, 1.1 g, or less than 1.1 g. Centrifugation of a fluidic medium containing transportates may occur for at least about 1 second (s), 10 s, 15 s, 30 s, 45 s, 1 minute (min), 5 mins, 10 mins, 15 mins, 20 mins, 30 mins, 60 mins, 1.5 hours, 2 hours, 3 hours, 6 hours, or more than 6 hours. Alternatively or additionally, centrifugation of a fluidic medium containing transportates may occur for no more than 6 hours, 3 hours, 2 hours, 1.5 hours, 60 mins, 30 mins, 20 mins, 15 mins, 10 mins, 5 mins, 1 min, 45 s, 30 s, 15 s, 10 s, 1 s, or less than 1 s.

Sedimentation of transportates toward a surface of a solid support may be further enhanced by electrically-driven or magnetically-driven mass transfer, such as by electrophoresis or magnetophoresis, respectively. Electrophoretic mass transfer or magnetophoretic mass transfer may utilize applied electrical fields and/or magnetic fields to induce a flux of particles in a direction determined by the shape of the applied field. In some cases, electrophoretic or magnetophoretic may utilize an intrinsic electrical or magnetic charge property of a moiety (e.g., an analyte or an anchoring moiety). For example, depending upon the ionic strength of a fluidic medium, a particle may have a net surface electrical charge that affects speed and distance of migration (i.e., an isoelectric point) within an electric field. In another example, moieties can have an intrinsic magnetic behavior (e.g., paramagnetism, diamagnetism, ferromagnetism, etc.). In other cases, electrically-charged or magnetic particles may be coupled to a transportate, thereby providing a desired electrical or magnetic behavior to the transportate. In some cases, one or more charged or magnetic particles may be coupled to a transportate by a releasable or separable coupling interaction, such as nucleic acid hybridization, a photocleavable linker, a chemically-cleavable linker, or an enzymatically-cleavable linker.

A method of forming an array of analytes may comprise altering a fluidic medium comprising a plurality of particles and/or macromolecules, thereby increasing a rate of mass transfer of the particles and/or macromolecules toward a surface of the array or increasing a concentration of the particles and/or macromolecules adjacent to the surface of the array. Altering a fluidic medium comprising a plurality of particles and/or macromolecules may comprise altering a pH and/or ionic strength of the fluidic medium. Altering a fluidic medium can comprise increasing the pH, decreasing the pH, increasing the ionic strength, or decreasing the ionic strength of the fluidic medium. Depending upon the chemical composition of the particles and/or macromolecules, the manner of altering a fluidic medium can differ. For example, increasing a concentration of negatively-charged nanoparticles (e.g., nucleic acid nanoparticles) may comprise decreasing the ionic strength of a fluidic medium comprising the negatively-charged nanoparticles.

In some cases, altering a fluidic medium may comprise contacting a first fluidic medium containing a plurality of particles and/or macromolecules with a second fluidic medium, in which the first fluidic medium comprises a first fluidic property (e.g., a first pH, a first ionic strength, a first composition) and the second fluidic medium comprises a second fluidic property, and in which the first fluidic property differs from the second fluidic property. FIG. 30 depicts a fluidic device that can effect mass transfer toward an array surface by contacting a first fluidic medium with a second fluidic medium. A first fluidic stream 3050 (e.g., a fluidic medium comprising a plurality of particles and/or macromolecules) is impinged by a second fluidic stream 3051, thereby forming an interface 3053 between the first fluidic stream 3050 and the second fluidic stream 3051. The first fluidic stream 3050 has a substantially homogeneous concentration of a salt, and the second fluidic stream 3051 has a substantially homogeneous concentration of a salt that is lower than the salt concentration of the first fluidic stream 3050. As the fluidic stream flow toward the right side of FIG. 30, salt diffuses from the first fluidic stream 3050 into the second fluidic stream 3051. Accordingly, a diffusion gradient is formed across the interface 3053 over a downstream surface 3052 (e.g., a surface comprising a plurality of array sites) of a solid support 3000. The diffusion gradient of salt toward the second fluidic stream 3051 may increase a transfer of particles and/or macromolecules in the first fluidic stream 3050 toward the surface 3052 of the solid support.

In an aspect, provided herein is a method, comprising: a) contacting a fluidic medium comprising a plurality of particles to a solid support, in which the solid support comprises a plurality of sites, in which the spatial distribution of the plurality of particles in the fluidic medium is substantially homogeneous, and in which each individual particle of the plurality of particles is coupled to a plurality of macromolecules, b) concentrating the plurality of particles adjacent to a surface of the solid support, c) after concentrating the plurality of particles adjacent to the surface of the solid support, separating macromolecules from particles of the plurality of particles, d) binding the macromolecules to sites of the plurality of sites of the solid support, and e) after separating the macromolecules from the plurality of particles, dispersing particles of the plurality of particles from adjacent to the surface of the solid support.

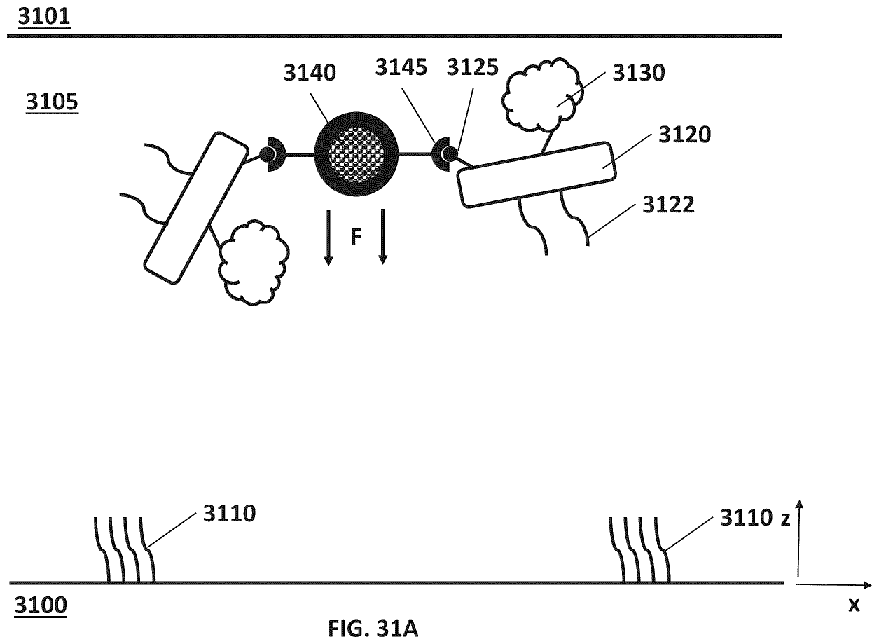

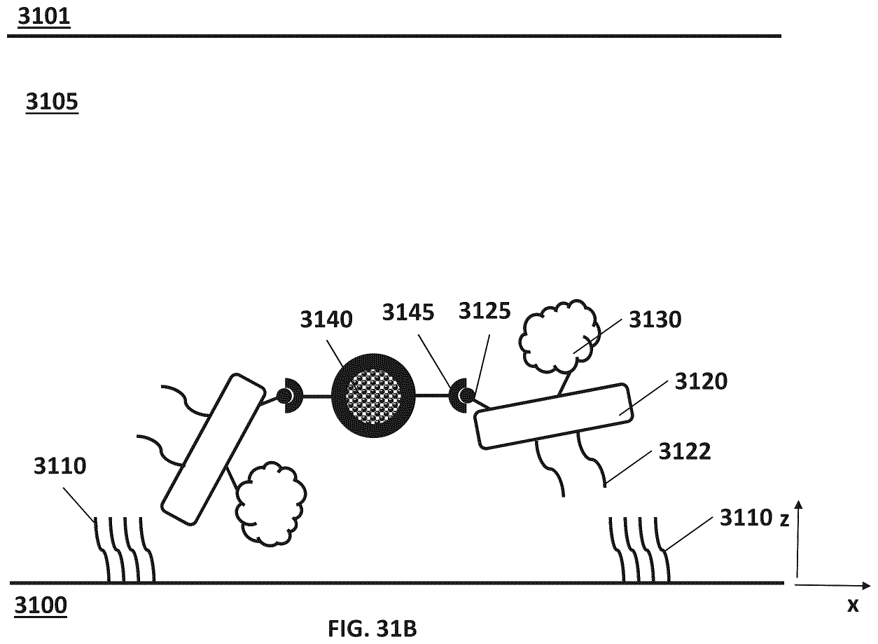

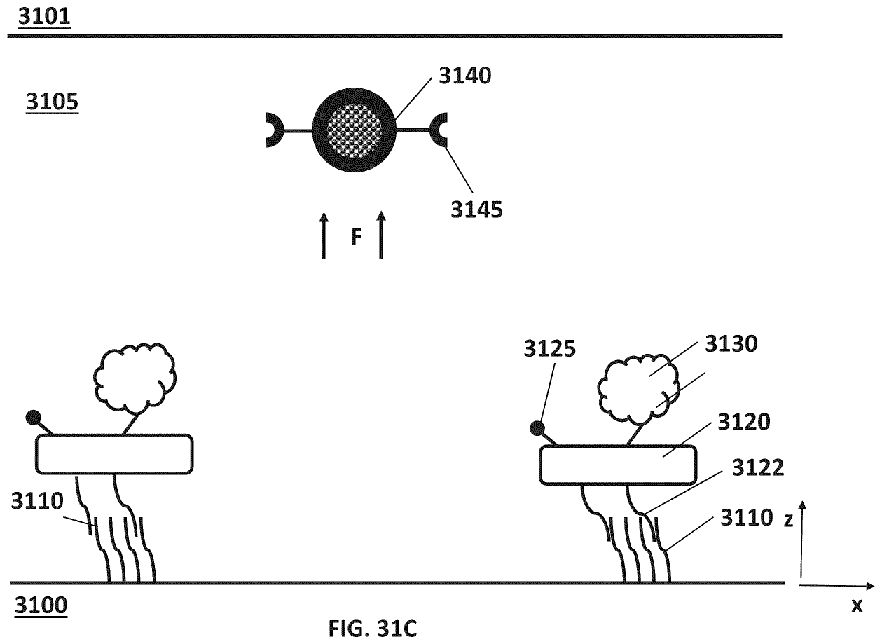

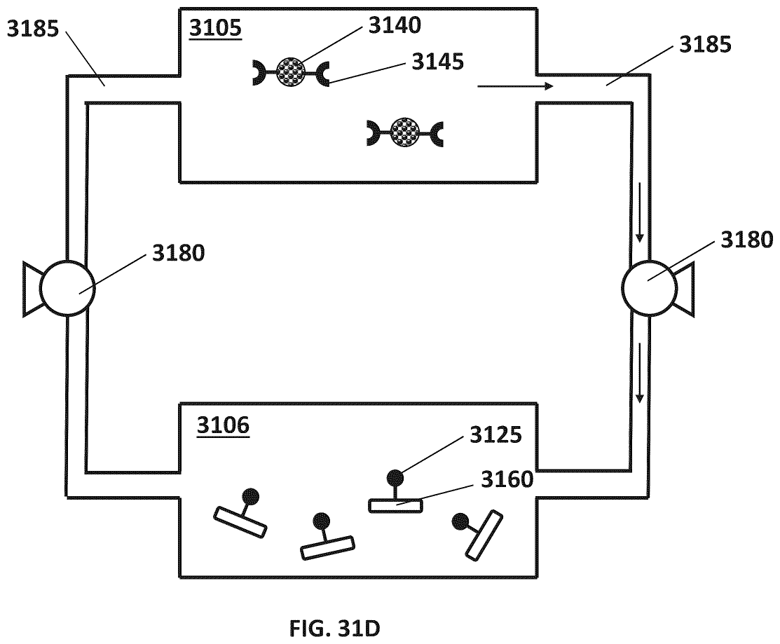

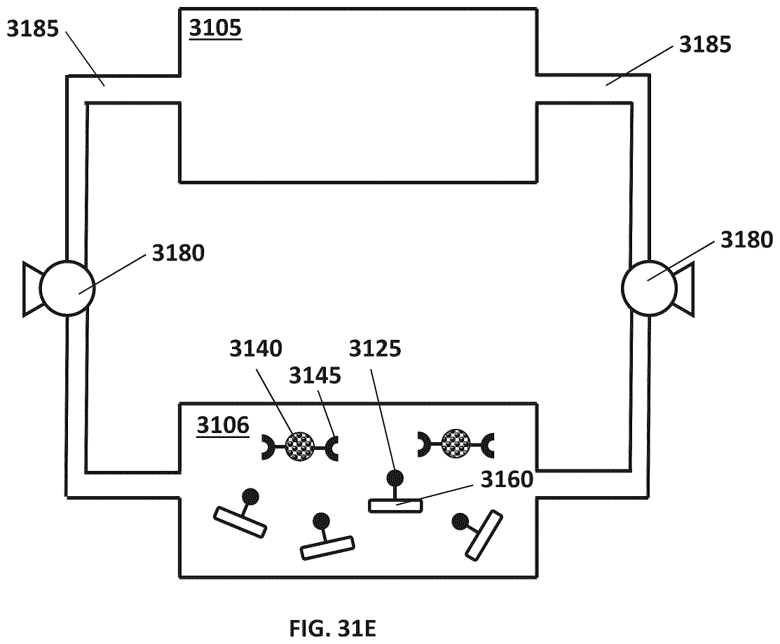

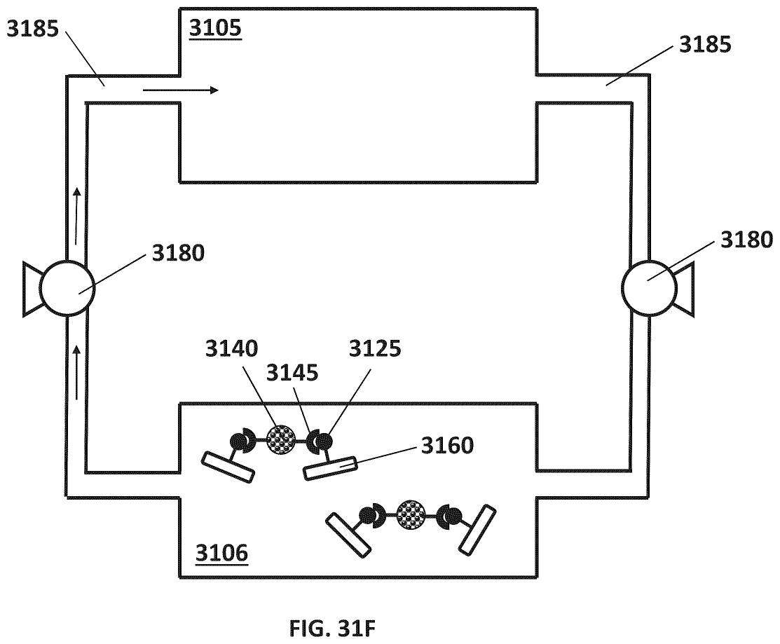

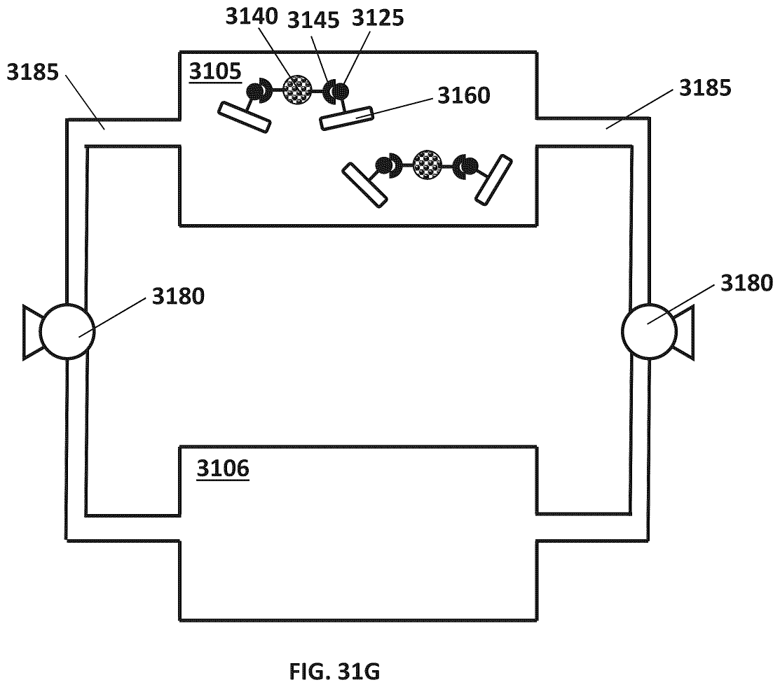

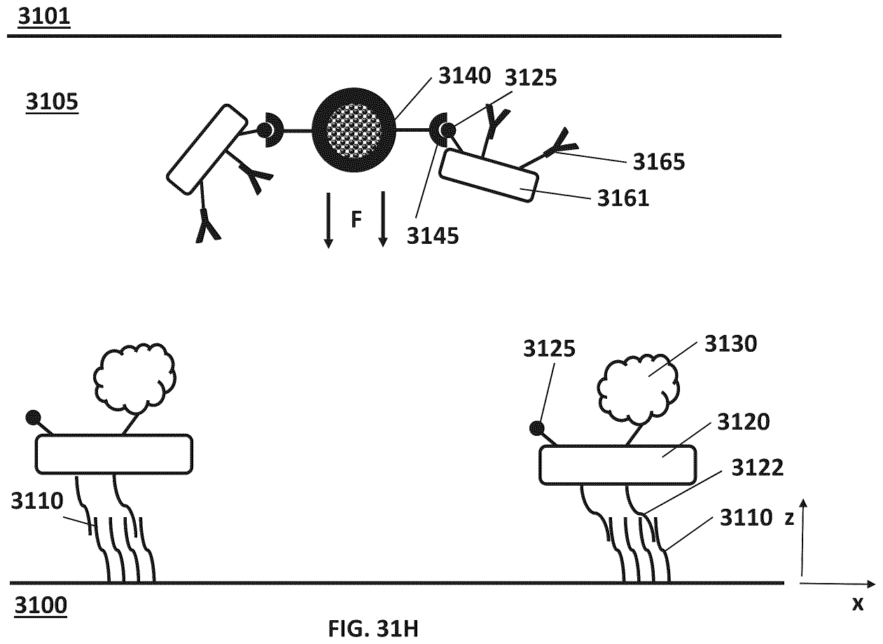

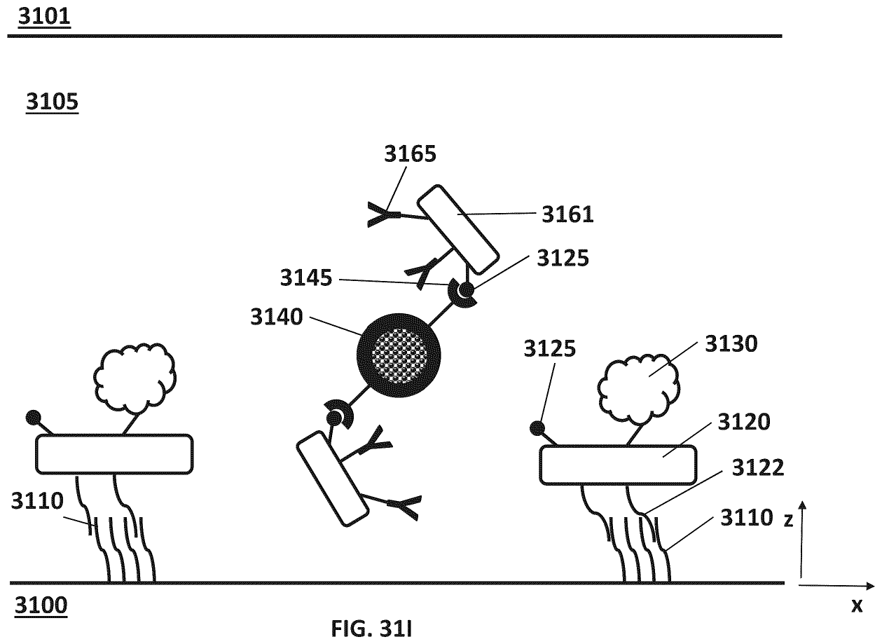

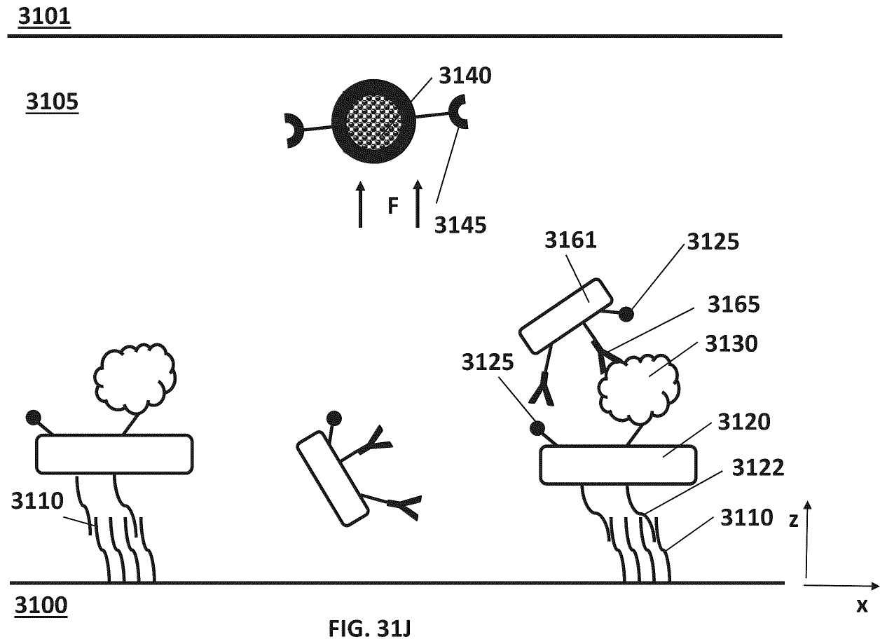

FIGS. 31A-31J depict aspects of a method of exerting directional forces on particles to drive mass transfer toward a surface of a solid support. FIGS. 31A-31C depict the use of particles to deliver assay agents (e.g., macromolecules, anchoring moieties, analytes, affinity agents, detectable probes, small molecules, etc.) to a surface of a solid support. FIG. 31A depicts an array system comprising a first solid support 3100 comprising array sites that individually contain pluralities of surface-coupled moieties 3110 and a second solid support 3101 that is offset from the first solid support 3100 to form a void 3105 between the two solid supports. The void contains a particle 3140 that is coupled to a plurality of macromolecules by interactions between particle-linked coupling moieties 3145 and macromolecule-linked coupling moieties 3125. Each macromolecule comprises an anchoring moiety 3120 coupled to an analyte 3130. The anchoring moieties comprise surface-coupling moieties 3122 that are configured to form interactions with the surface-coupled moieties 3110 of the array sites. A force F (e.g., a magnetic force, an electromotive force, a gravitational force, a centripetal force) is exerted on the particle complex. FIG. 31B depicts an altered position of the particle complex due to the force F translating the particle complex in the z-axis direction toward the surface of the first solid support 3100. FIG. 31C depicts the array system after the interactions between the macromolecules and the particles 3140 have been dissociated (i.e., photolytically dissociated, chemically dissociated, enzymatically dissociated, thermally dissociated, etc.). The macromolecules have been coupled to the array sites by binding of surface-coupling moieties 3122 to surface-coupled moieties 3110. Subsequent to release of the macromolecules from the particle 3140, the particle is translated toward the surface of the second solid support 3101 by a force F acting in an opposing direction to the force F exerted in FIG. 31A.

FIGS. 31D-31G depict a system for re-utilizing the particles 3140 of FIGS. 31A 31C. FIG. 31D depicts a configuration similar to that depicted in FIG. 31C, in which particles 3140 are present in a void 3105 after macromolecules have been separated from the particles 3140. The void 3105 is fluidically connected to a second void 3106 by a system of fluidic channels 3185 and fluid transfer devices 3180 (e.g., pumps). The second void 3106 comprises a second plurality of macromolecules 3160, in which each individual macromolecule comprises a particle-coupling moiety 3125. A flow has been induced that transfer particles from void 3105 to void 3106. FIG. 31E depicts a subsequent configuration, in which particles 3140 have been transferred to void 3106. FIG. 31F depicts a configuration in which macromolecules 3160 have been coupled to particles 3140 by binding of particle-coupled moieties 3145 with particle-coupling moieties 3125 to from particle complexes. A flow has been induced that transfers particle complexes toward void 3105. FIG. 31G depicts a configuration in which the particle complexes have been transferred from void 3106 to void 3105.

FIGS. 31H-31J depict a continuance of the method of FIGS. 31D-31G, in which the macromolecules 3160 are delivered to the surface of solid support 3100. FIG. 31H depicts the system of FIG. 31C, in which the particle complex has been transferred into void 3105. The particle complex comprises the particle 3140 coupled to a plurality of macromolecules, in which each macromolecule comprises a retaining group 3161 coupled to a plurality of affinity agents 3165. A force F (e.g., a magnetic force, an electromotive force, a gravitational force, a centripetal force) is exerted on the particle complex, thereby causing motion in the z-axis direction toward the surface of solid support 3100. FIG. 31I depicts a configuration in which the particle complex has been transferred closer to the surface of the first solid support 3100. FIG. 31J depicts the array system after the interactions between the macromolecules and the particles 3140 have been dissociated (i.e., photolytically dissociated, chemically dissociated, enzymatically dissociated, thermally dissociated, etc.). The macromolecule has been coupled to the analyte 3130 by binding of an affinity agent 3165 to the analyte 3130. Subsequent to release of the macromolecules from the particle 3140, the particle is translated toward the surface of the second solid support 3101 by a force F acting in an opposing direction to the force F exerted in FIG. 31H.

Methods of facilitating mass transfer to or across a surface of an array may be useful for transferring other assay reagents. A method may include the steps of: i) transferring an assay reagent by a mass transfer method, as set forth herein, and ii) modifying an analyte at an array site utilizing the assay reagent. The methods set forth herein may be useful for transferring assay reagents toward, across, and away from a surface of an array or solid support. For example, enzymes, peptides, nucleic acids, lipids, saccharides, metabolites, or other small molecules may be transferred to a surface of a solid support by a method set forth herein. In some cases, assay reagents may be delivered in vessels such as colloids, vesicles, liposomes, or emulsified liquids. In some cases, the particles 3140 depicted in FIGS. 31A-31J could be adapted to provide other assay agents to a surface. Certain reagents (e.g., small molecule compounds) may be delivered within vessels such as colloids, vesicles, or liposomes, in which vessels are attached to the particles 3140. Table III provides a non-exhaustive list of useful assay reagents that may be delivered to a surface of a solid support during a method set forth herein.

TABLE III
Assay Reagents for Analyte Modification

| | | Component | |
|---|---|---|---|
| Type | Activity/Effect | added | Examples |
| Enzyme | Add | Kinase | MAPK, ERK, ERK2, PKA, AKT |
| | phosphorylation | | |
| | to S, T, Y AAs | | |
| Enzyme | Remove | Phosphatase | Calf intestinal phosphatase, |
| | phosphorylation | | potato acid phosphatate, shrimp |
| | | | alkaline phosphatase |
| Enzyme | Cleave proteins | Protease | Trypsin, chymotrypsin, AspN, |
| | | | LysC, GluC, caspases, pepsin, |
| | | | methionine aminopeptidase, |
| | | | Factor Xa, pepsin, |
| | | | metalloproteases |
| Enzyme | Add glycan | Glycotransferase | |
| Enzyme | Remove | Deglycosylase | Glucosidases, EndoH, PNGaseF, |
| | glycans | | O-glycosidase, Neuraminidase |
| Enzyme | Acetylate | Acetylase | Histone acetyltransferases, N- |
| | proteins | | terminal acetyltransferases, |
| | | | lysine acetyltransferases |
| Enzyme | Remove | Deacetylase | Histone deacetylases, |
| | acetylation | | carboxylesterases, Zn-dependent |
| | | | amidohydrolases, sirtuins |
| Enzyme | Formylate | Formyltransferase | |
| | protein | | |
| Enzyme | Remove | Deformylase | Methionine aminopeptidase, |
| | formylation | | peptide deformylase |
| Enzyme | Add ubiquitn | Ubiquitin ligase | E3 ubiquitin ligases |
| Enzyme | Remove | Deubiquitinase | Trypsin, DUBs |
| | ubiquitin | | |
| Enzyme | Add SUMO | SUMO ligase | E3 SUMO ligase |
| Enzyme | Remove SUMO | SUMO protease | SENP enzymes |
| Enzyme | Add NEDD8 | NEDD8 ligase | E1 NEDD8 ligase, E2 NEDD8 |
| | | | ligase |
| Enzyme | Remove | NEDD8 protease | COP9 signalosome |
| | NEDD8 | | |
| Enzyme | Add methyl | Methyltransferase | Arginine methyltransferases, |
| | | | lysine methyltransferases |
| Enzyme | Remove methyl | Demethylase | Methylesterases, demethylases |
| Enzyme | Link peptides | Isopeptidase | Factor XIIIa, transglutaminase, |
| | with isopeptide | | transpeptidase |
| | bonds | | |
| Enzyme | Cleave | Isopeptidase | |
| | isopeptide | | |
| | bonds | | |
| Enzyme | Add hydrophobic groups | Lipid transferase | Farnesyltransferase, |
| | | | geranylgeranyltransferase |
| Enzyme | Isomerizes | Prolyl isomerase | |
| | proline | | |
| Enzyme | Add oxygen to | Hydroxylase | phenylalanine hydroxylase, |
| | tyrosine | | tyrosine hydroxylase |
| Enzyme | Adds NO group | Nitrosylase | |
| | to cys | | |
| Enzyme | Removes S- | Denitrosylases | Thioredoxin, nitrosyl reductase |
| | nitrosylation | | |
| Enzyme | Rearrange | Disulfide | |
| | disulfide bonds | isomerase | |
| Enzyme | Add succinyl | Succinylase | |
| | group to Lys | | |
| Enzyme | Remove | Desuccinylase | Sirtuins |
| | succinyl group | | |
| Enzyme | Add OH group | Hydroxylase | Dioxygenases, prolyl |
| | | | hydroxylase |
| Chemical | Cleave proteins | Chemical | Cyanogen bromide, BNPS- |
| | | treatments | skatole, hot acids |
| Chemical | Remove | Deglycosylase | TFMS, hydrazine |
| | glycans | | |
| Chemical | Inhibit | Small molecule | Aminoguanidine, metformin, |
| | glycation | | dydralzaine |
| Chemical | Inhibit | Small molecule | AEBSF, peptstatin, PMSF, |
| | proteases | inhibitors | EDTA, Leupeptin, benzamide |
| Chemical | Reductant | Reductant | DTT, DTNB, TCEP, beta |
| | | | mercaptoethanol |
| Chemical | Oxidant | Oxidant | Permanganate, ozone, hydrogen |
| | | | peroxide |
| Chemical | Inhibit | Small molecule | NaF, Sodium orthovanadate, |
| | phosphatases | inhibitors | beta-Glycerophosphate, sodium |
| | | | pyrophosphate, okadaic acid |
| Chemical | Inhibit kinases | Small molecule | Erlotinib, staurosporine, DAPK |
| | | inhibitors | inhibitor |
| Protein/Peptide | Inhibit kinases | Antibody; | Cetuximab, kinase inhibitor |
| | | Inhibiting Peptide | peptides, |
| Protein/peptide | Inhibit | Protein | BPTI/Aprotinin, STI, Bowman- |
| | proteases | | Birk inhibitor |
| Protein/peptide | Inhibit | Inhibiting peptide | phosphatase inhibitor peptides |
| | phosphatases | | |

A method of forming an array of analytes may comprise one or more steps of: i) within a fluidic medium, attaching a charged or magnetic particle to an analyte or anchoring moiety, ii) within the fluidic medium, attaching the analyte to the anchoring moiety, iii) contacting the fluidic medium to a solid support comprising a surface containing an array site in the presence of an electrical field or a magnetic field, and iv) optionally removing the charged or magnetic particle from the analyte or anchoring moiety.

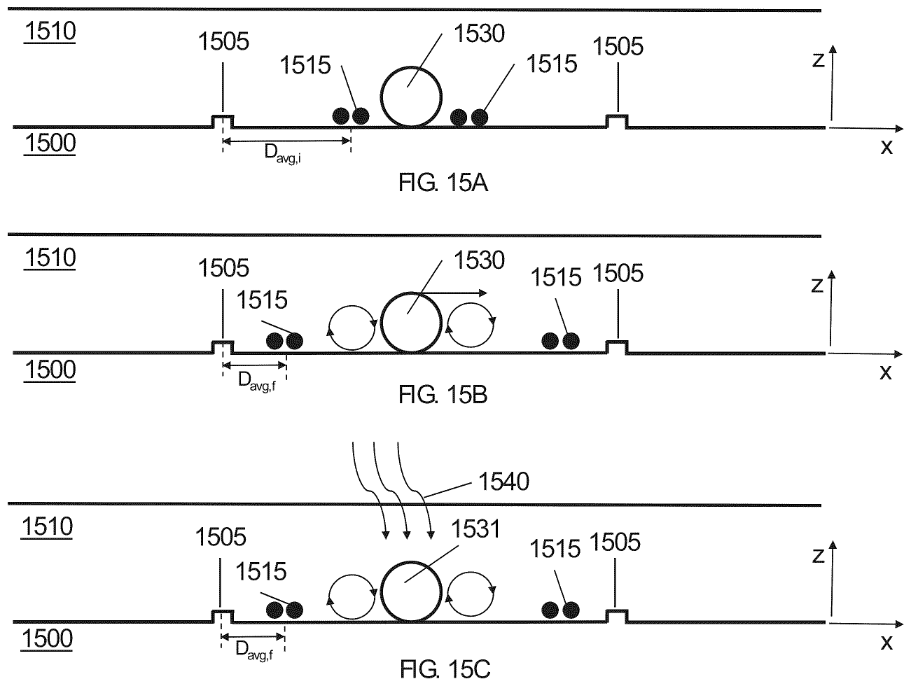

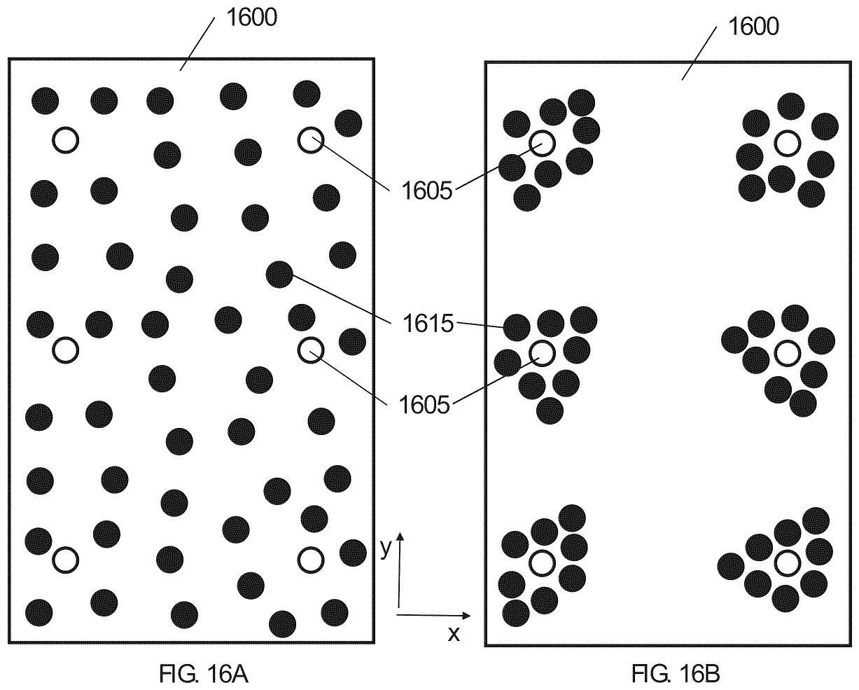

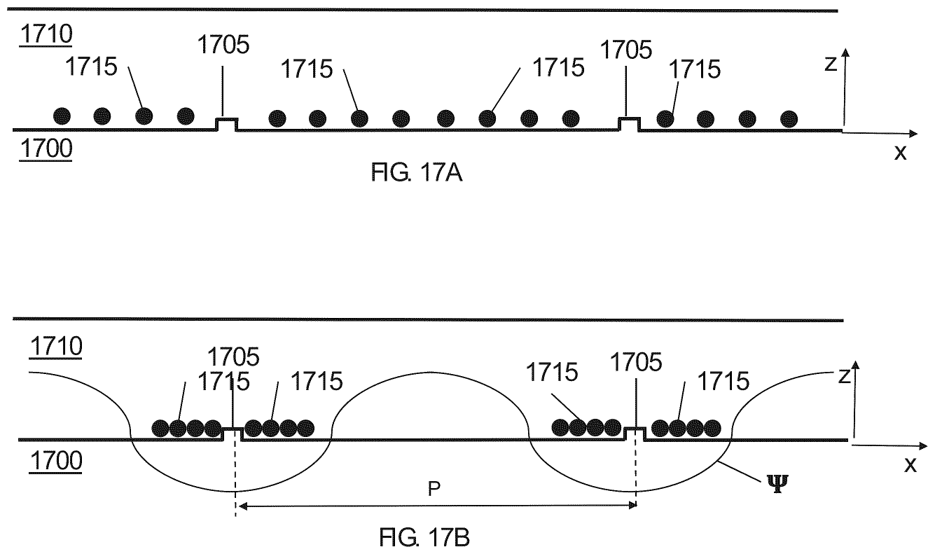

Unlike mass transfer methods such as sedimentation or centrifugation (in which moieties move in the direction of the gravitational force or centripetal acceleration, respectively, thereby limiting a position of an array of sites relative to analytes or anchoring moieties), electrophoretic or magnetophoretic mass transfer may be advantageous for multi-dimensional mass transfer. Electrical or magnetic fields can be shaped in one, two, or three dimensions to effect mass transfer in any conceivable direction. Moreover, electrical or magnetic field polarity can be reversed, thereby facilitating reversal in direction of mass transfer. FIGS. 12A-12C depict use of reversible electric or magnetic fields to facilitate array formation. FIG. 12A depicts a configuration in which a solid support 1200 containing a population of sites 1205 has been contacted with a fluidic medium 1210 containing a population of analytes and/or anchoring moieties 1215. An electrical field or magnetic field has facilitated migration of the analytes and/or anchoring moieties 1215 toward the surface of the solid support 1200. FIG. 12B depicts a second configuration, in which a polarity of the electrical field or magnetic field has been reversed, thereby facilitating migration of any unbound analytes and/or anchoring moieties 1215 away from the surface of the solid support 1200. Analytes and/or anchoring moieties 1215 bound to array sites 1205 do not migrate within the applied field. FIG. 12C depicts a third configuration, in which a polarity of the electrical field or magnetic field has been reverted back to the polarity of FIG. 12A, thereby facilitating migration of the analytes and/or anchoring moieties 1215 back toward the surface of the solid support 1200. The z-axis oscillatory mass transfer depicted in FIGS. 12A-12C, coupled with natural or induced transfer in the x-axis direction (and unseen y-axis direction) may facilitate increased binding of analytes and/or anchoring moieties 1215 to array sites 1205.

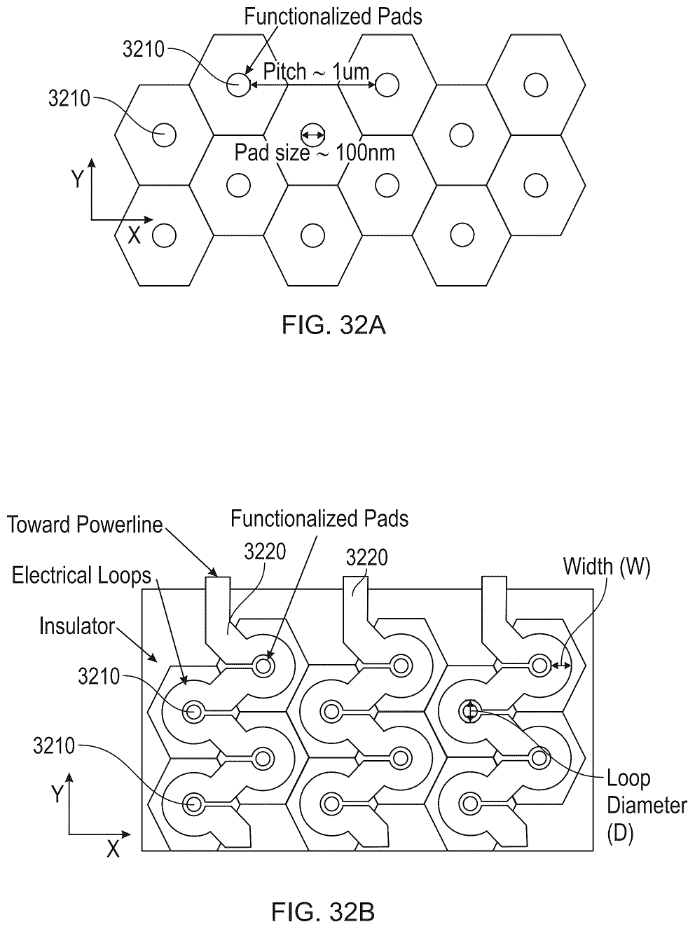

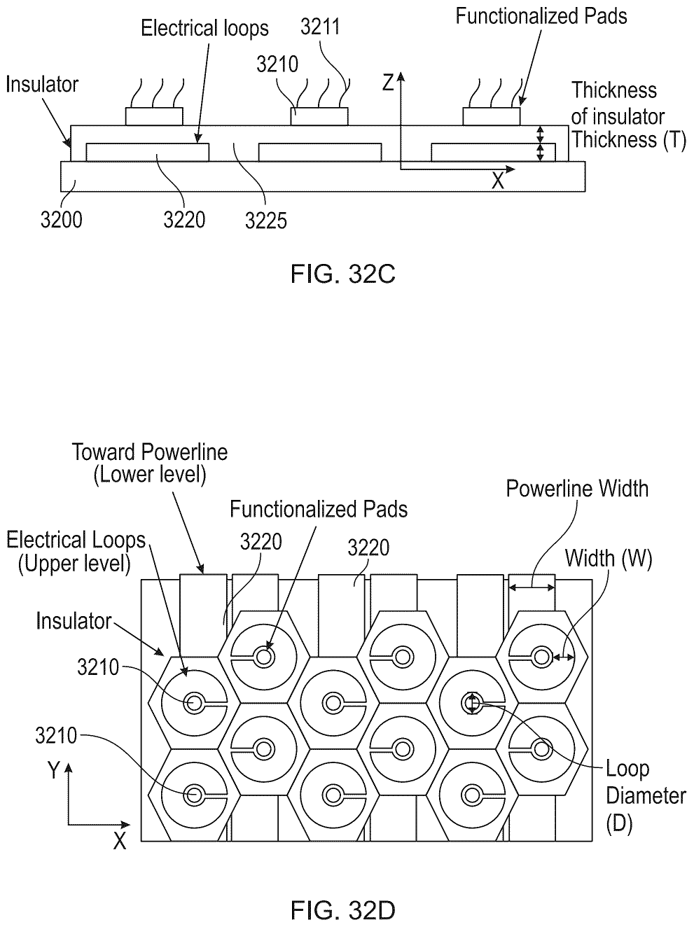

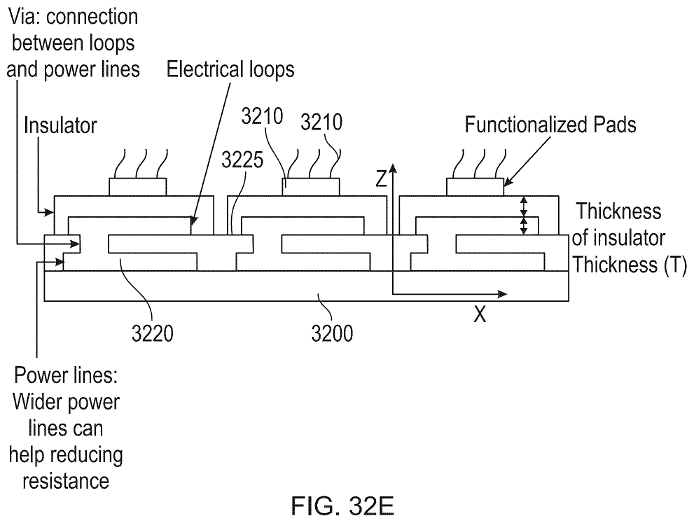

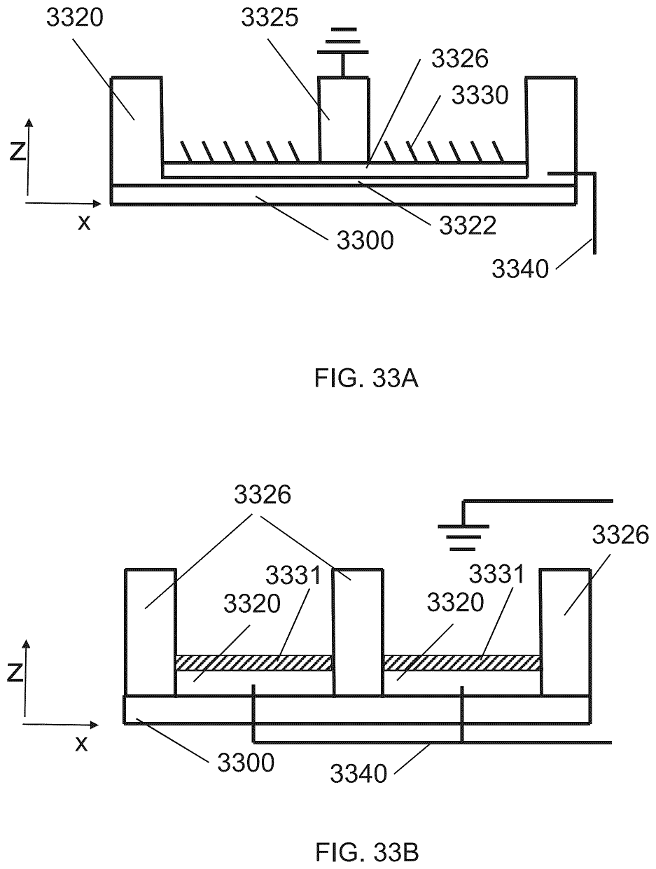

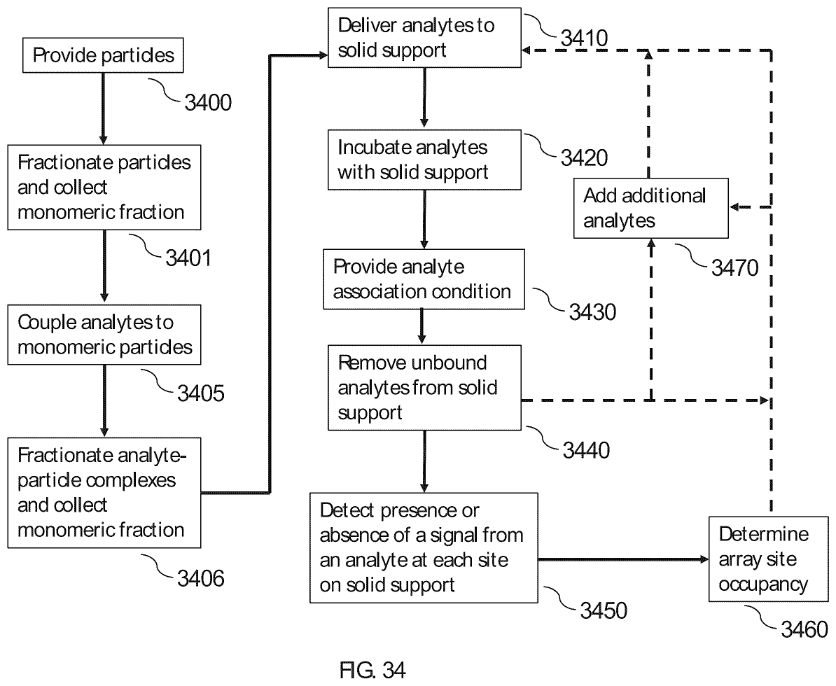

FIGS. 32A-32E and FIGS. 33A-33B depict array configurations that may be useful for producing magnetic or electrical fields that can effect mass transfer of transportates near array sites. FIGS. 32A-32E depict array configurations that include electromagnets coupled to array solid supports. FIG. 32A illustrates a top-down view of an array of sites 3210 with a hexagonal pattern of individual array sites 3210. FIG. 32B illustrates a top-down view of an array with a similar array pattern to that depicted in FIG. 32A. Conductive paths 3220 are patterned around each individual array site 3210 such that the conductive paths 3220 form circular paths around each array site 3210. Electrical current passed through the conductive paths 3220 can facilitate formation of a magnetic field in the vicinity of each individual array site 3210. FIG. 32C illustrates a cross-sectional view of the array depicted in FIG. 32B. The array of sites 3210 is disposed on a solid support 3200. The conductive paths are disposed in a single layer on the solid support 3200 and covered by an optional electrically-insulating material 3225. The conductive paths may be formed by lithographic processes. Array sites 3210 are patterned (e.g., lithographically) on the electrically-insulating material 3225. The array site may comprise an optional solid support material (e.g., metal, semiconductor, metal oxide, polymer) and a plurality of surface-coupled moieties 3211. FIG. 32D depicts a top-down view of a double-layer configuration of the array depicted in FIG. 32B. The conductive paths 3220 form about two loops adjacent to each individual array site, potentially facilitating formation of a stronger or less dispersed magnetic field at each individual array site. FIG. 32E depicts a cross-sectional view of the array of FIG. 32D. The conductive path can be formed in a substantially helical pattern within the electrically-insulating material 3225 on the solid support 3200.

FIGS. 33A and 33B illustrate array configurations for forming electrical fields in the vicinity of array sites. FIG. 33A depicts a cross-sectional view of an array configuration comprising well structures. Each individual well is bounded on a first side by a conductive material 3320 that is disposed on a solid support material 3300. Each individual well is also bounded on a second side by a second material 3325 that is electrically grounded and disposed on an insulating material 3326. A conductive bridge 3322 connects conductive walls of adjacent wells. Accordingly, supply of an electrical potential via a conductive line 3340 will generate an electrical potential between the conductive walls 3320 and the grounded walls 3325 of each well. Each individual well contains a plurality of surface-coupled moieties that provide attachment sites for binding analytes, anchoring moieties, or other particles within the well. When an electrical potential is applied to the array system, an electrical field will be formed adjacent to each well, thereby affecting movement of electrically-charged moieties with respect to the electric field. FIG. 33B depicts an array configuration in which the conductive material 3320 is provided in the bottoms of each well. Individual wells are bounded by walls formed of a non-conductive material or electrically-insulating material 3326. Each individual well further comprises a plurality of surface-coupled moieties or a surface coating (e.g., a polymer coating) that provides attachment sites for binding analytes, anchoring moieties, or other particles within the well. Electrical potential may be supplied to each individual well by conductive lines 3340 that contact the conductive material 3320 disposed in the bottom of each individual well. The electrical field formed in a well of the configuration of FIG. 33B may be substantially orthogonal to the directionality of the electrical field formed in the configuration of FIG. 33A.

Particles for delivering macromolecules and other moieties to array surfaces may have various useful configuration depending upon the mechanism of delivery of the particles to the surface. For example, advantageous particles for delivery by sedimentation or centrifugation can contain a high-density and/or solid material core (e.g., a metal, metal oxide, mineral, or semiconductor nanoparticle or microparticle). In another example, magnetic particles and particles with a non-neutral electrical charge can be useful for delivery by magnetophoresis and electrophoresis, respectively. Useful particles may comprise a core and shell structure, in which the core comprises an insoluble material and the shell comprises a coating or functionalized layer that provides a chemical characteristic to the particle, such as increasing the solubility and/or suspendibility of the particle (e.g., a hydrophilic layer to increase aqueous solubility of a metal or graphene particle), or providing electrically-charged surface moieties (e.g., an anionic, cationic, or zwitterionic polymer). A particle for delivery to an array surface may comprise an organic nanoparticle or an organic microparticle (e.g., a polymer particle, a graphene particle, a polypeptide, a nucleic acid particle, a polysaccharide particle, etc.). A particle for delivery to an array surface may comprise an inorganic nanoparticle or an inorganic microparticle (e.g., a metal particle, a metal oxide particle, a semiconductor particle, a magnetic nanoparticle, etc.).

It may be advantageous to deliver transportates to an array surface utilizing particles that can deliver a plurality of the transportates. Accordingly, particles may be chosen with a size that permits a plurality of transportates to be attached to them. Particle size (e.g., average length, average width, average diameter, average hydrodynamic radius, etc.) may depend, at least in part, on the characteristic dimension (e.g., average length, average width, average diameter, average hydrodynamic radius, etc.) of the transportates to be attached to the particle. A ratio of particle size to attached transportate size may be at least about 0.25, 0.5, 0.75, 1, 1.5, 2, 2.5, 3, 4, 5, 10, 20, 50, 100, or more than 100. Alternatively or additionally, a ratio of particle size to attached transportate size may be no more than about 100, 50, 20, 10, 5, 4, 3, 2.5, 2, 1.5, 1, 0.75, 0.5, 0.25, or less than 0.25. A particle may be attached to a plurality of transportates, such as at least about 2, 5, 10, 20, 50, 100, 500, 1000, 5000, 10000, 100000, 1000000, or more than 1000000 transportates. Alternatively or additionally, a particle may be attached to no more than about 1000000, 100000, 10000, 5000, 1000, 500, 100, 50, 20, 10, 5, or less than 5 transportates.

In some cases, a magnetic particle may be utilized for a method set forth herein. Magnetic particle may include ferromagnetic particle, diamagnetic particles, or paramagnetic particles. In some cases, a plurality of transportates may be coupled to a magnetic particle. In other cases, a plurality of transportates may be coupled to a non-magnetic particle, in which the non-magnetic particle is further coupled to one or more magnetic particles.

In some cases, a particle with a non-neutral electrical charge may be utilized for method set forth herein. A particle may be provided a non-neutral electrical charge by providing a layer or coating containing electrically-charged moieties (e.g., amines, carboxylic acids, etc.). The skilled person will readily recognize that electrical charge will depend in part on a fluidic medium in which a particle is provided. A medium comprising ions (e.g., dissolved salts) can partially or completely screen charges depending upon ionic strength of the medium. Further, effective electrical charge can be affected by pH-dependent protonation or deprotonation.

A particle may be coupled to a plurality of transportates. In some cases, a transportate of the plurality of transportates may comprise a biomolecule (e.g., a polypeptide, a nucleic acid, a polysaccharide, a lipid, a metabolite, or a combination thereof). In some cases, a transportate of the plurality of transportates may comprise an analyte. In some cases, a transportate of the plurality of transportates may comprise an affinity agent. In some cases, a transportate of the plurality of transportates may comprise a polymer (e.g., a biopolymer, a synthetic polymer, a linear chain polymer, a branched chain polymer, etc.). In some cases, a transportate of the plurality of transportates may comprise a nanoparticle (e.g., a nucleic acid nanoparticle, a polymer nanoparticle).

In a first embodiment, a particle may be coupled to a plurality of transportates, in which each individual transportate of the plurality of transportates comprises a nanoparticle (e.g., a nucleic acid nanoparticle, a polymer nanoparticle), and in which each individual nanoparticle is coupled to an analyte. Optionally, each individual nanoparticle may further comprise one or more surface-coupling moieties that are configured to bind the nanoparticle and/or analyte to an array site. Optionally, each transportate may further comprise one or more detectable labels (e.g., fluorophores, barcodes, etc.), for example coupled to the nanoparticle or analyte.

In a second embodiment, a particle may be coupled to a plurality of transportates, in which each individual transportate of the plurality of transportates comprises a nanoparticle (e.g., a nucleic acid nanoparticle, a polymer nanoparticle), and in which each individual nanoparticle is coupled to one or more affinity agents (e.g., antibodies, antibody fragments, aptamers, peptide binders, etc.). Optionally, each transportate may further comprise one or more detectable labels (e.g., fluorophores, luminophores, etc.), for example coupled to the nanoparticle or an affinity agent.

It may be preferable to couple transportates to particles to facilitate transfer of the macromolecules or other moieties to a surface of an array. In particular, it may be preferable to provide a separable coupling between a transportate and a particle such that the transportate can be controllably released from the particle at an advantageous time and/or location. In a particularly advantageous configuration, a particle may comprise a coupling moiety that facilitates repetitive attachment of transportates or other moieties to the particle. For example, attachment of transportates to particles by base pair interactions of complementary oligonucleotides facilitates association and dissociation of the transportates from the particle by nucleic acid hybridization/dehybridization. Accordingly, a particle may be provided with a plurality of coupling moieties, in which each individual coupling moiety of the plurality of coupling moieties is configured to form a binding interaction with a complementary coupling moiety of a transportate or other moiety.

In some cases, a particle of a plurality of particles can be coupled to a transportate or other moiety of a plurality of transportates or other moieties by a covalent bond. In some cases, a covalent bond can be a photolabile covalent bond (e.g., a reversible photolabile covalent bond). A method may comprise a step of separating a transportate or other moiety from a particle, in which separating the transportate or other moiety comprises cleaving a covalent bond coupling the transportate or other moiety to the particle. In some cases, cleaving a covalent bond coupling a transportate or other moiety comprises photolytically cleaving, chemically cleaving, or enzymatically cleaving the covalent bond. Photolytically cleaving a covalent bond may comprise a step of contacting the covalent bond with light. Chemically cleaving a covalent bond may comprise a step of performing a substitution reaction or an elimination reaction, thereby dissociating the transportate or other moiety from the particle. Enzymatically cleaving a covalent bond may comprise a step of contacting the covalent bond with an enzyme (e.g., a restriction enzyme, a protease, etc.).

In some cases, a particle of a plurality of particles can be coupled to a transportate or other moiety of a plurality of transportates or other moieties by a non-covalent interaction. Useful non-covalent interactions for coupling transportates or other moieties to particles can include nucleic acid hybridization, ligand-receptor binding (e.g., streptavidin/biotin, SpyCatchter/SpyTag, SnoopCatcher/SnoopTag, SdyCatcher/SdyTag, etc.), electrostatic interactions (e.g., a positively-charged moiety coupled to a negatively-charted moiety), a magnetic interaction (e.g., a first magnetic moiety coupled to a second magnetic moiety), or combinations thereof. A method may comprise a step of separating a transportate or other moiety from a particle of a plurality of particles, in which separating the transportate or other moiety from the particle comprises dissociating the non-covalent interaction. In some cases, dissociating the non-covalent interaction comprises contacting the particle with a dissociating agent, wherein the dissociating agent comprises a denaturant, a chaotrope, or a binding competitor. In some cases, dissociating the non-covalent interaction comprises heating the particle. In some cases, dissociating the non-covalent interaction comprises altering a pH or ionic strength of a fluidic medium.

In some cases, particles may be transported to a surface of a solid support comprising an array by applying a force (e.g., a gravitational force, a centripetal force, an electric force, a magnetic force, etc.) to the particle, thereby concentrating the particles adjacent to the surface of the solid support. In some cases, concentrating a plurality of particles adjacent to a surface of a solid support comprises forming an electric field or a magnetic field in the fluidic medium. An electric field may be formed with an average or peak magnitude of at least about 0.01 millivolt per meter (mV/m), 0.1 mV/m, 0.5 mV/m, 1 mV/m, 5 mV/m, 10 mV/m, 50 mV/m, 100 mV/m, 500 mV/m, 1000 mV/m, 5000 mV/m, 10000 mV/m, 100000 mV/m, 1000000 mV/m, or more than 1000000 mV/m. Alternatively or additionally, an electric field may be formed with an average or peak magnitude of no more than about 1000000 mV/m, 100000 mV/m, 10000 mV/m, 5000 mV/m, 1000 mV/m, 500 mV/m, 100 mV/m, 50 mV/m, 10 mV/m, 5 mV/m, 1 mV/m, 0.5 mV/m, 0.1 mV/m, 0.01 mV/m, or less than 0.01 mV/m. A magnetic field may be formed with an average or peak magnitude of at least about 1 Tesla per meter (T/m), 10 T/m, 50 T/m, 100 T/m, 500 T/m, 1000 T/m, 5000 T/m, 10000 T/m, 50000 T/m, 100000 T/m, 1000000 T/m, or more than 1000000 T/m. Alternatively or additionally, a magnetic field may be formed with an average or peak magnitude of no more than about 1000000 T/m, 100000 T/m, 50000 T/m, 10000 T/m, 5000 T/m, 1000 T/m, 500 T/m, 100 T/m, 10 T/m, 1 T/m, or less than 1 T/m.

After being transported to a surface of a solid support containing a plurality of sites, transportates may be bound to sites of the plurality of sites. In some cases, transportates may be separated from a particle before being bound to the sites of the plurality of sites. In other cases, transportates may be separated from a particle before after bound to the sites of the plurality of sites.

In some cases, a method may comprise a step of dispersing a particle or a plurality thereof from adjacent to a surface of a solid support (e.g., a solid support comprising a plurality of sites, a solid support that is substantially devoid of sites). Dispersing a particle may comprise a process that transfers the particle from a region with a higher concentration of particles to a region with a lower concentration of particles. Dispersion of particles can be a spontaneous process (e.g., a diffusion process) or a facilitated process (e.g., due to a gravitational force, a centripetal force, a magnetic force, an electric force, or a combination thereof). A particle may be dispersed from a surface after separating a transportate or other moiety from the particle. A particle may be dispersed from a surface before transferring the particle from a first void, reservoir, or chamber to a second void, reservoir, or chamber.

In some cases, a method may comprise a step of, after dispersing particles of a plurality of particles from adjacent to the surface of the solid support, coupling a second plurality of transportates to a particle of the particles. In some cases, coupling a second plurality of transportates to a particle of the particles can further comprise transferring the particles to a reservoir comprising the second plurality of transportates. In other cases, coupling a second plurality of transportates to a particle of the particles can further comprise providing the second plurality of transportates to the fluidic medium. For example, a second plurality of transportates may be provided to a fluidic medium within a void of chamber comprising an array of sites.

In an embodiment, a plurality of particles may be retained within a void or chamber comprising a plurality of sites. The void or chamber may comprise a first surface or first solid support comprising a plurality of sites, and a second surface or a second solid support that is substantially devoid of sites. In a particular embodiment, the first surface or solid support may be substantially opposed to the second surface or solid support (e.g., a lower face and upper face of a flow cell). Particles may be concentrated adjacent to the first surface or solid support when transferring transportates to the first surface or solid support, and may be concentrated at or bound to the second surface or solid support before or after transferring the transportates. This embodiment may be advantageous for performing fluid transfer into a void or chamber without removing the particles from the void or chamber. In some cases, the particles may be bound to or held in contact with the second surface or solid support by an electric or magnetic field. In some cases, a method may further comprise: i) binding the particles to a surface of a second solid support, ii) after binding the particles to the surface of the second solid support, removing the fluidic medium from the second solid support; and iii) providing a second fluidic medium to the second solid support, in which the second fluidic medium comprises a second plurality of transportates, and in which the second plurality of transportates are configured to bind to the particles.

In some cases, a method may comprise a step of dispersing particles of a plurality of particles from adjacent to a surface of a solid support, wherein dispersing the particles comprises forming an electric field or a magnetic field in the fluidic medium. In some cases, forming the electric field or the magnetic field in the fluidic medium can comprise reversing a polarity or a directionality of the electric field or the magnetic field in the fluidic medium. For example, reversing the direction of electricity flow through an electromagnet can reverse the polarity of a magnetic field. In some cases, dispersing particles of a plurality of particles from adjacent to a surface of a solid support can comprise applying a centripetal force to the particles of the plurality of particles (e.g., by centrifugation). In some cases, dispersing particles of a plurality of particles from adjacent to a surface of a solid support can comprise providing a turbulent flow in the fluidic medium.

Certain described methods of increasing mass transfer of transportates toward a surface of a solid support containing a population of array sites (e.g., altering a fluidic medium, sedimentation, centrifugation, electrophoretic transfer or magnetophoretic transfer) provide mechanisms for driving transportates toward the surface containing the array sites, but may not increase a likelihood that an transportate will contact the surface at an array site (e.g., as opposed to contacting the surface in an interstitial region). Accordingly, it may be advantageous to provide an array system that increases mass transfer of transportates toward a surface of a solid support containing a population of array sites, and increases a likelihood that transportates contact the surface of the solid support at or adjacent to an array site of the population of array sites.

In an aspect, provided herein is a method of forming an array, comprising: a) contacting an array comprising a plurality of sites with a layer of a fluidic medium, in which the fluidic medium comprises a population of transportates, in which the layer of the fluidic medium has an average thickness, in which sites of the population of sites comprise filamentous moieties, and in which the filamentous moieties have an average length of at least 10% the average thickness of the fluidic medium, and b) binding transportates to at least 70% of sites of the population of sites within 15 minutes of contacting the array with the fluidic medium.

An array site, as set forth herein, may comprise a filamentous moiety. An array site may comprise one and only one filamentous moiety. An array site may comprise a plurality of filamentous moieties. In some cases, an array site of a population of array sites may comprise zero filamentous moieties. In some cases, no more than 50% (e.g., no more than 40%, 30%, 25%, 20%, 15%, 10%, 5%, 1%, 0.5%, 0.1%, or less than 0.1%) of array sites of a population of array sites comprises zero filamentous moieties. A filamentous moiety can refer to any polymeric or molecular chain that is configured to form a binding interaction with a transportate. A filamentous moiety can be configured to tether an attached transportate to a surface while allowing the attached transportate to be positioned at a distance away from the surface. Thus, a transportate that is attached to a surface via a filamentous moiety can be positioned at a desired depth in a fluid that is in contact with the surface. The depth can be adjusted by adjusting the length of the filament between the surface and the point of attachment for the transportate. A filamentous moiety can comprise a linear chain or moiety, a branched chain or moiety, or a dendrimeric moiety. A filamentous moiety may be configured to form a binding interaction with a transportate. In some cases, a filamentous moiety may be configured to form a non-covalent binding interaction with a transportate. In other cases, a filamentous moiety may be configured to form a covalent binding interaction with a transportate.

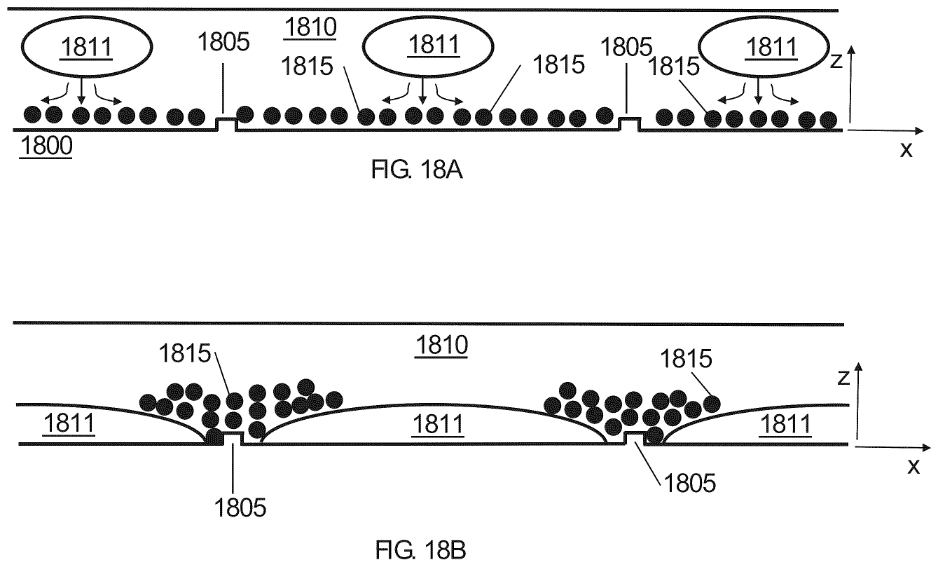

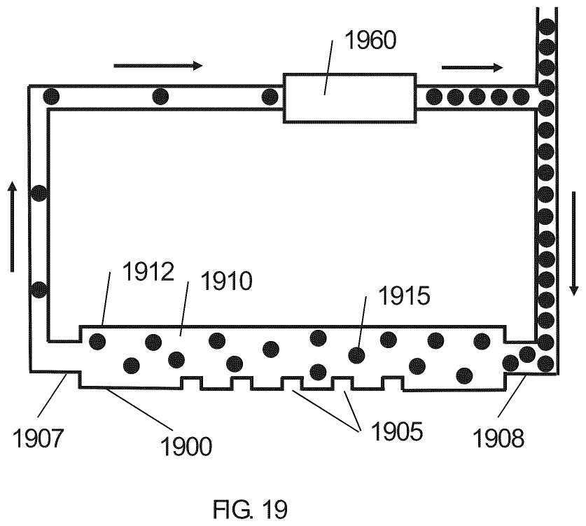

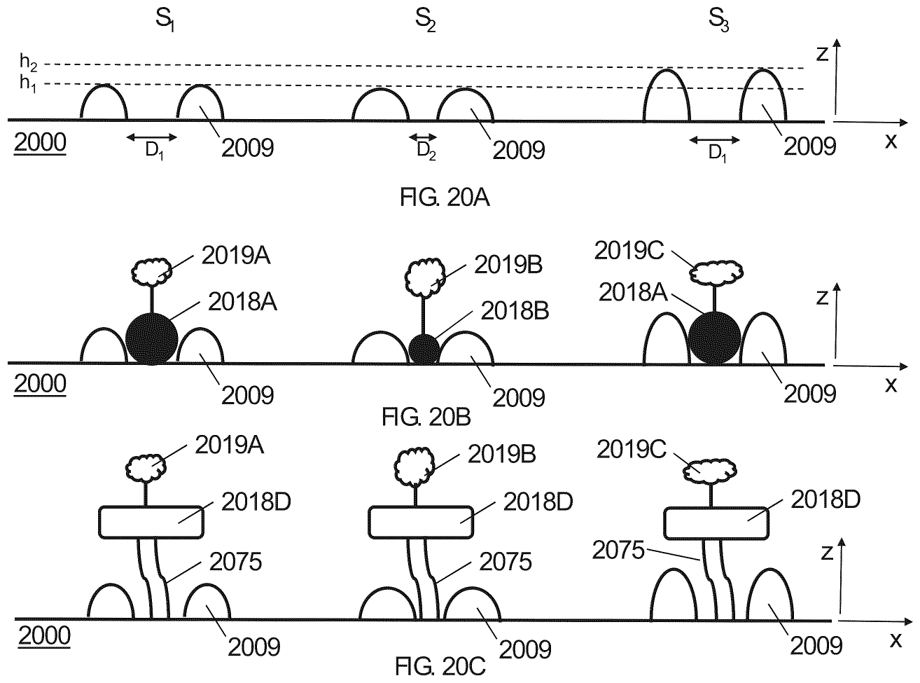

FIGS. 13A-13G illustrates aspects of array sites comprising filamentous moieties. FIG. 13A depicts a solid support 1300 comprising a population of array sites 1305, with each array site 1305 having a filamentous moiety 1306 coupled to the array site 1305 (e.g., covalently coupled or non-covalently coupled). The solid support 1300 is contacted with a fluidic medium 1310 of total depth or thickness Hₜₒₜ that produces an extended or non-globular state of the filamentous moieties 1306. In the extended or non-globular state, the filamentous moieties have an average length in the fluidic medium 1310 of H_{fm}. FIG. 13B depicts the configuration of FIG. 13A contacted with a population of analytes and/or anchoring moieties 1315. Analyte and/or anchoring moieties 1315 may be introduced by spiking them into the fluidic medium 1310 or by displacing a volume of the fluidic medium 1310 with a volume of fluidic medium 1310 containing the analytes and/or anchoring moieties 1315. The filamentous moieties 1306 may remain in an extended or non-globular state with an average length of H_{fm}, or may attain a different average length depending upon the effect of the presence of the analytes and/or anchoring moieties 1315. FIG. 13C depicts a configuration of the array system in which analytes and/or anchoring moieties 1315 have bound to the filamentous moieties 1306. Excess or unbound analytes and/or anchoring moieties 1315 may be removed from the system, for example by extraction, exchange, or rinsing of the fluidic medium 1310. Depending upon the nature of binding interactions between the filamentous moieties 1306 and the analytes and/or anchoring moieties 1315, the analytes and/or anchoring moieties 1315 may be bound to differing portions of the filamentous moieties 1306. FIG. 13D depicts a configuration of the array system at a time after the time depicted in FIG. 13C, in which all analytes and/or anchoring moieties 1315 have become bound to array sites 1305 after transferring along or adjacent to the filamentous moieties 1306.

FIGS. 13E-13G illustrate advantageous configurations of filamentous moieties. Such filamentous moieties 1306 may be configured to form one or more simultaneous and/or successive binding interactions with an analyte and/or anchoring moiety 1315. FIG. 13E depicts an electrically-charged filamentous moiety 1306A (e.g., a cationic polymer, an anionic polymer, a zwitterionic polymer, etc.) that is configured to form one or more electrostatic interactions with an electrically-charged anchoring moiety 1315A, in which the electrically-charged anchoring moiety 1315A is coupled to an analyte 1320. Breaking and forming of electrostatic binding interactions may facilitate “walking” of the electrically-charged anchoring moiety 1315A along the electrically-charged filamentous moiety 1306A, for example toward the portion of the electrically-charged filamentous moiety 1306A coupled to an array site. FIG. 13F depicts an oligonucleotide filamentous moiety 1306B. The oligonucleotide filamentous moiety 1306B comprises a repeated nucleotide sequences that binds to an oligonucleotide coupling moiety 1316 that is attached to the anchoring moiety 1315B. In some cases, the oligonucleotide coupling moiety may have a weakened binding interaction with the oligonucleotide filamentous moiety 1306B, for example by non-complete nucleotide sequence complementarity (as shown) or by a low dsDNA melting temperature (for example due to a short region of complementarity). In such a configuration, a complementary oligonucleotide coupling moiety 1316 may alternately de-hybridize and re-hybridize to the oligonucleotide filamentous moiety 1306B, thereby transferring along the oligonucleotide filamentous moiety 1306B. FIG. 13G depicts a filamentous moiety 1306C comprising a coupling moiety 1317A (e.g., a terminal coupling moiety or a branched coupling moiety) that is bound (e.g., covalently or non-covalently) to a complementary coupling moiety 1317B, in which the complementary coupling moiety 1317B is attached to an anchoring moiety 1315C. In some cases, the binding interaction between the coupling moiety 1317A and complementary coupling moiety 1317B may be non-dissociable over a timescale greater than or equal to the useful lifetime of an array of analytes (e.g., a time length of an assay). The coupling moiety 1317A and complementary coupling moiety 1317B can form a non-covalent binding interaction, such as by a receptor-ligand binding pair. The coupling moiety 1317A and complementary coupling moiety 1317B can form a covalent binding interaction, such as by a Click-type reaction pair. The configuration of FIG. 13G may be useful if filamentous moiety 1306C is configured to undergo a configuration change (e.g., polymer coiling, oligonucleotide or peptide folding) that brings the analyte 1320 and/or anchoring moiety 1315C closer to an array site to which the filamentous moiety 1306C is coupled.

A population of array sites may comprise a population of filamentous moieties, in which the population of filamentous moieties has an average length of at least about 50 nanometers (nm), 100 nm, 200 nm, 500 nm, 750 nm, 1 micron (μm), 2 μm, 3 μm, 4 μm, 5 μm, 10 μm, 20 μm, 50 μm, 100 μm, or more than 100 μm. Alternatively or additionally, a population of array sites may comprise a population of filamentous moieties, in which the population of filamentous moieties has an average length of no more than about 100 μm, 50 μm, 20 μm, 10 μm, 5 μm, 4 μm, 3 μm, 2 μm, 1 μm, 750 nm, 500 nm, 200 nm, 100 nm, 50 nm, or less than 50 nm. In some cases, average length can refer to an average length of filamentous moieties at their fully extended length. In other cases, average length can refer to an average length of filamentous moieties at their natural length within a fluidic medium (which may be a shorter length than a fully extended length). A population of filamentous moieties may comprise a range of moiety lengths, for example as characterized by a statistical distribution of lengths (e.g., a normal distribution, a bimodal distribution, a polymodal distribution, etc.). A filamentous moiety of a population of filamentous moieties may have a length of at least about 5 nanometers (nm), 10 nm, 50 nm, 100 nm, 200 nm, 500 nm, 750 nm, 1 micron (μm), 2 μm, 3 μm, 4 μm, 5 μm, 10 μm, 20 μm, 50 μm, 100 μm, 500 μm, or more than 500 μm. Alternatively or additionally, a filamentous moiety of a population of filamentous moieties may have a length of no more than about 500 μm, 100 μm, 50 μm, 20 μm, 10 μm, 5 μm, 4 μm, 3 μm, 2 μm, 1 μm, 750 nm, 500 nm, 200 nm, 100 nm, 50 nm, 10 nm, 5 nm, or less than 5 nm.

In some cases, a filamentous moiety may have a length, or a population of filamentous moieties may have an average length, that is extends a certain distance into a fluidic medium. It may be advantageous to have filamentous moieties extended into a fluidic medium, thereby increasing a likelihood of forming a binding interaction with a transportate within the fluidic medium. A filamentous moiety may have a length, or a population of filamentous moieties may have an average length of at least about 1%, 5%, 10%, 15%, 20%, 25%, 30%, 35%, 40%, 45%, 50%, or more than 50% of the thickness or depth of a layer of a fluidic medium, as set forth herein. Alternatively or additionally, a filamentous moiety may have a length, or a population of filamentous moieties may have an average length of no more than about 50%, 45%, 40%, 35%, 30%, 25%, 20%, 15%, 10%, 5%, 1%, or less than 1% of the thickness or depth of a layer of a fluidic medium, as set forth herein.

A method, as set forth herein may comprise one or more steps of: i) providing a population of filamentous moieties, in which the population of filamentous moieties is attached to a population of array sites, and in which at least a fraction (e.g., at least about 5%, 10%, 15%, 20%, 25%, 30%, 40%, 50%, 60%, 70%, 80%, 90%, 95%, or more than 95%) of the population of filamentous moieties have an extended or partially extended state (e.g., an average length of at least about 25%, 30%, 40%, 50%, 60%, 70%, 80%, 90%, 95%, or more than 95% of a maximum average length of the population of filamentous moieties), ii) binding transportates to filamentous moieties of the fraction of the population of filamentous moieties, iii) after binding the transportates to the filamentous moieties of the population of filamentous moieties, altering a configuration of the fraction of the population of filamentous moieties to a partially-coiled, coiled, or globular configuration (e.g., an average length of no more than about 25%, 20%, 15%, 10%, 5%, 1%, or less than 1% of a maximum average length of the population of filamentous moieties), and iv) after altering the configuration of the fraction of the population of filamentous moieties, binding the transportates to array sites of the population of array sites.

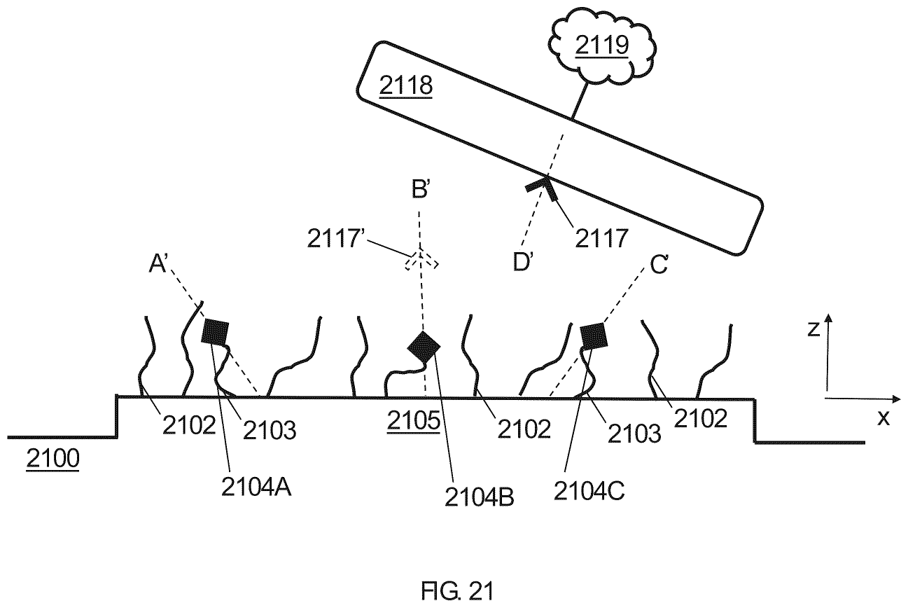

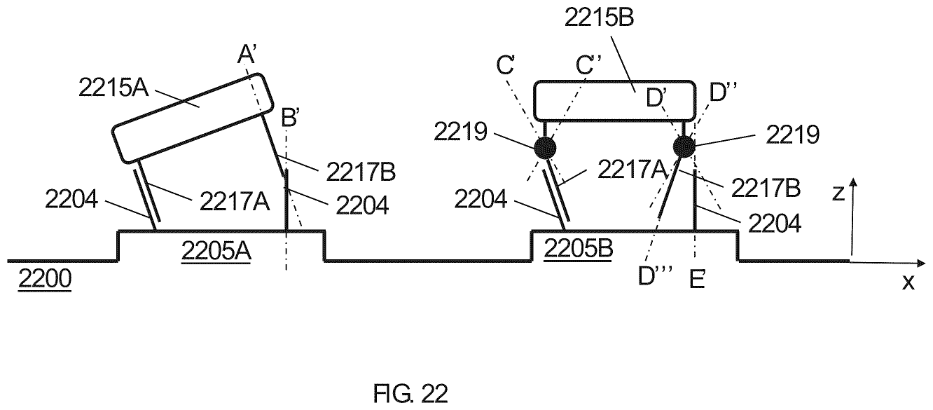

FIGS. 14A-14C depict differing methods of binding an analyte and/or anchoring moiety to an array site utilizing a filamentous moiety. FIG. 14A depicts a solid support 1400 containing an array site 1405, in which the array site comprises an attached filamentous moiety 1406. The filamentous moiety 1406 is bound to an anchoring moiety 1415 that is attached to an analyte 1420. FIG. 14B depicts a binding of the anchoring moiety 1415 and/or analyte 1420 to the array site 1405 by compacting the filamentous moiety, for example by coiling or folding the filamentous moiety. Compacting of a filamentous moiety may be achieved by any suitable method including changing a pH, ionic strength, or chemical composition of a fluidic medium (e.g., altering a concentration of a chaotrope, denaturant, or surfactant). In some cases, binding an analyte and/or anchoring moiety to an array site may comprise compacting (i.e., decreasing a length of) a filamentous moiety bound to the analyte and/or anchoring moiety. In some cases, binding an analyte and/or anchoring moiety to an array site may comprise: i) compacting a filamentous moiety bound to the analyte and/or anchoring moiety, and ii) after compacting the filamentous moiety, binding a coupling moiety of the analyte and/or anchoring moiety to a complementary coupling moiety of the array site. Optionally, a method may comprise a step of extending (i.e., increasing a length of) a filamentous moiety, for example by altering a composition, pH, or ionic strength of a fluidic medium. A filamentous moiety configuration like that of FIG. 13G may be advantageous for a compactable filamentous moiety due to the low likelihood of the analyte and/or anchoring moiety dissociating before it can be bound to the array site. FIG. 14C depicts an alternative “walking” mechanism in which an analyte 1420 or anchoring moiety 1415 can dissociate and re-associate to the filamentous moiety 1406 repeatedly, thereby being guided toward the array site by the chain of the filamentous moiety 1406. FIGS. 13A-13F depicts aspects of this method of utilizing a filamentous moiety. In some cases, the method of FIG. 14C may be combined with a method of altering a fluidic medium (e.g., pH, ionic strength, or composition change) that facilitates transfer of analytes 1420 and/or anchoring moieties 1415 toward a surface of a solid support 1400.

### Methods of Increasing Mass Transfer Across an Array Surface

A method, as set forth herein, may include a transportate association condition that increases a mass transfer of transportates across or adjacent to a surface of a solid support, in which the surface contains a population of array sites. A transportate association condition that increases a mass transfer of transportates across or adjacent to a surface of a solid support may be provided to increase site occupancy of an array of analytes, to decrease formation time of an array of analytes, to increase binding of assay agents at array sites, to decrease binding time of assay agents at array sites, or a combination thereof.

Methods described in this section may be advantageous when a solid support is configured with a large interstitial surface area relative to a surface area of array sites, or in which a total quantity of a population of array sites is greater than a total quantity of a population of transportates. In these situations, a likelihood or frequency of a transportate contacting an array site is reduced, thereby decreasing a likelihood or frequency of forming binding interactions between transportates and array sites.

Accordingly, methods are provided for: 1) increasing mass transfer of transportates across or adjacent to a surface of a solid support, or 2) concentrating transportates adjacent to array sites on a surface of a solid support. It will be understood that transportates that are adjacent to an array site are capable of binding, attaching or otherwise reacting with the array site. Mass transfer adjacent to a surface of a solid support may be limited due to quiescent flow or momentum transport in a boundary layer region adjacent to the surface. Accordingly, methods are provided for facilitating mass flux or fluid motion adjacent to a surface of a solid support.

In some cases, a transportate association condition may comprise agitation of a fluidic medium contacted with a surface of a solid support containing a population of array sites. Any conceivable method of agitating a fluidic medium may be utilized in an array-based system. Agitation of a fluidic medium may comprise providing a unidirectional or quasi-unidirectional flow to a fluidic medium (e.g., via peristaltic pumping of the fluidic medium). Agitation of a fluidic medium may comprise providing a bidirectional or quasi-bidirectional flow to a fluidic medium (e.g., via oscillatory pumping of the fluidic medium). In some cases, flow of a fluidic medium may have a direction that is substantially parallel to an average profile height of a surface of a solid support (e.g., a laminar flow profile). In other cases, flow of a fluidic medium may have a direction that is not parallel to an average profile height of a surface of a solid support (e.g., turbulent flow).

In some cases, control of fluidic motion may be achieved by disposing a population of array sites within a flow cell or fluidic cartridge. A flow cell or fluidic cartridge may provide control over fluid motion by limiting the locations where a fluidic medium can be injected or withdrawn into a reservoir, chamber, or channel containing a population of array sites. In some cases, a flow cell may comprise ports or manifolds that facilitate fluid ingress and egress from a reservoir, chamber, or channel containing a population of array sites. In some cases, a method of agitating a fluidic medium may comprise the steps of: i) delivering a first volume of a fluidic medium into a first port of a flow cell or fluidic cartridge, and ii) withdrawing a second volume of a fluidic medium out of a second port of the flow cell or fluidic cartridge. In other cases, a method of agitating a fluidic medium may comprise the steps of: i) delivering a first volume of a fluidic medium into a first port of a flow cell or fluidic cartridge, and ii) withdrawing a second volume of a fluidic medium out of the first port of the flow cell or fluidic cartridge.

Agitating a fluidic medium may comprise delivering (e.g. injecting) and/or withdrawing fluid from a flow cell or fluidic cartridge, in which the flow cell or fluidic cartridge contains a reservoir, chamber, or channel comprising a population of array sites. In some cases, agitating a fluidic medium in a volume comprising a population of array sites can comprise displacing the fluidic medium from the reservoir, chamber, or channel, for example by injecting a volume of a fluid that is greater than or equal to a volume of the fluidic medium or a volume of the reservoir, chamber, or channel containing the population of array sites. In some cases, agitating a fluidic medium in a volume comprising a population of array sites can comprise displacing a portion of the fluidic medium from the reservoir, chamber, or channel, for example by injecting a volume of a fluid that is less than a volume of the fluidic medium or a volume of the reservoir, chamber, or channel containing the population of array sites. A volume of fluid delivered to a reservoir, chamber, or channel containing a population of array sites may be substantially devoid of transportates.

In some cases, a method of agitating a fluidic medium may comprise providing a fluidic medium comprising a population of transportates to a flow cell or fluidic cartridge, in which a volume of the fluidic medium is greater than a volume of a reservoir, chamber, or channel of the flow cell or fluidic cartridge, and in which the reservoir, chamber, or channel of the flow cell or fluidic cartridge comprises a population of array sites. In some cases, a method may further comprise passing at least about 50%, 60%, 70%, 80%, 90%, 95%, 99%, or substantially 100% of the volume of the fluidic medium through the reservoir, chamber, or channel (e.g., by an oscillatory flow or by a peristaltic flow).

FIG. 19 depicts a system that is configured to provide a flow cell or fluidic cartridge with a volume of a fluidic medium comprising analytes and/or anchoring moieties, in which the volume of the fluidic medium is greater than a volume of the flow cell or fluidic cartridge. A flow cell 1912 comprises a solid support 1900 containing a population of array sites 1905. The fluidic medium 1910 is continuously circulated through the flow cell 1912 by injection trough an entrance port 1908 and discharge through an exit port 1907. Continuous circulation may inhibit deposition of analytes and/or anchoring moieties 1915 on or adjacent to a surface of the solid support 1900. Discharged fluidic medium 1910 contains a reduced concentration of analytes and/or anchoring moieties 1915 (due to deposition on array sites 1905). The fluidic medium 1910 with the reduced concentration of analytes and/or anchoring moieties 1915 is passed through a concentrating unit 1960 that discharges a stream of fluidic medium with an increased concentration of analytes and/or anchoring moieties 1915. The stream of fluidic medium 1910 with the increased concentration of analytes and/or anchoring moieties 1915 is returned to the flow cell 1912 by delivery through the entrance port 1908.

In some cases, a method of agitating a fluidic medium may comprise providing a fluidic medium that is substantially devoid of transportates to a flow cell or fluidic cartridge. In other cases, a method of agitating a fluidic medium may comprise providing a fluidic medium that contains transportates to a flow cell or fluidic cartridge.

In some cases, agitating a fluidic medium in a flow cell or fluidic cartridge may comprise forming a turbulent flow in the fluidic medium in a reservoir, chamber, or channel of the flow cell or fluidic cartridge. The skilled person will recognize that development of turbulent flow is a function of fluidic properties (e.g., density, viscosity, local or average fluid velocity) as well as a shape of a conduit (e.g., a reservoir, chamber, or channel of a flow cell or fluidic cartridge). Development of turbulent flow may be impacted by an average or local characteristic aspect ratio (e.g., ratio of height to width or ratio of height to length) of a reservoir, chamber, or channel of a flow cell or fluidic cartridge. A reservoir, chamber, or channel of a flow cell or fluidic cartridge may be provided with a characteristic aspect ratio of at least about 0.000001:1, 0.00001:1, 0.0001:1, 0.001:1, 0.01:1, 0.1:1, 0.5:1, 1:1, 2:1, 5:1, 10:1, 50:1, 100:1, 1000:1, 10000:1, 100000:1, 1000000:1, or more than 1000000:1. Alternatively or additionally, a reservoir, chamber, or channel of a flow cell or fluidic cartridge may be provided with a characteristic aspect ratio of no more than about 1000000:1, 100000:1, 10000:1, 1000:1, 100:1, 50:1, 10:1, 5:1, 2:1, 1:1, 0.5:1, 0.1:1, 0.01:1, 0.001:1, 0.0001:1, 0.00001:1, 0.000001:1, or less than 0.000001:1.

In some cases, particles other than transportates may be provided in a fluidic medium containing a population of transportates. Particles may be provided to facilitate generation of flow (e.g., turbulent flow) or to facilitate displacement of transportates on a surface of a solid support. In some cases, particles provided to a fluidic medium may comprise an electrical charge and/or a magnetic polarity. For example, electrically-charge particles may be used to induce electrokinetic flow in a fluidic medium that is exposed to an electric field (see for example Wang, G. R., et al. “There Can Be Turbulence in Microfluidics at Low Reynolds Numbers,” Lab on a Chip, 8, 2014, which is incorporated by reference in its entirety), or magnetic particles may be used to stir or circulate fluid via particle rotation in a magnetic field (see for example Willis, A. J., et al. “Rotating Magnetic Nanoparticle Clusters as Microdevices for Drug Delivery,” Int J Nanomedicine, 15, 2020, which is incorporated by reference in its entirety).

In some cases, particles may be provided to a fluidic medium, in which the particles have a higher density than a density of the fluidic medium. Accordingly, such particles may settle or sediment onto a surface of a solid support. FIGS. 15A-15C illustrate aspects of utilizing settled particles to generate flow near or adjacent to a surface of a solid support. FIG. 15A depicts a solid support 1500 comprising a population of array sites 1505, in which the solid support 1500 is contacted with a fluidic medium 1510. The fluidic medium contains a plurality of analytes and/or anchoring moieties 1515 of a population of analytes and/or anchoring moieties 1515 adjacent to the surface of the solid support 1500. The plurality of analytes and/or anchoring moieties 1515 has an average x-axis displacement from a nearest array site 1505 of D_{avg,i}. The fluidic medium further comprises a particle 1530 adjacent to the surface of the solid support 1500. FIG. 15B depicts a configuration of the solid support 1500 after motion has been induced in the particle 1530 (e.g., rolling or saltation caused by flow, translation caused by flow, magnetokinesis, or electrokinesis). Motion of the particle 1530 can induce velocity fields in the fluidic medium 1510, thereby displacing analytes and/or anchoring moieties 1515 in the x-axis direction (and possibly the z-axis direction). Depending upon the nature of the flow field formed by particle 1530 motion, the average x-axis displacement of analytes and/or anchoring moieties 1515 from a nearest array site 1505 may decrease to D_{avg,f}. FIG. 15C depicts a configuration of the solid support 1500 after convective fluid motion (i.e., buoyancy-driven flow) has been induced adjacent to the surface of the solid support 1500 by illumination of a photo-absorbing particle 1531 in a light field 1540 (alternatively, convective circulation can be induced by direct heating of the solid support 1500). Convective flow fields caused by heating of the particle 1531 can displace analytes and/or anchoring moieties 1515 in the x-axis direction (and possibly the z-axis direction). Depending upon the nature of the flow field formed by convective heat transfer, the average x-axis displacement of analytes and/or anchoring moieties 1515 from a nearest array site 1505 may decrease to D_{avg,f}.

It may be useful to provide a transportate association condition, in which the transportate association condition facilitates formation of an anisotropic analyte and/or anchoring moiety spatial distribution (e.g., surface density) on or adjacent to a surface of a solid support containing a population of array sites. FIGS. 16A-16B illustrate aspects of analyte and/or anchoring moiety 1615 spatial distributions on solid supports 1600 containing populations of array sites 1605. FIG. 16A depicts a solid support 1600 with a substantially uniform spatial distribution of analytes and/or anchoring moieties 1615 with respect to average surface density (e.g., analytes and/or anchoring moieties per unit of surface area). FIG. 16B depicts a solid support 1600 with an anisotropic spatial distribution of analytes and/or anchoring moieties 1615. A surface density of analytes and/or anchoring moieties 1615 in an interstitial region adjacent to an array site, or in contact with an array site, 1605 is greater than a surface density of an interstitial region at a region away from array sites (e.g., at a region centered halfway between two array sites).

In some cases, an anisotropic distribution of transportates may be formed on or adjacent to a surface of a solid support utilizing a vibrational, piezoelectric, or acoustic wave. A vibrational, piezoelectric, or acoustic wave may be provided to a solid support, thereby inducing motion of any particles, analytes, and/or anchoring moieties on or adjacent to a surface of the solid support. A vibrational, piezoelectric, or acoustic wave may be provided to a solid support, thereby inducing motion of a fluidic medium containing particles, analytes, and/or anchoring moieties that is contacted to a surface of the solid support. Vibration of a solid support or any material contacted thereto may occur in a steady-state fashion (i.e., a standing wave). For example, a standing wave may be formed that clusters together surface-adjacent transportates in a spatial distribution similar to that depicted in FIG. 16B. Vibration of a solid support or any material contacted thereto may occur in a transient fashion (i.e., a propagating wave). For example, acoustic waves may move clusters of transportates across a surface of a solid support (e.g., net motion in the x-axis and/or y-axis directions relative to FIG. 16A).

FIGS. 17A-17B illustrate the use of vibrational, piezoelectric, or acoustic waves to transfer particles, analytes, and/or anchoring moieties across a surface of a solid support. FIG. 17A depicts a solid support 1700 containing a population of array sites 1705 that is contacted with a fluidic medium 1710. The fluidic medium comprises a plurality of analytes and/or anchoring moieties 1715 of a population of analytes and/or anchoring moieties 1715 that are adjacent to a surface of the solid support 1700. The plurality of analytes and/or anchoring moieties 1715 has a substantially uniform spatial distribution along the direction of the x-axis. As shown in FIG. 17B, a standing acoustic or vibrational wave T with a wavelength P (substantially the same as the inter-site pitch) is applied to the solid support 1700, thereby displacing analytes and/or anchoring moieties 1715 toward array sites 1705.

In some cases, a transportate association condition may comprise depositing a fluid or material on an interstitial region of a solid support containing a population of array sites. In some cases, a method may comprise depositing a fluid or material on an interstitial region of a solid support containing a population of array sites, in which the fluid or material and the interstitial region have a mutual chemical property (e.g., substantially similar hydrophobicity, hydrophilicity, electrical charge, polarity, etc.), and in which the fluid or material and an array site have a dissimilar chemical property (substantially dissimilar hydrophobicity, hydrophilicity, electrical charge, polarity, etc.). For example, an array may be contacted with a hydrophobic oil that selectively binds to hydrophobic interstitial regions (e.g., regions only comprising an adhesion promoter like HMDS) and does not deposit on array sites comprising hydrophilic surface-coupled moieties. In some cases, a fluid or material may be deposited on an interstitial region of a solid support before the solid support is contacted with a fluidic medium comprising transportates. In other cases, a fluid or material may be deposited on an interstitial region of a solid support after the solid support is contacted with a fluidic medium comprising transportates.

Exemplary fluids or materials are numerous, depending upon the desired chemical property or properties of the fluid or material, and can include liquids (e.g., oils, halogenated alkanes, mercury, etc.) and solids (e.g., nanoparticles, polymers, halogenated polymers, etc.).

FIGS. 18A-18B illustrate mass transfer of transportates by deposition of a fluid or material on an interstitial region of a solid support. FIG. 18A depicts a solid support 1800 comprising a population of array sites 1805 that is contacted with a fluidic medium 1810. The fluidic medium comprises a plurality of analytes and/or anchoring moieties 1815 of a population of analytes and/or anchoring moieties 1815 that are adjacent to an interstitial region of the solid support 1800. The plurality of analytes and/or anchoring moieties 1815 has a substantially uniform spatial distribution in the x-axis direction. The fluidic medium 1810 further comprises a dispersion (e.g., a suspension, an emulsion, etc.) of a fluid or material 1850. The dispersion of fluid or material 1850 is translating toward the surface of the solid support 1800 (e.g., due to settling, sedimentation, emulsion breaking, centrifugation, precipitation, etc.), thereby producing a non-quiescent fluid flow field that can displace analytes and/or anchoring moieties 1815. FIG. 18B depicts a configuration of the solid support 1815 after deposition of the fluid or material 1850 on interstitial regions of the solid support 1850. The fluid or material 1850 has displaced the plurality of analytes and/or anchoring moieties 1815, thereby forming an anisotropic spatial distribution of analytes and/or anchoring moieties 1815, with highest concentration of analytes and/or anchoring moieties 1815 on or adjacent to array sites 1805. As shown in FIG. 18B, deposited fluid or material 1850 may form a surface or boundary that effectively forms wells adjacent to array sites. Such wells may favor concentration of analytes and/or anchoring moieties 1815 on or adjacent to array sites due to gravitational phenomena such as settling, sedimentation, centrifugation, etc. Further, the surface or boundary of the fluid or material 1850 may have a chemical property (e.g., hydrophobicity, hydrophilicity, electrical charge, polarity, etc.) that is dissimilar to a chemical property of an analyte and/or anchoring moiety 1815, thereby favoring mass transfer of the analyte and/or anchoring moiety 1815 toward an array site.

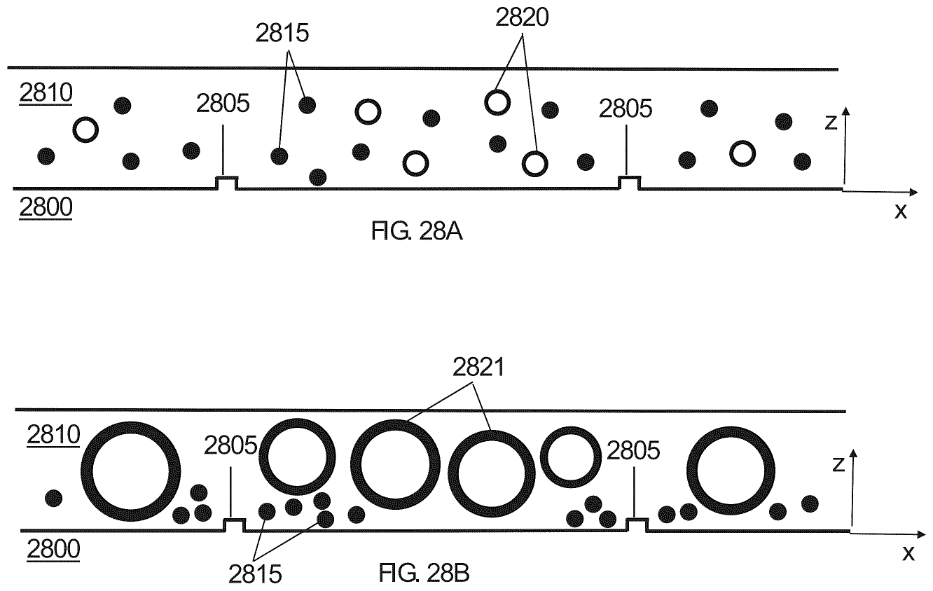

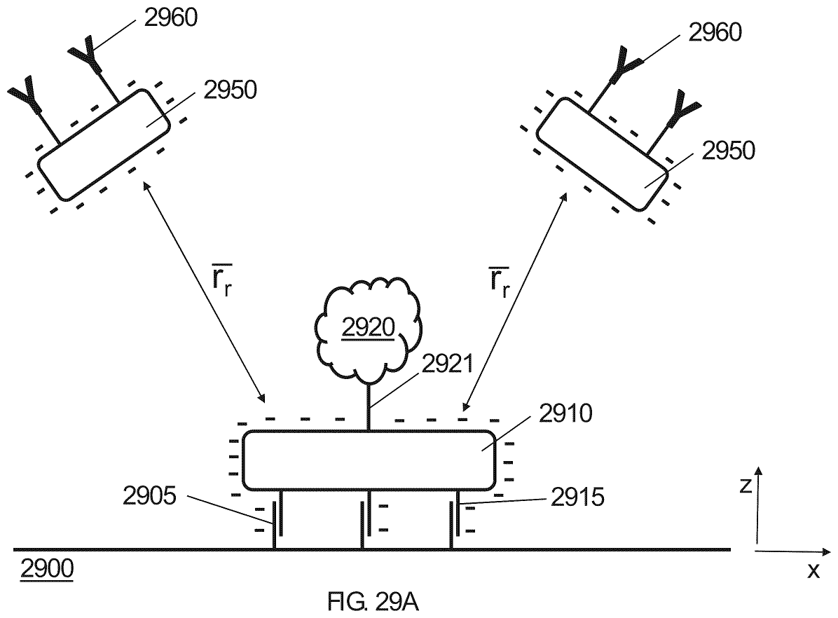

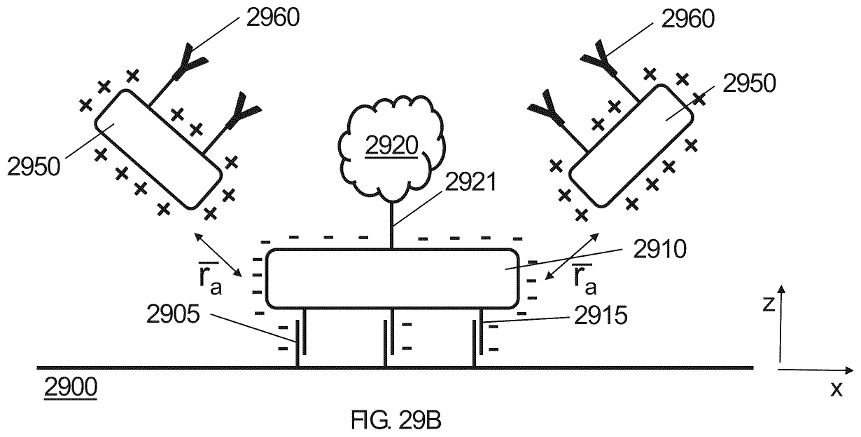

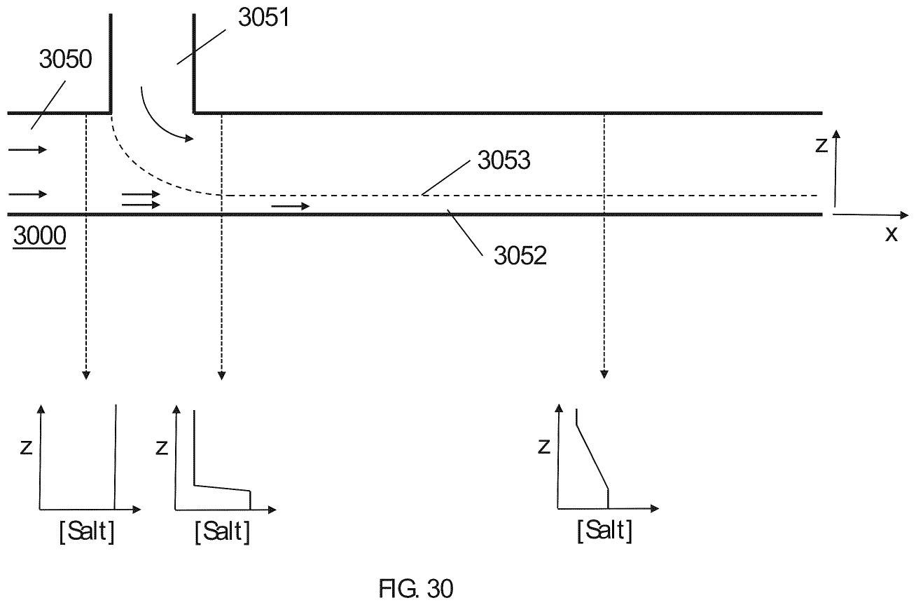

FIGS. 28A and 28B depict use of expanding particles to facilitate mass transfer toward an array surface. FIG. 28A depicts a first configuration in which a solid support 2800 comprising a plurality of array sites 2805 has been contacted with a fluidic medium 2810 comprising a plurality of particles 2815 (e.g., analytes, anchoring moieties, affinity agents, detectable probes, etc.). The fluidic medium 2810 further comprises a plurality of expanding particles 2820 in a smaller volumetric state. FIG. 28B depicts a second configuration in which the plurality of expanding particles 2820 have been induced to swell or expand to a larger volumetric state (e.g., by change in pH or ionic strength of the fluidic medium 2810). The increased volumes occupied by the expanding particles 2820 concentrates the particles 2815, thereby increasing the concentration of the particles 2815 adjacent to a surface of the solid support 2800 containing the array sites 2805. Optionally, the fluidic medium can be altered to return the expanding particles 2820 to the original smaller volumetric state before removing them from contact with the solid support 2800. Exemplary expanding particles can include certain polymer nanoparticles (e.g., polyacrylate expanding particles).

Interactions between particles contacted to arrays (e.g., anchoring moieties, analytes, affinity agents, detectable probes, etc.) and array surfaces or moieties bound thereto may affect the ability of the particles to contact and/or become bound at an array site. FIGS. 29A and 29B illustrate the impact of particle surface electrical charge on array interactions adjacent to an array surface. FIG. 29A depicts a solid support 2900 with an analyte 2920 that is attached to the solid support 2900 by an anchoring moiety 2910 (e.g., a nucleic acid nanoparticle). The anchoring moiety 2910 comprises a plurality of surface-coupling moieties 2915 (e.g., oligonucleotides, receptor-ligand binding components, etc.) that couples the anchoring moiety to a plurality of surface-coupled moieties 2905 (e.g., complementary components to the surface-coupling moieties 2915). The analyte 2920 is attached to the anchoring moiety 2910 by a linker 2921 (e.g., a nucleic acid linker, a polymer linker, etc.). The anchoring moiety 2910 has a negative net surface electrical charge. The solid support 2900 is contacted with a plurality of detectable probes comprising a retaining component 2950 (e.g., a nucleic acid nanoparticle, a polymer nanoparticle, etc.) attached to a plurality of affinity agents 2960. The retaining component 2950 has a negative net surface electrical charge. Repulsion interactions between the retaining component 2950 and the anchoring moiety 2910 increase an average distance rᵣ between the detectable probes and the array site. FIG. 29B depicts a substantially similar system to the system depicted in FIG. 29A, but the retaining components 2950 of the detectable probes have been modified to have a net positive surface electrical charge (e.g., by attaching amines to the surface of the retaining components 2950). Attractive interactions between the retaining component 2950 and the anchoring moiety 2910 decrease an average distance rₐ between the detectable probes and the array site such that rₐ<rᵣ. The skilled person will readily recognize that a fluidic medium can be modified to affect the relative attraction or repulsion between charge particles, for example by tuning fluidic ionic strength to increase or decrease bridging by ions.

In some cases, a method may further comprise separating a fluid or material from a solid support or an interstitial region thereof. A fluid or material may be separated from a solid support or an interstitial region thereof by any suitable method, such as solvent stripping or mechanical separation.

### Improving Binding Competency of Analytes or Anchoring Moieties

Methods for forming arrays of analytes via controlled deposition of the analytes at array sites are provided herein. In some cases, methods are provided for binding an analyte to an array site by a binding interaction between a coupling moiety attached to the analyte or an anchoring moiety coupled to the analyte with a complementary coupling moiety attached to an array site. In a preferable case, binding an analyte to an array site may comprise binding a polyvalent analyte or anchoring moiety (i.e., having two or more coupling moieties) to a polyvalent array site (i.e., having two or more complementary coupling moieties), thereby forming a plurality of binding interactions between the analyte and/or anchoring moiety and the array site. For example, an analyte may be attached to an anchoring moiety, in which the anchoring moiety contains about ten pendant oligonucleotide coupling moieties. The anchoring moiety may be contacted to an array site containing at least ten surface-coupled complementary oligonucleotides. Accordingly, up to ten binding interactions may form between the anchoring moiety and the array site.

Those skilled in the art will be able to provide analytes, anchoring moieties, and/or array sites with coupling moieties based on the teachings set forth herein. Accordingly, to the skilled person can determine whether an analyte or anchoring moiety will bind to an array site based upon the configurations of each entity. The rate at which an analyte or anchoring moiety will bind to an array site can be determined. It may take a longer amount of time to form an array of analytes with a high array site occupancy than expected; conversely, an array of analytes may have a lower than expected array site occupancy given a fixed amount of analyte deposition time.

FIGS. 20A-20C illustrate aspects of array site and analyte and/or anchoring moiety configuration that may affect a rate of binding interactions occurring between an array site and an analyte or anchoring moiety. FIG. 20A depicts a solid support 2000 containing a population of array sites (S₁, S₂, and S₃). Due to inherent variability in an array site formation process (e.g., photolithography or nanoimprint lithography), site diameters differ, with array sites S₁ and S₃ each having a diameter of about D₁, and array site S₂ having a diameter of about D₂. Each array site has a boundary material 2009 adjacent to the edge of the array site. The boundary material 2009 of each array site has a height that exceeds a height of a surface of the solid support 2000 or a height of the array site to which it is adjacent, thereby giving the sites a 3-D morphology (assuming the array site has an unseen y-axis dimension like FIG. 3). Due to inherent variability in an array site formation process, boundary material heights differ, with array sites S₁ and S₂ each having a boundary material 2009 height of about h₁, and array site S₃ having a boundary material 2009 height of about h₂.

FIG. 20B depicts anchoring moieties with differing binding competencies being utilized to facilitate analyte coupling to array sites. Two species of anchoring moieties are utilized, with the species distinguished by differing diameters or footprints (e.g., project surface areas of a face of the anchoring moiety). Arrays sites S₁ and S₃ bind anchoring moiety 2018A, which has a larger diameter or footprint but is sufficiently sized to fit into sites of diameter D₁. Array site S₂ binds anchoring moiety 2018B, which has a smaller diameter than anchoring moiety 2018A, and is able to fit into sites of diameter D₂. Due to binding of anchoring moieties 2018A and 2018B, analytes 2019A, 2019B, and 2019C are coupled to array sites S₁, S₂, and S₃, respectively. Of note, if an anchoring moiety, such as 2018A or 2018B was positioned adjacent to an array site but separated by a boundary material 2009 (like a configuration shown in FIG. 2), the anchoring moiety may be gravitationally inhibited from binding to the array site. Accordingly, it may be necessary to provide an analyte association condition, as set forth herein, that separates the anchoring moiety from the surface of the solid support, thereby facilitating contact between the anchoring moiety and the array site.

FIG. 20C depicts an alternative approach to anchoring moiety design, in which a single species of anchoring moiety has a range of binding competencies (e.g., is able to bind to more than one type or species of array site). The anchoring moiety 2018D has a diameter or footprint that optionally exceeds a diameter or footprint of an array site, but comprises coupling moieties 2075 of sufficient length and/or flexibility as to facilitate coupling of the coupling moieties to facilitate coupling of the anchoring moiety 2018D to array sites of varying diameter and/or boundary material 2009 height. Accordingly, due to binding of anchoring moieties 2018D, analytes 2019A, 2019B, and 2019C are coupled to array sites S₁, S₂, and S₃, respectively.

A rate of binding between an array site and an analyte and/or anchoring moiety, or pluralities thereof, may be affected by aspects of array site configuration and/or analyte or anchoring moiety configuration. Formation of a binding interaction between a coupling moiety attached to an analyte or anchoring moiety and a complementary coupling moiety attached to an array site can be a complex phenomenon that is influenced by simple co-location of the coupling moiety and the complementary coupling moiety, among other factors. FIG. 21 illustrates a binding scenario for an anchoring moiety 2118 with an attached coupling moiety 2117 and an attached analyte 2119. The anchoring moiety is approaching an array site 2105 of a solid support 2100, in which the array site contains a plurality of surface-coupled moieties, including passivating moieties 2102 (e.g., PEG, dextrans, etc.) and complementary coupling moieties 2104A, 2104B, and 2104C, each of which is attached to the array site 2105 by a passivating linker 2103 (e.g., PEG, dextran, etc.). At the instant depicted in FIG. 21, the coupling moiety 2117 has an orientation along axis D′, relative to the z- and x-axes. Likewise, complementary coupling moieties 2104A, 2104B, and 2104C have orientations along axes A′, B′, and C′, respectively. Because the complementary coupling moieties 2104A, 2104B, and 2104C, and the anchoring moiety 2118 and/or coupling moiety 2117 have degrees of freedom to alter their position or orientation, the depicted configuration can change temporally and spatially. To form a binding interaction between the coupling moiety 2117 and a complementary coupling moiety (e.g., 2104B), it may be necessary to achieve a proper orientation of both moieties (for example, receptor-ligand binding pairs can be sensitive to proper alignment of a ligand to a binding region of the receptor). An outline of a hypothetical coupling moiety 2117′ is shown to demonstrate a necessary alignment of the coupling moiety 2117 to the complementary coupling moiety 2104B to form a binding interaction. The two coupling moieties must have orientation axes that are substantially colinear to form a binding interaction. Moreover, at any given instant, motion of passivating moieties 2102 can partially or fully occlude complementary coupling moieties 2104A, 2104B, or 2104C, thereby inhibiting the ability of the occluded complementary coupling moiety to participate in a binding interaction. Given possible motion of the anchoring moiety 2118, coupling moiety 2117, and complementary coupling moieties 2104A, 2104B, and 2104C, a likelihood of forming a binding interaction during any instant of contact between the binding entities may be relatively low; rather, binding interactions can occur stochastically through repeated rearrangement of configurations of binding entities. Although FIG. 21 is exemplified with a coupling system like a receptor-ligand binding pair, the same considerations may hold true for other coupling approaches, like oligonucleotide hybridization or covalent bond formation.

Formation of binding interactions between array sites and analytes and/or anchoring moieties can be described individually through a statistical or stochastic description (e.g., a likelihood of a binding interaction forming for a given contact between the two binding entities), or at a continuum level through a binding kinetics description (e.g., a rate of binding interaction formation). A likelihood or rate of binding interaction formation may be dependent upon one or more aspects of array site configuration. A likelihood or rate of binding interaction formation may be dependent upon one or more aspects of analyte or anchoring moiety configuration.

In some cases, inherent variability of manufacturing workflows (e.g., lithography workflows or surface chemistry workflows) may produce arrays with variability in array site configuration. Such inherent variability may be a substantially fixed aspect of array behavior. For example, two random arrays from a same manufacturing run may be expected to have similar or even substantially identical array site binding competency profiles, suggesting similar or substantially identical outcomes in distributions of array site configurations and/or binding competencies. There may be a greater parameter space for design flexibility in analytes and/or anchoring moieties.

In an aspect, provided herein is a binding entity (e.g., analytes, anchoring moieties) comprising a plurality of coupling moieties, in which coupling moieties of the plurality of coupling moieties are coupled to the binding entity by a single-stranded linker. In another aspect, provided herein is a binding entity (e.g., analytes, anchoring moieties) comprising a plurality of coupling moieties, in which coupling moieties of the plurality of coupling moieties are coupled to the binding entity by a non-rigid linker.

FIG. 22 illustrates an advantage of incorporating non-rigid linkers into polyvalent binding entities (e.g., analytes, anchoring moieties). The system is simplified to depict a situation in which an anchoring moiety 2215 is configured to form two binding interactions with an array site (e.g., a bivalent binding entity), although the design concept is readily extended to an increased number of binding interactions. FIG. 22 depicts a solid support 2200 containing two array sites 2205A and 2205B. Each array site comprises surface-coupled, single-stranded complementary oligonucleotide coupling moieties 2204. Array site 2205A is contacted with an anchoring moiety 2215A (e.g., a nucleic acid nanoparticle) comprising two oligonucleotide coupling moieties 2217A and 2217B. Oligonucleotide coupling moiety 2217A has hybridized to a complementary coupling moiety 2204, forming a rigid double-stranded structure that coupled the anchoring moiety to array site 2205A. Due to the rigidity of the double-stranded DNA coupling interaction, the second oligonucleotide coupling moiety 2217B is not positioned in an optimal orientation to form a coupling interaction with the second complementary oligonucleotide coupling moiety 2204 (e.g., axis A′ being substantially parallel to axis B′). Accordingly, the rigidity of the double-stranded coupling interaction may inhibit anchoring moiety 2215A from forming a polyvalent binding interaction with the array site 2205A.

Continuing with FIG. 22, array site 2205B is contacted with an anchoring moiety 2215B that comprises two comprising two oligonucleotide coupling moieties 2217A and 2217B, each of which is attached to the anchoring moiety 2215B by a non-rigid linker 2219 (e.g., a polymer chain, a single-stranded peptide, a single-stranded nucleic acid). Oligonucleotide coupling moiety 2217A has hybridized to a complementary coupling moiety 2204, forming a rigid double-stranded structure that coupled the anchoring moiety to array site 2205B. Due to the presence of the non-rigid linker 2219, the anchoring moiety 2215 has increased degrees of freedom (as shown by the region formed between axes C′ and C″) to reorient its position. Moreover, the second oligonucleotide coupling moiety 2217B has increased degrees of freedom (as shown by the region formed between axes D′ and D″) to alter its position or conformation relative to the second surface-coupled complementary oligonucleotide coupling moiety 2204. Accordingly, there is an increased likelihood that the second oligonucleotide coupling moiety 2217B can become positioned in an optimal orientation to form a coupling interaction with the second complementary oligonucleotide coupling moiety 2204 (e.g., axis D′″ being substantially parallel to axis E′), thereby forming a polyvalent binding interaction between the anchoring moiety 2215B and array site 2205B.

A binding entity (e.g., an analyte, an anchoring moiety) may comprise a plurality of coupling moieties. Surprisingly, there may be an optimum total quantity of coupling moieties attached to a binding entity, beyond which there is no discernible increase in binding strength of the binding entity to an array site, and beyond which there is a discernible decrease in rate of binding or likelihood of binding to an array site during a fixed period of time. In some cases, increasing a total quantity of coupling moieties attached to a binding entity can increase the surface density of coupling moieties attached to a face of the binding entity. A decrease in binding rate may be due to occlusion of binding of a first coupling moiety by a second coupling moiety. A binding entity may comprise no more than about 1000, 500, 250, 200, 150, 125, 100, 75, 50, 40, 30, 29, 28, 27, 26, 25, 24, 23, 22, 21, 20, 19, 18, 17, 16, 15, 14, 13, 12, 11, 10, 9, 8, 7, 6, 5, 4, 3, or less than 3 coupling moieties. Alternatively or additionally, a binding entity may comprise at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 75, 100, 125, 150, 200, 250, 500, 1000, or more than 1000 coupling moieties.

In some cases, a binding entity (e.g., an analyte or an anchoring moiety) may comprise a face, in which the face comprises an average projected surface area (i.e., a surface area when the footprint of the face is projected orthogonally onto a planar surface), and wherein the face comprises a surface, moiety, or plurality of moieties that provide a substantially similar orientation to any moiety attached to the face. In particular cases, a binding entity may comprise two or more faces. In other cases, a binding entity may comprise no defined faces (e.g., a spherical or oblate particle). A binding entity may comprise a total effective surface area (i.e., a surface area of a boundary that completely surrounds the external portions of the binding entity). An average coupling moiety surface density may be calculated for a binding entity or a face thereof based upon the total effective surface area or the average project surface area, respectively. A binding entity or a face thereof may have an average coupling moiety surface density of no more than about 10¹⁸ coupling moieties/cm², 10¹⁷ coupling moieties/cm², 10¹⁶ coupling moieties/cm², 10¹⁵ coupling moieties/cm², 10¹⁴ coupling moieties/cm², 10¹¹ coupling moieties/cm², 5×10¹² coupling moieties/cm², 10¹² coupling moieties/cm², 5×10¹¹ coupling moieties/cm², 10¹¹ coupling moieties/cm², 5×10¹⁰ coupling moieties/cm², 10¹⁰ coupling moieties/cm², 10⁹ coupling moieties/cm², 10⁸ coupling moieties/cm², 10⁷ coupling moieties/cm², 10⁶ coupling moieties/cm², or less than 10⁶ coupling moieties/cm². Alternatively or additionally, a binding entity or a face thereof may have an average coupling moiety surface density of at least about 10⁶ coupling moieties/cm², 10⁷ coupling moieties/cm², 10⁸ coupling moieties/cm², 10⁹ coupling moieties/cm², 10¹⁰ coupling moieties/cm², 5×10¹⁰ coupling moieties/cm², 10¹¹ coupling moieties/cm², 5×10¹¹ coupling moieties/cm², 10¹² coupling moieties/cm², 5×10¹² coupling moieties/cm², 10¹¹ coupling moieties/cm², 10¹⁴ coupling moieties/cm², 10¹⁵ coupling moieties/cm², 10¹⁶ coupling moieties/cm², 10¹⁷ coupling moieties/cm², 10¹⁸ coupling moieties/cm², or more than 10¹⁸ coupling moieties/cm².

A coupling moiety may be attached to a binding entity (e.g., an analyte or an anchoring moiety) by a linker or linking moiety. In some cases, a linker or linking moiety may comprise a non-rigid linker. A non-rigid linker may comprise any suitable chemical moiety that provides at least one degree of translational freedom and one degree of rotational freedom to a coupling moiety with respect to a binding entity to which the non-rigid linker couples the coupling moiety. A non-rigid linker may comprise a polymeric chain (i.e., a homogeneous or heterogeneous chain of covalently bonded monomers). A non-rigid linker may comprise a linear polymeric chain. A non-rigid linker may be substantially free of branching (e.g., a polymeric chain coupled to a binding entity at a first terminus and coupled to a coupling moiety at a second terminus, and further comprising an attached polymeric chain at a residue or monomer between the first and second termini). A non-rigid linker may be substantially free of unsaturated covalent bonds (e.g., alkenyl or alkynyl moieties, carbonyl moieties, etc.). A non-rigid linker may be substantially devoid of pendant polyatomic functional groups (e.g., alkyl groups, hydroxyl groups, carboxyl groups, amine groups, etc). A non-rigid linker may comprise a monatomic bridge (e.g., an ether linkage, a sulfide linkage). A non-rigid linker may comprise an alkyl chain or moiety as a segment of a polymeric chain (e.g., an ethyl or propyl group).

A non-rigid linker may comprise a polymeric chain, in which the polymeric chain can comprise a plurality of covalently bonded monomers. A non-rigid linker may comprise a homopolymer moiety (e.g., polyethylene glycol, a single-nucleotide repeat, a single amino acid repeat, etc.). For example, a non-rigid linker may comprise a linear polyethylene glycol chain. In another example, a non-rigid linker may comprise an oligonucleotide containing a poly-T, poly-A, poly-G, or poly-C repeat. A non-rigid linker may comprise a heteropolymer moiety (e.g., a copolymer, a varying sequence of nucleotides, a varying sequence of amino acids. In some cases, a polymeric chain may comprise a monomer sequence that does not have self-complementarity. For example, a nucleotide sequence of an oligonucleotide linker or an amino acid sequence of a peptide chain may be designed to avoid self-complementarity. In some cases, a polymeric chain may comprise a monomer sequence that does not form a secondary and/or tertiary structure. For example, an oligonucleotide linker may contain a sequence that does not form a hairpin or loop structure due to self-complementarity. In some cases, a non-rigid linker comprising a polymeric chain may form an ordered structure (e.g., secondary structure, tertiary structure, quaternary structure) in the presence of a first fluidic medium (e.g., a buffer that is substantially devoid of a chaotrope or denaturant), and may be substantially devoid of the ordered structure in the presence of a second fluidic medium (a buffer containing a chaotrope or denaturant).

A linker or linking moiety may comprise a particular number of residues (e.g., polymeric monomers, nucleotides, amino acids, saccharides, etc.), such as at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, or more than 100 residues. A linker or linking moiety may comprise no more than about 100, 90, 80, 70, 60, 50, 45, 40, 35, 30, 29, 28, 27, 26, 25, 24, 23, 22, 21, 20, 19, 18, 17, 16, 15, 14, 13, 12, 11, 10, 9, 8, 7, 6, 5, 4, 3, 2, or less than 2 residues. Alternatively or additionally, a linker or linking moiety may have a length (e.g., in an extended state, based upon an average residue length, etc.) of at least about 0.1 nanometers (nm), 0.5 nm, 1 nm, 5 nm, 10 nm, 15 nm, 20 nm, 25 nm, 30 nm, 35 nm, 40 nm, 45 nm, 50 nm, or more than 50 nm. A linker or linking moiety may have a length of no more than about 50 nm, 45 nm, 40 nm, 35 nm, 30 nm, 25 nm, 20 nm, 15 nm, 10 nm, 5 nm, 1 nm, 0.5 nm, 0.1 nm, or less than 0.1 nm.

A linker or linking moiety may be characterized by a persistence length. A persistence length may be a suitable quantitative proxy for rigidity of a linker or linking moiety. For example, the persistence length of double-stranded DNA is about 2 orders of magnitude greater than the persistence length of single-stranded DNA due to the increased rigidity of the double-stranded secondary structure. A linker or linking moiety may have a persistence length of no more than about 50 nm, 25 nm, 10 nm, 5 nm, 4 nm, 3 nm, 2 nm, 1 nm, 0.9 nm, 0.8 nm, 0.7 nm, 0.6 nm, 0.5 nm, 0.4 nm, 0.3 nm, 0.2 nm, 0.1 nm, or less than 0.1 nm. Alternatively or additionally, a linker or linking moiety may have a persistence length of at least about 0.1 nm, 0.2 nm, 0.3 nm, 0.4 nm, 0.5 nm, 0.6 nm, 0.7 nm, 0.8 nm, 0.9 nm, 1 nm, 2 nm, 3 nm, 4 nm, 5 nm, 10 nm, 25 nm, 50 nm, or more than 50 nm.

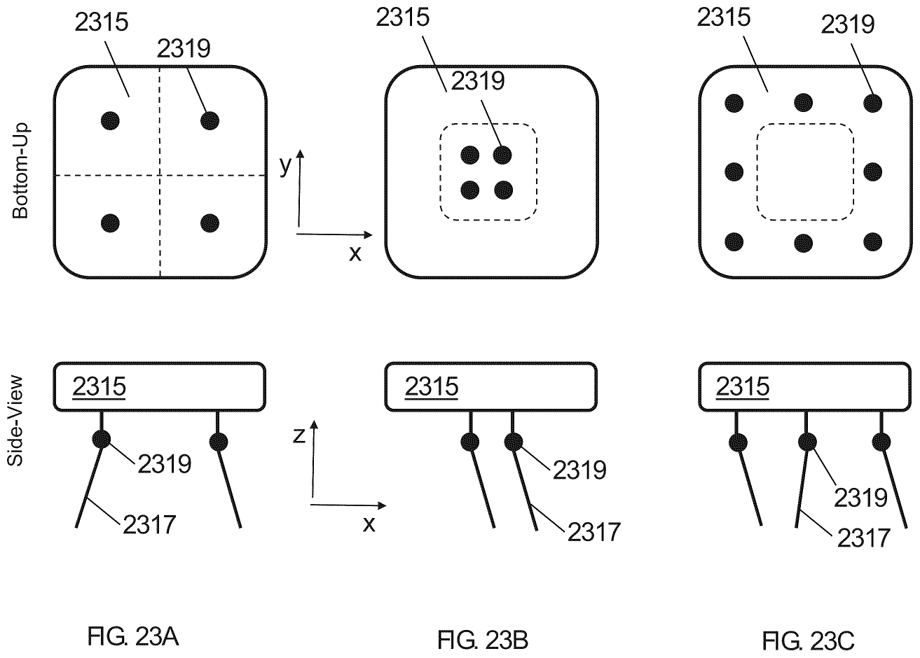

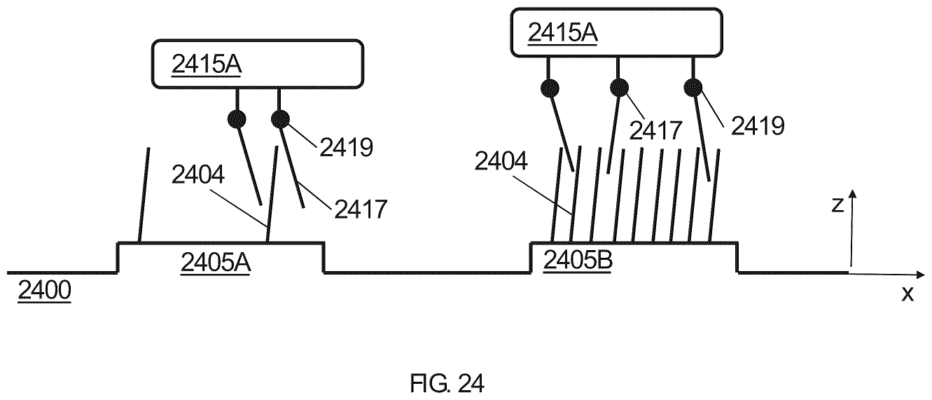

A plurality of coupling moieties may be affixed to a face of a binding entity (e.g., an analyte, an anchoring moiety) with a spatial distribution that favors formation of polyvalent binding interactions. FIGS. 23A-23C and FIG. 24 illustrate aspects of spatial distribution of coupling moieties on binding entities that may influence a rate or likelihood of forming binding interactions between a binding entity and an array site. In some cases, nucleic acid nanoparticles, such as nucleic acid origami, may be advantageous due to the tunable nature of nucleic acid origami. A linker may attach a coupling moiety to a specific location or may orient a coupling moiety in a specific direction on a face of a nucleic acid nanoparticle.

FIGS. 23A-23C depict different possible spatial distributions of linkers 2319 (e.g., non-rigid linkers) on a face of a binding entity 2315. The upper figures display a bottom-up view of the face of the binding entity 2315 to which the linkers 2319 are attached. The lower figures display a cross-sectional or side face view of the binding entity 2315 with coupling moieties 2317 attached to the binding entity 2315 by the linkers 2319. FIG. 23A depicts a substantially uniform spatial distribution of the linkers 2319 on the face of the binding entity. Accordingly, the average distance from a linker 2319 to a nearest linker 2319 is substantially the same for all linkers 2319 attached to the face of the binding entity 2319. Alternatively, the face of the binding entity 2315 is shown divided into four equal quadrants, in which each quadrant contains an identical or similar quantity of coupling moieties. FIG. 23B depicts a clustered spatial distribution, in which the linkers 2319 are confined within a limited area of the face of the binding entity 2315 (shown by the dashed box), and few or no linkers 2319 are located on areas of the face outside the limited area. The limited area is depicted as being centered, but in some cases the region can be located away from the center of the face of the binding entity 2315. FIG. 23C depicts a configuration with a flanking spatial distribution, in which the linkers 2319 are located near edges or boundaries of the face of the binding entity 2315. The face of the binding entity 2315 may comprise an internal region (shown by the dashed box) that contains few or no linkers 2319 located within the internal region.

FIG. 24 illustrates aspects of polyvalent binding interactions that may influence location and/or orientation of coupling moieties on binding entities. A solid support 2400 contains a first array site 2405A, and a second array site 2405B. The first array site 2405A comprises a low surface density of complementary coupling moieties 2404. The second array site 2405B contains a higher surface density of complementary coupling moieties than array site 2405A. Array site 2405A is contacted with binding entity 2415A, which has a binding competency for array sites with low surface density of complementary coupling moieties 2404. Binding entity 2415A comprises a similar structure as shown in FIG. 23B, in which coupling moieties 2417 are attached to binding entity 2415A by linkers 2419 that are attached to a face of the binding entity 2415A with a clustered spatial distribution. This spatial arrangement of coupling moieties 2417 may be advantageous for low surface densities of complementary coupling moieties 2404 because a new binding interaction between a coupling moiety 2417 and a complementary coupling moiety 2404 can rapidly form if a prior binding interaction dissociates (i.e., the binding entity 2415A displays multiple potential binding partners to the complementary coupling moiety 2404, thereby permitting repeated dissociation and re-association of the binding entity 2415A to the array site 2405A before the binding entity 2415A can diffuse away). Array site 2405B is contacted with binding entity 2415B, which has a binding competency for array sites with higher surface densities of complementary coupling moieties 2404. Binding entity 2415A comprises a similar structure as shown in FIG. 23C, in which coupling moieties 2417 are attached to binding entity 2415A by linkers 2419 that are attached to a face of the binding entity 2415A with a flanking spatial distribution. This spatial arrangement of coupling moieties 2417 may be advantageous for higher surface densities of complementary coupling moieties 2404 each coupling moiety 2417 has multiple complementary coupling moieties 2404 with which it can form a binding interaction, and because the relatively large spatial separation between each coupling moiety 2417 provides a greater likelihood that the orientation of each coupling moiety 2417 can adjust to facilitate formation of a binding interaction with one of the plurality of complementary coupling moieties 2404.

A site may have an average coupling moiety surface density of no more than about 10¹⁸ coupling moieties/cm², 10¹⁷ coupling moieties/cm², 10¹⁶ coupling moieties/cm², 10¹⁵ coupling moieties/cm², 10¹⁴ coupling moieties/cm², 10¹³ coupling moieties/cm², 5×10¹² coupling moieties/cm², 10¹² coupling moieties/cm², 5×10¹¹ coupling moieties/cm², 10¹¹ coupling moieties/cm², 5×10¹⁰ coupling moieties/cm², 10¹⁰ coupling moieties/cm², 10⁹ coupling moieties/cm², 10⁸ coupling moieties/cm², 10⁷ coupling moieties/cm², 10⁶ coupling moieties/cm², or less than 10⁶ coupling moieties/cm². Alternatively or additionally, a site may have an average coupling moiety surface density of at least about 106 coupling moieties/cm², 10⁷ coupling moieties/cm², 10⁸ coupling moieties/cm², 10⁹ coupling moieties/cm², 10¹⁰ coupling moieties/cm², 5×10¹⁰ coupling moieties/cm², 10¹¹ coupling moieties/cm², 5×10¹¹ coupling moieties/cm², 10¹² coupling moieties/cm², 5×10¹² coupling moieties/cm², 10¹³ coupling moieties/cm², 10¹⁴ coupling moieties/cm², 10¹⁵ coupling moieties/cm², 10¹⁶ coupling moieties/cm², 10¹⁷ coupling moieties/cm², 10¹⁸ coupling moieties/cm², or more than 10¹⁸ coupling moieties/cm².

A site may comprise a plurality of surface-coupled moieties. The surface-coupled moieties may comprise passivating moieties. The surface-coupled moieties may comprise coupling moieties. In some cases, a surface-coupled moiety may comprise a passivating moiety and a coupling moiety (e.g., an oligonucleotide attached to a PEG linker). In some cases, a site may be provided with a plurality of surface-coupled moieties, in which substantially all surface-coupled moieties comprise passivating moieties (e.g., PEG moieties, alkyl moieties, etc.). In some cases, a site may be provided with a plurality of surface-coupled moieties, in which substantially all surface-coupled moieties comprise coupling moieties. Alternatively, a site may be provided with a plurality of surface-coupled moieties, in which a fraction of the surface-coupled moieties comprise a coupling moiety. A plurality of surface-coupled moieties may comprise a ratio of molecules comprising a coupling moiety to molecules not comprising a coupling moiety (e.g., passivating moieties) of at least about 1:1000000, 1:100000, 1:10000, 1:1000, 1:500, 1:200, 1:100, 1:50, 1:20, 1:10, 1:5, 1:2, 1:1, 2:1, 5:1, 10:1, 100:1, 1000:1, or more than 1000:1. Alternatively or additionally, a plurality of surface-coupled moieties may comprise a ratio of molecules comprising a coupling moiety to molecules not comprising a coupling moiety (e.g., passivating moieties) of no more than about 1000:1, 100:1, 10:1, 5:1, 2:1, 1:1, 1:2, 1:5, 1:10, 1:20, 1:50, 1:100, 1:200, 1:500, 1:1000, 1:10000, 1:100000, 1:1000000, or less than 1:1000000.

In a useful configuration, analytes or anchoring moieties comprising at least one surface-coupling moiety may be bound to a site comprising a surface-coupled moiety, in which the surface-coupled moiety comprises a complementary coupling moiety that is configured to bind to the surface-coupling moiety, and in which the surface-coupling moiety and the surface-coupled moiety each individually comprise a linker moiety. Preferably, the surface-coupling moiety and the surface-coupled moiety each individually comprise a flexible linker moiety (e.g., a PEG moiety).

### Polypeptide Assays

The present disclosure provides compositions, apparatus and methods that can be useful for characterizing sample components, such as proteins, nucleic acids, cells or other species, by obtaining multiple separate and non-identical measurements of the sample components. In particular configurations, the individual measurements may not, by themselves, be sufficiently accurate or specific to make the characterization, but an aggregation of the multiple non-identical measurements can allow the characterization to be made with a high degree of accuracy, specificity and confidence. For example, the multiple separate measurements can include subjecting the sample to reagents that are promiscuous with regard to recognizing multiple components of the sample. Accordingly, a first measurement carried out using a first promiscuous reagent may perceive a first subset of sample components without distinguishing one component from another. A second measurement carried out using a second promiscuous reagent may perceive a second subset of sample components, again, without distinguishing one component from another. However, a comparison of the first and second measurements can distinguish: (i) a sample component that is uniquely present in the first subset but not the second; (ii) a sample component that is uniquely present in the second subset but not the first; (iii) a sample component that is uniquely present in both the first and second subsets; or (iv) a sample component that is uniquely absent in the first and second subsets. The number of promiscuous reagents used, the number of separate measurements acquired, and degree of reagent promiscuity (e.g. the diversity of components recognized by the reagent) can be adjusted to suit the component diversity expected for a particular sample.

The present disclosure provides assays that are useful for detecting one or more analytes. Exemplary assays are set forth herein in the context of detecting proteins. Those skilled in the art will recognize that methods, compositions and apparatus set forth herein can be adapted for use with other analytes such as nucleic acids, polysaccharides, metabolites, vitamins, hormones, enzyme co-factors and others set forth herein or known in the art. Particular configurations of the methods, apparatus and compositions set forth herein can be made and used, for example, as set forth in U.S. Pat. No. 10,473,654 or US Pat. App. Pub. Nos. 2020/0318101 A1 or 2020/0286584 A1, each of which is incorporated herein by reference. Exemplary methods, systems and compositions are set forth in further detail below.

A composition, apparatus or method set forth herein can be used to characterize an analyte, or moiety thereof, with respect to any of a variety of characteristics or features including, for example, presence, absence, quantity (e.g. amount or concentration), chemical reactivity, molecular structure, structural integrity (e.g. full length or fragmented), maturation state (e.g. presence or absence of pre- or pro-sequence in a protein), location (e.g. in an analytical system, subcellular compartment, cell or natural environment), association with another analyte or moiety, binding affinity for another analyte or moiety, biological activity, chemical activity or the like. An analyte can be characterized with regard to a relatively generic characteristic such as the presence or absence of a common structural feature (e.g. amino acid sequence length, overall charge or overall pKa for a protein) or common moiety (e.g. a short primary sequence motif or post-translational modification for a protein). An analyte can be characterized with regard to a relatively specific characteristic such as a unique amino acid sequence (e.g. for the full length of the protein or a motif), an RNA or DNA sequence that encodes a protein (e.g. for the full length of the protein or a motif), or an enzymatic or other activity that identifies a protein. A characterization can be sufficiently specific to identify an analyte, for example, at a level that is considered adequate or unambiguous by those skilled in the art.

In particular configurations, a protein can be detected using one or more affinity agents having known or measurable binding affinity for the protein. For example, an affinity agent can bind a protein to form a complex and a signal produced by the complex can be detected. A protein that is detected by binding to a known affinity agent can be identified based on the known or predicted binding characteristics of the affinity agent. For example, an affinity agent that is known to selectively bind a candidate protein suspected of being in a sample, without substantially binding to other proteins in the sample, can be used to identify the candidate protein in the sample merely by observing the binding event. This one-to-one correlation of affinity agent to candidate protein can be used for identification of one or more proteins. However, as the protein complexity (i.e. the number and variety of different proteins) in a sample increases, or as the number of different candidate proteins to be identified increases, the time and resources to produce a commensurate variety of affinity agents having one-to-one specificity for the proteins approaches limits of practicality.

Methods set forth herein, can be advantageously employed to overcome these constraints. In particular configurations, the methods can be used to identify a number of different candidate proteins that exceeds the number of affinity agents used. For example, the number of candidate proteins identified can be at least 5×, 10×, 25×, 50×, 100× or more than the number of affinity agents used. This can be achieved, for example, by (1) using promiscuous affinity agents that bind to multiple different candidate proteins suspected of being present in a given sample, and (2) subjecting the protein sample to a set of promiscuous affinity agents that, taken as a whole, are expected to bind each candidate protein in a different combination, such that each candidate protein is expected to be encoded by a unique profile of binding and non-binding events. Promiscuity of an affinity agent is a characteristic that can be understood relative to a given population of proteins. Promiscuity can arise due to the affinity agent recognizing an epitope that is known to be present in a plurality of different candidate proteins suspected of being present in the given population of unknown proteins. For example, epitopes having relatively short amino acid lengths such as dimers, trimers, or tetramers can be expected to occur in a substantial number of different proteins in the human proteome. Alternatively or additionally, a promiscuous affinity agent can recognize different epitopes (e.g. epitopes differing from each other with regard to amino acid composition or sequence), the different epitopes being present in a plurality of different candidate proteins. For example, a promiscuous affinity agent that is designed or selected for its affinity toward a first trimer epitope may bind to a second epitope that has a different sequence of amino acids when compared to the first epitope.

Although performing a single binding reaction between a promiscuous affinity agent and a complex protein sample may yield ambiguous results regarding the identity of the different proteins to which it binds, the ambiguity can be resolved when the results are combined with other identifying information about those proteins. The identifying information can include characteristics of the protein such as length (i.e. number of amino acids), hydrophobicity, molecular weight, charge to mass ratio, isoelectric point, chromatographic fractionation behavior, enzymatic activity, presence or absence of post translational modifications or the like. The identifying information can include results of binding with other promiscuous affinity agents. For example, a plurality of different promiscuous affinity agents can be contacted with a complex population of proteins, wherein the plurality is configured to produce a different binding profile for each candidate protein suspected of being present in the population. In this example, each of the affinity agents can be distinguishable from the other affinity agents, for example, due to unique labeling (e.g. different affinity agents having different luminophore labels), unique spatial location (e.g. different affinity agents being located at different addresses in an array), and/or unique time of use (e.g. different affinity agents being delivered in series to a population of proteins). Accordingly, the plurality of promiscuous affinity agents produces a binding profile for each individual protein that can be decoded to identify a unique combination of epitopes present in the individual protein, and this can in turn be used to identify the individual protein as a particular candidate protein having the same or similar unique combination of epitopes. The binding profile can include observed binding events as well as observed non-binding events and this information can be evaluated in view of the expectation that particular candidate proteins produce a similar binding profile, for example, based on presence and absence of particular epitopes in the candidate proteins.

In some configurations, distinct and reproducible binding profiles may be observed for one or more unknown proteins in a sample. However, in many cases one or more binding events produces inconclusive or even aberrant results and this, in turn, can yield ambiguous binding profiles. For example, observation of binding outcome for a single-molecule binding event can be particularly prone to ambiguities due to stochasticity in the behavior of single molecules when observed using certain detection hardware. The present disclosure provides methods that provide accurate protein identification despite ambiguities and imperfections that can arise in many contexts. In some configurations, methods for identifying, quantitating or otherwise characterizing one or more proteins in a sample utilize a binding model that evaluates the likelihood or probability that one or more candidate proteins that are suspected of being present in the sample will have produced an empirically observed binding profile. The binding model can include information regarding expected binding outcomes (e.g. binding or non-binding) for binding of one or more affinity reagent with one or more candidate proteins. The information can include an apriori characteristic of a candidate protein, such as presence or absence of a particular epitope in the candidate protein or length of the candidate protein. Alternatively or additionally, the information can include empirically determined characteristics such as propensity or likelihood that the candidate protein will bind to a particular affinity reagent. Accordingly, a binding model can include information regarding the propensity or likelihood of a given candidate protein generating a false positive or false negative binding result in the presence of a particular affinity reagent, and such information can optionally be included for a plurality of affinity reagents.

Methods set forth herein can be used to evaluate the degree of compatibility of one or more empirical binding profiles with results computed for various candidate proteins using a binding model. For example, to identify an unknown protein in a sample of many proteins, an empirical binding profile for the protein can be compared to results computed by the binding model for many or all candidate proteins suspected of being in the sample. In some configurations of the methods set forth herein, identity for the unknown protein is determined based on a likelihood of the unknown protein being a particular candidate protein given the empirical binding pattern or based on the probability of a particular candidate protein generating the empirical binding pattern. Optionally a score can be determined from the measurements that are acquired for the unknown protein with respect to many or all candidate proteins suspected of being in the sample. A digital or binary score that indicates one of two discrete states can be determined. In particular configurations, the score can be non-digital or non-binary. For example, the score can be a value selected from a continuum of values such that an identity is made based on the score being above or below a threshold value. Moreover, a score can be a single value or a collection of values. Particularly useful methods for identifying proteins using promiscuous reagents, serial binding measurements and/or decoding with binding models are set forth, for example, in U.S. Pat. No. 10,473,654 US Pat. App. Pub. No. 2020/0318101 A1 or Egertson et al., BioRxiv (2021), DOI: 10.1101/2021.10.11.463967, each of which is incorporated herein by reference.

The present disclosure provides compositions, apparatus and methods for detecting one or more proteins. A protein can be detected using one or more affinity agents having binding affinity for the protein. The affinity agent and the protein can bind each other to form a complex and, during or after formation, the complex can be detected. The complex can be detected directly, for example, due to a label that is present on the affinity agent or protein. In some configurations, the complex need not be directly detected, for example, in formats where the complex is formed and then the affinity agent, protein, or a label component that was present in the complex is detected.

Many protein detection methods, such as enzyme linked immunosorbent assay (ELISA), achieve high-confidence characterization of one or more protein in a sample by exploiting high specificity binding of antibodies, aptamers or other binding agents to the protein(s) and detecting the binding event while ignoring all other proteins in the sample. ELISA is generally carried out at low plex scale (e.g. from one to a hundred different proteins detected in parallel or in succession) but can be used at higher plexity. ELISA methods can be carried out by detecting immobilized binding agents and/or proteins in multiwell plates, on arrays, or on particles in microfluidic devices. Exemplary plate-based methods include, for example, the MULTI-ARRAY technology commercialized by MesoScale Diagnostics (Rockville, Maryland) or Simple Plex technology commercialized by Protein Simple (San Jose, CA). Exemplary, array-based methods include, but are not limited to those utilizing Simoa® Planar Array Technology or Simoa® Bead Technology, commercialized by Quanterix (Billerica, MA). Further exemplary array-based methods are set forth in U.S. Pat. Nos. 9,678,068; 9,395,359; 8,415,171; 8,236,574; or 8,222,047, each of which is incorporated herein by reference. Exemplary microfluidic detection methods include those commercialized by Luminex (Austin, Texas) under the trade name xMAP® technology or used on platforms identified as MAGPIX®, LUMINEX® 100/200 or FEXMAP 3D®.

Other detection methods that can also be used, for example at low plex scale, include procedures that employ SOMAmer reagents and SOMAscan assays commercialized by Soma Logic (Boulder, CO). In one configuration, a sample is contacted with aptamers that are capable of binding proteins with specificity for the amino acid sequence of the proteins. The resulting aptamer-protein complexes can be separated from other sample components, for example, by attaching the complexes to beads (or other solid support) that are removed from other sample components. The aptamers can then be isolated and, because the aptamers are nucleic acids, the aptamers can be detected using any of a variety of methods known in the art for detecting nucleic acids, including for example, hybridization to nucleic acid arrays, PCR-based detection, or nucleic acid sequencing. Exemplary methods and compositions are set forth in U.S. Pat. Nos. 7,855,054; 7,964,356; 8,404,830; 8,945,830; 8,975,026; 8,975,388; 9,163,056; 9,938,314; 9,404,919; 9,926,566; 10,221,421; 10,239,908; 10,316,321 10,221,207 or 10,392,621, each of which is incorporated herein by reference.

In some detection assays, a protein can be cyclically modified and the modified products from individual cycles can be detected. In some configurations, a protein can be sequenced by a sequential process in which each cycle includes steps of detecting the protein and removing one or more terminal amino acids from the protein. Optionally, one or more of the steps can include adding a label to the protein, for example, at the amino terminal amino acid or at the carboxy terminal amino acid. In particular configurations, a method of detecting a protein can include steps of (i) exposing a terminal amino acid on the protein; (ii) detecting a change in signal from the protein; and (iii) identifying the type of amino acid that was removed based on the change detected in step (ii). The terminal amino acid can be exposed, for example, by removal of one or more amino acids from the amino terminus or carboxyl terminus of the protein. Steps (i) through (iii) can be repeated to produce a series of signal changes that is indicative of the sequence for the protein.

In a first configuration of a cyclical protein detection method, one or more types of amino acids in the protein can be attached to a label that uniquely identifies the type of amino acid. In this configuration, the change in signal that identifies the amino acid can be loss of signal from the respective label. For example, lysines can be attached to a distinguishable label such that loss of the label indicates removal of a lysine. Alternatively or additionally, other amino acid types can be attached to other labels that are mutually distinguishable from lysine and from each other. For example, lysines can be attached to a first label and cysteines can be attached to a second label, the first and second labels being distinguishable from each other. Exemplary compositions and techniques that can be used to remove amino acids from a protein and detect signal changes are those set forth in Swaminathan et al., Nature Biotech. 36:1076-1082 (2018); or U.S. Pat. Nos. 9,625,469 or 10,545,153, each of which is incorporated herein by reference. Methods and apparatus under development by Erisyon, Inc. (Austin, TX) may also be useful for detecting proteins.

In a second configuration of a cyclical protein detection method, a terminal amino acid of a protein can be recognized by an affinity agent that is specific for the terminal amino acid or specific for a label moiety that is present on the terminal amino acid. The affinity agent can be detected on the array, for example, due to a label on the affinity agent. Optionally, the label is a nucleic acid barcode sequence that is added to a primer nucleic acid upon formation of a complex. For example, a barcode can be added to the primer via ligation of an oligonucleotide having the barcode sequence or polymerase extension directed by a template that encodes the barcode sequence. The formation of the complex and identity of the terminal amino acid can be determined by decoding the barcode sequence. Multiple cycles can produce a series of barcodes that can be detected, for example, using a nucleic acid sequencing technique. Exemplary affinity agents and detection methods are set forth in US Pat. App. Pub. No. 2019/0145982 A1; 2020/0348308 A1; or 2020/0348307 A1, each of which is incorporated herein by reference. Methods and apparatus under development by Encodia, Inc. (San Diego, CA) may also be useful for detecting proteins.

Cyclical removal of terminal amino acids from a protein can be carried out using an Edman-type sequencing reaction in which a phenyl isothiocyanate reacts with a N-terminal amino group under mildly alkaline conditions (e.g. about pH 8) to form a cyclical phenylthiocarbamoyl Edman complex derivative. The phenyl isothiocyanate may be substituted or unsubstituted with one or more functional groups, linker groups, or linker groups containing functional groups. An Edman-type sequencing reaction can include variations to reagents and conditions that yield a detectable removal of amino acids from a protein terminus, thereby facilitating determination of the amino acid sequence for a protein or portion thereof. For example, the phenyl group can be replaced with at least one aromatic, heteroaromatic or aliphatic group which may participate in an Edman-type sequencing reaction, non-limiting examples including: pyridine, pyrimidine, pyrazine, pyridazoline, fused aromatic groups such as naphthalene and quinoline), methyl or other alkyl groups or alkyl group derivatives (e.g., alkenyl, alkynyl, cyclo-alkyl). Under certain conditions, for example, acidic conditions of about pH 2, derivatized terminal amino acids may be cleaved, for example, as a thiazolinone derivative. The thiazolinone amino acid derivative under acidic conditions may form a more stable phenylthiohydantoin (PTH) or similar amino acid derivative which can be detected. This procedure can be repeated iteratively for residual protein to identify the subsequent N-terminal amino acid. Many variations of Edman-type degradation have been described and may be used including, for example, a one-step removal of an N-terminal amino acid using alkaline conditions (Chang, J. Y., FEBS LETTS., 1978, 91(1), 63-68). In some cases, Edman-type reactions may be thwarted by N-terminal modifications which may be selectively removed, for example, N-terminal acetylation or formylation (e.g., see Gheorghe M. T., Bergman T. (1995) in Methods in Protein Structure Analysis, Chapter 8: Deacetylation and internal cleavage of Proteins for N-terminal Sequence Analysis. Springer, Boston, MA. https://doi.org/10.1007/978-1-4899-1031-8_8).

Non-limiting examples of functional groups for substituted phenyl isothiocyanate may include ligands (e.g. biotin and biotin analogs) for known receptors, labels such as luminophores, or reactive groups such as click functionalities (e.g. compositions having an azide or acetylene moiety). The functional group may be a DNA, RNA, peptide or small molecule barcode or other tag which may be further processed and/or detected.

The removal of an amino terminal amino acid using Edman-type processes can utilize at least two main steps, the first step includes reacting an isothiocyanate or equivalent with protein N-terminal residues to form a relatively stable Edman complex, for example, a phenylthiocarbamoyl complex. The second step can include removing the derivatized N-terminal amino acid, for example, via heating. The protein, now having been shortened by one amino acid, may be detected, for example, by contacting the protein with a labeled affinity agent that is complementary to the amino terminus and examining the protein for binding to the agent, or by detecting loss of a label that was attached to the removed amino acid.

Edman-type processes can be carried out in a multiplex format to detect, characterize or identify a plurality of proteins. A method of detecting a protein can include steps of (i) exposing a terminal amino acid on a protein at an address of an array; (ii) binding an affinity agent to the terminal amino acid, where the affinity agent includes a nucleic acid tag, and where a primer nucleic acid is present at the address; (iii) extending the primer nucleic acid, thereby producing an extended primer having a copy of the tag; and (iv) detecting the tag of the extended primer. The terminal amino acid can be exposed, for example, by removal of one or more amino acids from the amino terminus or carboxyl terminus of the protein. Steps (i) through (iv) can be repeated to produce a series of tags that is indicative of the sequence for the protein. The method can be applied to a plurality of proteins on the array and in parallel. Whatever the plexity, the extending of the primer can be carried out, for example, by polymerase-based extension of the primer, using the nucleic acid tag as a template. Alternatively, the extending of the primer can be carried out, for example, by ligase- or chemical-based ligation of the primer to a nucleic acid that is hybridized to the nucleic acid tag. The nucleic acid tag can be detected via hybridization to nucleic acid probes (e.g. in an array), amplification-based detections (e.g. PCR-based detection, or rolling circle amplification-based detection) or nuclei acid sequencing (e.g. cyclical reversible terminator methods, nanopore methods, or single molecule, real time detection methods). Exemplary methods that can be used for detecting proteins using nucleic acid tags are set forth in US Pat. App. Pub. No. 2019/0145982 A1; 2020/0348308 A1; or 2020/0348307 A1, each of which is incorporated herein by reference.

A protein can optionally be detected based on its enzymatic or biological activity. For example, a protein can be contacted with a reactant that is converted to a detectable product by an enzymatic activity of the protein. In other assay formats, a first protein having a known enzymatic function can be contacted with a second protein to determine if the second protein changes the enzymatic function of the first protein. As such, the first protein serves as a reporter system for detection of the second protein. Exemplary changes that can be observed include, but are not limited to, activation of the enzymatic function, inhibition of the enzymatic function, attenuation of the enzymatic function, degradation of the first protein or competition for a reactant or cofactor used by the first protein. Proteins can also be detected based on their binding interactions with other molecules such as proteins, nucleic acids, nucleotides, metabolites, hormones, vitamins, small molecules that participate in biological signal transduction pathways, biological receptors or the like. For example, a protein that participates in a signal transduction pathway can be identified as a particular candidate protein by detecting binding to a second protein that is known to be a binding partner for the candidate protein in the pathway.

The presence or absence of post-translational modifications (PTM) can be detected using a composition, apparatus or method set forth herein. A PTM can be detected using an affinity agent that recognizes the PTM or based on a chemical property of the PTM. Exemplary PTMs that can be detected, identified or characterized include, but are not limited to, myristoylation, palmitoylation, isoprenylation, prenylation, farnesylation, geranylgeranylation, lipoylation, flavin moiety attachment, Heme C attachment, phosphopantetheinylation, retinylidene Schiff base formation, dipthamide formation, ethanolamine phosphoglycerol attachment, hypusine, beta-Lysine addition, acylation, acetylation, deacetylation, formylation, alkylation, methylation, C-terminal amidation, arginylation, polyglutamylation, polyglyclyation, butyrylation, gamma-carboxylation, glycosylation, glycation, polysialylation, malonylation, hydroxylation, iodination, nucleotide addition, phosphoate ester formation, phosphoramidate formation, phosphorylation, adenylylation, uridylylation, propionylation, pyrolglutamate formation, S-glutathionylation, S-nitrosylation, S-sulfenylation, S-sulfinylation, S-sulfonylation, succinylation, sulfation, glycation, carbamylation, carbonylation, isopeptide bond formation, biotinylation, carbamylation, oxidation, reduction, pegylation, ISGylation, SUMOylation, ubiquitination, neddylation, pupylation, citrullination, deamidation, elminylation, disulfide bridge formation, proteolytic cleavage, isoaspartate formation, racemization, and protein splicing.

PTMs may occur at particular amino acid residues of a protein. For example, the phosphate moiety of a particular proteoform can be present on a serine, threonine, tyrosine, histidine, cysteine, lysine, aspartate or glutamate residue of the protein. In other examples, an acetyl moiety can be present on the N-terminus or on a lysine; a serine or threonine residue can have an O-linked glycosyl moiety; an asparagine residue can have an N-linked glycosyl moiety; a proline, lysine, asparagine, aspartate or histidine amino acid can be hydroxylated; an arginine or lysine residue can be methylated; or the N-terminal methionine or at a lysine amino acid can be ubiquitinated.

In some configurations of the apparatus and methods set forth herein, one or more proteins can be detected on a solid support. For example, protein(s) can be coupled to a support, the support can be contacted with detection agents (e.g. affinity agents) in solution, the agents can interact with the protein(s), thereby producing a detectable signal, and then the signal can be detected to determine the presence of the protein(s). In multiplexed versions of this approach, different proteins can be coupled to different addresses in an array, and the probing and detection steps can occur in parallel. In another example, affinity agents can be coupled to a solid support, the support can be contacted with proteins in solution, the proteins can interact with the affinity agents, thereby producing a detectable signal, and then the signal can be detected to determine presence, quantity or characteristics of the proteins. This approach can also be multiplexed by coupling different affinity agents to different addresses of an array.

Proteins, affinity agents or other objects of interest can be coupled to a solid support via covalent or non-covalent bonds. For example, a linker can be used to covalently couple a protein or other object of interest to an array. A particularly useful linker is a structured nucleic acid particle such as a nucleic acid nanoball (e.g. a concatemeric amplicon produced by rolling circle replication of a circular nucleic acid template) or a nucleic acid origami. For example, a plurality of proteins can be conjugated to a plurality of structured nucleic acid particles, such that each protein-conjugated particle forms an address in the array. Exemplary linkers for coupling proteins, or other objects of interest, to an array or other solid support are set forth in US Pat. App. Pub. No. 2021/0101930 A1, which is incorporated herein by reference.

A protein can be detected based on proximity of two or more affinity agents. For example, the two affinity agents can include two components each: a receptor component and a nucleic acid component. When the affinity agents bind in proximity to each other, for example, due to ligands for the respective receptors being on a single protein, or due to the ligands being present on two proteins that associate with each other, the nucleic acids can interact to cause a modification that is indicative of the two ligands being in proximity. Optionally, the modification can be polymerase catalyzed extension of one of the nucleic acids using the other nucleic acid as a template. As another option, one of the nucleic acids can form a template that acts as splint to position other nucleic acids for ligation to an oligonucleotide. Exemplary methods are commercialized by Olink Proteomics AB (Uppsala Sweden) or set forth in U.S. Pat. Nos. 7,306,904; 7,351,528; 8,013,134; 8,268,554 or 9,777,315, each of which is incorporated herein by reference.

A method or apparatus of the present disclosure can optionally be configured for optical detection (e.g. luminescence detection). Analytes or other entities can be detected, and optionally distinguished from each other, based on measurable characteristics such as the wavelength of radiation that excites a luminophore, the wavelength of radiation emitted by a luminophore, the intensity of radiation emitted by a luminophore (e.g. at particular detection wavelength(s)), luminescence lifetime (e.g. the time that a luminophore remains in an excited state) or luminescence polarity. Other optical characteristics that can be detected, and optionally used to distinguish analytes, include, for example, absorbance of radiation, resonance Raman, radiation scattering, or the like. A luminophore can be an intrinsic moiety of a protein or other analyte to be detected, or the luminophore can be an exogenous moiety that has been synthetically added to a protein or other analyte.

A method or apparatus of the present disclosure can use a light sensing device that is appropriate for detecting a characteristic set forth herein or known in the art. Particularly useful components of a light sensing device can include, but are not limited to, optical sub-systems or components used in nucleic acid sequencing systems. Examples of useful sub systems and components thereof are set forth in US Pat. App. Pub. No. 2010/0111768 A1 or U.S. Pat. Nos. 7,329,860; 8,951,781 or 9,193,996, each of which is incorporated herein by reference. Other useful light sensing devices and components thereof are described in U.S. Pat. Nos. 5,888,737; 6,175,002; 5,695,934; 6,140,489; or 5,863,722; or US Pat. Pub. Nos. 2007/007991 A1, 2009/0247414 A1, or 2010/0111768; or WO2007/123744, each of which is incorporated herein by reference. Light sensing devices and components that can be used to detect luminophores based on luminescence lifetime are described, for example, in U.S. Pat. Nos. 9,678,012; 9,921,157; 10,605,730; 10,712,274; 10,775,305; or 10,895,534, each of which is incorporated herein by reference.

Luminescence lifetime can be detected using an integrated circuit having a photodetection region configured to receive incident photons and produce a plurality of charge carriers in response to the incident photons. The integrated circuit can include at least one charge carrier storage region and a charge carrier segregation structure configured to selectively direct charge carriers of the plurality of charge carriers directly into the charge carrier storage region based upon times at which the charge carriers are produced. See, for example, U.S. Pat. Nos. 9,606,058, 10,775,305, and 10,845,308, each of which is incorporated herein by reference. Optical sources that produce short optical pulses can be used for luminescence lifetime measurements. For example, a light source, such as a semiconductor laser or LED, can be driven with a bipolar waveform to generate optical pulses with FWHM durations as short as approximately 85 ps having suppressed tail emission. See, for example, in U.S. Pat. No. 10,605,730, which is incorporated herein by reference.

For configurations that use optical detection (e.g. luminescent detection), one or more analytes (e.g. proteins) may be immobilized on a surface, and this surface may be scanned with a microscope to detect any signal from the immobilized analytes. The microscope itself may include a digital camera or other luminescence detector configured to record, store, and analyze the data collected during the scan. A luminescence detector of the present disclosure can be configured for epiluminescent detection, total internal reflection (TIR) detection, waveguide assisted excitation, or the like.

A light sensing device may be based upon any suitable technology, and may be, for example, a charged coupled device (CCD) sensor that generates pixilated image data based upon photons impacting locations in the device. It will be understood that any of a variety of other light sensing devices may also be used including, but not limited to, a detector array configured for time delay integration (TDI) operation, a complementary metal oxide semiconductor (CMOS) detector, an avalanche photodiode (APD) detector, a Geiger-mode photon counter, a photomultiplier tube (PMT), charge injection device (CID) sensors, JOT image sensor (Quanta), or any other suitable detector. Light sensing devices can optionally be coupled with one or more excitation sources, for example, lasers, light emitting diodes (LEDs), arc lamps or other energy sources known in the art.

An optical detection system can be configured for single molecule detection. For example, waveguides or optical confinements can be used to deliver excitation radiation to locations of a solid support where analytes are located. Zero-mode waveguides can be particularly useful, examples of which are set forth in U.S. Pat. Nos. 7,181,122, 7,302,146, or 7,313,308, each of which is incorporated herein by reference. Analytes can be confined to surface features, for example, to facilitate single molecule resolution. For example, analytes can be distributed into wells having nanometer dimensions such as those set forth in U.S. Pat. Nos. 7,122,482 or 8,765,359, or US Pat. App. Pub. No 2013/0116153 A1, each of which is incorporated herein by reference. The wells can be configured for selective excitation, for example, as set forth in U.S. Pat. No. 8,798,414 or 9,347,829, each of which is incorporated herein by reference. Analytes can be distributed to nanometer-scale posts, such as high aspect ratio posts which can optionally be dielectric pillars that extend through a metallic layer to improve detection of an analyte coupled to the pillar. See, for example, U.S. Pat. Nos. 8,148,264, 9,410,887 or 9,987,609, each of which is incorporated herein by reference. Further examples of nanostructures that can be used to detect analytes are those that change state in response to the concentration of analytes such that the analytes can be quantitated as set forth in WO 2020/176793 A1, which is incorporated herein by reference.

An apparatus or method set forth herein need not be configured for optical detection. For example, an electronic detector can be used for detection of protons or charged labels (see, for example, US Pat. App. Pub. Nos. 2009/0026082 A1; 2009/0127589 A1; 2010/0137143 A1; or 2010/0282617 A1, each of which is incorporated herein by reference in its entirety). A field effect transistor (FET) can be used to detect analytes or other entities, for example, based on proximity of a field disrupting moiety to the FET. The field disrupting moiety can be due to an extrinsic label coupled to an analyte or affinity agent, or the moiety can be intrinsic to the analyte or affinity agent being used. Surface plasmon resonance can be used to detect binding of analytes or affinity agents at or near a surface. Exemplary sensors and methods for coupling molecules to sensors are set forth in US Pat. App. Pub. Nos. 2017/0240962 A1; 2018/0051316 A1; 2018/0112265 A1; 2018/0155773 A1 or 2018/0305727 A1; or U.S. Pat. Nos. 9,164,053; 9,829,456; 10,036,064, each of which is incorporated herein by reference.

In some configurations of the compositions, apparatus and methods set forth herein, one or more proteins can be present on a solid support, where the proteins can optionally be detected. For example, a protein can be coupled to a solid support, the solid support can be contacted with a detection agent (e.g. affinity agent) in solution, the affinity agent can interact with the protein, thereby producing a detectable signal, and then the signal can be detected to determine the presence, absence, quantity, a characteristic or identity of the protein. In multiplexed versions of this approach, different proteins can be coupled to different addresses in an array, and the detection steps can occur in parallel, such that proteins at each address are detected, quantified, characterized or identified. In another example, detection agents can be coupled to a solid support, the support can be contacted with proteins in solution, the proteins can interact with the detection agents, thereby producing a detectable signal, and then the signal can be detected to determine the presence of the proteins. This approach can also be multiplexed by coupling different probes to different addresses of an array.

In multiplexed configurations, different proteins can be coupled to different unique identifiers (e.g. addresses in an array), and the proteins can be manipulated and detected in parallel. For example, a fluid containing one or more different affinity agents can be delivered to an array such that the proteins of the array are in simultaneous contact with the affinity agent(s). Moreover, a plurality of addresses can be observed in parallel allowing for rapid detection of binding events. A plurality of different proteins can have a complexity of at least 5, 10, 100, 1×10³, 1×10⁴, 1×10⁵ or more different native-length protein primary sequences. Alternatively or additionally, a proteome, proteome subfraction or other protein sample that is analyzed in a method set forth herein can have a complexity that is at most 1×10⁵, 1×10⁴, 1×10³, 100, 10, 5 or fewer different native-length protein primary sequences. The total number of proteins of a sample that is detected, characterized or identified can differ from the number of different primary sequences in the sample, for example, due to the presence of multiple copies of at least some protein species. Moreover, the total number of proteins of a sample that is detected, characterized or identified can differ from the number of candidate proteins suspected of being in the sample, for example, due to the presence of multiple copies of at least some protein species, absence of some proteins in a source for the sample, or loss of some proteins prior to analysis.

A protein can be coupled to a unique identifier using any of a variety of means. The coupling can be covalent or non-covalent. Exemplary covalent attachments include chemical linkers such as those achieved using click chemistry or other linkages known in the art or described in U.S. patent application Ser. No. 17/062,405, which is incorporated herein by reference. Non-covalent attachment can be mediated by receptor-ligand interactions (e.g. (strept)avidin-biotin, antibody-antigen, or complementary nucleic acid strands), for example, wherein the receptor is attached to the unique identifier and the ligand is attached to the protein or vice versa. In particular configurations, a protein is coupled to a solid support (e.g. an address in an array) via a structured nucleic acid particle (SNAP). A protein can be coupled to a SNAP and the SNAP can interact with a solid support, for example, by non-covalent interactions of the DNA with the support and/or via covalent linkage of the SNAP to the support. Nucleic acid origami or nucleic acid nanoballs are particularly useful. The use of SNAPs and other moieties to couple proteins to unique identifiers such as tags or addresses in an array are set forth in U.S. patent application Ser. Nos. 17/062,405 and 63/159,500, each of which is incorporated herein by reference.

The methods, compositions and apparatus of the present disclosure are particularly well suited for use with proteins. Although proteins are exemplified throughout the present disclosure, it will be understood that other analytes can be similarly used. Exemplary analytes include, but are not limited to, biomolecules, polysaccharides, nucleic acids, lipids, metabolites, hormones, vitamins, enzyme cofactors, therapeutic agents, candidate therapeutic agents or combinations thereof. An analyte can be a non-biological atom or molecule, such as a synthetic polymer, metal, metal oxide, ceramic, semiconductor, mineral, or a combination thereof.

One or more proteins that are used in a method, composition or apparatus herein, can be derived from a natural or synthetic source. Exemplary sources include, but are not limited to biological tissues, fluids, cells or subcellular compartments (e.g. organelles). For example, a sample can be derived from a tissue biopsy, biological fluid (e.g. blood, sweat, tears, plasma, extracellular fluid, urine, mucus, saliva, semen, vaginal fluid, synovial fluid, lymph, cerebrospinal fluid, peritoneal fluid, pleural fluid, amniotic fluid, intracellular fluid, extracellular fluid, etc.), fecal sample, hair sample, cultured cell, culture media, fixed tissue sample (e.g. fresh frozen or formalin-fixed paraffin-embedded) or product of a protein synthesis reaction. A protein source may include any sample where a protein is a native or expected constituent. For example, a primary source for a cancer biomarker protein may be a tumor biopsy sample or bodily fluid. Other sources include environmental samples or forensic samples.

Exemplary organisms from which proteins or other analytes can be derived include, for example, a mammal such as a rodent, mouse, rat, rabbit, guinea pig, ungulate, horse, sheep, pig, goat, cow, cat, dog, primate, non-human primate or human; a plant such as Arabidopsis thaliana, tobacco, corn, sorghum, oat, wheat, rice, canola, or soybean; an algae such as Chlamydomonas reinhardtii; a nematode such as Caenorhabditis elegans; an insect such as Drosophila melanogaster, mosquito, fruit fly, honey bee or spider; a fish such as zebrafish; a reptile; an amphibian such as a frog or Xenopus laevis; a dictyostelium discoideum; a fungi such as Pneumocystis carinii, Takifugu rubripes, yeast, Saccharomyces cerevisiae or Schizosaccharomyces pombe; or a Plasmodium falciparum. Proteins can also be derived from a prokaryote such as a bacterium, Escherichia coli, staphylococci or Mycoplasma pneumoniae; an archae; a virus such as Hepatitis C virus, influenza virus, coronavirus, or human immunodeficiency virus; or a viroid. Proteins can be derived from a homogeneous culture or population of the above organisms or alternatively from a collection of several different organisms, for example, in a community or ecosystem.

In some cases, a protein or other biomolecule can be derived from an organism that is collected from a host organism. For example, a protein may be derived from a parasitic, pathogenic, symbiotic, or latent organism collected from a host organism. A protein can be derived from an organism, tissue, cell or biological fluid that is known or suspected of being linked with a disease state or disorder (e.g., cancer). Alternatively, a protein can be derived from an organism, tissue, cell or biological fluid that is known or suspected of not being linked to a particular disease state or disorder. For example, the proteins isolated from such a source can be used as a control for comparison to results acquired from a source that is known or suspected of being linked to the particular disease state or disorder. A sample may include a microbiome or substantial portion of a microbiome. In some cases, one or more proteins used in a method, composition or apparatus set forth herein may be obtained from a single source and no more than the single source. The single source can be, for example, a single organism (e.g. an individual human), single tissue, single cell, single organelle (e.g. endoplasmic reticulum, Golgi apparatus or nucleus), or single protein-containing particle (e.g., a viral particle or vesicle).

A method, composition or apparatus of the present disclosure can use or include a plurality of proteins having any of a variety of compositions such as a plurality of proteins composed of a proteome or fraction thereof. For example, a plurality of proteins can include solution-phase proteins, such as proteins in a biological sample or fraction thereof, or a plurality of proteins can include proteins that are immobilized, such as proteins coupled to a particle or solid support. By way of further example, a plurality of proteins can include proteins that are detected, analyzed or identified in connection with a method, composition or apparatus of the present disclosure. The content of a plurality of proteins can be understood according to any of a variety of characteristics such as those set forth below or elsewhere herein.

A plurality of proteins can be characterized in terms of total protein mass. The total mass of protein in a liter of plasma has been estimated to be 70 g and the total mass of protein in a human cell has been estimated to be between 100 pg and 500 pg depending upon cells type. See Wisniewski et al. Molecular & Cellular Proteomics 13:10.1074/mcp.M113.037309, 3497-3506 (2014), which is incorporated herein by reference. A plurality of proteins used or included in a method, composition or apparatus set forth herein can include at least 1 pg, 10 pg, 100 pg, 1 ng, 10 ng, 100 ng, 1 mg, 10 mg, 100 mg, 1 mg, 10 mg, 100 mg or more protein by mass. Alternatively or additionally, a plurality of proteins may contain at most 100 mg, 10 mg, 1 mg, 100 mg, 10 mg, 1 mg, 100 ng, 10 ng, 1 ng, 100 pg, 10 pg, 1 pg or less protein by mass.

A plurality of proteins can be characterized in terms of percent mass relative to a given source such as a biological source (e.g. cell, tissue, or biological fluid such as blood). For example, a plurality of proteins may contain at least 60%, 75%, 90%, 95%, 99%, 99.9% or more of the total protein mass present in the source from which the plurality of proteins was derived. Alternatively or additionally, a plurality of proteins may contain at most 99.9%, 99%, 95%, 90%, 75%, 60% or less of the total protein mass present in the source from which the plurality of proteins was derived.

A plurality of proteins can be characterized in terms of total number of protein molecules. The total number of protein molecules in a Saccharomyces cerevisiae cell has been estimated to be about 42 million protein molecules. See Ho et al., Cell Systems (2018), DOI: 10.1016/j.cels.2017.12.004, which is incorporated herein by reference. A plurality of proteins used or included in a method, composition or apparatus set forth herein can include at least 1 protein molecule, 10 protein molecules, 100 protein molecules, 1×10⁴ protein molecules, 1×10⁶ protein molecules, 1×10⁸ protein molecules, 1×10¹⁰ protein molecules, 1 mole (6.02214076×10²³ molecules) of protein, 10 moles of protein molecules, 100 moles of protein molecules or more. Alternatively or additionally, a plurality of proteins may contain at most 100 moles of protein molecules, 10 moles of protein molecules, 1 mole of protein molecules, 1×10¹⁰ protein molecules, 1×10⁸ protein molecules, 1×10⁶ protein molecules, 1×10⁴ protein molecules, 100 protein molecules, 10 protein molecules, 1 protein molecule or less.

A plurality of proteins can be characterized in terms of the variety of full-length primary protein structures in the plurality. For example, the variety of full-length primary protein structures in a plurality of proteins can be equated with the number of different protein-encoding genes in the source for the plurality of proteins. Whether or not the proteins are derived from a known genome or from any genome at all, the variety of full-length primary protein structures can be counted independent of presence or absence of post translational modifications in the proteins. A human proteome is estimated to have about 20,000 different protein-encoding genes such that a plurality of proteins derived from a human can include up to about 20,000 different primary protein structures. See Aebersold et al., Nat. Chem. Biol. 14:206-214 (2018), which is incorporated herein by reference. Other genomes and proteomes in nature are known to be larger or smaller. A plurality of proteins used or included in a method, composition or apparatus set forth herein can have a complexity of at least 2, 5, 10, 100, 1×10³, 1×10⁴, 2×10⁴, 3×10⁴ or more different full-length primary protein structures. Alternatively or additionally, a plurality of proteins can have a complexity that is at most 3×10⁴, 2×10⁴, 1×10⁴, 1×10³, 100, 10, 5, 2 or fewer different full-length primary protein structures.

In relative terms, a plurality of proteins used or included in a method, composition or apparatus set forth herein may contain at least one representative for at least 60%, 75%, 90%, 95%, 99%, 99.9% or more of the proteins encoded by the genome of a source from which the sample was derived. Alternatively or additionally, a plurality of proteins may contain a representative for at most 99.9%, 99%, 95%, 90%, 75%, 60% or less of the proteins encoded by the genome of a source from which the sample was derived.

A plurality of proteins can be characterized in terms of the variety of primary protein structures in the plurality including transcribed splice variants. The human proteome has been estimated to include about 70,000 different primary protein structures when splice variants ae included. See Aebersold et al., Nat. Chem. Biol. 14:206-214 (2018), which is incorporated herein by reference. Moreover, the number of the partial-length primary protein structures can increase due to fragmentation that occurs in a sample. A plurality of proteins used or included in a method, composition or apparatus set forth herein can have a complexity of at least 2, 5, 10, 100, 1×10³, 1×10⁴, 7×10⁴, 1×10⁵, 1×10⁶ or more different primary protein structures. Alternatively or additionally, a plurality of proteins can have a complexity that is at most 1×10⁶, 1×10⁵, 7×10⁴, 1×10⁴, 1×10³, 100, 10, 5, 2 or fewer different primary protein structures.

A plurality of proteins can be characterized in terms of the variety of protein structures in the plurality including different primary structures and different proteoforms among the primary structures. Different molecular forms of proteins expressed from a given gene are considered to be different proteoforms. Protoeforms can differ, for example, due to differences in primary structure (e.g. shorter or longer amino acid sequences), different arrangement of domains (e.g. transcriptional splice variants), or different post translational modifications (e.g. presence or absence of phosphoryl, glycosyl, acetyl, or ubiquitin moieties). The human proteome is estimated to include hundreds of thousands of proteins when counting the different primary structures and proteoforms. See Aebersold et al., Nat. Chem. Biol. 14:206-214 (2018), which is incorporated herein by reference. A plurality of proteins used or included in a method, composition or apparatus set forth herein can have a complexity of at least 2, 5, 10, 100, 1×10³, 1×10⁴, 1×10⁵, 1×10⁶, 5×10⁶, 1×10⁷ or more different protein structures. Alternatively or additionally, a plurality of proteins can have a complexity that is at most 1×10⁷, 5×10⁶, 1×10⁶, 1×10⁵, 1×10⁴, 1×10³, 100, 10, 5, 2 or fewer different protein structures.

A plurality of proteins can be characterized in terms of the dynamic range for the different protein structures in the sample. The dynamic range can be a measure of the range of abundance for all different protein structures in a plurality of proteins, the range of abundance for all different primary protein structures in a plurality of proteins, the range of abundance for all different full-length primary protein structures in a plurality of proteins, the range of abundance for all different full-length gene products in a plurality of proteins, the range of abundance for all different proteoforms expressed from a given gene, or the range of abundance for any other set of different proteins set forth herein. The dynamic range for all proteins in human plasma is estimated to span more than 10 orders of magnitude from albumin, the most abundant protein, to the rarest proteins that have been measured clinically. See Anderson and Anderson Mol Cell Proteomics 1:845-67 (2002), which is incorporated herein by reference. The dynamic range for plurality of proteins set forth herein can be a factor of at least 10, 100, 1×10³, 1×10⁴, 1×10⁶, 1×10⁸, 1×10¹⁰, or more. Alternatively or additionally, the dynamic range for plurality of proteins set forth herein can be a factor of at most 1×10¹⁰, 1×10⁸, 1×10⁶, 1×10⁴, 1×10³, 100, 10 or less.

A method set forth herein can be carried out in a fluid phase or on a solid phase. For fluid phase configurations, a fluid containing one or more proteins can be mixed with another fluid containing one or more affinity agents. For solid phase configurations one or more proteins or affinity agents can be coupled to a solid support. One or more components that will participate in a binding event can be contained in a fluid and the fluid can be delivered to a solid support, the solid support being coupled to one or more other component that will participate in the binding event.

A method of the present disclosure can be carried out at single analyte resolution.

Alternatively to single-analyte resolution, a method can be carried out at ensemble-resolution or bulk-resolution. Bulk-resolution configurations acquire a composite signal from a plurality of different analytes or affinity agents in a vessel or on a surface. For example, a composite signal can be acquired from a population of different protein-affinity agent complexes in a well or cuvette, or on a solid support surface, such that individual complexes are not resolved from each other. Ensemble-resolution configurations acquire a composite signal from a first collection of proteins or affinity agents in a sample, such that the composite signal is distinguishable from signals generated by a second collection of proteins or affinity agents in the sample. For example, the ensembles can be located at different addresses in an array. Accordingly, the composite signal obtained from each address will be an average of signals from the ensemble, yet signals from different addresses can be distinguished from each other.

A composition, apparatus or method set forth herein can be configured to contact one or more proteins (e.g. an array of different proteins) with a plurality of different affinity agents. For example, a plurality of affinity agents (whether configured separately or as a pool) may include at least 2, 5, 10, 25, 50, 100, 250, 500 or more types of affinity agents, each type of affinity agent differing from the other types with respect to the epitope(s) recognized. Alternatively or additionally, a plurality of affinity agents may include at most 500, 250, 100, 50, 25, 10, 5, or 2 types of affinity agents, each type of affinity agent differing from the other types with respect to the epitope(s) recognized. Different types of affinity agents in a pool can be uniquely labeled such that the different types can be distinguished from each other. In some configurations, at least two, and up to all, of the different types of affinity agents in a pool may be indistinguishably labeled with respect to each other. Alternatively or additionally to the use of unique labels, different types of affinity agents can be delivered and detected serially when evaluating one or more proteins (e.g. in an array).

A method of the present disclosure can be performed in a multiplex format. In multiplexed configurations, different proteins can be coupled to different unique identifiers (e.g. the proteins can be coupled to different addresses in an array). Multiplexed proteins can be manipulated and detected in parallel. For example, a fluid containing one or more different affinity agents can be delivered to a protein array such that the proteins of the array are in simultaneous contact with the affinity agent(s). Moreover, a plurality of addresses can be observed in parallel allowing for rapid detection of binding events. A plurality of different proteins can have a complexity of at least 5, 10, 100, 1×10³, 1×10⁴, 2×10⁴, 3×10⁴ or more different native-length protein primary sequences. Alternatively or additionally, a proteome or proteome subfraction that is analyzed in a method set forth herein can have a complexity that is at most 3×10⁴, 2×10⁴, 1×10⁴, 1×10³, 100, 10, 5 or fewer different native-length protein primary sequences. The plurality of proteins can constitute a proteome or subfraction of a proteome. The total number of proteins that is detected, characterized or identified can differ from the number of different primary sequences in the sample from which the proteins are derived, for example, due to the presence of multiple copies of at least some protein species. Moreover, the total number of proteins that are detected, characterized or identified can differ from the number of candidate proteins suspected of being present, for example, due to the presence of multiple copies of at least some protein species, absence of some proteins in a source for the proteins, or loss of some proteins prior to analysis.

A particularly useful multiplex format uses an array of proteins and/or affinity agents. A polypeptide, anchoring group, polypeptide composite or other analyte can be coupled to a unique identifier, such as an address in an array, using any of a variety of means. The coupling can be covalent or non-covalent. Exemplary covalent attachments include chemical linkers such as those achieved using click chemistry or other linkages known in the art or described in US Pat. App. Pub. No. 2021/0101930 A1, which is incorporated herein by reference. Non-covalent attachment can be mediated by receptor-ligand interactions (e.g. (strept)avidin-biotin, antibody-antigen, or complementary nucleic acid strands), for example, in which the receptor is attached to the unique identifier and the ligand is attached to the protein or vice versa. In particular configurations, a protein is coupled to a solid support (e.g. an address in an array) via a structured nucleic acid particle (SNAP). A protein can be coupled to a SNAP and the SNAP can interact with a solid support, for example, by non-covalent interactions of the DNA with the support and/or via covalent linkage of the SNAP to the support. Nucleic acid origami or nucleic acid nanoballs are particularly useful. The use of SNAPs and other moieties to couple proteins to unique identifiers such as tags or addresses in an array are set forth in US Pat. App. Pub. No. 2021/0101930 A1, which is incorporated herein by reference.

A solid support or a surface thereof may be configured to display an analyte or a plurality of analytes. A solid support may contain one or more patterned, formed, or prepared surfaces that contain at least one address for displaying an analyte. In some cases, a solid support may contain one or more patterned, formed, or prepared surfaces that contain a plurality of addresses, with each address configured to display one or more analytes. Accordingly, an array as set forth herein may comprise a plurality of analytes coupled to a solid support or a surface thereof. In some configurations, a solid support or a surface thereof may be patterned or formed to produce an ordered or patterned array of addresses. The deposition of analytes on the ordered or patterned array of addresses may be controlled by interactions between the solid support and the analytes such as, for example, electrostatic interactions, magnetic interactions, hydrophobic interactions, hydrophilic interactions, covalent interactions, or non-covalent interactions. Accordingly, the coupling of an analyte at each address of an array may produce an ordered or patterned array of analytes whose average spacing between analytes is determined based upon the tolerance of the ordering or patterning of the solid support and the size of an analyte-binding region for each address. An ordered or patterned array of analytes may be characterized as having a regular geometry, such as a rectangular, triangular, polygonal, or annular grid. In other configurations, a solid support or a surface thereof may be non-patterned or non-ordered. The deposition of analytes on the non-ordered or non-patterned array of addresses may be controlled by interactions between the solid support and the analytes, or inter-analyte interactions such as, for example, steric repulsion, electrostatic repulsion, electrostatic attraction, magnetic repulsion, magnetic attraction, covalent interactions, or non-covalent interactions.

A solid support or a surface thereof may contain one or more structures or features. A structure or feature may comprise an elevation, profile, shape, geometry, or configuration that deviates from an average elevation, profile, shape, geometry, or configuration of a solid support or surface thereof. A structure or feature may be a raised structure or feature, such as a ridge, post, pillar, or pad, if the structure or feature extends above the average elevation of a surface of a solid support. A structure or feature may be a depressed structure, such as a channel, well, pore, or hole, if the structure or feature extends below the average elevation of a surface of a solid support. A structure or feature may be an intrinsic structure or feature of a substrate (i.e., arising due to the physical or chemical properties of the substrate, or a physical or chemical mechanism of formation), such as surface roughness structures, crystal structures, or porosity. A structure or feature may be formed by a method of processing a solid support. In some configurations, a solid support or a surface may be processed by a lithographic method to form one or more structures or features. A solid support or a surface thereof may be formed by a suitable lithographic method, including, but not limited to photolithography, Dip-Pen nanolithography, nanoimprint lithography, nanosphere lithography, nanoball lithography, nanopillar arrays, nanowire lithography, immersion lithography, neutral particle lithography, plasmonic lithography, scanning probe lithography, thermochemical lithography, thermal scanning probe lithography, local oxidation nanolithography, molecular self-assembly, stencil lithography, laser interference lithography, soft lithography, magnetolithography, stereolithography, deep ultraviolet lithography, x-ray lithography, ion projection lithography, proton-beam lithography, or electron-beam lithography.

A solid support or surface may comprise a plurality of structures or features. A plurality of structures or features may comprise an ordered or patterned array of structures or features. A plurality of structures or features may comprise an non-ordered, non-patterned, or random array of structures or features. A structure or feature may have an average characteristic dimension (e.g., length, width, height, diameter, circumference, etc.) of at least about 1 nanometer (nm), 5 nm, 10 nm, 20 nm, 30 nm, 40 nm, 50 nm, 75 nm, 100 nm, 150 nm, 200 nm, 250 nm, 300 nm, 400 nm, 500 nm, 750 nm, 1000 nm, or more than 1000 nm. Alternatively or additionally, a structure or feature may have an average characteristic dimension of no more than about 1000 nm, 750 nm, 500 nm, 400 nm, 300 nm, 250 nm, 200 nm, 150 nm, 100 nm, 75 nm, 50 nm, 40 nm, 30 nm, 20 nm, 10 nm, 5 nm, 1 nm, or less than 1 nm. An array of structures or features may have an average pitch, in which the pitch is measured as the average separation between respective centerpoints of neighboring structures or features. An array may have an average pitch of at least about 1 nm, 5 nm, 10 nm, 20 nm, 30 nm, 40 nm, 50 nm, 75 nm, 100 nm, 150 nm, 200 nm, 250 nm, 300 nm, 400 nm, 500 nm, 750 nm, 1 micron (μm), 2 μm, 5 μm, 10 μm, 50 μm, 100 μm, or more than 100 μm. Alternatively or additionally, an array may have an average pitch of no more than about 100 μm, 50 μm, 10 μm, 5 μm, 1 μm, 750 nm, 500 nm, 400 nm, 300 nm, 250 nm, 200 nm, 150 nm, 100 nm, 75 nm, 50 nm, 40 nm, 30 nm, 20 nm, 10 nm, 5 nm, 1 nm, or less than 1 nm.

A solid support or a surface thereof may include a base substrate material and, optionally, one or more additional materials that are contacted or adhered with the substrate material. A solid support may comprise one or more additional materials that are deposited, coated, or inlayed onto the substrate material. Additional materials may be added to the substrate material to alter the properties of the substrate material. For example, materials may be added to alter the surface chemistry (e.g., hydrophobicity, hydrophilicity, non-specific binding, electrostatic properties), alter the optical properties (e.g., reflective properties, refractive properties), alter the electrical or magnetic properties (e.g., dielectric materials, conducting materials, electrically-insulating materials), or alter the heat transfer characteristics of the substrate material. Additional materials contacted or adhered with a substrate material may be ordered or patterned onto the substrate material to, for example, locate the additional material at addresses or locate the additional material at interstitial regions between addresses. Exemplary additional materials may include metals (e.g., gold, silver, copper, etc.), metal oxides (e.g., titanium oxide, silicon dioxide, alumina, iron oxides, etc.), metal nitrides (e.g., silicon nitride, aluminum nitride, boron nitride, gallium nitride, etc.), metal carbides (e.g., tungsten carbide, titanium carbide, iron carbide, etc.), metal sulfides (e.g., iron sulfide, silver sulfide, etc.), and organic moieties (e.g., polyethylene glycol (PEG), dextrans, chemically-reactive functional groups, etc.).

A method of the present disclosure can include the step of coupling one or more analytes to a solid support or a surface thereof prior to performing a detection step set forth herein. The coupling of one or more analytes to a solid support surface may include covalent or non-covalent coupling of the one or more analytes to the solid support. Covalent coupling of an analyte to a solid support can include direct covalent coupling of an analyte to a solid support (e.g., formation of coordination bonds) or indirect covalent coupling between a reactive functional group of the analyte and a reactive functional group that is coupled to the solid support (e.g., a CLICK-type reaction). Non-covalent coupling can include the formation of any non-covalent interaction between an analyte and a solid support, including electrostatic or magnetic interactions, or non-covalent bonding interactions (e.g., ionic bonds, van der Waals interactions, hydrogen bonding, etc.). The skilled person will readily recognize that the particular analyte and the choice of solid support can affect the selection of a coupling chemistry for the compositions and methods set forth herein.

Accordingly, a coupling chemistry may be selected based upon the criterium that it provides a sufficiently stable coupling of an analyte to a solid support for a time scale that meets or exceeds the time scale of a method as set forth herein. For example, a polypeptide identification method can require a coupling of the analyte to the solid support for a sufficient amount of time to permit a series of empirical measurements of the analyte to occur. An analyte may be continuously coupled to a solid support for an observable length of time such as, for example, at least about 1 minute, 1 hour (hr), 3 hrs, 6 hrs, 12 hrs, 1 day, 1.5 days, 2 days, 3 days, 1 week (wk), 2 wks, 3 wks, 1 month, or more. The coupling of an analyte to a solid support can occur with a solution-phase chemistry that promotes the deposition of the analyte on the solid support. Coupling of an analyte to a solid support may occur under solution conditions that are optimized for any conceivable solution property, including solution composition, species concentrations, pH, ionic strength, solution temperature, etc. Solution composition can be varied by chemical species, such as buffer type, salts, acids, bases, and surfactants. In some configurations, species such as salts and surfactants may be selected to facilitate the formation of interactions between an analyte and a solid support. Covalent coupling methods for coupling an analyte to a solid support may include species such as catalyst, initiators, and promoters to facilitate particular reactive chemistries.

Coupling of an analyte to a solid support may be facilitated by a mediating group. A mediating group may modify the properties of the analyte to facilitate the coupling. Useful mediating groups have been set forth herein (e.g., structured nucleic acid particles). In some configurations, a mediating group can be coupled to an analyte prior to coupling the analyte to a solid support. Accordingly, the mediating group may be chosen to increase the strength, control, or specificity of the coupling of the analyte to the solid support. In other configurations, a mediating group can be coupled to a solid support prior to coupling an analyte to the solid support. Accordingly, the mediating group may be chosen to provide a more favorable coupling chemistry than can be provided by the solid support alone.

Also provided herein are array-based systems for implementing methods, as set forth herein, including methods of forming arrays of analytes. An array-based system can include arrays, as set forth herein, including arrays disposed within flow cells or fluidic cartridges. An array-based system may further comprise a fluidic system that is configured to transport fluidic media to and from an array. A fluidic system may comprise ancillary equipment for transfer and storage of fluidic media, including transfer lines, pumps, valves, reservoirs, etc. A flow cell or fluidic cartridge may comprise one or more ports and/or manifolds that facilitate injection and removal of a fluidic medium from the flow cell or fluidic cartridge. A fluidic system may further comprise components that facilitate mass transfer of analytes, anchoring moieties, or other particles in an array-based system. Additional components can include magnetic field generators (e.g., magnets, electromagnets), electric field generators (e.g., electrodes, capacitors, resistors, voltage sources, etc.), and heat transfer devices such as heaters (e.g., dissipative heaters, radiative heaters) and cooling devices (e.g., heat exchangers, fans or blowers, cooling fins, Pelletier devices). An array-based system may further comprise a detection system, such as a light-sensing device. A detection system may be configured to detect addresses that produce a detectable signal, such as an address containing an analyte, anchoring moiety, or affinity agent, as set forth herein.

An array or an assay agent (e.g., an anchoring moiety, analyte, or other particle), as set forth herein, may be provided as a component of a kit. A kit may comprise an anchoring moiety, as set forth herein, that is configured to be coupled to an analyte of interest. A kit may be provided with an array or assay agent, as set forth herein, or a plurality thereof. A collection kit may be specific to a particular assay to be performed on a sample. For example, a collection kit for a polypeptide assay may include polypeptide-specific reagents to protect and/or preserve polypeptides within a sample. A collection kit may include one or more sample vessels, one or more reagents, instructions for use of the sample collection kit and optionally intermediate sample vessels, a sealant for the vessel(s), a label for the vessel(s) such as a barcode or radio frequency identification device (RFID), or packaging for transport and/or storage of the sample vessel(s). A kit may include one or more reagents for any of a variety of purposes, including sample preservation, sample stability, sample quality control, processing and/or purification, and sample storage. A kit may include reagents such as buffers, acids, bases, solvents, denaturants, surfactants, detergents, reactants, labels (e.g., fluorophores, radiolabels), indicator dyes, enzymes, enzyme inhibitors, oxygen scavengers, water scavengers, humectants, affinity reagents (e.g., antibodies), or other capture agents (e.g., biotinylated particles). A kit may include one or more reagents in liquid or solid form. A kit may include one or more separate reagents and/or internal standards that are added to a sample vessel before or after sample preparation. A kit may include one or more reagents, control reagents, and/or internal standards that are provided within a sample collection vessel. For example, reagents and/or internal standards may be provided in a crystallized or coated form on a surface of a collection vessel, or may be in a liquid solution within the collection vessel. In some configurations, a kit may further comprise an array or solid support, as set forth herein. An array or solid support may be provided in a kit with one or more assay agents (e.g., e.g., anchoring moieties, analytes, or other particles) present within or deposited on the array or solid support.

A kit for an assay or other process may be utilized according to a provided set of instructions. The instructions may be directed to use of assay agents and/or arrays in accordance with teachings set forth herein. A kit may provide instructions for coupling an analyte of interest to an anchoring moiety, for example by a method as set forth herein. A kit may provide instructions for depositing an assay agent, as set forth herein, to an array or solid support, as set forth herein. A kit may be utilized by a technician or self-collecting subject. A technician utilizing a kit may be specifically trained in the proper utilization of the kit. A kit protocol may employ one or more intermediate steps before preparation of a sample is complete. Intermediate steps during sample preparation may be performed in a vessel or in a separate medium (provided with the kit or provided by the collector). For example, a blood sample may be fractionated by a phlebotomist, with only the red blood cell or plasma fraction saved for preparation. A kit may include indicator dyes, litmus strips, or other methods of confirming successful sample collection and/or preparation. A kit may include a sealant (e.g., an adhesive or sticker) to ensure that a sample has not been tampered with or damaged during storage or transport. A kit may include a label for sample tracking by the collector or the analysis facility. A label for a vessel may include a serial number, RFID, bar code or QR code. A label for a vessel may be pre-printed or pre-applied to a vessel, or may be placed by a collector.

### Example 1. Multi-Cycle Anchoring Moiety Deposition

Anchoring moieties were deposited on an array containing a patterned array of sites. Details of anchoring moieties and patterned arrays can be found in U.S. Pat. No. 11,505,796. In brief, each anchoring moiety comprised a DNA origami tile, with each origami tile containing a plurality of pendant surface-coupling oligonucleotides. The array comprised a hexagonally-patterned silicon wafer, with each array site containing a plurality of surface-coupled oligonucleotides. The surface-coupled oligonucleotides were configured to be complementary to the surface-coupling oligonucleotides of the anchoring moieties.

Each cycle of anchoring moiety deposition comprised the steps of: 1) contacting a fluid containing a 2 nanomolar (nM) concentration of anchoring moieties with the patterned array of sites for 1 hour, 2) after contacting the fluid with the array, rinsing the fluid from the array with a rinsing buffer, and 3) imaging the array via confocal laser scanning microscopy to detect presence or absence at each array site of fluorescent dyes coupled to anchoring moieties. Up to 7 cycles were performed.

Microscope images were analyzed to determine total site occupancy after each cycle, as well as total site co-occupancy after each cycle. Site occupancy was determined based upon the presence of a signal above a background signal at an array site. Site co-occupancy was determined based upon the presence of a signal with a greater signal magnitude compared to an average single-occupancy site.

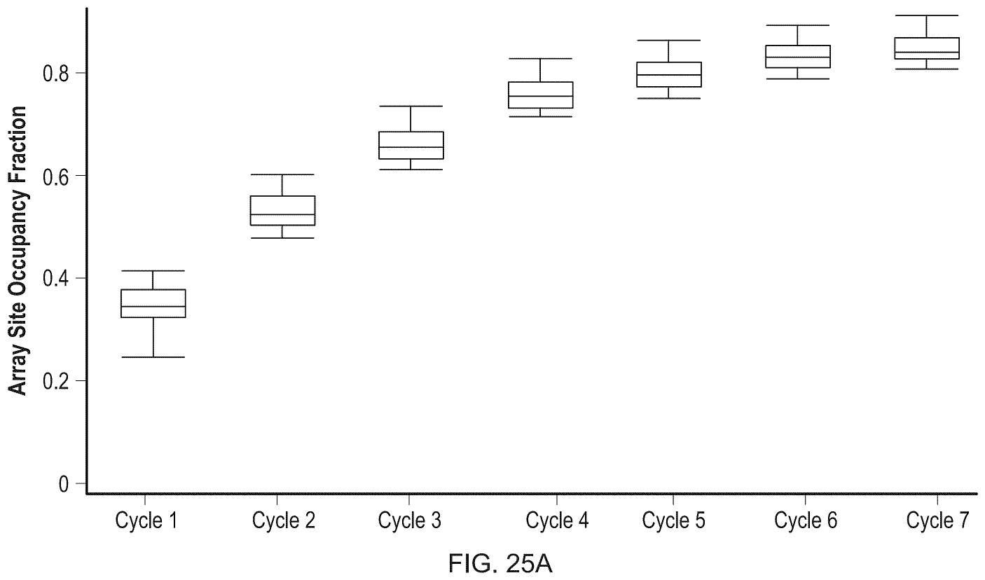

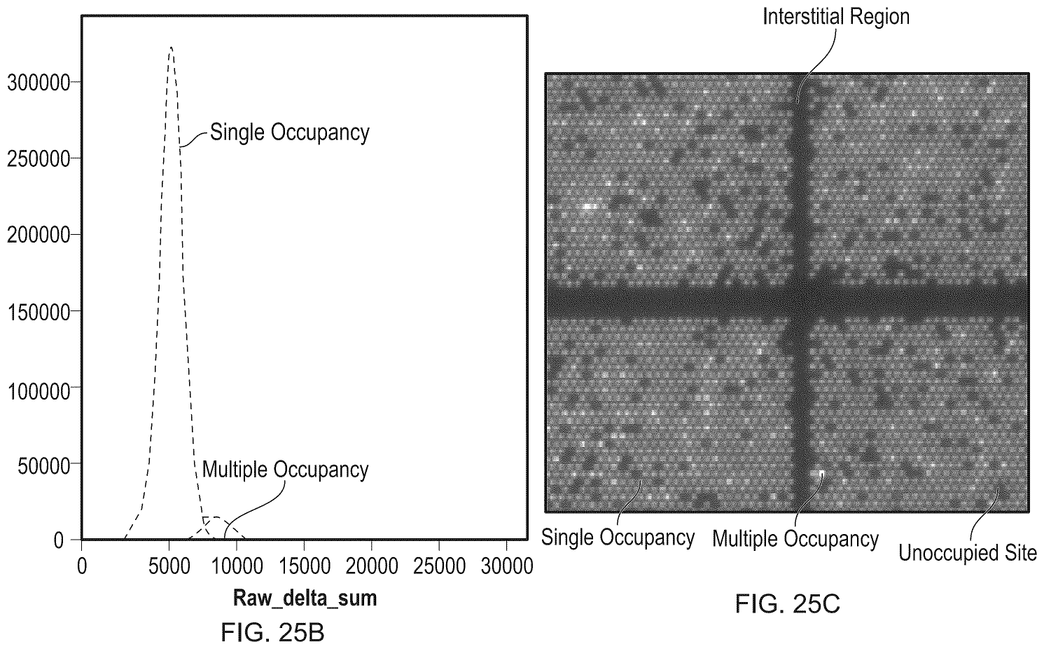

FIG. 25A displays the array site occupancy in fractional form as a function of cycle number. The array site occupancy rate can be seen to increase with each successive cycle of deposition, with an initial occupancy fraction after cycle 1 of about 0.35 and a final occupancy fraction after cycle 7 of about 0.84. FIG. 25B depicts a signal intensity histogram for the array after cycle 7, indicating about 80% of sites with single occupancy of anchoring moieties, and about 4% of sites with anchoring moiety co-occupancy (two or more anchoring moieties present at the same array site). FIG. 25C depicts an exemplary microscopy image of an array site after cycle 7, showing the high level of site occupancy (based upon observed fluorescent signals) and a low level of array site co-occupancy (based upon high-intensity fluorescent signals).

### Example 2. Anchoring Moiety Design Parameters

Anchoring moieties were varied to assess impact of surface-coupling oligonucleotide design on array site occupancy. Details of anchoring moieties and patterned arrays can be found in U.S. Pat. No. 11,505,796. Differing designs of anchoring moiety were deposited on arrays according to the method of Example 1, with only a single cycle of deposition utilized.

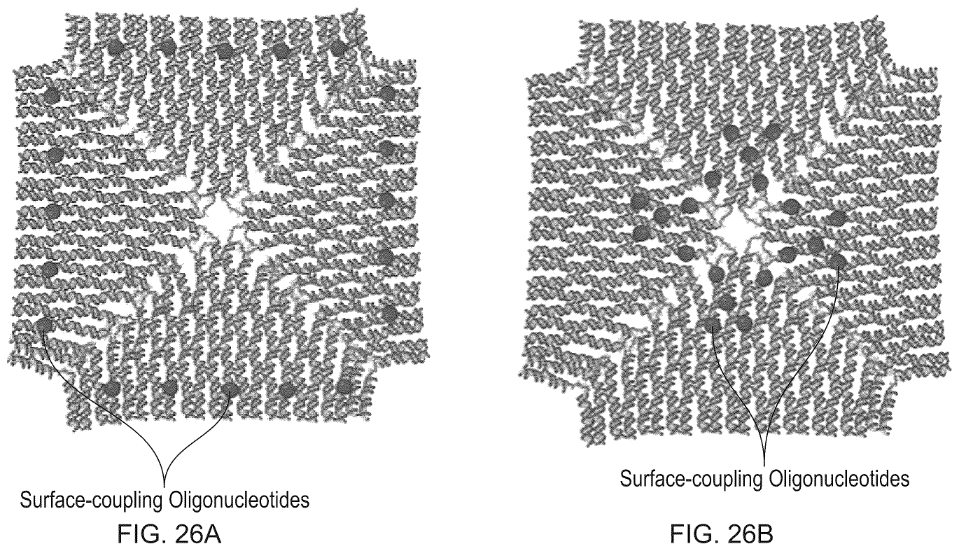

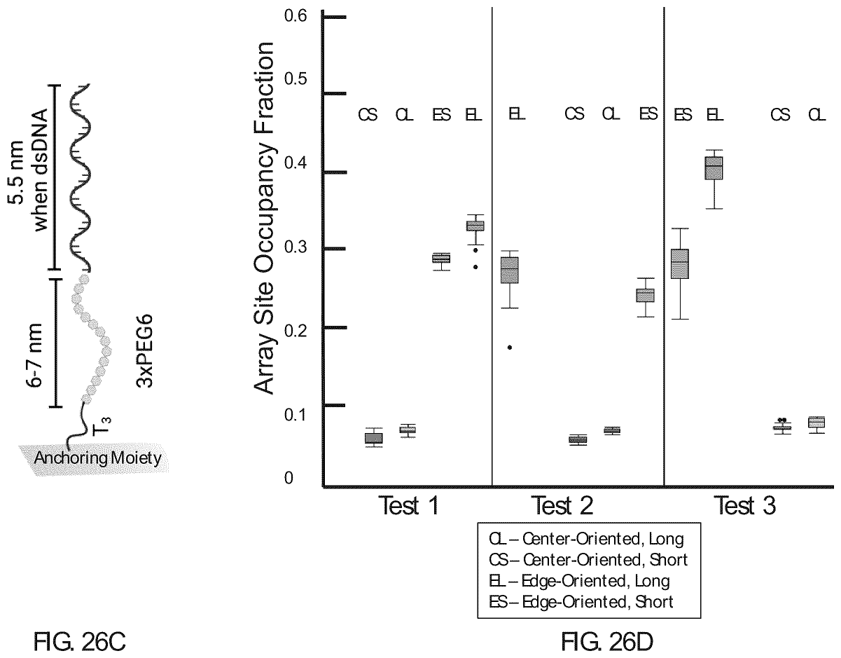

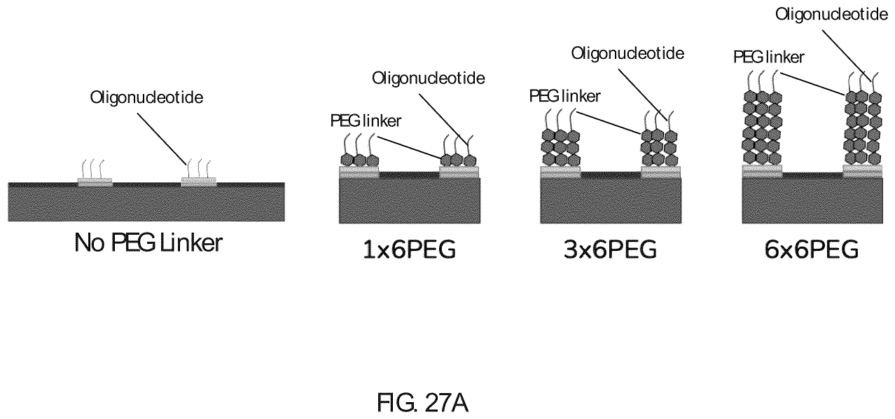

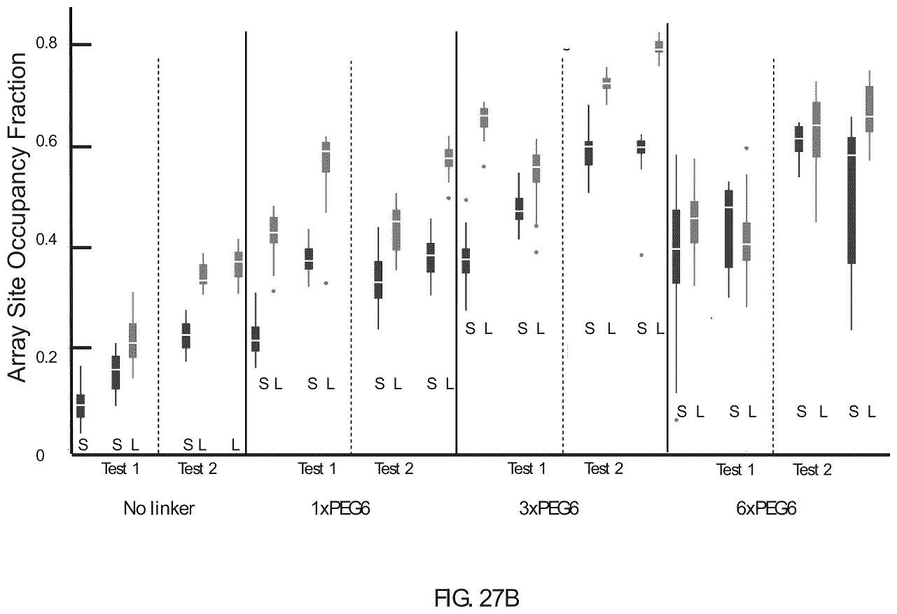

FIGS. 26A and 26B depict differing designs of anchoring moiety tiles. The design of FIG. 26A includes 20 surface-coupling oligonucleotides, with 4 sets of 5 oligonucleotides, and with each set oriented along an edge of a face of the tile. The design of FIG. 26B includes 20 surface-coupling oligonucleotides, with all oligonucleotides clustered together near the center of the face of the tile. FIG. 26C depicts an additional design parameter of moiety length. “Long” coupling moieties were designed to incorporate an 18-monomer PEG linker between the tile and the surface-coupling oligonucleotide. “Short” coupling moieties were designed to exclude the 18-monomer PEG linker.

FIG. 26D depicts array site occupancy fraction for different anchoring moiety designs. Each data set represents a replicate of the deposition experiment. In general, higher array site occupancy is observed for anchoring moiety designs with the long coupling moieties versus the short coupling moieties. Further, higher array site occupancy is observed for anchoring moieties with edge-oriented coupling moieties versus center-oriented coupling moieties.

### Example 3. Array Site Design Parameters

Array site configurations were varied to assess impact of surface-coupled oligonucleotide design on array site occupancy. Details of anchoring moieties and patterned arrays can be found in U.S. Pat. No. 11,505,796. The “long” and “short” edge-oriented designs of anchoring moieties described in Example 2 were deposited on array with differing array site configurations, with only a single cycle of deposition utilized.

FIG. 27A depicts differing tested array site configurations. Surface-coupled oligonucleotides were varied by incorporation of PEG linkers. Tested PEG linker lengths were 0 monomers, 6 monomers, 18 monomers, or 36 monomers. The anchoring moieties were deposited on each array configuration and analyzed by the method described in Example 1. Two replicate experiments were performed for each array configuration.

FIG. 27B displays array site occupancy fraction for each tested condition. Array site occupancy fraction is observed to increase with increasing length of PEG linker on the surface-coupled oligonucleotides. There does not appear to be substantial increase in loading beyond the 18 monomer linker, but loading variability does increase at the longest PEG linker length. Greater array site occupancy is also observed with the “long” surface-coupling oligonucleotides for the anchoring moieties, as compared to the “short” anchoring moiety designs.

### Example 4. Single and Multiple Occupancy of Array Sites

Rates of single and multiple occupancy of nucleic acid nanoparticles on single-molecule arrays were determined over a variety of deposition conditions. Nucleic acid nanoparticles and array compositions were prepared as described in U.S. Pat. No. 11,505,796. Two types of nucleic acid nanoparticles were tested. The first type of nucleic acid nanoparticle was a substantially square tile with sides of 83 nanometers in length and a first face that was configured to bind to an analyte and a substantially opposed second face containing 20 pendant surface-coupling single-stranded oligonucleotides. The second type of nucleic acid nanoparticle was a rectangular block with a 57 nm×53 nm face containing 20 pendant surface-coupling single-stranded oligonucleotides and a substantially opposed second face containing a protruding post of 30 nm length comprising multiple double-stranded helices. The protruding post was configured to bind to an analyte. Each nucleic acid nanoparticle was coupled to a plurality of fluorescent dyes to facilitate detection when bound to array sites.

Deposition of each type of nucleic acid nanoparticle was tested under a variety of deposition conditions, including variations in array lot and variations in deposition buffer. Deposition buffer was varied with respect to buffer composition, ionic strength, surfactant type, and surfactant concentration. Nucleic acid nanoparticles were incubated with the arrays at 150 picomolar (pM) concentration for 30 mins. After incubation, unbound nucleic acid nanoparticles were rinsed from array surfaces, then arrays were imaged by confocal fluorescent microscopy. Individual images were analyzed to quantify counts of dark array sites (no occupancy), moderate fluorescent signal intensity array sites (single occupancy), and high fluorescent signal intensity array sites (multiple occupancy). Total array site occupancy was calculated as the percentage of total number of sites with detected single or multiple occupancy divided by the total number of sites observed. Single array site occupancy was calculated as the percentage of total number of sites with detected single occupancy divided by the total number of sites observed. Multiple array site occupancy was calculated as the percentage of total number of sites with detected multiple occupancy divided by the total number of sites observed.

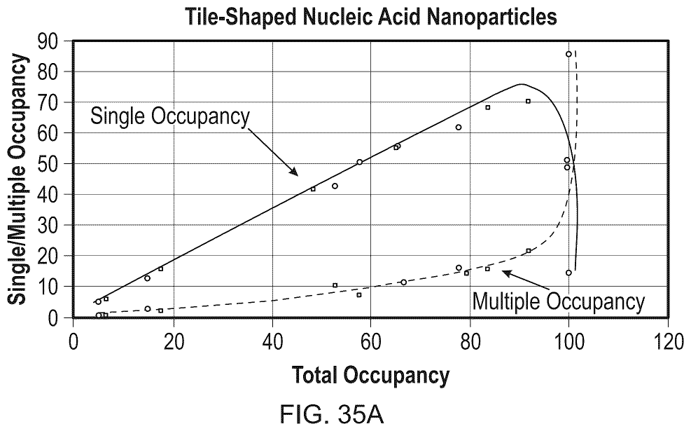

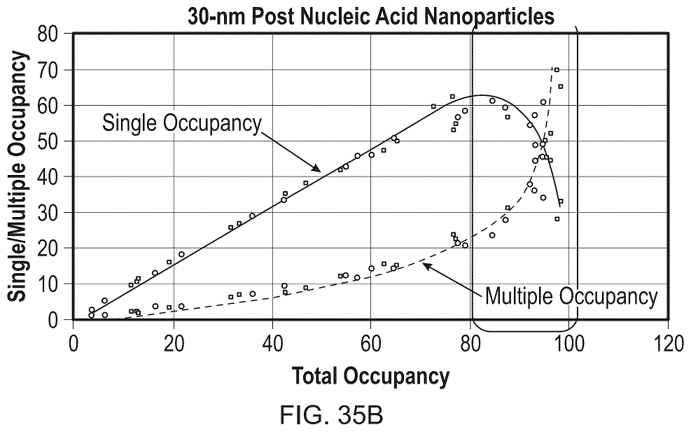

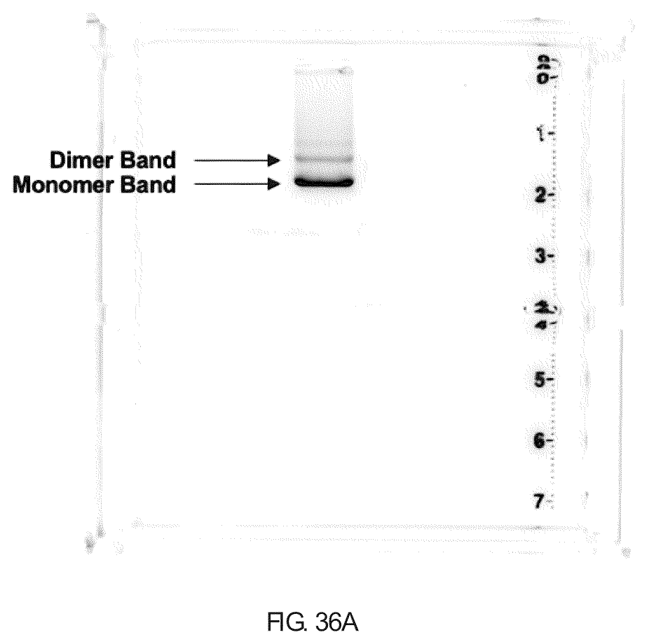

FIGS. 35A and 35B display aggregated data showing single occupancy and multiple occupancy as a function of total occupancy over all the conditions observed. FIG. 35A displays aggregated data for the tile-shaped nucleic acid nanoparticles; FIG. 35B displays aggregated data for the nucleic acid nanoparticles with protruding posts. For both types of nucleic acid nanoparticles, the percentages of sites having single and multiple occupancies was observed to increase linearly as total occupancy increases. Accordingly, a ratio of sites with single occupancy to sites with multiple occupancy was essentially constant until very high total occupancy percentages was achieved. This effect was observed to occur independent of the deposition buffer formulation and array lot.

### Example 5. Fractionation of Particles Before Array Deposition

Nucleic acid nanoparticles were fractionated prior to array deposition to determine the effect of fractionation on single and multiple occupancy percentages of arrays. Nucleic acid nanoparticles and array compositions were prepared as described in U.S. Pat. No. 11,505,796. The fractionated nucleic acid nanoparticles are described in Example 4 as the type of nucleic acid nanoparticle with a 30 nanometer post that was configured to attach an analyte.

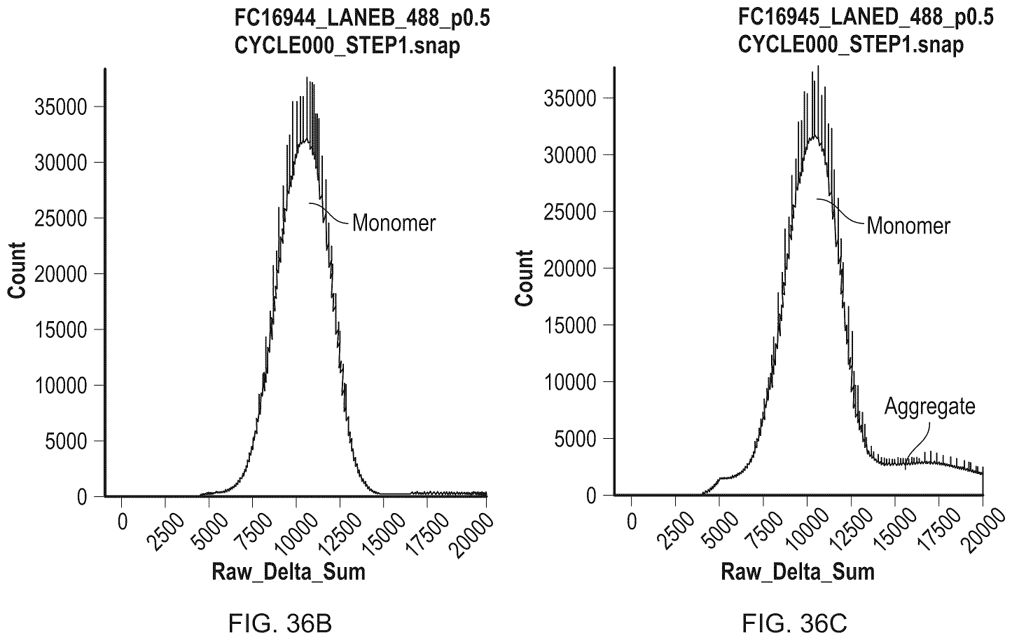

After forming nucleic acid nanoparticles, the nanoparticles were separated on a 1% agarose gel with 0.5×TBE buffer, 11 mM magnesium chloride and 5 microliters of SYBR SAFE dye. Gels were run for 2 hours at 70 V. FIG. 36A depicts images of separated nucleic acid nanoparticles. Samples were observed to have a first band at the molecular weight corresponding to monomeric nucleic acid nanoparticles, and a second band at a higher molecular weight, corresponding to aggregates or multimeric nanoparticle complexes. A portion of agarose gel containing the low-molecular weight band was isolated and purified to recover a fraction containing the monomeric particles.

The purified monomeric particle fraction was deposited on a first array at 150 picomolar concentration with a 30 min deposition time. As a control, a non-fractionated sample (i.e., a sample of nucleic acid particles that had not been separated on the agarose gel) was deposited on a second array at 150 picomolar concentration with a 30 min deposition time. Arrays were imaged as described in Example 4.

FIG. 36B depicts a fluorescent intensity histogram for the imaged array of fractionated nucleic acid nanoparticles. The histogram was observed to have substantially a single peak, indicating most all observed occupied sites had single occupancy. FIG. 36C depicts a fluorescent intensity histogram for the imaged control array. The histogram was observed to have a peak at lower signal intensities and a broader second peak at higher signal intensities, indicating a mixture of sites having single occupancy and multiple occupancy. Fractionation of the nucleic acid nanoparticles to enrich for a monomeric fraction appeared to increase the quantity of sites with single-molecule occupancy.

Notwithstanding the appended claims, the disclosure set forth herein is also defined by the following clauses:
1) A method of forming an array of analytes, comprising:
   a) providing a solid support comprising a population of sites, wherein the population of sites is characterized by an array site binding competency profile;
   b) providing a fluidic medium comprising a population of analytes, wherein the composition of the population of analytes is based upon the array site binding competency profile; and
   c) contacting the fluidic medium to the solid support, thereby binding analytes of the population of analytes to sites of the population of sites to form the array of analytes, wherein the array of analytes has a site occupancy of at least 80%, and wherein the array of analytes has a site co-occupancy of no more than 10%.
2) The method of clause 1, wherein an analyte of the population of analytes is linked to one and only one anchoring moiety of a population of anchoring moieties.
3) The method of clause 2, wherein array site binding competency for an array site of the population of array sites comprises a measure of binding propensity for an anchoring moiety.
4) The method of clause 3, wherein the measure of binding propensity comprises a deterministic measure of binding propensity of the array site for the anchoring moiety.
5) The method of clause 3 or 4, wherein the measure of binding propensity comprises a deterministic measure of binding propensities of the array site for anchoring moieties of a population of anchoring moieties.
6) The method of clause 3, wherein the measure of binding propensity comprises a probabilistic measure of binding propensity of the array site for the anchoring moiety.
7) The method of clause 3 or 6, wherein the measure of binding propensity comprises a probabilistic measure of binding propensities of the array site for anchoring moieties of a population of anchoring moieties.
8) The method of clause 5 or 7, wherein the population of anchoring moieties comprises two or more species of anchoring moieties.
9) The method of clause 8, wherein a first species of anchoring moiety of the two or more species of anchoring moiety differs from a second species of anchoring moiety of the two or more species of anchoring moiety with respect to an anchoring moiety binding property.
10) The method of clause 9, wherein the anchoring moiety binding property is selected from the group consisting of: anchoring moiety dimension, binding entity face dimension, binding entity effective surface area, binding entity face effective surface area, binding entity hydrodynamic radius, binding entity weight, total coupling moiety quantity, coupling moiety species quantity, total coupling moiety surface density, coupling moiety species surface density, total passivating moiety surface density, passivating moiety species surface density, total coupling moiety quantity, coupling moiety species quantity, total passivating moiety quantity, passivating moiety species quantity, total coupling moiety functional quantity, coupling moiety species functional quantity, coupling moiety linker length, coupling moiety linker chemical composition, binding entity net surface electrical charge density, binding entity surface electrical charge anisotropy, binding entity isoelectric point, and binding entity Zeta potential.
11) The method of clause 9 or 10, wherein the first species of anchoring moiety of the two or more species of anchoring moiety differs from the second species of anchoring moiety of the two or more species of anchoring moiety with respect to two or more anchoring moiety binding properties.
12) The method of any one of clauses 2-11, wherein the population of analytes is characterized by an anchoring moiety binding competency profile.
13) The method of clause 12, wherein the anchoring moiety binding competency profile comprises a statistical distribution of anchoring moiety quantity as a function of anchoring moiety binding competency for anchoring moieties of the population of anchoring moieties.
14) The method of clause 11, wherein anchoring moiety binding competency for an anchoring moiety site of the population of anchoring moieties comprises a measure of binding propensity for an array site.
15) The method of clause 14, wherein the measure of binding propensity comprises a deterministic measure of binding propensity of the anchoring moiety for the array site.
16) The method of clause 14 or 15, wherein the measure of binding propensity comprises a deterministic measure of binding propensities of the anchoring moiety for array sites of the population of array sites.
17) The method of clause 14, wherein the measure of binding propensity comprises a probabilistic measure of binding propensity of the anchoring moiety for the array site.
18) The method of clause 14 or 17, wherein the measure of binding propensity comprises a probabilistic measure of binding propensities of the anchoring moiety for array sites of the population of array sites.
19) The method of clause 16 or 18, wherein the population of array sites comprises two or more species of array sites.
20) The method of clause 19, wherein a first species of array site of the two or more species of array sites differs from a second species of array site of the two or more species of array sites with respect to an array site binding property.
21) The method of clause 20, wherein the array site binding property is selected from the group consisting of: site dimension, site surface area, site aspect ratio, site pitch, site depth, site height, coating or layer thickness, total molecular surface density, total coupling moiety surface density, coupling moiety species surface density, total passivating moiety surface density, passivating moiety species surface density, total surface moiety quantity, total coupling moiety quantity, coupling moiety species quantity, total passivating moiety quantity, passivating moiety species quantity, total coupling moiety functional quantity, coupling moiety species functional quantity, passivating layer thickness, passivating layer volume, boundary material height, boundary material width, boundary material volume, passivating layer chemical composition, boundary layer chemical composition, defect surface density, and array site net surface electrical charge density.
22) The method of clause 20 or 21, wherein the first species of array site of the two or more species of array sites differs from the second species of array site of the two or more species of array sites with respect to two or more array site binding properties.
23) The method of any one of clauses 2-22, wherein providing the fluidic medium comprising the population of analytes comprises: e) attaching analytes of the population of analytes to anchoring moieties of the population of anchoring moieties; and f) disposing the population of analytes and the population of anchoring moieties within the fluidic medium.
24) The method of clause 23, wherein step e) comprises covalently attaching analytes of the population of analytes to anchoring moieties of the population of anchoring moieties.
25) The method of any one of clauses 2-24, wherein the fluidic medium comprises an analyte of the population of analytes that is not attached to an anchoring moiety of the population of anchoring moieties.
26) The method of any one of clauses 2-25, wherein the fluidic medium comprises an anchoring moiety of the population of anchoring moieties that is not attached to an analyte of the population of analytes.
27) The method of any one of clauses 2-26, wherein providing the fluidic medium comprising the population of analytes comprises: g) providing a quantity of analytes of the population of analytes based upon the array site binding competency profile of the population of array sites.
28) The method of clause 27, wherein providing the quantity of analytes of the population of analytes comprises: h) identifying a quantity of sites comprising a same array site binding competency based upon the array site binding competency profile; and i) providing the quantity of analytes to the fluidic medium, wherein each analyte of the quantity of analytes comprises a binding competency for the quantity of sites.
29) The method of clause 28, wherein the quantity of analytes is greater than the quantity of sites.
30) The method of clause 28, wherein the quantity of analytes is less than or equal to the quantity of sites.
31) The method of any one of clauses 28-30, wherein providing the quantity of analytes of the population of analytes comprises: h) identifying two or more subpopulations of array sites, wherein each subpopulation of array sites comprises a quantity of sites comprising a same array site binding competency based upon the array site binding competency profile; and i) providing two or more subpopulations of analytes to the fluidic medium, wherein each subpopulation of analytes comprises a quantity of analytes, and wherein each analyte of a quantity of analytes of a subpopulation of analytes comprises a binding competency for the quantity of sites of a corresponding subpopulation of sites.
32) The method of any one of clauses 2-31, wherein binding analytes of the population of analytes to array sites of the population of array sites comprises binding an anchoring moiety to an array site of the population of array sites, wherein the anchoring moiety is attached to an analyte.
33) The method of clause 32, wherein the anchoring moiety comprises a non-covalent coupling moiety.
34) The method of clause 33, wherein binding the anchoring moiety to the array site comprises non-covalently binding the non-covalent coupling moiety of the anchoring moiety to a non-covalent coupling moiety of the array site.
35) The method of any one of clauses 32-34, wherein the anchoring moiety comprises a covalent coupling moiety.
36) The method of clause 35, wherein binding the anchoring moiety to the array site comprises covalently binding the covalent coupling moiety of the anchoring moiety to a covalent coupling moiety of the array site.
37) The method of any one of clauses 2-36, wherein the array of analytes is formed by contacting the fluidic medium comprising the population of analytes only once.
38) The method of clause 37, wherein the no additional analytes are added to the fluidic medium during the contacting of the fluidic medium to the solid support.
39) The method of any one of clauses 2-36, wherein the array of analytes is formed by further contacting a second fluidic medium to the solid support, wherein the second fluidic medium comprises a second population of analytes.
40) The method of any one of clauses 1-39, wherein the fluidic medium is in contact with the solid support for no more than 1 hour.
41) The method of clause 40, wherein the fluidic medium is in contact with the solid support for no more than 30 minutes.
42) The method of clause 41, wherein the fluidic medium is in contact with the solid support for no more than 15 minutes.
43) The method of any one of clauses 1-42, further comprising detecting at each site of the population of sites a presence or an absence of an analyte of the population of analytes.
44) The method of clause 43, wherein detecting the presence or the absence of the analyte of the plurality of analytes comprises detecting a signal from the analyte or an anchoring moiety attached to the analyte.
45) The method of any one of clauses 1-44, further comprising characterizing at least 50% of the analytes of the array of analytes.
46) The method of clause 45, wherein characterizing the at least 50% of the analytes of the array of analytes comprises: i) binding a first plurality of affinity agents to a first fraction of the analytes of the array; ii) detecting affinity agents of the first plurality of affinity agents bound to the first fraction of the analytes at a first fraction of sites of the population of sites; and iii) optionally binding a second plurality of affinity agents to a second fraction of the analytes of the array, wherein the first fraction of analytes differs from the second fraction of analytes; and iv) optionally detecting affinity agents of the second plurality of affinity agents bound to the second fraction of the analytes at a second fraction of sites of the population of sites, wherein the first fraction of sites differs from the second fraction of sites.
47) The method of clause 46, further comprising: v) identifying an affinity reagent binding profile for each analyte of the array of analytes.
48) The method of clause 47, further comprising: vi) determining an identity of an analyte based upon an affinity reagent binding profile.
49) The method of clause 45, wherein characterizing the at least 50% of the analytes of the array of analytes comprises performing a fluorosequencing assay.
50) The method of clause 49, wherein the fluorosequencing assay comprises an Edman-type degradation reaction.
51) The method of clause 1, wherein the site quality control parameter is selected from the group consisting of: average site dimension, maximum site dimension, minimum site dimension, average site aspect ratio, maximum site aspect ratio, minimum site aspect ratio, average site surface area, maximum site surface area, minimum site surface area, average site accessible surface area, maximum site accessible surface area, minimum site accessible surface area, average site functional group surface density, maximum site functional group surface density, minimum site functional group surface density, average site functional group surface quantity, maximum site functional group surface quantity, and minimum site functional group surface quantity.
52) A method of forming an array of analytes, comprising:
   a) providing a solid support comprising a population of sites, wherein the population of sites has a characterized site binding competency profile;
   b) providing a fluidic medium comprising a population of analytes, wherein the population of analytes has a characterized analyte binding competency profile, and wherein the characterized analyte binding competency profile is a function of an analyte quality control parameter;
   c) contacting the fluidic medium to the solid support, thereby binding analytes of the population of analytes to sites of the population of sites to form the array of analytes, wherein the array of analytes has a site occupancy of at least 80%, and wherein binding the analytes of the population of analytes to sites of the population of sites occurs in no more than 1 hour.
53) A method of preparing a population of anchoring moieties, comprising:
   a) providing a solid support comprising a population of sites;
   b) measuring a quality control parameter for each site of the population of sites;
   c) based upon the quality control parameter, determining a site binding competency profile for the population of sites; and
   d) based upon the site binding competency profile of the population of sites, preparing a population of anchoring moieties, wherein the population of anchoring moieties has an anchoring moiety binding competency profile, and wherein the anchoring moiety binding competency profile is complementary to the site binding competency profile of the population of sites.
54) A method of forming an array of analytes, comprising:
   a) providing a solid support with a surface, wherein the surface is substantially planar, and wherein a population of sites is disposed on the surface;
   b) contacting the array with a fluidic medium comprising a population of anchoring moieties, wherein each anchoring moiety is attached to an analyte; and
   c) after contacting the array with the fluidic medium, providing a first analyte association condition, wherein the first analyte association condition forms a concentration gradient of anchoring moieties in the fluidic medium, and wherein the concentration gradient of anchoring moieties is characterized by an increasing concentration of anchoring moieties as distance to the surface of the solid support decreases in a direction orthogonal to the surface of the solid support, whereby anchoring moieties of the plurality of anchoring moieties bind to sites of the population of sites, wherein the single-pass site occupancy is at least 75% of the population of sites within no more than 30 minutes of contacting the solid support with the fluidic medium.
55) A method of forming an array of analytes on a solid support, comprising:
   a) providing a solid support comprising a surface, wherein the surface is substantially planar, wherein a quantity of sites is disposed on the surface, wherein each site is separated from each other site of the quantity of sites by an interstitial region, and wherein each site of the quantity of sites is optically resolvable from each other site;
   b) contacting the array with a fluidic medium comprising a plurality of nanoparticles, wherein each nanoparticle is attached to an analyte;
   c) after contacting the array with the fluidic medium, providing a first analyte association condition, wherein the first analyte association condition forms a concentration gradient of nanoparticles in the fluidic medium, and wherein the concentration of nanoparticles increases as a distance from the surface of the solid support decreases in a direction orthogonal to the surface of the solid support;
   d) after contacting the array with the fluidic medium, providing a first analyte association condition, wherein the first analyte association condition forms a concentration gradient of nanoparticles in the fluidic medium, and wherein the concentration of nanoparticles increases as a distance from the surface of the solid support decreases in a direction orthogonal to the surface of the solid support;
   e) after contacting the array with the fluidic medium, providing a second analyte association condition, wherein the second analyte association condition forms an anisotropic surface density of nanoparticles on the interstitial region of the solid support, whereby nanoparticles of the plurality of nanoparticles bind to sites of the quantity of sites, wherein the single-pass global site occupancy is at least 75% of the quantity of sites within no more than 15 minutes of contacting the solid support with the fluidic medium.
56) The method of clause 55, wherein providing the first analyte association condition comprises altering the fluidic medium.
57) The method of clause 56, wherein altering the fluidic medium comprises altering a pH of the fluidic medium.
58) The method of clause 56 or 57, wherein altering the fluidic medium comprises altering an ionic strength of a salt species of the fluidic medium.
59) The method of any one of clauses 56-58, wherein altering the fluidic medium comprises altering a concentration of a surfactant species of the fluidic medium.
60) The method of any one of clauses 56-59, further comprising altering a temperature of the fluidic medium.
61) The method of clause 56, wherein contacting the array with a fluidic medium comprises contacting the array with a fluidic medium comprising the plurality of nanoparticles and a plurality of small solutes.
62) The method of clause 61, wherein the fluidic medium has a first concentration of small solutes.
63) The method of clause 62, wherein altering the fluidic medium comprises: i) contacting the fluidic medium with a second fluidic medium, wherein the fluidic medium is contacted with the second fluidic medium at a surface distal to the surface of the solid support, and wherein the second fluidic medium has a second concentration of small solutes, wherein the second concentration of small solutes is less than the first concentration of small solutes; ii) forming a concentration gradient of small solutes in the fluidic medium, wherein the concentration of small solutes in the fluidic medium increases as the distance from the surface of the solid support increases in a direction orthogonal to the surface of the solid support; and iii) forming the concentration gradient of nanoparticles in the fluidic medium.
64) The method of clause 63, wherein steps ii) and iii) occur simultaneously.
65) The method of clause 63 or 64, wherein a concentration of small solutes in the second fluidic medium is substantially zero before contacting the fluidic medium with the second fluidic medium.
66) The method of any one of clauses 55-65, wherein providing the first analyte association condition comprises increasing a sedimentation rate of the nanoparticles.
67) The method of clause 66, wherein increasing the sedimentation rate of the nanoparticles comprises coupling a weighted particle to a nanoparticle of the plurality of nanoparticles.
68) The method of clause 67, wherein a ratio of a weight of the weighted particle to a weight of the nanoparticle is at least 1.
69) The method of clause 68, wherein the ratio of the weight of the weighted particle to the weight of the nanoparticle is at least 10.
70) The method of any one of clauses 55-69, wherein providing the first analyte association condition comprises, after contacting the fluidic medium to the solid support, centrifuging the fluidic medium and the solid support.
71) The method of any one of clauses 55-70, wherein providing the second analyte association condition comprises agitating the fluidic medium.
72) The method of clause 71, wherein agitating the fluidic medium comprises generating oscillatory flow in the fluidic medium in a direction substantially parallel to the surface of the solid support.
73) The method of any one of clauses 55-72, wherein providing the second analyte association condition comprises providing acoustic waves to the solid support.
74) The method of any one of clauses 55-73, wherein the interstitial region comprises a hydrophobic layer.
75) The method of clause 74, wherein providing the second analyte association condition comprises coupling a hydrophobic material to the hydrophobic layer.
76) The method of clause 75, wherein the hydrophobic material comprises a hydrophobic liquid.
77) The method of clause 75, wherein the hydrophobic material comprises hydrophobic particles.
78) The method of any one of clauses 55-77, wherein the interstitial region comprises a photon-absorbing material.
79) The method of clause 78, wherein providing the second analyte association condition comprises: i) heating the photon-absorbing material with an incident light field; and ii) producing a convective flow of nanoparticles away from the photon-absorbing material.
80) A method of forming an array, comprising:
   a) providing an array comprising a plurality of sites, wherein the sites are substantially devoid of analytes, wherein the plurality of sites has a characteristic property, and wherein the characteristic binding property of the plurality of sites has a standard deviation with a magnitude of at least 10% of the characteristic property; b) binding a plurality of analytes to sites of the plurality of sites; and
   c) after binding the plurality of analytes to the sites, detecting a presence or absence of an analyte at each site of the plurality of sites, wherein at least 70% of sites of the active fraction of sites comprise an analyte of the plurality of analytes, and wherein no more than 10% of sites of the active fraction of sites comprises two or more analytes of the plurality of analytes.
81) A method, comprising:
   a) providing a first array of a plurality of arrays, wherein each array of the plurality of arrays comprises a plurality of sites, and wherein the plurality of sites for each array has a known binding characteristic;
   b) binding analytes to the plurality of sites of the first array with a first analyte association condition;
   c) after binding the analytes to the first array, determining a first analyte site occupancy for the first array;
   d) after determining the first analyte site occupancy for the first array, determining a second analyte association condition for a second array of the plurality of arrays based upon a comparison of a first known binding characteristic of the first array to a second known binding characteristic of the second array; and
   e) binding analytes to the plurality of sites of the second array with the second analyte association condition.
82) A method of forming an array, comprising:
   a) contacting an array comprising a plurality of sites with a layer of a fluidic medium, wherein the fluidic medium comprises a plurality of analytes, wherein the layer of the fluidic medium has an average thickness, wherein sites of the plurality of sites comprise filamentous moieties, and wherein the filamentous moieties have an average length of at least 10% the thickness of the fluidic medium; and
   b) binding analytes to at least 70% of sites of the plurality of sites within 15 minutes of contacting the array with the fluidic medium.
83) The method of clause 82, wherein a site of the plurality of sites comprises one and only one filamentous moiety of the filamentous moieties.
84) The method of clause 82 or 83, wherein a site of the plurality of sites comprises two or more filamentous moieties of the filamentous moieties.
85) The method of any one of clauses 82-84, wherein a site of the plurality of sites comprises zero filamentous moieties of the filamentous moieties.
86) The method of clause 85, wherein no more than about 10% of sites of the plurality of sites comprise zero filamentous moieties of the filamentous moieties.
87) The method of any one of clauses 82-86 wherein a filamentous moiety of the filamentous moieties has a length of at least 1 micron.
88) The method of clause 87, wherein the filamentous moiety of the filamentous moieties has a length of at least 10 microns.
89) The method of any one of clauses 82-88, wherein a filamentous moiety of the filamentous moieties has a length of at least 25% of the thickness of the fluidic medium.
90) The method of clause 89, wherein the filamentous moiety of the filamentous moieties has a length of at least 50% of the thickness of the fluidic medium.
91) The method of any one of clauses 82-90, wherein a filamentous moiety comprises a non-zero net electrical charge.
92) The method of clause 91, wherein the non-zero net electrical charge comprises a positive electrical charge or a negative electrical charge.
93) The method of any one of clauses 82-92, wherein a filamentous moiety of the filamentous moieties comprises an oligonucleotide.
94) The method of clause 93, wherein an analyte of the plurality of analytes comprises a second oligonucleotide.
95) The method of clause 94, wherein the second oligonucleotide comprises a complementary nucleic acid sequence to the oligonucleotide of the filamentous moiety.
96) The method of clause 93 or 94, wherein the oligonucleotide comprises a plurality of nucleic acid sequences that are complementary to the second oligonucleotide of the analyte of the plurality of analytes.
97) The method of any one of clauses 82-96, further comprising contacting the filamentous moieties with a second layer of a second fluidic medium, thereby increasing a length of the filamentous moieties relative to a surface of the array.
98) The method of clause 97, further comprising, after increasing the length of the filamentous moieties, binding analytes of the plurality of analytes to filamentous moieties.
99) The method of clause 98, further comprising contacting the array with a third fluidic medium, thereby decreasing the length of the filamentous moieties.
100) A method, comprising:
   a) contacting a fluidic medium comprising a plurality of particles to a solid support, wherein the solid support comprises a plurality of sites, wherein the spatial distribution of the plurality of particles in the fluidic medium is substantially homogeneous, and wherein each individual particle of the plurality of particles is coupled to a plurality of macromolecules;
   b) concentrating the plurality of particles adjacent to a surface of the solid support;
   c) after concentrating the plurality of particles adjacent to the surface of the solid support, separating macromolecules from particles of the plurality of particles;
   d) binding the macromolecules to sites of the plurality of sites of the solid support; and
   e) after separating the macromolecules from the plurality of particles, dispersing particles of the plurality of particles from adjacent to the surface of the solid support.
101) The method of clause 100, wherein a particle of the plurality of particles comprises an organic nanoparticle or an inorganic nanoparticle.
102) The method of clause 100 or 101, wherein the particle of the plurality of particles comprises a magnetic nanoparticle.
103) The method of clause 102, wherein the magnetic particle is coupled to the particle of the plurality of particles.
104) The method of clause 100 or 101, wherein the particle of the plurality of particles comprises a non-neutral electrical charge.
105) The method of clause 104, wherein the particle of the plurality of particles comprises a nanoparticle, wherein an electrically-charged moiety is coupled to the nanoparticle.
106) The method of any one of clauses 100-105, wherein a macromolecule of the plurality of macromolecules comprises a biomolecule.
107) The method of clause 106, wherein the biomolecule of the plurality of macromolecules comprises a polypeptide, a nucleic acid, a polysaccharide, or a combination thereof.
108) The method of any one of clauses 100-107, wherein a macromolecule of the plurality of macromolecules comprises a polymer.
109) The method of any one of clause 100-108, wherein a particle of the plurality of particles is coupled to a macromolecule of the plurality of macromolecules by a covalent bond.
110) The method of clause 109, wherein separating the macromolecule from the particle of the plurality of particles comprises cleaving the covalent bond.
111) The method of clause 110, wherein cleaving the covalent bond comprises photolytically cleaving, chemically cleaving, or enzymatically cleaving the covalent bond.
112) The method of any one of clauses 100-108, wherein a particle of the plurality of particles is coupled to a macromolecule of the plurality of macromolecules by a non-covalent interaction.
113) The method of clause 112, wherein separating the macromolecule from the particle of the plurality of particles comprises dissociating the non-covalent interaction.
114) The method of clause 113, wherein dissociating the non-covalent interaction comprises contacting the particle with a dissociating agent, wherein the dissociating agent comprises a denaturant, a chaotrope, or a binding competitor.
115) The method of any one of clauses 100-114, wherein concentrating the plurality of particles adjacent to the surface of the solid support comprises forming an electric field or a magnetic field in the fluidic medium.
116) The method of clause 115, wherein forming the electric field comprises forming an electric field of at least 1 milliVolt per meter (mV/m).
117) The method of clause 115, wherein forming the magnetic field comprises forming a magnetic field of at least 100 Tesla per meter (T/m).
118) The method of any one of clauses 100-114, wherein concentrating the plurality of particles adjacent to the surface of the solid support comprises applying a centripetal force to the plurality of particles.
119) The method of any one of clauses 100-118, wherein binding the macromolecules to the sites of the plurality of sites of the solid support comprises covalently coupling the macromolecules to the sites of the plurality of sites.
120) The method of any one of clauses 100-118, wherein binding the macromolecules to the sites of the plurality of sites of the solid support comprises non-covalently coupling the macromolecules to the sites of the plurality of sites.
121) The method of clause 120, wherein non-covalently coupling a macromolecule of the macromolecules to a site of the sites comprises binding the macromolecule to an analyte, wherein the analyte is coupled to the site of the plurality of sites.
122) The method of clause 121, wherein the macromolecule comprises an affinity agent, wherein binding the macromolecule to the analyte comprises binding the affinity agent to the analyte.
123) The method of any one of clauses 100-118, wherein a site of the plurality of sites comprises a surface-coupled moiety, wherein a macromolecule of the macromolecules comprises a surface-coupling moiety, and wherein binding the macromolecule to the site comprises binding the surface-coupled moiety to the surface-coupling moiety.
124) The method of clause 123, wherein the site of the plurality of sites comprises a plurality of surface-coupled moieties, wherein the macromolecule of the macromolecules comprises a plurality of surface-coupling moieties, and wherein binding the macromolecule to the site comprises binding the plurality of surface-coupled moieties to the plurality of surface-coupling moieties.
125) The method of any one of clauses 100-124, further comprising: f) after dispersing the particles of the plurality of particles from adjacent to the surface of the solid support, coupling a second plurality of macromolecules to a particle of the particles.
126) The method of clause 125, wherein step f) further comprises transferring the particles to a reservoir comprising the second plurality of macromolecules.
127) The method of clause 125, wherein step f) further comprises, before coupling the second plurality of macromolecules to the particle of the particles, providing the second plurality of macromolecules to the fluidic medium.
128) The method of clause 125, wherein step f) further comprises: i) binding the particles to a surface of a second solid support, ii) after binding the particles to the surface of the second solid support, removing the fluidic medium from the solid support; and iii) providing a second fluidic medium to the solid support, wherein the second fluidic medium comprises the second plurality of macromolecules.
129) The method of any one of clauses 100-128, wherein dispersing the particles of the plurality of particles from adjacent to the surface of the solid support comprises forming an electric field or a magnetic field in the fluidic medium.
130) The method of clause 129, wherein forming the electric field or the magnetic field in the fluidic medium comprises reversing a polarity or a directionality of the electric field or the magnetic field in the fluidic medium.
131) The method of any one of clauses 100-128, wherein dispersing the particles of the plurality of particles from adjacent to the surface of the solid support comprises applying a centripetal force to the particles of the plurality of particles.
132) The method of any one of clauses 100-128, wherein dispersing the particles of the plurality of particles from adjacent to the surface of the solid support comprises providing a turbulent flow in the fluidic medium.

What is claimed is:

1. A method, comprising:
a) contacting a plurality of analytes to a solid support comprising a plurality of sites, wherein each individual analyte is coupled to one and only one particle of a plurality of particles, wherein each particle is attached to a plurality of oligonucleotides, and wherein each individual site of the plurality of sites comprises a plurality of complementary oligonucleotides;
b) hybridizing oligonucleotides of the plurality of oligonucleotides of particles of the plurality of particles to complementary oligonucleotides of sites of the plurality of sites, thereby coupling analytes of the plurality of analytes to the solid support; and
c) after contacting the plurality of analytes to the solid support, detecting a presence of one and only one analyte for at least 50% of sites of the plurality of sites, and detecting a presence of two or more analytes for no more than 10% of sites of the plurality of sites;
wherein the contacting occurs for no more than 1 hour; and
wherein a molar ratio of analytes of the plurality of analytes to sites of the plurality of sites is no more than 10.

2. The method of claim 1, wherein each individual oligonucleotide of the plurality of oligonucleotides comprises a linker moiety.

3. The method of claim 2, wherein the linker moiety is at least 1 nanometer (nm) in length.

4. The method of claim 2 or 3, wherein the linker moiety comprises a non-rigid moiety.

5. The method of claim 4, wherein the non-rigid moiety comprises a non-nucleic acid polymeric chain.

6. The method of claim 5, wherein the non-nucleic acid polymeric chain comprises polyethylene glycol, a peptide, or a polysaccharide.

7. The method of any one of claims 1-6, wherein each complementary oligonucleotide comprises a linker moiety.

8. The method of claim 7, wherein the linker moiety is at least 1 nanometer (nm) in length.

9. The method of claim 7 or 8, wherein the linker moiety comprises a non-rigid moiety.

10. The method of claim 9, wherein the non-rigid moiety comprises a non-nucleic acid polymeric chain.

11. The method of any one of claims 1-10, wherein contacting the plurality of analytes to the solid support comprises contacting a fluidic medium containing the plurality of analytes to the solid support.

12. The method of claim 11, further comprising agitating the fluidic medium while the fluidic medium is contacted to the solid support.

13. The method of claim 11 or 12, further comprising, during the no more than 1 hour: (i) removing the fluidic medium from contact with the solid support; and (ii) after removing the fluidic medium, again contacting the fluidic medium to the solid support.

14. The method of claim 13, wherein steps (i) and (ii) are repeated at least once.

15. The method of claim 13 or 14, further comprising, between steps (i) and (ii), adding additional analytes to the fluidic medium.

16. The method of any one of claims 1-15, wherein each individual particle of the plurality of particles comprises a nucleic acid nanoparticle.

17. The method of claim 16, wherein the individual analyte is attached to a first face of the nucleic acid nanoparticle and the plurality of oligonucleotides is attached to a second face of the nucleic acid nanoparticles.

18. The method of claim 17, wherein the plurality of oligonucleotides is attached to the second face adjacent to one or more edges of the second face.

19. The method of any one of claims 1-18, wherein contacting the plurality of analytes to the solid support further comprises contacting a plurality of fiducial elements to the solid support, wherein each individual fiducial element is attached to a plurality of oligonucleotides.

20. The method of claim 19, further comprising hybridizing oligonucleotides of the plurality of oligonucleotides of fiducial elements to complementary oligonucleotides of sites of the plurality of sites, thereby coupling fiducial elements of the plurality of fiducial elements to the solid support.
